(19) United States
(12) Patent Application Publication
(10) Pub. No.: US 2024/0264170 A1
(43) Pub. Date: Aug. 8, 2024

(54) BIOMARKER PAIRS AND TRIPLETS FOR PREDICTING PRETERM BIRTH

(71) Applicant: Sera Prognostics, Inc., Salt Lake City, UT (US)

(72) Inventors: Ryan Michael TREACY, Salt Lake City, UT (US); Ashoka Dharmapriya POLPITIYA, Sandy, UT (US); Md Bahadur BADSHA, Salt Lake City, UT (US); Julja BURCHARD, Ventura, CA (US); Tracey Cristine FLEISCHER, Sandy, UT (US); John Jay BONIFACE, Park City, UT (US)

(21) Appl. No.: 18/560,831

(22) PCT Filed: May 20, 2022
(86) PCT No.: PCT/US22/30387

§ 371 Date: Nov. 14, 2023

Related U.S. Application Data
(60) Provisional application No. 63/191,666, filed on May 21, 2021.
(60) Provisional application No. 63/256,367, filed on Oct. 15, 2021.

(51) Int. Cl. G01N 33/68; C12N 9/02

(52) U.S. Cl. CPC G01N 33/689; C12N 9/0071; G01N 33/6848; C12Y 114/11029; G01N 2333/42; G01N 2333/471; G01N 2333/4713; G01N 2333/5756; G01N 2333/65; G01N 2800/368

(57) ABSTRACT

The disclosure provides a reversal group of biomarkers comprising a reversal pair and a reversal triplet, wherein the reversal pair and reversal triplet exhibit a change in reversal value between pregnant females at risk for pre-term birth and term controls. Also provided is a method of determining probability for preterm birth in a pregnant female, the method comprising measuring in a biological sample obtained from the pregnant female, a reversal value for a reversal pair and a reversal triplet to determining the probability of preterm birth in the pregnant female.

### CROSS-REFERENCE TO RELATED APPLICATIONS

This application claims the benefit of U.S. Provisional Patent Application No. 63/191,666, filed May 21, 2021, and U.S. Provisional Patent Application No. 63/256,367, filed Oct. 15, 2021, the entire contents of each of which are incorporated by reference herein.

The invention relates generally to the field of precision medicine and, more specifically to compositions and methods for determining the probability for preterm birth in a pregnant female.

### BACKGROUND

According to the World Health Organization, an estimated 15 million babies are born preterm (before 37 completed weeks of gestation) every year. In almost all countries with reliable data, preterm birth rates are increasing. See, World Health Organization; March of Dimes; The Partnership for Maternal, Newborn & Child Health; Save the Children, Born too soon: the global action report on preterm birth, ISBN 9789241503433(2012). An estimated 1 million babies die annually from preterm birth complications. Globally, preterm birth is the leading cause of newborn deaths (babies in the first four weeks of life) and the second leading cause of death after pneumonia in children under five years. Many survivors face a lifetime of disability, including learning disabilities and visual and hearing problems.

Across 184 countries with reliable data, the rate of preterm birth ranges from 5% to 18% of babies born. Blencowe et al., “National, regional and worldwide estimates of preterm birth.” The Lancet, 9; 379(9832):2162-72 (2012). While over 60% of preterm births occur in Africa and south Asia, preterm birth is nevertheless a global problem. Countries with the highest numbers include Brazil, India, Nigeria and the United States of America. Of the 11 countries with preterm birth rates over 15%, all but two are in sub-Saharan Africa. In the poorest countries, on average, 12% of babies are born too soon compared with 9% in higher-income countries. Within countries, poorer families are at higher risk. More than three-quarters of premature babies can be saved with feasible, cost-effective care, for example, antenatal steroid injections given to pregnant women at risk of preterm labor to strengthen the babies' lungs.

Infants born preterm are at greater risk than infants born at term for mortality and a variety of health and developmental problems. Complications include acute respiratory, gastrointestinal, immunologic, central nervous system, hearing, and vision problems, as well as longer-term motor, cognitive, visual, hearing, behavioral, social-emotional, health, and growth problems. The birth of a preterm infant can also bring considerable emotional and economic costs to families and have implications for public-sector services, such as health insurance, educational, and other social support systems. The greatest risk of mortality and morbidity is for those infants born at the earliest gestational ages. However, those infants born nearer to term represent the greatest number of infants born preterm and also experience more complications than infants born at term.

To prevent preterm birth in women who are less than 24 weeks pregnant with an ultrasound showing cervical opening, a surgical procedure known as cervical cerclage can be employed in which the cervix is stitched closed with strong sutures. For women less than 34 weeks pregnant and in active preterm labor, hospitalization may be necessary as well as the administration of medications to temporarily halt preterm labor and/or promote the fetal lung development. If a pregnant woman is determined to be at risk for preterm birth, health care providers can implement various clinical strategies that may include preventive medications, for example, 17-α hydroxyprogesterone caproate (Makena) injections and/or vaginal progesterone gel, cervical pessaries, restrictions on sexual activity and/or other physical activities, and alterations of treatments for chronic conditions, such as diabetes and high blood pressure, that increase the risk of preterm labor.

There is a great need to identify and provide women at risk for preterm birth with proper antenatal care. Women identified as high-risk can be scheduled for more intensive antenatal surveillance and prophylactic interventions. Current strategies for risk assessment are based on the obstetric and medical history and clinical examination, but these strategies are only able to identify a small percentage of women who are at risk for preterm delivery. Prior history of spontaneous PTB (sPTB) is currently the single strongest predictor of subsequent PTB. After one prior sPTB the probability of a second PTB is 30-50%. Other maternal risk factors include: black race, low maternal body-mass index, and short cervical length. Amniotic fluid, cervicovaginal fluid, and serum biomarker studies to predict sPTB suggest that multiple molecular pathways are aberrant in women who ultimately deliver preterm. Reliable early identification of risk for preterm birth would enable planning appropriate monitoring and clinical management to prevent preterm delivery. Such monitoring and management might include: more frequent prenatal care visits, serial cervical length measurements, enhanced education regarding signs and symptoms of early preterm labor, lifestyle interventions for modifiable risk behaviors such as smoking cessation, cervical pessaries and progesterone treatment. Finally, reliable antenatal identification of risk for preterm birth also is crucial to cost-effective allocation of monitoring resources.

Despite intense research to identify at-risk women, PTB prediction algorithms based solely on clinical and demographic factors or using measured serum or vaginal biomarkers have seldom resulted in clinically useful tests. More accurate methods to identify women at risk during their first pregnancy and sufficiently early in gestation are needed to allow for clinical intervention. The present invention addresses this need by providing compositions and methods for determining whether a pregnant woman is at risk for preterm birth. Related advantages are provided as well.

### SUMMARY

The present invention provides biomarkers, compositions and methods for predicting the probability of preterm birth in a pregnant female.

In one aspect, provided herein is a composition comprising at least three pairs of biomarkers selected from the group consisting of IBP4/SHBG, IBP4/TETN, EGLN/SHBG, EGLN/TETN, PRL/SHBG, and PRL/TETN, wherein said pairs of biomarkers exhibit a change in reversal value between pregnant females at risk for pre-term birth and term controls.

In one aspect, provided herein is a composition comprising a reversal group of isolated biomarkers comprising: (a) a reversal pair of isolated biomarkers consisting of IBP4/SHBG; and (b) a reversal triplet of isolated biomarkers consisting of (EGLN+PRL)/TETN wherein said reversal group of isolated biomarkers exhibits a change in combined reversal value between pregnant females at risk for pre-term birth and term controls.

In one aspect, provided herein is a composition comprising a reversal group of surrogate peptides for a reversal group of biomarkers comprising: (a) a reversal pair of biomarkers consisting of IBP4/SHBG; and (b) a reversal triplet of biomarkers consisting of (EGLN+PRL)/TETN, wherein said reversal group of biomarkers exhibits a change in combined reversal value between pregnant females at risk for pre-term birth and term controls.

In one aspect, provided herein is a composition comprising a reversal group of isolated biomarkers comprising (a) a reversal triplet of isolated biomarkers consisting of (EGLN+IBP4)/SHBG or (PAPP2+IBP4)/SHBG, wherein said reversal group of isolated biomarkers exhibits a change in combined reversal value between pregnant females at risk for pre-term birth and term controls.

In one aspect, provided herein is a composition comprising a reversal group of surrogate peptides for a reversal group of biomarkers comprising a reversal triplet of isolated biomarkers consisting of (EGLN+IBP4)/SHBG or (PAPP2+IBP4)/SHBG, wherein said reversal group of biomarkers exhibits a change in combined reversal value between pregnant females at risk for pre-term birth and term controls.

In one aspect, provided herein is a composition comprising at least two pairs of biomarkers, wherein the at least two pairs of biomarkers comprise a first reversal pair of IBP4/SHBG; and a second reversal pair selected from Table 22, wherein said pairs of biomarkers exhibit a change in reversal value between pregnant females at risk for pre-term birth and term controls.

In some embodiments, the composition comprises a third reversal pair, wherein the third reversal pair is a reversal pair of isolated biomarkers selected from Table 22, except the third reversal pair and the second reversal pair are not the same.

In one aspect, provided herein is a composition comprising a reversal group of isolated biomarkers comprising: (a) a first reversal pair of isolated biomarkers consisting of IBP4/SHBG; and (b) a second reversal pair of isolated biomarkers selected from Table 22, wherein said reversal group of isolated biomarkers exhibits a change in combined reversal value between pregnant females at risk for pre-term birth and term controls

In one aspect, provided herein is a composition comprising a reversal group of surrogate peptides for a reversal group of biomarkers comprising: (a) a first reversal pair of isolated biomarkers consisting of IBP4/SHBG; and (b) a second reversal pair of isolated biomarkers selected from Table 22, wherein said reversal group of biomarkers exhibits a change in combined reversal value between pregnant females at risk for pre-term birth and term controls.

In some embodiments, the composition comprising a reversal group of isolated biomarkers further comprise a third reversal pair, wherein the third reversal pair is a reversal pair of isolated biomarkers is selected from Table 22, except the third reversal pair and the second reversal pair are not the same.

In some embodiments, the composition further comprises stable isotope labeled standard peptides (SIS peptides) corresponding to each of the surrogate peptides.

In one aspect, provided herein is a panel comprising at least three pairs of biomarkers selected from the group consisting of IBP4/SHBG, IBP4/TETN, EGLN/SHBG, EGLN/TETN, PRL/SHBG, and PRL/TETN, wherein said pairs of biomarkers exhibit a change in reversal value between pregnant females at risk for pre-term birth and term controls.

In one aspect, provided herein is a panel of biomarkers comprising a reversal group of biomarkers comprising: (a) a reversal pair of biomarkers consisting of IBP4/SHBG; and (b) a reversal triplet of biomarkers consisting of (EGLN+PRL)/TETN, wherein said reversal group of biomarkers exhibits a change in combined reversal value between pregnant females at risk for pre-term birth and term controls.

In one aspect, provided herein is a panel of surrogate peptides comprising a reversal group of surrogate peptides for a reversal group of biomarkers comprising: (a) a reversal pair of biomarkers consisting of IBP4/SHBG; and (b) a reversal triplet of biomarkers consisting of (EGLN+PRL)/TETN, wherein said reversal group of biomarkers exhibits a change in combined reversal value between pregnant females at risk for pre-term birth and term controls.

In one aspect, provided herein is a panel of biomarkers comprising a reversal group of biomarkers comprising: a reversal triplet of isolated biomarkers consisting of (EGLN+IBP4)/SHBG or (PAPP2+IBP4)/SHBG, wherein said reversal group of isolated biomarkers exhibits a change in combined reversal value between pregnant females at risk for pre-term birth and term controls.

In one aspect, provided herein is a panel of surrogate peptides comprising a reversal group of surrogate peptides for a reversal group of biomarkers comprising: a reversal triplet of isolated biomarkers consisting of (EGLN+IBP4)/SHBG or (PAPP2+IBP4)/SHBG, wherein said reversal group of isolated biomarkers exhibits a change in combined reversal value between pregnant females at risk for pre-term birth and term controls.

In one aspect, provided herein is a panel comprising at least two pairs of biomarkers, wherein the at least two pairs of biomarkers comprise a first reversal pair of IBP4/SHBG; and a second reversal pair selected from Table 22, wherein said pairs of biomarkers exhibits a change in reversal value between pregnant females at risk for pre-term birth and term controls.

In some embodiments, the panel comprises a third pair of biomarkers, wherein the third pair of biomarkers is a reversal pair of isolated biomarkers selected from Table 22, except the third reversal pair and the second reversal pair are not the same.

In one aspect, provided herein is a panel of biomarkers comprising a reversal group of biomarkers comprising: a first reversal pair of isolated biomarkers consisting of IBP4/SHBG; and a second reversal pair of biomarkers selected from Table 22, wherein said reversal group of biomarkers exhibits a change in combined reversal value between pregnant females at risk for pre-term birth and term controls.

In one aspect, provided herein is a panel of surrogate peptides comprising a reversal group of surrogate peptides for a reversal group of biomarkers comprising: a first reversal pair of isolated biomarkers consisting of IBP4/SHBG; and a second reversal pair of biomarkers selected from Table 22, wherein said reversal group of biomarkers exhibits a change in combined reversal value between pregnant females at risk for pre-term birth and term controls.

In some embodiments, the panel comprising the reversal group further comprises a third reversal pair, wherein the third reversal pair is a reversal pair of isolated biomarkers selected from Table 22, except the third reversal pair and the second reversal pair are not the same.

In some embodiments, the panel further comprises SIS peptides corresponding to each of the surrogate peptides.

In one aspect, provided herein is a method of determining probability for preterm birth in a pregnant female, the method comprising measuring in a biological sample obtained from said pregnant female, at least three pairs of biomarkers selected from the group consisting of IBP4/SHBG, IBP4/TETN, EGLN/SHBG, EGLN/TETN, PRL/SHBG, and PRL/TETN, wherein said pairs of biomarkers exhibit a change in reversal value between pregnant females at risk for pre-term birth and term controls.

In one aspect, provided herein is a method of determining probability for preterm birth in a pregnant female, the method comprising measuring in a biological sample obtained from said pregnant female a reversal group of biomarkers comprising: (a) a reversal pair of biomarker consisting of IBP4/SHBG; and (b) a reversal triplet of biomarkers consisting of (EGLN+PRL)/TETN, wherein said reversal group of biomarkers exhibits a change between pregnant females at risk for pre-term birth and term controls.

In one aspect, provided herein is a method of determining probability for preterm birth in a pregnant female, the method comprising measuring in a biological sample obtained from said pregnant female a reversal group of surrogate peptides for a reversal group of biomarkers comprising: (a) a reversal pair of biomarkers consisting of IBP4/SHBG; and (b) a reversal triplet of biomarkers consisting of (EGLN+PRL)/TETN, wherein said reversal group of biomarkers exhibits a change between pregnant females at risk for pre-term birth and term controls.

In one aspect, provided herein is a method of determining probability for preterm birth in a pregnant female, the method comprising: (a) obtaining a biological sample from the pregnant female; (b) measuring a reversal group of biomarkers comprising: (i) a reversal pair of biomarkers consisting of IBP4/SHBG; and (ii) a reversal triplet of biomarkers consisting of (EGLN+PRL)/TETN; and (c) determining the reversal value of said reversal group, wherein said reversal value of said reversal group of biomarkers exhibits a change between pregnant females at risk for pre-term birth and term controls.

In one aspect, provided herein is a method of determining probability for preterm birth in a pregnant female, the method comprising: (a) obtaining a biological sample from the pregnant female; (b) measuring a reversal pair of biomarkers consisting of IBP4/SHBG; (c) determining a first reversal value of said reversal pair; (d) measuring a reversal triplet of biomarkers consisting of (EGLN+PRL)/TETN; (e) determining a second reversal value of said reversal triplet; (f) combining the first reversal value and the second reversal value into a combined reversal value, wherein said combined reversal value exhibits a change between pregnant females at risk for pre-term birth and term controls.

In one aspect, provided herein is a method of determining probability for preterm birth in a pregnant female, the method comprising measuring in a biological sample obtained from said pregnant female a reversal group of biomarkers comprising: a reversal triplet of isolated biomarkers consisting of (EGLN+IBP4)/SHBG or (PAPP2+IBP4)/SHBG, wherein said reversal group of biomarkers exhibits a change between pregnant females at risk for pre-term birth and term controls.

In one aspect, provided herein is a method of determining probability for preterm birth in a pregnant female, the method comprising measuring in a biological sample obtained from said pregnant female a reversal group of surrogate peptides for a reversal group of biomarkers comprising: (a) a reversal triplet of isolated biomarkers consisting of (EGLN+IBP4)/SHBG or (PAPP2+IBP4)/SHBG, wherein said reversal group of biomarkers exhibits a change between pregnant females at risk for pre-term birth and term controls.

In one aspect, provided herein is a method of determining probability for preterm birth in a pregnant female, the method comprising: (a) obtaining a biological sample from the pregnant female; (b) measuring a reversal group of biomarkers comprising: (i) a reversal triplet of isolated biomarkers consisting of (EGLN+IBP4)/SHBG or (PAPP2+IBP4)/SHBG; and (c) determining a combined reversal value of said reversal group, wherein said combined reversal value of said reversal group of biomarkers exhibits a change between pregnant females at risk for pre-term birth and term controls.

In one aspect, provided herein is a method of determining probability for preterm birth in a pregnant female, the method comprising: (a) obtaining a biological sample from the pregnant female; (b) measuring a reversal triplet of isolated biomarkers consisting of (EGLN+IBP4)/SHBG or (PAPP2+IBP4)/SHBG; and (c) determining a combined reversal value of said reversal triplet; wherein said combined reversal value exhibits a change between pregnant females at risk for pre-term birth and term controls.

In one aspect, provided herein is a method of determining probability for preterm birth in a pregnant female, the method comprising measuring a biological sample obtained from said pregnant female, at least two pairs of biomarkers, wherein the at least two pairs of biomarkers comprise a first reversal pair of IBP4/SHBG; and a second reversal pair selected from Table 22, wherein said pairs of biomarkers exhibit a change in reversal value between pregnant females at risk for pre-term birth and term controls.

In some embodiments, the method comprises measuring a third pair of biomarkers, wherein the third pair of biomarkers is a reversal pair of isolated biomarkers selected from Table 22, except the third reversal pair and the second reversal pair are not the same.

In one aspect, provided herein is a method of determining probability for preterm birth in a pregnant female, the method comprising measuring a biological sample obtained from said pregnant female a reversal group of biomarkers comprising: (a) a first reversal pair of isolated biomarkers consisting of IBP4/SHBG; and (b) a second reversal pair of isolated biomarkers selected from Table 22; wherein said reversal group of biomarkers exhibits a change between pregnant females at risk for pre-term birth and term controls.

In one aspect, provided herein is a method of determining probability for preterm birth in a pregnant female, the method comprising measuring in a biological sample obtained from said pregnant female a reversal group of surrogate peptides for a reversal group of biomarkers comprising: (a) a first reversal pair of isolated biomarkers consisting of IBP4/SHBG; and (b) a second reversal pair of isolated biomarkers selected from Table 22; wherein said reversal group of biomarkers exhibits a change between pregnant females at risk for pre-term birth and term controls.

In one aspect, provided herein is a method of determining probability for preterm birth in a pregnant female, the method comprising: (a) obtaining a biological sample from the pregnant female; (b) measuring a reversal group of biomarkers comprising: (i) a first reversal pair of isolated biomarkers consisting of IBP4/SHBG; and (ii) a second reversal pair of isolated biomarkers selected from Table 22; and (c) determining the reversal value of said reversal group, wherein said reversal value of said reversal group of biomarkers exhibits a change between pregnant females at risk for pre-term birth and term controls.

In one aspect, provided herein is a method of determining probability for preterm birth in a pregnant female, the method comprising: (a) obtaining a biological sample from the pregnant female; (b) measuring a reversal group of biomarkers comprising a first reversal pair of biomarkers consisting of IBP4/SHBG; (c) determining a first reversal value of said first reversal pair; (d) measuring a second reversal pair of biomarkers selected from Table 22; (e) determining a second reversal value of said second reversal pair; (f) combining the first reversal value and the second reversal value into a combined reversal value, wherein said combined reversal value exhibits a change between pregnant females at risk for pre-term birth and term controls.

In some embodiments, the method comprising the reversal group of biomarkers further comprises a third reversal pair, wherein the third reversal pair is a reversal pair of isolated biomarkers selected from Table 22, except the third reversal pair and the second reversal pair are not the same.

In some embodiments, the method further comprises a step of determining gestational age at blood draw (GABD). In some embodiments, the step of determining GABD is performed before said obtaining a biological sample.

In some embodiments, the method further comprises a step of determining body mass index (BMI). In some embodiments, the step of determining BMI is performed before said obtaining a biological sample.

In some embodiments, the method further comprises an initial step of detecting a measurable feature for one or more risk indicia. In some embodiments, the one or more risk indicia are combined with the measurement of said pairs of biomarkers into a test score that, when compared to a reference score, exhibits a change in score between pregnant females at risk for pre-term birth and term controls. In other embodiments, the one or more risk indicia are combined with the measurement of said reversal group of biomarkers into a test score that, when compared to a reference score, exhibits a change in score between pregnant females at risk for pre-term birth and term controls. In further embodiments, the one or more risk indicia are combined with said reversal value into a test score that, when compared to a reference score, exhibits a change in score between pregnant females at risk for pre-term birth and term controls. In further embodiments, the one or more risk indicia are combined with said combined reversal value into a test score that, when compared to a reference score, exhibits a change in score between pregnant females at risk for pre-term birth and term controls. In further embodiments, the one or more risk indicia is selected from the group consisting of prior preterm birth, short cervical length, prior miscarriage, prior stillbirth, Body Mass Index (BMI), maternal age, parity, gravidity, fetal gender, height and weight separately from BMI, race and socioeconomic status. In specific embodiments, the risk indicium is BMI.

In some embodiments, the method further comprises prediction of gestational age at birth (GAB) prior to said determining the probability for preterm birth.

In some embodiments of the method, the existence of a change in reversal value, combined reversal value, or final reversal value between the pregnant female and a term control indicates the probability for preterm birth in the pregnant female.

In some embodiments of the method, measuring comprises measuring surrogate peptides of said reversal pair and reversal triplet in the biological sample obtained from said pregnant female. In other embodiments of the method, measuring further comprises measuring stable isotope labeled standard peptides (SIS peptides) for each of the surrogate peptides.

In some embodiments of the method, probability is expressed as a risk score.

In some embodiments of the method, the biological sample is selected from the group consisting of whole blood, plasma, serum, saliva, urine, amniotic fluid, cervical vaginal fluid. In particular embodiments, the biological sample is serum.

In some embodiments of the method, measuring comprises mass spectrometry (MS). In some embodiments, MS is selected from the group consisting of matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n. In other embodiments, MS comprises co-immunoprecipitation-mass spectrometry (co-IP MS). In other embodiments, MS comprises liquid chromatography-mass spectrometry (LC-MS). In other embodiments, MS comprises multiple reaction monitoring (MRM) or selected reaction monitoring (SRM).

In some embodiments of the method, measuring comprises an assay that utilizes a capture agent. In some embodiments, the capture agent is selected from the group consisting of and antibody, antibody fragment, nucleic acid-based protein binding reagent, small molecule or variant thereof. In further embodiments, the assay is selected from the group consisting of enzyme immunoassay (EIA), enzyme-linked immunosorbent assay (ELISA), and radioimmunoassay (RIA).

In one aspect, provided herein is a method of detecting at least three pairs of biomarkers selected from the group consisting of IBP4/SHBG, IBP4/TETN, EGLN/SHBG, EGLN/TETN, PRL/SHBG, and PRL/TETN in a pregnant female, said method comprising, (a) obtaining a biological sample from said pregnant female; and (b) detecting whether the at least three pairs of biomarkers is present in the biological sample, wherein said pairs of biomarkers exhibit a change in reversal value between pregnant females at risk for pre-term birth and term controls.

In one aspect, provided herein is a method of detecting at least two pairs of biomarkers in a pregnant female, wherein the at least two pairs of biomarkers comprise a first reversal pair of IBP4/SHBG and a second reversal pair selected from Table 22, said method comprising, (a) obtaining a biological sample from said pregnant female; and (b) detecting whether the at least two pairs of biomarkers is present in the biological sample, wherein said pairs of biomarkers exhibit a change in reversal value between pregnant females at risk for pre-term birth and term controls.

In some embodiments, the method comprises detecting a third biomarker pair, wherein the third biomarker pair is a biomarker pair selected from Table 22, except the third biomarker pair and the second biomarker pair are not the same.

In some embodiments, the method further comprises a step of determining gestational age at blood draw (GABD). In other embodiments, the method further comprises a step of determining body mass index (BMI).

In some embodiments, the method of determining GABD is performed before obtaining a biological sample from pregnant female. In other embodiments, the step of determining BMI is performed before obtaining a biological sample from a pregnant female.

In some embodiments, the method further comprises an initial step of detecting a measurable feature for one or more risk indicia. In some embodiments, the one or more risk indicia are combined with the measurement of said pairs of biomarkers into a test score that, when compared to a reference score, exhibits a change in score between pregnant females at risk for pre-term birth and term controls. In other embodiments, the one or more risk indicia are combined with the measurement of said reversal group of biomarkers into a test score that, when compared to a reference score, exhibits a change in score between pregnant females at risk for pre-term birth and term controls. In further embodiments, the one or more risk indicia are combined with said reversal value or combined reversal value into a test score that, when compared to a reference score, exhibits a change in score between pregnant females at risk for pre-term birth and term controls. In further embodiments, the one or more risk indicia are combined with said combined reversal value into a test score that, when compared to a reference score, exhibits a change in score between pregnant females at risk for pre-term birth and term controls. In some embodiments, the one or more risk indicia is selected from the group consisting of prior preterm birth, short cervical length, prior miscarriage, prior stillbirth, Body Mass Index (BMI), maternal age, parity, gravidity, fetal gender, height and weight separately from BMI, race and socioeconomic status. In particular embodiments, the risk indicium is BMI.

In some embodiments, the method further comprises prediction of gestational age at birth (GAB) prior to said determining the probability for preterm birth.

In some embodiments of the method, existence of a change in reversal value, combined reversal value, or final reversal value between the pregnant female and a term control indicates the probability for preterm birth in the pregnant female.

In some embodiments of the method, measuring comprises measuring surrogate peptides of said reversal pair and reversal triplet in the biological sample obtained from said pregnant female. In some embodiments of the method, measuring further comprises measuring stable isotope labeled standard peptides (SIS peptides) for each of the surrogate peptides.

In some embodiments of the method, probability is expressed as a risk score.

In some embodiments, the method comprises a biological sample selected from the group consisting of whole blood, plasma, serum, saliva, urine, amniotic fluid, cervical vaginal fluid. In particular embodiments, the biological sample is serum.

In some embodiments of the method, measuring comprises mass spectrometry (MS). In some embodiments, MS is selected from the group consisting of matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n. In other embodiments, MS comprises co-immunoprecipitation-mass spectrometry (co-IP MS). In some embodiments, MS comprises liquid chromatography-mass spectrometry (LC-MS). In some embodiments, MS comprises multiple reaction monitoring (MRM) or selected reaction monitoring (SRM).

In some embodiments of the method, measuring comprises an assay that utilizes a capture agent. In other embodiments, the capture agent is selected from the group consisting of and antibody, antibody fragment, nucleic acid-based protein binding reagent, small molecule or variant thereof. In other embodiments of the method, the assay is selected from the group consisting of enzyme immunoassay (EIA), enzyme-linked immunosorbent assay (ELISA), and radioimmunoassay (RIA).

In one aspect, provided herein is a method of detecting a reversal group of biomarkers comprising a reversal pair of biomarkers consisting of IBP4/SHBG and a reversal triplet of biomarkers consisting of (EGLN+PRL)/TETN in a pregnant female, said method comprising: (a) obtaining a biological sample from said pregnant female; and (b) detecting whether the reversal group of biomarkers is present in the biological sample, wherein said detecting comprises subjecting said sample to a proteomics work-flow comprised of mass spectrometry (MS) quantification.

In one aspect, provided herein is a method of detecting a reversal group of biomarkers comprising a reversal pair of biomarkers consisting of IBP4/SHBG and a reversal triplet consisting of (EGLN+PRL)/TETN in a pregnant female, said method comprising: (a) obtaining a biological sample from the pregnant female; (b) first detecting whether the reversal pair of biomarkers is present in the biological sample; and (c) second detecting whether the reversal triplet of biomarkers is present in the biological sample, wherein said first detecting step and/or said second detecting step comprises subjecting said sample to a proteomics work-flow comprised of mass spectrometry (MS) quantification.

In one aspect, provided herein is a method of detecting at least three pairs of biomarkers selected from the group consisting of IBP4/SHBG, IBP4/TETN, EGLN/SHBG, EGLN/TETN, PRL/SHBG, and PRL/TETN in a pregnant female, said method comprising, (a) obtaining a biological sample from said pregnant female; and (b) detecting whether the pair of isolated biomarkers is present in the biological sample by contacting the biological sample with a first capture agent that specifically binds a first member of said pair and a second capture agent that specifically binds a second member of said pair; and, (c) detecting binding between the first biomarker of said pair and the first capture agent and between the second member of said pair and the second capture agent, wherein said detecting comprises an assay that utilizes the capture agent.

In one aspect, provided herein is a method of detecting a reversal group of biomarkers comprising a reversal pair of biomarkers consisting of IBP4/SHBG and a reversal triplet of biomarkers consisting of (EGLN+PRL)/TETN in a pregnant female, said method comprising: (a) obtaining a biological sample from the pregnant female; (b) detecting whether the reversal group of biomarkers is present in the biological sample by contacting the biological sample with a capture agent that specifically binds an individual biomarker of the reversal group; and (c) detecting binding between the capture agent and the individual biomarker of the reversal group wherein said detecting comprises an assay that utilizes the capture agent.

In one aspect, provided herein is a method of detecting a reversal group of biomarkers comprising a reversal triplet of isolated biomarkers consisting of (EGLN+IBP4)/SHBG or (PAPP2+IBP4)/SHBG in a pregnant female, said method comprising (a) obtaining a biological sample from said pregnant female; and (b) detecting whether the reversal group of biomarkers is present in the biological sample, wherein said detecting comprises subjecting said sample to a proteomics work-flow comprised of mass spectrometry (MS) quantification.

In one aspect, provided herein is a method of detecting a reversal group of biomarkers comprising a reversal triplet of biomarkers consisting of (EGLN+IBP4)/SHBG or (PAPP2+IBP4)/SHBG in a pregnant female, said method comprising: (a) obtaining a biological sample from the pregnant female; (b) detecting whether the reversal group of biomarkers is present in the biological sample by contacting the biological sample with a capture agent that specifically binds an individual biomarker of the reversal group; and (c) detecting binding between the capture agent and the individual biomarker of the reversal group, wherein said detecting comprises an assay that utilizes the capture agent.

In one aspect, provided herein is a detecting a reversal group of biomarkers in a pregnant female comprising a first reversal pair of biomarkers consisting of IBP4/SHBG and a second reversal pair of biomarkers selected from Table 22, said method comprising: (a) obtaining a biological sample from said pregnant female; and (b) detecting whether the reversal group of biomarkers is present in the biological sample, wherein said detecting comprises subjecting said sample to a proteomics work-flow comprised of mass spectrometry (MS) quantification.

In one aspect, provided herein is a method of detecting a reversal group of biomarkers in a pregnant female comprising a first reversal pair of biomarkers consisting of IBP4/SHBG and a second reversal pair of biomarkers selected from Table 22, said method comprising: (a) obtaining a biological sample from the pregnant female; (b) first detecting whether the first reversal pair of biomarkers is present in the biological sample; and (c) second detecting whether the second reversal pair of biomarkers is present in the biological sample, wherein said first detecting step and/or said second detecting step comprises subjecting said sample to a proteomics work-flow comprised of mass spectrometry (MS) quantification.

In one aspect, provided herein is a method of detecting at least two pairs of biomarkers in a pregnant female, wherein the at least two pairs of biomarkers comprise a first reversal pair of IBP4/SHBG and a second reversal pair selected from Table 22, said method comprising, (a) obtaining a biological sample from said pregnant female; and (b) detecting whether the pairs of isolated biomarkers are present in the biological sample by contacting the biological sample with a first capture agent that specifically binds a first member of said pairs and a second capture agent that specifically binds a second member of said pairs; and, (c) detecting binding between the first biomarker of said pairs and the first capture agent and between the second member of said pairs and the second capture agent, wherein said detecting comprises an assay that utilizes the capture agent

In some embodiments, the method comprises detecting a third pair of biomarkers, wherein the third pair of biomarkers is a reversal pair of isolated biomarkers selected from Table 22, except the third reversal pair and the second reversal pair are not the same.

In one aspect, provided herein is a method of detecting a reversal group of biomarkers comprising a first reversal pair of biomarkers consisting of IBP4/SHBG and a second reversal pair of biomarkers selected from Table 22 in a pregnant female, said method comprising: (a) obtaining a biological sample from the pregnant female; (b) detecting whether the reversal group of biomarkers is present in the biological sample by contacting the biological sample with a capture agent that specifically binds an individual biomarker of the reversal group; and (c) detecting binding between the capture agent and the individual biomarker of the reversal group, wherein said detecting comprises an assay that utilizes the capture agent.

In some embodiments, the reversal group of biomarkers detected in the method comprise a third reversal pair, wherein the third reversal pair is a reversal pair of isolated biomarkers selected from Table 22, except the third reversal pair and the second reversal pair are not the same.

In some embodiments, the method further comprises an initial step of determining gestational age at blood draw (GABD). In other embodiments, the method further comprises an initial step of determining Body Mass Index (BMI).

In other embodiments, the method further comprises detecting a measurable feature for one or more risk indicia. In some embodiments, the risk indicium is selected from the group consisting of prior preterm birth, short cervical length, prior miscarriage, prior stillbirth, body mass index (BMI), maternal age, parity, gravidity, fetal gender, height and weight separately from BMI, race, and low socioeconomic status. In particular embodiments, the risk indicium is BMI.

In some embodiments, the method further comprises measuring a reversal value or combined reversal value for each of said pair of biomarkers or said reversal group of biomarkers.

In some embodiments of the method, existence of a change in said reversal value or combined reversal value between the pregnant female and a term control indicates the probability for preterm birth in the pregnant female.

In some embodiments of the method, probability is expressed as a risk score.

In some embodiments of the method, the biological sample is selected from the group consisting of whole blood, plasma, serum, saliva, urine, amniotic fluid, cervical vaginal fluid. In particular embodiments, the biological sample is serum.

In some embodiments of the method, detecting comprises mass spectrometry (MS). In other embodiments of the method, detecting comprises an assay that utilizes a capture agent.

In some embodiments, the capture agent is selected from the group consisting of and antibody, antibody fragment, nucleic acid-based protein binding reagent, small molecule or variant thereof. In other embodiments, the assay is selected from the group consisting of enzyme immunoassay (EIA), enzyme-linked immunosorbent assay (ELISA), and radioimmunoassay (RIA).

In some embodiments of the method, MS is selected from the group consisting of matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n. In other embodiments, MS comprises affinity-capture MS (AC-MS). In other embodiments, MS comprises co-immunoprecipitation-mass spectrometry (co-IP MS). In other embodiments, MS comprises liquid chromatography-mass spectrometry (LC-MS). In other embodiments, MS comprises multiple reaction monitoring (MRM) or selected reaction monitoring (SRM).

In one aspect, provided herein is a method of treating or preventing preterm birth in a pregnant female the method comprising: (a) obtaining a biological sample from said pregnant female; (b) detecting a reversal group of biomarkers in said sample; (c) providing a risk score for said pregnant female; (d) prognosing said pregnant female as having an increased risk of preterm birth; and (e) administering one or more therapies to said pregnant female to prevent preterm birth.

In some embodiments, the reversal group comprises a reversal pair of and a reversal triplet of biomarkers. In some embodiments, the reversal pair comprises IBP4/SHBG. In some embodiments, the reversal triplet comprises (EGLN+PRL)/TETN.

In some embodiments, the reversal group comprises a reversal triplet of biomarkers consisting of (EGLN+IBP4)/SHBG or (PAPP2+IBP4)/SHBG.

In some embodiments, the reversal group comprises at least two reversal pairs of biomarkers, wherein the at least two reversal pairs comprise a first reversal pair and a second reversal pair of biomarkers. In some embodiments, the first reversal pair comprises IBP4/SHBG. In some embodiments, the second reversal pair comprises a reversal group of biomarkers selected from Table 22. In some embodiments, the reversal group comprises a third reversal pair, wherein the third reversal pair is a reversal pair of isolated biomarkers selected from Table 22, except the third reversal pair and the second reversal pair are not the same.

In some embodiments, therapies comprise cervical cerclage, administration of 17-α hydroxyprogesterone caproate, vaginal progesterone gel, antenatal corticosteroids, cervical pessaries, or elevated care.

In some embodiments, the method further comprises prediction of gestational age at birth (GAB) before administering one or more therapies to prevent preterm birth.

In some embodiments, the method further comprises an initial step of determining gestational age at blood draw (GABD). In particular embodiments, the step of determining GABD is performed before administering one or more therapies to prevent preterm birth.

In some embodiments, the method further comprises an initial step of determining Body Mass Index (BMI). In particular embodiments, the step of determining BMI is performed before administering one or more therapies to prevent preterm birth.

In some embodiments, the method further comprises detecting a measurable feature for one or more risk indicia. In particular embodiments, the one or more risk indicia are incorporated into said test risk score and said reference risk score. In some embodiments, the risk indicium is selected from the group consisting of prior preterm birth, short cervical length, prior miscarriage, prior stillbirth, body mass index (BMI), maternal age, parity, gravidity, fetal gender, height and weight separately from BMI, race, and low socioeconomic status. In particular embodiments, the risk indicium is BMI.

In some embodiments, the method further comprises measuring a reversal value or combined reversal value for said pair of biomarkers or said reversal group of biomarkers.

In some embodiments of the method, the existence of a change in said reversal value or combined reversal value between the pregnant female and a term control indicates the probability for preterm birth in the pregnant female.

In some embodiments, the biological sample is selected from the group consisting of whole blood, plasma, serum, saliva, urine, amniotic fluid, cervical vaginal fluid. In particular embodiments, the biological sample is serum.

In some embodiments of the method, detecting comprises an assay that utilizes a capture agent. In particular embodiments, the capture agent is selected from the group consisting of and antibody, antibody fragment, nucleic acid-based protein binding reagent, small molecule or variant thereof. In some embodiments, the assay is selected from the group consisting of enzyme immunoassay (EIA), enzyme-linked immunosorbent assay (ELISA), and radioimmunoassay (RIA).

In some embodiments of the method, detecting comprises mass spectrometry (MS). In some embodiments, MS is selected from the group consisting of matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n. In other embodiments, MS comprises affinity-capture MS (AC-MS). In other embodiments, MS comprises co-immunoprecipitation-mass spectrometry (co-IP MS). In other embodiments, MS comprises liquid chromatography-mass spectrometry (LC-MS). In other embodiments, MS comprises multiple reaction monitoring (MRM) or selected reaction monitoring (SRM).

In one aspect, provided herein is a method of treating and/or preventing preterm birth, the method comprising: (a) obtaining a biological sample from a pregnant female; (b) detecting a reversal group of biomarkers in said sample; (c) providing a test risk score for said pregnant female based at least in part on the detected level of said reversal group in said sample; and (d) administering one or more preterm birth interventions to said pregnant female when the risk score exceeds a reference risk score.

In some embodiments, the reversal group comprises a reversal pair of and a reversal triplet of biomarkers. In some embodiments, the reversal pair comprises IBP4/SHBG. In some embodiments, the reversal triplet comprises (EGLN+PRL)/TETN.

In some embodiments, the reversal group comprises a reversal triplet of biomarkers consisting of (EGLN+IBP4)/SHBG or (PAPP2+IBP4)/SHBG.

In some embodiments, the reversal group comprises at least two reversal pairs of biomarkers, wherein the at least two reversal pairs comprise a first reversal pair and a second reversal pair of biomarkers. In some embodiments, the first reversal pair comprises IBP4/SHBG. In some embodiments, the second reversal pair comprises a reversal group of biomarkers selected from Table 22. In some embodiments, the reversal group comprises a third reversal pair of biomarkers, wherein the third reversal pair of biomarkers is a reversal pair of isolated biomarkers selected from Table 22, except the third reversal pair and the second reversal pair are not the same.

In some embodiments of the method, preterm interventions comprise cervical cerclage, administration of 17-α hydroxyprogesterone caproate, vaginal progesterone gel, antenatal corticosteroids, cervical pessaries, or elevated care.

In some embodiments, the method further comprises prediction of gestational age at birth (GAB) prior administering one or more preterm birth interventions to said pregnant female.

In some embodiments, the method further comprises a step of determining gestational age at blood draw (GABD). In some embodiments, the step of determining GABD is performed before obtaining a biological sample from a pregnant female.

In some embodiments, the method further comprises a step of determining Body Mass Index (BMI). In some embodiments of the method, the step of determining BMI is performed before administering one or more preterm birth interventions to said pregnant female.

In some embodiments, the method further comprises detecting a measurable feature for one or more risk indicia. In some embodiments, the one or more risk indicia are incorporated into said test risk score and said reference risk score. In some embodiments, the risk indicium is selected from the group consisting of prior preterm birth, short cervical length, prior miscarriage, prior stillbirth, body mass index (BMI), maternal age, parity, gravidity, fetal gender, height and weight separately from BMI, race, and low socioeconomic status. In particular embodiments, the risk indicium is BMI.

In some embodiments, the method further comprises measuring a reversal value or combined reversal value for said pair of biomarkers or said reversal group of biomarkers. In further embodiments, the existence of a change in said reversal value or combined reversal value between the pregnant female and a term control indicates the probability for preterm birth in the pregnant female.

In some embodiments of the method, the biological sample is selected from the group consisting of whole blood, plasma, serum, saliva, urine, amniotic fluid, cervical vaginal fluid. In particular embodiments, the biological sample is serum.

In some embodiments, detecting comprises an assay that utilizes a capture agent. In some embodiments, the capture agent is selected from the group consisting of and antibody, antibody fragment, nucleic acid-based protein binding reagent, small molecule or variant thereof. In some embodiments, the assay is selected from the group consisting of enzyme immunoassay (EIA), enzyme-linked immunosorbent assay (ELISA), and radioimmunoassay (RIA).

In some embodiments, said detecting comprises mass spectrometry (MS). In some embodiments, MS is selected from the group consisting of matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n. In other embodiments, MS comprises affinity-capture MS (AC-MS). In other embodiments, MS comprises co-immunoprecipitation-mass spectrometry (co-IP MS). In other embodiments, MS comprises liquid chromatography-mass spectrometry (LC-MS). In other embodiments, MS comprises multiple reaction monitoring (MRM) or selected reaction monitoring (SRM).

### BRIEF DESCRIPTION OF THE DRAWINGS

FIG. 1 shows the rate of births for the top 15% of those with high scores in the IBP4/SHBG+GABD*BMI+(EGLN+PRL)/TETN predictor (high risk) vs. low scores (low score) by Kaplan-Meier analysis. Horizontal lines indicate delivery at 34 (238 days) and 37 weeks (259 days).

FIG. 2 shows data taken from a causal network analysis showing relationships of proteins measured by MRM-MS and clinical variables to preterm birth (sPTB, center circle). Grey gradients represent different clusters. Arrows indicate causal connections and point from parent to child, with bidirectional arrows denoting indeterminate causality. Proteins are represented by small circles while clinical variables are represented by triangles denoting conditions present before blood draw. Instances where a protein is depicted more than once (denoted by _1 and _2), indicate the protein was measured based on two distinct peptides. “Prior preterm birth” represents multigravidas with a prior PTB and “Primigravida” represents women without a history of PTB as this is their first pregnancy, both of which are compared to the baseline category, multigravidas without prior PTB.

### DETAILED DESCRIPTION

The present disclosure is based, generally, on the discovery that certain proteins and peptides in biological samples obtained from a pregnant female are differentially expressed in pregnant females that have an increased risk of preterm birth relative to controls. The present disclosure is further specifically based, in part, on the unexpected discovery that a reversal group of biomarkers disclosed herein can be utilized in methods, kits, compositions, and systems involving determining the probability for preterm birth in a pregnant female with high sensitivity and specificity. The proteins and peptides disclosed herein as components of ratios and/or reversal pairs or reversal triplets serve as biomarkers for treating pregnant patients (e.g., patients with an elevated predicted probability of preterm birth), classifying test samples, predicting probability of preterm birth, predicting probability of term birth, predicting gestational age at birth (GAB), predicting time to birth (TTB) and/or monitoring of progress of preventative therapy in a pregnant female at risk for PTB, either individually, in ratios, reversal pairs, reversal triplets or in panels of biomarkers, reversal pairs or reversal triplets. The invention lies, in part, in the selection of particular biomarkers that, when combined as a reversal pair and/or reversal triplet, can predict the probability of pre-term birth based on reversal values or combined reversal values. In some embodiments, such reversal pairs or triplets, or the reversal values or combined reversal values, can predict the probability of preterm-birth in nulliparous women (women who had not had a previous birth, also referred to herein as “nullips”) or multiparous women (women with one or more births, also referred to herein as “multips”). In some embodiments, such reversal pairs or triplets, or the reversal values or combined reversal values, can predict the probability of preterm-birth in only nulliparous women. In some embodiments, such reversal pairs or triplets, or the reversal values or combined reversal values, can predict the probability of preterm-birth in only multiparous women. In some embodiments, such reversal pairs or triplets, or the reversal values or combined reversal values, can predict the probability of preterm-birth in both nulliparous women and multiparous women. One major advantage of the present disclosure is that risk of developing preterm birth can be assessed early during pregnancy so that appropriate monitoring and clinical management to prevent preterm delivery can be initiated in a timely fashion. Accordingly, the invention lies, in part, on human ingenuity in selecting the specific biomarkers that are informative upon being paired in novel reversals or triplets that underlies the present invention.

It must be noted that, as used herein, the singular forms “a”, “an” and “the” include plural references, unless the content clearly dictates otherwise and are used interchangeably with “at least one” and “one or more.” Thus, for example, reference to “a biomarker” includes a mixture of two or more biomarkers, and the like.

The term “about,” particularly in reference to a given quantity, is meant to encompass deviations of plus or minus five percent.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but can include other elements not expressly listed or inherent to such process, method, product-by-process, or composition of matter.

As used herein, the term “panel” refers to an array or a collection, comprising two or more biomarkers. The term can also refer to a profile or index of expression patterns of two or more biomarkers described herein. The number of biomarkers useful for a biomarker panel can, in some embodiments, be based on the sensitivity and specificity value for the particular combination of biomarker values. Biomarkers in a panel, or the panel as a unit, can be combined in some embodiments of the invention with other markers, including other biomarkers and/or clinical or demographic variables (e.g., BMI, GABD). Such combinations can be used to derive combined scores predictive of, and which can in turn be used to treat, pregnancy complications (e.g., preterm birth).

As used herein, and unless otherwise specified, the terms “isolated” and “purified” generally describes a composition of matter that has been removed from its native environment (e.g., the natural environment if it is naturally occurring), and thus is altered by the hand of man from its natural state so as to possess markedly different characteristics with regard to at least one of structure, function or property. An isolated protein or nucleic acid is distinct from the way it exists in nature and includes synthetic peptides and proteins.

The term “biomarker” refers to a biological molecule, or a fragment of a biological molecule, the change and/or the detection of which can be correlated with a particular physical condition or state. The terms “marker” and “biomarker” are used interchangeably throughout the disclosure. For example, the biomarkers of the present invention are correlated with an increased likelihood of preterm birth. Such biomarkers include any suitable analyte, but are not limited to, biological molecules comprising nucleotides, nucleic acids, nucleosides, amino acids, sugars, fatty acids, steroids, metabolites, peptides, polypeptides, proteins, carbohydrates, lipids, hormones, antibodies, regions of interest that serve as surrogates for biological macromolecules and combinations thereof (e.g., glycoproteins, ribonucleoproteins, lipoproteins). The term also encompasses portions or fragments of a biological molecule, for example, peptide fragment of a protein or polypeptide that comprises at least 5 consecutive amino acid residues, at least 6 consecutive amino acid residues, at least 7 consecutive amino acid residues, at least 8 consecutive amino acid residues, at least 9 consecutive amino acid residues, at least 10 consecutive amino acid residues, at least 11 consecutive amino acid residues, at least 12 consecutive amino acid residues, at least 13 consecutive amino acid residues, at least 14 consecutive amino acid residues, at least 15 consecutive amino acid residues, at least 5 consecutive amino acid residues, at least 16 consecutive amino acid residues, at least 17 consecutive amino acid residues, at least 18 consecutive amino acid residues, at least 19 consecutive amino acid residues, at least 20 consecutive amino acid residues, at least 21 consecutive amino acid residues, at least 22 consecutive amino acid residues, at least 23 consecutive amino acid residues, at least 24 consecutive amino acid residues, at least 25 consecutive amino acid residues, or more consecutive amino acid residues.

As used herein, the term “surrogate peptide” refers to a peptide that is selected to serve as a surrogate for quantification of a biomarker of interest in an MRM assay configuration. Quantification of surrogate peptides is best achieved using stable isotope labeled standard surrogate peptides (“SIS surrogate peptides” or “SIS peptides”) in conjunction with the MRM detection technique. Such SIS surrogate peptides can be synthetic or the endogenous peptide that is derived from the biomarker of interest. A SIS surrogate peptide can be synthesized with heavy labels for example, in the Arginine or Lysine residue at the C-terminus of tryptic peptides, or any other amino acid of the peptide to serve as an internal standard in the MRM assay. An SIS surrogate peptide is not a naturally occurring peptide and has a markedly different mass, detectable by a mass spectrometer, but similar structure and properties to its naturally occurring counterpart. The detectably different mass of a SIS peptide, together with its similar structure and properties makes it an ideal standard.

As used herein, the term “reversal” refers to the ratio of the measured value of one or more analytes (or some conversion or representation thereof) over that of one or more analytes, wherein the analytes provide some partially independent information. In some embodiments, the analyte value (or its representation) is itself a ratio of the peak area of the endogenous analyte over that of the peak area of a corresponding standard (e.g., stable isotopic standard analyte), referred to herein as: response ratio or relative ratio. For example, as exemplified herein, IBP4/SHBG is a ratio of an up-regulated protein in the numerator and a down-regulated protein in the denominator, which is defined herein as a “reversal pair.” As another example, as exemplified herein, (EGLN+PRL)/TETN is a ratio of two up-regulated proteins in the numerator and one down-regulated protein in the denominator, which is within the definition of a “reversal triplet” defined herein. As yet another example, (EGLN+IBP4)/SHBG and (PAPP2+IBP4)/SHBG are ratios of two up-regulated proteins in the numerator and one-down regulated protein in the denominator, which also fit within the definition of “reversal triplets”. In some embodiments, “/” is inclusive of but not limited to division, taking a root or difference. In other embodiments, “+” is inclusive of but not limited to addition, taking a power or multiplication. In all instances, one protein in a reversal may serve to normalize another protein (e.g. decrease variability due to biomedical conditions not of interest, or sample pre-analytical or analytical variability). In the particular case of a ratio that is a “reversal” both amplification of diagnostic signal and normalization are possible. It is understood, that the methods of the invention are not limited to the subset of reversals, but also encompass ratios of biomarkers.

As used herein, the term “reversal pair” refers to biomarkers in pairs where a mathematical combination of the values of the pair exhibits a change in value between the classes being compared. The detection of a reversal pair in protein concentrations or gene expression levels may eliminate the need for data normalization or the establishment of population-wide thresholds, and requires only that the levels of two or more biomarkers change relative to each other. In some embodiments, the reversal pair is a pair of isolated biomarkers IBP4/SHBG, wherein the reversal pair exhibits a change in reversal value between pregnant females at risk for pre-term birth compared to term controls. In a further embodiment, the reversal pair IBP4/SHBG exhibits a higher ratio in pregnant females at risk for pre-term birth compared to term controls. Encompassed within the definition of any reversal pair is the corresponding reversal pair wherein individual biomarkers are switched between the numerator and denominator. One skilled in the art will appreciate that such a corresponding reversal pair is equally informative with regard to its predictive power.

The term “reversal triplet” refers to three biomarkers that exhibit a change in value between the classes being compared. The detection of a reversal triplet in protein concentrations or gene expression levels may eliminate the need for data normalization or the establishment of population-wide thresholds and requires only that the levels of two or more biomarkers change relative to each other. In some embodiments, the reversal triplet is three isolated biomarkers EGLN, PRL and TETN, wherein two biomarkers are in the numerator (e.g., EGLN+PRL) and one biomarker is in the denominator (e.g., TETN). For example, a reversal triplet can be (EGLN+PRL)/TETN. In some embodiments, the reversal triplet is three isolated biomarkers EGLN, IBP4, and SHBG, wherein two biomarkers are in the numerator (e.g., EGLN+IBP4), and one biomarker is in the denominator (e.g., SHBG). For example, a reversal triplet can be (EGLN+IBP4)/SHBG. In some embodiments, the reversal triplet is three isolated biomarkers PAPP2, IBP4, and SHBG, wherein two biomarkers are in the numerator (e.g., PAPP2+IBP4), and one biomarker is in the denominator (e.g., SHBG). For example, a reversal triplet can be (PAPP2+IBP4)/SHBG. In other embodiments, the reversal triplet is three isolated biomarkers EGLN, TETN and APOC3, wherein one biomarker is in the numerator (e.g., EGLN) and two biomarkers are in the denominator (e.g., TETN+APOC3). For example, a reversal triplet can be EGLN/(TETN+APOC3). In some embodiments, the reversal triplet exhibits a change in combined reversal value between pregnant females at risk for pre-term birth compared to term controls. In a further embodiment, the reversal triplet (EGLN+PRL)/TETN exhibits a higher ratio in pregnant females at risk for pre-term birth compared to term controls. Similarly, in some embodiments, the reversal triplets (EGLN+IBP4)/SHBG and (PAPP2+IBP4)/SHBG each exhibit a higher ratio in pregnant females at risk for pre-term birth compared to term controls. Encompassed within the definition of any reversal triplet is the corresponding reversal triplet wherein individual biomarkers are switched between the numerator and denominator. One skilled in the art will appreciate that such a corresponding reversal triplet is equally informative with regard to its predictive power.

The term “reversal group” refers to a group of one or more isolated biomarkers, reversal pairs and reversal triplets combined to make a group of biomarkers that exhibit a change in value between the classes being compared. The term reversal group can also further refer to a group of one or more isolated biomarkers, reversal pairs, and reversal triplets combined with clinical and/or demographic variables. In some embodiments, the clinical variables include body mass index (BMI), gestational age at blood draw (GABD), prior preterm birth, progesterone treatment, or a shortened cervix. In some embodiments, combination of biomarkers, reversal pairs, reversal triplets and clinical and/or demographic variables include combination by addition, multiplication, subtraction or division.

In some embodiments one or more isolated biomarkers, reversal pairs, and/or reversal triplets are combined with clinical and/or demographic variables into a numerical combined score (sometimes called a classifier herein) that can be trained on a dataset to predict a patient's probability of a specific clinical outcome (e.g., preterm birth). In some embodiments the combined score includes one or more molecular components selected from the group consisting of isolated biomarkers (e.g., measured level of isolated biomarkers in a sample), reversal pairs, and reversal triplets (either individually or themselves combined into a molecular score) and one or more clinical components selected from the group consisting of body mass index (BMI), gestational age at blood draw (GABD), ObRisk (prior miscarriage for nulliparous women, or prior history of preterm birth for multiparous women), current progesterone treatment, shortened cervix, and zero or more additional risk indicia as described herein (either individually or themselves combined into a clinical score). In some embodiments the combined score can be the combination of the molecular score and the clinical score. Some components of the combined score (e.g., measured levels of a biomarker, reversal values or combined reversal values, GABD, BMI) can be continuous numeric variables. Some components of the combined score can be binary or categorical variables, which can be expressed numerically. For example, the clinical component of prior history of preterm birth can be included in the combined score as a binary variable, with 0 representing no history of preterm birth and 1 representing a history of preterm birth (or vice versa). Any categorical variable can be decomposed into pairwise binary contrasts in which 0 represents one level and 1 represents a second level. As is known in the art, all but one of all possible pairs are so coded for complete representation. Any component of the combined score may be multiplied by a coefficient or otherwise mathematically converted (e.g., logarithm). Some components with continuous possible values can be expressed in a binary fashion. For example, GABD can be expressed as (<19 weeks or >20 weeks)=0 and (19 or 20 weeks)=1. In some embodiments the total combined score can be multiplied by a coefficient or otherwise mathematically converted, e.g., to scale the score to run from 0 to 100. Such combined scores can be used as test scores or test values (or correspondingly reference scores or reference values) in any methods or systems of the invention.

In some embodiments the combined score is calculated according to any of the following formulae:

$$Combined score = ( A \times Biomarker level ( s ) ) + ( B \times GABD ) + ( C \times BMI ) + G \times GABD \times BMI ) + [ \text{} one or more of ( D \times priori history of preterm birth [ 0 or 1 ] ) . ( E \times current progesterone treatment [ 0 or 1 ] ) , ( F \times shortened cervix [ 0 or 1 ] ) ] \qquad ( 1 )$$

In some cases, a formula may not have all of the specified coefficients or have the value of 0 for one or more of the coefficients (and thus not incorporate the corresponding variable(s)) or 1 for a coefficient. For example, in formula (3) A is 1.23, B is −0.00130, C is 1.52, D is 0.401, E is −2.08, and F of formula (4) would not be applicable in this example (i.e., the coefficient for F would be 0). As mentioned above, in some embodiments one or more of the clinical variables or components (e.g., BMI and/or GABD) can be combined into a clinical score, which can then be combined with one or more of the molecular variables or components to yield a combined score according to the following more generalized formula:

$$Combined score = {A}^{′} \times ( molecular score ) + {B}^{′} \times ( clinical score ) \qquad ( 2 )$$

In some embodiments, the first term in formula (1) (A×Biomarker level(s)) comprises multiple individual biomarkers, each of which has its own coefficient—e.g., ln(IBP4/SHBG), ln(EGLN), ln(PRL), ln(TETN)—all of which can be combined additively or multiplicatively. In some embodiments, the term “ln([BIOMARKER])” is the natural log of the measured level or concentration of the biomarker in a sample. In some of these embodiments, new formulae can be used to calculate the combined score that predicts probability of preterm birth:

$$Combined score = ( A \times ln ( IBP 4 / SHBG ) ) + ( B \times GABD * BMI ) + ( C \times ln ( EGLN ) ) + ( D \times ln ( PRL ) ) + ( E \times ln ( TETN ) ) \qquad ( 3 )$$

$$Combined score = ( A \times ln ( IBP 4 / SHBG ) ) + ( B \times GABD * BMI ) + ( C \times ln ( EGLN ) ) + ( D \times ln ( PRL ) ) + ( E \times ln ( TETN ) ) + ( F \times PriorPTB [ 0 or 1 ] ) \qquad ( 4 )$$

In some embodiments of each of formula (3) and formula (4), additional clinical factors can be included in the combined score as follows: (G×Short cervix [0 or 1]) and/or (H×Progesterone treatment [0 or 1]). In some embodiments involving formula (3) or formula (4), the coefficients are as follows in Table 1.

TABLE 1
Coefficients associated with formula (3) or formula (4).

| | Model | | | |
|---|---|---|---|---|
| | sPTB = ln(IBP4/SHBG)~ + | | sPTB = ln(IBP4/SHBG)~ + | |
| | GABD*BMI + ln(EGLN) + ln(PRL) + | | GABD*BMI + PriorPTB + ln(EGLN) + | |
| | ln(TETN) | | ln(PRL) + ln(TETN) | |
| Feature | Coefficient | Range | Coefficient | Range |
| ln(EGLN) | 1.52 | 0.3603 to 2.73 | 1.54 | 0.3698 to 2.76 |
| ln(PRL) | 0.401 | 0.0862 to 0.729 | 0.379 | 0.06197 to 0.7104 |
| ln(TETN) | −2.08 | −3.31 to −0.902 | −2.03 | −3.27 to −0.851 |
| ln(IBP4/SHBG) | 1.23 | 0.354 to 2.134 | 1.20 | 0.316 to 2.12 |
| PriorPTB | | | 1.63 | 0.159 to 3.37 |
| BMI | 0.0804 | −1.32 to 1.51 | 0.0570 | −1.37 to 1.52 |
| GABD | −0.0127 | −0.253 to 0.233 | −0.0139 | −0.258 to 0.236 |
| BMI × GABD | −0.00130 | −0.0126 to 0.0096 | −0.00118 | −0.0127 to 0.00995 |

In some embodiments, the coefficient for short cervix is 1.6 and/or the coefficient for progesterone treatment is −0.16.

In some embodiments involving formula (3) or formula (4), A, B, C, and/or D, E, F, G or H is within rounding of these values (e.g., A is between 1.2250.445 and 1.2340.454, etc.). In some cases, a formula may not have all of the specified coefficients or have the value of 0 for one or more of the coefficients (and thus not incorporate the corresponding variable(s)) or 1 for a coefficient. For example, one of the embodiments mentioned previously may incorporate formula (1) where A in formula (1) is 0.95 and B in formula (2) is 0.61. C and D would not be applicable in this example (i.e., the coefficient would be 0 for each).

In some embodiments, A is between 1.2 and 1.25, 1.15 and 1.25, 1.1 and 1.25, 1.050.4 and 1.25, 10.5, 0.4 and 1.25, 0.95 and 1.25, 0.9 and 1.25, 0.85 and 1.25, 0.8 and 1.25, 0.75 and 1.25, 0.7 and 1.25, 0.65 and 1.25, 0.6 and 1.25, 0.55 and 1.250.49, 0.54 and 1.25, 1.20.45, 0.35 and 1.3, 1.20.45, 0.36 and 1.35, 1.20.45, 0.37 and 1.4, 1.20.45, 0.38 and 10.45, 1.20.39 and 1.5, 1.20.45, 0.35 and 1.55, 1.20.4, 0.3 and 1.6, 1.20.45, 0.3 and 1.65, 1.20.4, 0.3 and 1.7, 1.20.45, 0.25 and 1.75, 1.20.49, 0.25 and 1.8, 1.20.45, 0.25 and 1.85, 1.20.4, 0.25 and 1.9, 1.2 and 1.950.35, or between 1.20.25 and 20.3. In some embodiments B is between −0.00125 and −0.00135, −0.0012 and −0.00135, −0.00115 and −0.00135, −0.0011 and −0.00135, −0.00105 and −0.00135, −0.001 and −0.00135, −0.00095 and −0.00135, −0.0009 and −0.00135, −0.00125 and −0.0014, −0.00125 and −0.00145, −0.00125 and −0.0015, −0.00125 and −0.00155, −0.00125 and −0.0016, −0.00125 and −0.00165, −0.00125 and −0.00170.35 and 1, 0.40 and 0.99, 0.45 and 0.95, 0.45 and 0.8, 0.45 and 0.7, 0.45 and 0.65, 0.50 and 0.63, or between −0.0012550 and −0.0017554. In some embodiments C is between 1.5 and 1.55, 1.45 and 1.55, 1.4 and 1.55, 1.35 and 1.55, 1.3 and 1.55, 1.25 and 1.55, 1.2 and 1.55, 1.15 and 1.55, 1.5 and 1.6, 1.5 and 1.65, 1.5 and 1.7, 1.5 and 1.75, 1.5 and 1.8, 1.5 and 1.85, 1.5 and 1.9, 1.5 and 10.10 and 1, 0.15 and 0.95, or between 1.5 and 2.0.20 and 0.90, 0.25 and 0.8, 0.30 and 0.7, 0.35 and 0.65, 0.40 and 0.60, or between 0.45 and 0.55. In some embodiments D is between 0.4 and 0.405, 0.395 and 0.405, 0.39 and 0.405, 0.385 and 0.405, 0.38 and 0.405, 0.375 and 0.405, 0.37 and 0.405, 0.365 and 0.405, 0.4 and 0.41, 0.4 and 0.415, 0.4 and 0.42, 0.4 and 0.425, 0.4 and 0.43, 0.4 and 0.435, 0.4 and 0.44, or between 0.4 and 0.445. In some embodiments E is between −2.05 and −2.1, −2 and −2.1, −120 and 1, 0.25 and 0.95, 0.30 and −2.1, −1.90.90, 0.35 and −2.1, −10.85, 0.40 and −2.1, −1.80.80, 0.45 and −2.1, −10.75, 0.50 and −2.1, −1.70.70, or between 0.55 and −2.1, −2.050.65. In some embodiments D is between 0.20 and −2.15, −2.051, 0.25 and −2.2, −2.050.75, 0.30 and −2.25, −2.050.65, 0.35 and −2.3, −2.050.55, 0.40 and −2.35, −2.05 and −2.4, −2.05 and −2.450.50, or between −2.050.45 and −2.50.50. In some embodiments FE is between 1.6 and 1.65, 1.55 and 1.65, 1.5 and 1.65, 1.45 and 1.65, 1.4 and 1.65, 1.35 and 1.65, 1.3 and 1.65, 1.6 and 1.7, 1.6 and 1.75, 1.6 and 1.8, 1.6 and 1.85, 1.6 and 1.9, 1.6 and 10.20 and 1, 0.25 and 0.95, 0.30 and 0.90, 0.35 and 0.85, 0.40 and 0.80, 0.45 and 0.75, 0.50 and 0.70, or between 1.6 and 20.55 and 0.65. In some embodiments E is between 0.20 and 1, 0.30 and 0.95, 0.30 and 0.90, 0.40 and 0.85, 0.50 and 0.80, 0.60 and 0.75, or between 0.70 and 0.75. In some embodiments F is between 0.001 and 0.2, 0.005 and 0.18, 0.01 and 0.16, 0.02 and 0.14, 0.04 and 0.12, 0.06 and 0.11, or between 0.08 and 0.10.

In some embodiments involving formula (3) or formula (4), A, B, C, D, E, F, G or H is within the range indicated in the above table (e.g., A is between 0.3603 and 2.73, etc.). In some embodiments A is between 1.2 and 1.25, 1.3, 1.35, 1.4, 1.45, 1.5, 1.55, 1.6, 1.65, 1.7, 1.75, 1.8, 1.85, 1.9, 1.95, 2, 2.05, 2.15, 2.2, 2.25, 2.3, 2.35, 2.4, 2.45, 2.5, 2.55, 2.6, 2.65, or 2.7; or between 0.3605, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, or 1.2, and 1.25; B is between −0.0122 and 0.01, 0.009, 0.008, 0.007, 0.006, 0.005, 0.004, 0.003, 0.002, 0.001, 0, −0.001, or −0.00130; or between −0.00130, −0.002, −0.001, −0, 0.001, 0.002, 0.003, 0.004, 0.005, 0.006, 0.007, 0.008, or 0.009 and 0.0096; and/or C is between 0.3603 and 0.4, 0.5, 0.6, 0.7, 0.8, 0.9, 1, 1.1, 1.2, 1.3, 1.4, 1.5, or 1.52; or between 1.52, 1.6, 1.7, 1.8, 1.9, 2, 2.1, 2.2, 2.3, 2.4, 2.5, 2.6, or 2.7 and 2.73; and/or D is between 0.0862, 0.095, 0.1, 0.15, 0.2, 0.25, 0.3, 0.35, or 0.4 and 0.401; or between 0.401 and 0.45, 0.5, 0.55, 0.6, 0.65, 0.7 or 0.729; and/or E is between −3.31, −3.2, −3.1, −3, −2.9, −2.8, −2.7, −2.6, −2.5, −2.4, −2.3, −2.2, or −2.1 and −2.08; or between −2.08, −2, −1.9, −1.8, −1.7, −1.6, −1.5, −1.4, −1.3, −1.2, −1.1, or −1 and −0.902; and/or F is between 0.159, 0.2, 0.3, 0.4, 0.5, 0.6, 0.7, 0.8, 0.9, 1.1, 1.2, 1.3, 1.4, 1.5, or 1.6 and 1.63; or between 1.63 and 1.7, 1.8, 1.9, 2, 2.1, 2.2, 2.3, 2.4, 2.5, 2.6, 2.7, 2.8, 2.9, 3, 3.1, 3.2, 3.3 or 3.37.

In some embodiments, the combined score is calculated according to formula (5)(i) or (5)(ii).

$$b 1 * log ( IBP 4 / SHBG ) + b 2 * ObRisk + b 3 * log ( AnalyteA ) - b 4 * log ( AnalyteB ) for nulliparous women [ new reversal : A / C ] \qquad ( 5 ) ( i )$$

$$b 5 * log ( IBP 4 / SHBG ) + b 6 * ObRisk + b 7 * log ( AnalyteA ) - b 8 * log ( AnalyteC ) for nulliparous women [ new reversal : A / C ] \qquad ( 5 ) ( ii )$$

In some embodiments, the combined score is calculated according to any one of formula (6)(i) to (6)(iii).
(6))(i)
   Tree step: separate nullips vs multips
   Calculation step:

$$\begin{matrix}Nullips : combined score = EGLN / TETN + ObRisk + IBP 4 / SHBG \\ Mutips : combined score = EGLN / AnalyteC + ObRisk + IBP 4 / SHBG\end{matrix}$$

(6) (ii)
   Tree step: separate nullips vs multips
   Calculation step:

$$\begin{matrix}Nullips : combined score = EGLN / AnalyteC + ObRisk + IBP 4 / SHBG \\ Mutips : combined score = EGLN / AnalyteC + ObRisk + IBP 4 / SHBG\end{matrix}$$

(6)(iii)
   Tree step: separate nullips vs multips
   Calculation step:

$$\begin{matrix}Nullips : combined score = EGLN + ObRisk + IBP 4 / SHBG \\ Mutips : combined score = EGLN + ObRisk + IBP 4 / SHBG\end{matrix}$$

As an example of the coefficients that can be used to calculate a combined score using formula (5)(i), (5)(ii), and (6)(i), the coefficients for an exemplary combination of pairs of biomarkers are shown in Table 1.1. It is recognized that coefficients may be varied depending upon the biomarkers used, the way the biomarkers are assayed, and the population to which they are applied for calculating the combined score. Coefficients can be derived from training on a sample of a population, by applying assay specific conversion factors to coefficiences exemplified herein, or a priori (e.g., assigning a coefficient of 1 to all components that have a positive range and assigning a coeffieinct of −1 to all components that have a negative range).

TABLE 1.1
Coefficients associated with formula (6)(i)

| | Model | | | |
|---|---|---|---|---|
| | PTB = ln(IBP4/SHBG) + ObRisk + | | sPTB = ln(IBP4/SHBG) + ObRisk + | |
| | (ln(EGLN) + ln(TETN)): Nulliparous + | | (ln(EGLN) + ln(TETN)): Nulliparous + | |
| | (ln(EGLN) + ln(ALS)): Multiparous | | (ln(EGLN) + ln(ALS)): Multiparous | |
| Feature | Coefficient | Range | Coefficient | Range |
| ln(EGLN): | 1.33 | ≥0 | 1.33 | ≥0 |
| nulliparous | | | | |
| ln(TETN): | −1.50 | ≤0 | −1.50 | ≤0 |
| nulliparous | | | | |
| ln(EGLN): | 1.03 | ≥0 | 1.03 | ≥0 |
| multiparous | | | | |
| ln(ALS): | −0.536 | ≤0 | −0.536 | ≤0 |
| multiparous | | | | |
| ln(IBP4/SHBG): | 0.763 | ≥0 | 0.763 | ≥0 |
| nulliparous | | | | |
| ln(IBP4/SHBG): | 0.713 | ≥0 | 0.713 | ≥0 |
| multiparous | | | | |
| ObRisk: | 0.819 | ≥0 | 0.819 | ≥0 |
| nulliparous | | | | |
| ObRisk: | 2.03 | ≥0 | 2.03 | ≥0 |
| multiparous | | | | |

The detection of a reversal group in protein concentrations or gene expression levels may eliminate the need for data normalization or the establishment of population-wide thresholds and requires only that the levels of two more biomarkers change relative to each other and requires only that the levels of two or more biomarkers change relative to each other. In some embodiments, the reversal group is three or more isolated biomarkers PRL, IBP4 and SHBG, wherein one biomarker is in the numerator (e.g., IBP4), one biomarker is in the denominator (e.g., SHBG) and one biomarker is added to same. For example, a group can be PRL+IBP4/SHBG. Other examples of reversal groups can be found in Tables 6-19, and Tables 21-22. In some embodiments, the reversal group includes, or is supplemented by, one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP, any of which may be useful for methods, kits, compositions, or systems described herein for determining the probability for preterm birth in a pregnant female. For example, in some instances one or more of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP, are used in combination with a reversal group for enhanced prediction of preterm birth. In other instances, any one or more of the biomarkers disclosed can be used individually (e.g., without a reversal group) for the prediction of preterm birth. In some embodiments, the triplet group exhibits a change in combined reversal value between pregnant females at risk for pre-term birth compared to term controls. In other embodiments, the reversal group is isolated biomarkers EGLN, PRL, TETN, IBP4 and SHBG, wherein the group is (EGLN+PRL)/TETN+IBP4/SHBG. Other examples of reversal groups can be found in Tables 6-19, and Tables 21-22. In some embodiments, the reversal group exhibits a change in reversal value or combined reversal value between pregnant females at risk for pre-term birth compared to term controls. In a further embodiment, the reversal group (EGLN+PRL)/TETN+IBP4/SHBG exhibits a higher ratio in pregnant females at risk for pre-term birth compared to term controls. In another embodiment, the reversal groups (EGLN+IBP4)/SHBG and (PAPP2+IBP4)/SHBG each exhibit a higher ratio in pregnant females at risk for pre-term birth compared to term controls. Encompassed within the definition of any reversal pair is the corresponding reversal pair wherein individual biomarkers are switched between the numerator and denominator. One skilled in the art will appreciate that such a corresponding reversal pair is equally informative with regard to its predictive power.

The term “reversal value” refers to the ratio of the abundance of two analytes and serves to both normalize variability and amplify diagnostic signal. In some embodiments, a reversal value refers to the ratio of the abundance of an up-regulated (interchangeably referred to as “over-abundant,” up-regulation as used herein simply refers to an observation of abundance) analyte over a down-regulated analyte (interchangeably referred to as “under-abundant,” down-regulation as used herein simply refers to an observation of relative abundance). In some embodiments, a reversal value refers to the ratio of an up-regulated analyte over an up-regulated analyte, where one analyte differs in the degree of up-regulation relative the other analyte. In some embodiments, a reversal value refers to the ratio a down-regulated analyte over a down-regulated analyte, where one analyte differs in the degree of down-regulation relative the other analyte. In some embodiments a reversal value refers to the ratio of a regulated analyte (up or down) and an analyte that is un-regulated. In this case the un-regulated analyte can still serve to normalize. In some embodiments, a reversal value refers to the ratio of two analytes that are un-regulated or whose directions of regulation are unknown. In this case, the un-regulated analytes can still serve to normalize each other and to reveal a diagnostic signal.

The term “combined reversal value” as used herein refers to the combination (e.g., combined value) of values (e.g., relative abundance) characterizing two or more reversal pairs or analytes (e.g., reversal values, which each can represent the ratio of two biomarkers forming a reversal pair), which like reversal value, serves to normalize variability and/or amplify diagnostic signal. In some embodiments, a combined reversal value refers to the combination of values characterizing two or more reversal pairs or analytes, where the two or more reversal pairs share one of the same analytes. In some embodiments, a combined reversal value refers to the combination of values characterizing two or more reversal pairs or analytes, where the two or more reversal pairs each refer to different analytes. In some embodiments, a combined reversal value refers to the combination of values characterizing the abundance of two or more up-regulated analytes over one or more down-regulated analytes. In some embodiments, a combined reversal value refers to the combination of values characterizing the abundance of one or more up-regulated analytes over other two or more down-regulated analytes. In some embodiments, a combined reversal value refers to the combination of values characterizing the abundance of two or more up-regulated analytes over other two or more down-regulated analytes. In some embodiments, a combined reversal value refers to the combination of values characterizing two or more up-regulated analytes over other one or more up-regulated analytes, where at least one of the analytes differ in the degree of up-regulation relative to the other analyte(s). In some embodiments, a combined reversal value refers to the combination of values characterizing two or more down-regulated analytes over other one or more down-regulated analytes, where at least one of the analytes differ in the degree of down-regulation relative to the other analyte(s). In some embodiments, a combined reversal value refers to the combination of values characterizing two or more regulated analytes (up or down) and one or more analytes that are un-regulated. In this case, the un-regulated analyte(s) can still serve to normalize. In some embodiments, a combined reversal value can be structured according to the models and formulas of Example 3.

One advantageous aspect of a reversal or combined reversal is the presence of complementary information in the two analytes or pair(s) of analytes, so that the combination of the two analytes or pair(s) of analyztes is more diagnostic of the condition of interest than either one alone. Preferably the combination of the two analytes or pair(s) of analytes increases signal-to-noise ratio by compensating for biomedical conditions not of interest, pre-analytic variability and/or analytic variability. Out of all the possible reversals within a narrow gestational age window, a subset can be selected based on individual univariate performance. Additionally, a subset can be selected based on bivariate or multivariate performance in a training set, with testing on held-out data or on bootstrap iterations. For example, logistic or linear regression models can be trained, optionally with parameter shrinkage by L1 or L2 or other penalties, and tested in leave-one-out, leave-pair-out or leave-fold-out cross-validation, or in bootstrap sampling with replacement, or in a held-out data set. As disclosed herein, the ratio of the abundance of two analytes, for example, the ratio of an up-regulated biomarker over a down-regulated biomarker, referred herein as a reversal value, can be used to identify robust and accurate classifiers and predict a pregnant female's risk of developing placental dysfunction in the pregnancy. Similarly, as disclosed herein, the ratio of the abundance of more than one reversal pairs or analytes, for example, the ratio of one or more up-regulated biomarkers over other one or more down-regulated biomarker(s), referred herein as a combined reversal value, can be used to identify robust and accurate classifiers and predict a pregnant female's risk of developing placental dysfunction in the pregnancy.

Use of a ratio of biomarkers in the methods disclosed herein corrects, among others, for variability that is the result of human manipulation after the removal of the biological sample from the pregnant female. Such variability can be introduced, for example, during sample collection, processing, depletion, digestion or any other step of the methods used to measure the biomarkers present in a sample and is independent of how the biomarkers behave in nature. Use of a ratio of biomarkers can also correct for multiple influences on each biomarker within the pregnant female. For example, if at least one marker is influenced by two biological processes and the other marker or markers are influenced by two biological processes, one common and one distinct, the combination of the two or more markers can reinforce measurement of the common process and distinguish it from other processes. Accordingly, the invention generally encompasses the use of one or more reversal pairs in a method of diagnosis or prognosis to reduce variability and/or amplify, normalize or clarify diagnostic signal.

While the terms reversal value and combined reversal value can refer to the ratio of the abundance of one or more up regulated analytes over other one or more down regulated analytes and serve to both normalize variability and amplify diagnostic signal, it is also contemplated that one or more pairs of biomarkers of the invention could be treated in a classifier by any other means, for example, by subtraction, addition, exponentiation or multiplication of abundances. In addition, it is contemplated that a value can be mathematically converted to a different value and used to determine a ratio. For example, as disclosed herein, reversals can be constructed as the ratios of the logarithm (log) values. Similarly, ratios can be mathematically converted, for example, as the log of the ratioed values. The methods disclosed herein encompass the measurement of biomarker pairs by such other means. A person skilled in the art will readily understand suitable data transformations that can be applied to identify biomarkers predictive of preterm birth and/or placental dysfunction, including the data transformations disclosed herein. Exemplary transformations include, but are not limited to, box-cox, root, inverse, rank and log. Such data transformations are well known in the art, for example, root (where the root transformation is selected as appropriate for the data set, such as 2, 3, 4, and higher, as appropriate), inverse (1/X), rank (assigning to an ordered list based on appropriate criteria), and so forth, as is well known in the art.

This method is advantageous because, in some embodiments, it provides a simple classifier that can function independently of data normalization, help to avoid overfitting, and result in a very simple experimental test that is easy to implement in the clinic. In some uses of the term “reversal” it refers to the identification of analyte pairs where the relative expression (rank order) of each member of a pair reverses in the two or more conditions studied (e.g. cancer vs not cancer, placental dysfunction vs not). Reversal, as it is used here, allows for there to be opposing regulation of the two or more members of the pair (e.g., up or down), but does not require that their rank order in abundance to “reverse” in the different clinical conditions. The use of marker pairs based on changes in reversal values (or combined reversal values) that are independent of data normalization enabled the development of the clinically relevant biomarkers disclosed herein. Because quantification of any single protein is subject to uncertainties caused by measurement variability, normal fluctuations, and individual related variation in baseline expression, identification of pairs of markers that may be under coordinated, systematic regulation enables robust methods for diagnosis and prognosis.

IBP4 is a member of a family of insulin-like growth factor binding proteins (IBP) that negatively regulate the insulin-like growth factors IGF1 and IGF2. (Forbes et al. Insulin-like growth factor I and II regulate the life cycle of trophoblast in the developing human placenta. Am J Physiol, Cell Physiol. 2008; 294(6):C1313-22). IBP4 is expressed by syncytiotrophoblasts (Crosley et al., IGFBP-4 and -5 are expressed in first-trimester villi and differentially regulate the migration of HTR-8/SVneo cells. Reprod Biol Endocrinol. 2014; 12(1):123) and is the dominant IBP expressed by extravillous trophoblasts (Qiu et al. Significance of IGFBP-4 in the development of fetal growth restriction. J Clin Endocrinol Metab. 2012; 97(8):E1429-39). Compared to term pregnancies, maternal IBP4 levels in early pregnancy are higher in pregnancies complicated by fetal growth restriction and preeclampsia. (Qiu et al., supra, 2012)

SHBG regulates the availability of biologically active unbound steroid hormones. Hammond G L. Diverse roles for sex hormone-binding globulin in reproduction. Biol Reprod. 2011; 85(3):431-41. Plasma SHBG levels increase 5-10 fold during pregnancy (Anderson D C. Sex-hormone-binding globulin. Clin Endocrinol (Oxf). 1974; 3(1):69-96) and evidence exists for extra-hepatic expression, including placental trophoblastic cells. (Larrea et al. Evidence that human placenta is a site of sex hormone-binding globulin gene expression. J Steroid Biochem Mol Biol. 1993; 46(4):497-505) Physiologically, SHBG levels negatively correlate with triglycerides, insulin levels and BMI. (Simó et al. Novel insights in SHBG regulation and clinical implications. Trends Endocrinol Metab. 2015; 26(7):376-83) BMI's effect on SHBG levels may explain, in part, the improved predictive performance with BMI stratification.

Intra-amniotic infection and inflammation have been associated with PTB, as has increased levels of proinflammatory cytokines including TNF-α and IL1-β. (Mendelson C R. Minireview: fetal-maternal hormonal signaling in pregnancy and labor. Mol Endocrinol. 2009; 23(7):947-54; Gomez-Lopez et al. Immune cells in term and preterm labor. Cell Mol Immunol. 2014; 11(6):571-81). SHBG transcription in liver is suppressed by IL1-β and NF-kB mediated TNF-α signaling (Simó et al. Novel insights in SHBG regulation and clinical implications. Trends Endocrinol Metab. 2015; 26(7):376-83), a pathway implicated in initiation of normal and abnormal labor (Lindström T M, Bennett P R. The role of nuclear factor kappa B in human labour. Reproduction. 2005; 130(5):569-81). Lower levels of SHBG in women destined for sPTB may be a result of infection and/or inflammation. Hence, SHBG may be critical for control of androgen and estrogen action in the placental-fetal unit in response to upstream inflammatory signals.

Endoglin (EGLN), a transmembrane coreceptor for TGFβ, is an anti-angiogenic factor expressed by the placenta that inhibits trophoblast migration (Jones et al. TGF-beta superfamily expression and actions in the endometrium and placenta. (1470-1626 (Print); and Cheifetz et al., Endoglin is a component of the transforming growth factor-beta receptor system in human endothelial cells. J Biol Chem. 1992; 267(27):19027-30). EGLN may be important for cell differentiation, migration, and angiogenesis. (Gregory et al., Review: the enigmatic role of endoglin in the placenta. Placenta. 2014; 35 Suppl:S93-9; and Duff et al., CD105 is important for angiogenesis: evidence and potential applications. Faseb j. 2003; 17(9):984-92; Caniggia et al. Endoglin Regulates Trophoblast Differentiation along the Invasive Pathway in Human Placental Villous Explants. Endocrinology. 1997; 138(11):4977-4988). Many independent studies and a meta-analysis support an association of elevated circulating EGLN levels with development of preeclampsia (PE) (Margioula-Siarkou et al. Soluble EGLN concentration in maternal blood as a diagnostic biomarker of preeclampsia: A systematic review and meta-analysis. Eur J Obstet Gynecol Reprod Biol. 2021 Jan. 26; 258:366-381; Chaiworapongsa et al., The use of angiogenic biomarkers in maternal blood to identify which SGA fetuses will require a preterm delivery and mothers who will develop pre-eclampsia. J Matem Fetal Neonatal Med. 2016; 29(8):1214-28). and in placentas following delivery (Jeyabalan et al. Circulating and placental EGLN concentrations in pregnancies complicated by intrauterine growth restriction and preeclampsia. Placenta. 2008 June; 29(6):555-63). EGLN has been proposed as a biomarker to predict PE, particularly in combinations with other soluble angiogenesis factors, sFLT and PLGF (Levine et al. Soluble EGLN and other circulating antiangiogenic factors in preeclampsia. N Engl J Med. 2006 Sep. 7; 355(10):992-1005), and with uterine artery doppler lowest pulsatility index (Foidart et al. Maternal plasma soluble EGLN at 11-13 weeks' gestation in pre-eclampsia. Ultrasound Obstet Gynecol. 2010 June; 35(6):680-7). EGLN may also be a marker for other pregnancy complications: sEGLN levels were elevated in women delivering small for gestational age infants (Romero et al. A longitudinal study of angiogenic (placental growth factor) and anti-angiogenic (soluble EGLN and soluble vascular endothelial growth factor receptor-1) factors in normal pregnancy and patients destined to develop preeclampsia and deliver a small for gestational age neonate. J Matern Fetal Neonatal Med. 2008 January; 21(1):9-23; Conroy et al. Altered angiogenesis as a common mechanism underlying preterm birth, small for gestational age, and stillbirth in women living with HIV. (1097-6868 (Electronic)) preterm (Conroy et al. Altered angiogenesis as a common mechanism underlying preterm birth, small for gestational age, and stillbirth in women living with HIV. (1097-6868 (Electronic); Chaiworapongsa et al A subset of patients destined to develop spontaneous preterm labor had an abnormal angiogenic/anti-angiogenic profile in maternal plasma: evidence in support of pathophysiologic heterogeneity of preterm labor derived from a longitudinal study. (1476-4954 (Electronic)), or in pregnancies complicated by intraamniotic infection Kim et al. EGLN in amniotic fluid as a risk factor for the subsequent development of bronchopulmonarv dysplasia. Am J Reprod Immunol. 2013; 69(2):105-123. However, the utility of EGLN to predict sPTB, as opposed to preeclampsia or medically indicated PTB from serum at 17-19 weeks gestation is unexpected.

PRL regulates mammary gland development and milk production and thus increases 10- to 20-fold during pregnancy, during which it is the dominant growth factor responsible for development of mammary glands and milk production (Al-Chalabi et al. Physiology, prolactin Treasure Island, FL: StatPearls Publishing; Updated 2020 Jul. 10. Available from: https://www.ncbi.nlm.nih.gov/books/NBK507829/). However, as a polypeptide hormone produced by the pituitary gland, and the decidua during pregnancy (Soares. The prolactin and growth hormone families: Pregnancy-specific hormones/cytokines at the maternal-fetal interface. Reproductive Biology and Endocrinology. 2004 2004/07/05; 2(1):51), it has many roles in homeostasis (Al-Chalabi et al. Physiology, Prolactin Treasure Island, FL: StatPearls Publishing; Updated 2020 Jul. 10. Available from: https://www.ncbi.nlm.nih.gov/books/NBK507829/) and has been shown to modulate immune response, both positively and negatively (Bachelot. Reproductive role of prolactin. Reproduction. 2007 February; 133(2):361-9). In pregnancy, PRL may help regulate insulin resistance to facilitate transport of glucose and other nutrients across the placenta (Lopez-Vicchi et al. Metabolic functions of prolactin: Physiological and pathological aspects. J Neuroendocrinol. 2020 November; 32(11):e12888). PRL may also play a role in placental angiogenesis: the full-length protein has angiogenic properties and a cleaved N-terminal fragment has anti-angiogenic and VEGF inhibitory activity (Struman et al. Opposing actions of intact and N-terminal fragments of the human prolactin/growth hormone family members on angiogenesis: an efficient mechanism for the regulation of angiogenesis. Proc Natl Acad Sci USA. 1999 Feb. 16; 96(4):1246-51). Circulating and urine PRL levels (full length and anti-angiogenic fragment) were higher in severe vs mild PE, and predicted adverse maternal and fetal outcomes, such as small for gestational age, even better than soluble fins-like tyrosine kinase/placental growth factor (sFLT/PlGF) or sEGLN (Leaños-Miranda et al. Circulating Angiogenic Factors and Urinary Prolactin as Predictors of Adverse Outcomes in Women With Preeclampsia. Hypertension. 2013 2013/05/01; 61(5):1118-1125). PRL has also been implicated in preterm birth: one study measured cervicovaginal fluid levels of PRL in women showing symptoms of preterm labor, and found detectable levels in 50% of subjects vs. 5% for asymptomatic women (O'Brien et al. Cervicovaginal prolactin: A marker for spontaneous preterm delivery. American Journal of Obstetrics and Gynecology. 1994 1994/10/01/; 171(4):1107-1111. However, the ability of PRL to predict sPTB as a blood-based biomarker has not been reported.

TETN regulates extracellular matrix remodeling and fibrinolysis via interactions with plasminogen and fibrin. Clemmensen et al. Purification and characterization of a novel, oligomeric, plasminogen kringle 4 binding protein from human plasma: tetranectin, Eur. J. Biochem. 1986; 156(2):327-333; Kluft et al. Calcium-dependent binding of tetranectin to fibrin. Thrombosis Research. 1989; 55(2):233-238. Lower levels of TETN in the serum or plasma are associated with various disease states including cancer, particularly of metastatic disease (Hogdall et al. Prognostic value of serum tetranectin in patients with metastatic breast cancer. Acta Oncol. 1993; 32(6):631-6; Jensen et al. Plasma tetranectin is reduced in cancer and related to metastasia. Cancer. 1988 Sep. 1; 62(5):869-72; Hogdall et al. The prognostic value of tetranectin immunoreactivity and plasma tetranectin in patients with ovarian cancer. Cancer. 1993 Oct. 15; 72(8):2415-22; Hogdall et al. Serum tetranectin and CA-125 used to monitor the course of treatment in ovarian cancer patients. Eur J Obstet Gynecol Reprod Biol. 1994 December; 57(3):175-8), arthritis (Kamper E F, Kopeikina L T, Trontzas P, et al. Comparative study of tetranectin levels in serum and synovial fluid of patients with rheumatoid arthritis, seronegative spondylarthritis and osteoarthritis. Clin Rheumatol. 1998; 17(4):318-24; Kamper E F, Kopeikina L T, Koutsoukos V, et al. Plasma tetranectin levels and disease activity in patients with rheumatoid arthritis. J Rheumatol. 1997 February; 24(2):262-8), heart failure (McDonald et al. Tetranectin, a potential novel diagnostic biomarker of heart failure, is expressed within the myocardium and associates with cardiac fibrosis. Scientific Reports. 2020 2020/05/05; 10(1):7507), and preeclampsia (Murphy et al. Alterations to the maternal circulating proteome after preeclampsia. (1097-6868 (Electronic)). Further, TETN was shown to reduce VEGF secretion and in vitro model angiogenesis (Dai et al. Downregulation of exosomal CLEC3B in hepatocellular carcinoma promotes metastasis and angiogenesis via AMPK and VEGF signals. (1478-811X (Electronic)). Moreover, in both amniotic fluid and fetal serum, correlation between TETN levels and gestational age has been reported, suggesting a role in fetal maturation (Hogdall et al., Tetranectin in amniotic fluid, maternal serum and fetal fluids. Scand J Clin Lab Invest. 1991; 51(5):411-5). TETN is reported to be negatively regulated by TGFβ (Iba et al., Transforming growth factor-01 downregulates dexamethasone-indunced tetranectin gene expression during the in vitro mineralization of the human osteoblastic cell line SV-HFO. FEBS Letters. 1995; 373(1):1-4), a pathway of importance in decidualization and placentation (Adu-Gyamfi et al., Regulation of placentation by the transforming growth factor beta superfamily. Biol Reprod. 2020; 102(1):18-26; Godkin et al., Transforming growth factor beta and the endometrium. Rev Reprod. 1998; 3(1):1-6; Ni et al., TGFβ superfamily signaling and uterine decidualization. Reprod Biol Endocrinol. 2017; 15(1):84). Accordingly, TETN may be involved in trophoblast invasion.

PAPP2, also known as pregnancy-associated plasma protein A2 and a homologue of PAPP-A, encodes a member of the pappalysin family of metzincin metalloproteinases. The encoded protein cleaves insulin-like growth factor-binding protein 5 (IGFBP-5), IGFBP-3, and is believed to be a local regulator of insulin-like growth factor (IGF) bioavailability (Overgaard M T et al. J Biol Chem 2001 June; 276(24)21849-21853). Diseases associated with PAPP2 include Hellp Syndrome, Short Stature, and Dauber-Argente Type, and more (“PAPPA2 Gene—Pappalysin.” GeneCards, PAPPA2Gene—GeneCards|PAPP2 Protein|PAPP2 Antibody). Related pathways include metabolism of proteins, regulation of IGF transport and uptake by IGF binding proteins (IGFBPs). PAPP-A, a paralog of PAPP2, has been shown to specifically cleave IGFBP-4, one of six modulators of IGF-I and -II activity. Cleavage of IGFBP-4 causes release of bound IGF that, in turn, interacts with its cellular receptor. It has been previously established that PAPP-A is the IGFBP-4 proteinase secreted from fibroblasts, osteoblasts, marrow stromal cells, and vascular smooth muscle cells, and is present in pregnancy serum and ovarian follicular fluid. PAPP2, an active metalloproteinase, specifically cleaves IGFBP-5 (as well as IGFBP-3) and functions in the same growth regulatory system as PAPP-A. Like PAPP-A and IGFBP-4, the pair of PAPP2 and IGFBP-5 plays an analogous role in a number of the tissues above. Furthermore, PAPP2 has been shown to have a higher expression in the placenta than in other tissues, with levels increasing with advancing gestation (Xi Chen et al. “The potential role of pregnancy-associated plasma protein-A2 in angiogenesis and development of preeclampsia,” Hypertension Research, 42; 970-980 (2019)).

A “measurable feature” is any property, characteristic or aspect that can be determined and correlated with the probability for preterm birth in a subject. The term further encompasses any property, characteristic or aspect that can be determined and correlated in connection with a prediction of GAB, a prediction of term birth, or a prediction of time to birth in a pregnant female. For a biomarker, such a measurable feature can include, for example, the presence, absence, or concentration of the biomarker, or a fragment thereof, in the biological sample, an altered structure, such as, for example, the presence or amount of a post-translational modification, such as oxidation at one or more positions on the amino acid sequence of the biomarker or, for example, the presence of an altered conformation in comparison to the conformation of the biomarker in term control subjects, and/or the presence, amount, or altered structure of the biomarker as a part of a profile of more than one biomarker.

As used herein, the term “risk score” refers to a score that can be assigned based on comparing one or more test scores or values (e.g., the amount of one or more biomarkers or reversal values (or combined reversal values) in a biological sample obtained from a pregnant female) to a standard or reference score or value that represents an average score or value (e.g., average amount of the one or more biomarkers calculated from biological samples) obtained from a random pool of pregnant females. In some embodiments the risk score is calculated by the association of the test score with outcomes, and the degree of the separation between outcomes (e.g., PTB vs term birth) as a result of each women having a test score. That risk can be expressed in a variety of ways (e.g., PPV, probability).

As exemplified herein in Tables 2 and 5-18, the enhanced IBP4/SHBG Classifier is defined as the natural log of the SIS normalized intensities of the IBP4, SHBG, EGLN, PRL and TETN peptide transitions. Exemplary m/z values for peptides/transitions for the top predicted IBP4, SHBG, EGLN, PRL, TETN, ALS, CSH1/CSH2, IBP3, FA5, and PAPP2 are shown in Table 2. Score=ln(P1n/P2n)+ln(P3n+P4n)/P5n, where P1n-P5n denote the SIS normalized peak area values for the IBP4, SHBG, EGLN, PRL and TETN transitions, respectively. SIS normalization is defined as the relative ratio of the endogenous peak area divided by the corresponding SIS peak area: e.g. P1n=P1e/P1SIS, where P1e=the peak area for the IBP4 endogenous transition and P1S1S=the peak area for IBP4 SIS transition.

TABLE 2
m/z values for SIS peptides and peptides derived from endogenous
protein

| Protein | | | |
|---|---|---|---|
| Description | Peptide | SIS Transition | Endogenous Transition |
| SHBG_HUMAN | IALGGLLFPASNLR | 484.63 m/z→667.38 m/z | |
| | (SEQ ID NO: 1) | | |
| SHBG_HUMAN | IALGGLLFPASNLR | 484.63 m/z→412.26 m/z | |
| | (SEQ ID NO: 1) | | |
| SHBG_HUMAN | IALGGLLFPASNLR | | 481.29 m/z→657.37 m/z |
| | (SEQ ID NO: 1) | | |
| SHBG_HUMAN | IALGGLLFPASNLR | | 481.29 m/z→412.26 m/z |
| | (SEQ ID NO: 1) | | |
| IBP4_HUMAN | QCHPALDGQR (SEQ ID | 397.86 m/z→485.23 m/z | |
| | NO: 2) | | |
| IBP4_HUMAN | QCHPALDGQR (SEQ ID | 397.86 m/z→370.21 m/z | |
| | NO: 2) | | |
| IBP4_HUMAN | QCHPALDGQR (SEQ ID | | 394.52 m/z→475.23 m/z |
| | NO: 2) | | |
| IBP4_HUMAN | QCHPALDGQR (SEQ ID | | 394.52 m/z→360.2 m/z |
| | NO: 2) | | |
| EGLN_HUMAN | GPITSAAELNDPQSILLR | 635.68 m/z→836.52 m/z | |
| | (SEQ ID NO: 3) | | |
| EGLN_HUMAN | GPITSAAELNDPQSILLR | 635.68 m/z→418.77 m/z | |
| | (SEQ ID NO: 3) | | |
| EGLN_HUMAN | GPITSAAELNDPQSILLR | | 632.35 m/z→826.51 m/z |
| | (SEQ ID NO: 3) | | |
| EGLN_HUMAN | GPITSAAELNDPQSILLR | | 632.35 m/z→413.76 m/z |
| | (SEQ ID NO: 3) | | |
| EGLN_HUMAN | TQILEWAAER (SEQ ID | 613.82 m/z→997.53 m/z | |
| | NO: 4) | | |
| EGLN_HUMAN | TQILEWAAER (SEQ ID | 613.82 m/z→884.45 m/z | |
| | NO: 4) | | |
| EGLN_HUMAN | TQILEWAAER (SEQ ID | | 608.82 m/z→987.53 m/z |
| | NO: 4) | | |
| EGLN_HUMAN | TQILEWAAER (SEQ ID | | 608.82 m/z→874.44 m/z |
| | NO: 4) | | |
| PRL_HUMAN | LSAYYNLLHCLR (SEQ | 511.6 m/z→710.36 m/z | |
| | ID NO: 5) | | |
| PRL_HUMAN | LSAYYNLLHCLR (SEQ | 511.6 m/z→666.84 m/z | |
| | ID NO: 5) | | |
| PRL_HUMAN | LSAYYNLLHCLR (SEQ | | 508.27 m/z→705.35 m/z |
| | ID NO: 5) | | |
| PRL_HUMAN | LSAYYNLLHCLR (SEQ | | 508.27 m/z→661.84 m/z |
| | ID NO: 5) | | |
| PRL_HUMAN | SWNEPLYHLVTEVR | 584.97 m/z→740.4 m/z | |
| | (SEQ ID NO: 6) | | |
| PRL_HUMAN | SWNEPLYHLVTEVR | 584.97 m/z→618.85 m/z | |
| | (SEQ ID NO: 6) | | |
| PRL_HUMAN | SWNEPLYHLVTEVR | | 581.63 m/z→735.39 m/z |
| | (SEQ ID NO: 6) | | |
| PRL_HUMAN | SWNEPLYHLVTEVR | | 581.63 m/z→613.85 m/z |
| | (SEQ ID NO: 6) | | |
| TETN_HUMAN | CFLAFTQTK (SEQ ID | 562.29 m/z→816.47 m/z | |
| | NO: 7) | | |
| TETN_HUMAN | CFLAFTQTK (SEQ ID | 562.29 m/z→485.28 m/z | |
| | NO: 7) | | |
| TETN_HUMAN | CFLAFTQTK (SEQ ID | | 558.28 m/z→808.46 m/z |
| | NO: 7) | | |
| TETN_HUMAN | CFLAFTQTK (SEQ ID | | 558.28 m/z→477.27 m/z |
| | NO: 7) | | |
| TETN_HUMAN | LDTLAQEVALLK (SEQ | 661.39 m/z→1093.67 | |
| | ID NO: 8) | m/z | |
| TETN_HUMAN | LDTLAQEVALLK (SEQ | 661.39 m/z→879.54 m/z | |
| | ID NO: 8) | | |
| TETN_HUMAN | LDTLAQEVALLK (SEQ | | 657.39 m/z→1085.66 |
| | ID NO: 8) | | m/z |
| TETN_HUMAN | LDTLAQEVALLK (SEQ | | 657.39 m/z→871.52 m/z |
| | ID NO: 8) | | |
| ALS_HUMAN | IRPHTFTGLSGLR (SEQ | 488.9 m/z→555.3 m/z | |
| | ID NO: 19) | | |
| ALS_HUMAN | IRPHTFTGLSGLR (SEQ | 488.9 m/z→442.3 m/z | |
| | ID NO: 19) | | |
| ALS_HUMAN | IRPHTFTGLSGLR (SEQ | | 485.6 m/z→545.3 m/z |
| | ID NO: 19) | | |
| ALS_HUMAN | IRPHTFTGLSGLR (SEQ | | 485.6 m/z→432.3 m/z |
| | ID NO: 19) | | |
| CSH1_HUMAN; | AHQLAIDTYQEFEETYI | 768.7 m/z→634.4 m/z | |
| CSH2_HUMAN | PK (SEQ ID NO: 22) | | |
| CSH1_HUMAN; | AHQLAIDTYQEFEETYI | 768.7 m/z→521.3 m/z | |
| CSH2_HUMAN | PK (SEQ ID NO: 22) | | |
| CSH1_HUMAN; | AHQLAIDTYQEFEETYI | 768.71 m/z→365.26 m/z | |
| CSH2_HUMAN | PK (SEQ ID NO: 22) | | |
| CSH1_HUMAN; | AHQLAIDTYQEFEETYI | 768.71 m/z→252.18 m/z | |
| CSH2_HUMAN | PK (SEQ ID NO: 22) | | |
| CSH1_HUMAN; | AHQLAIDTYQEFEETYI | | 766 m/z→634.4 m/z |
| CSH2_HUMAN | PK (SEQ ID NO: 22) | | |
| CSH1_HUMAN; | AHQLAIDTYQEFEETYI | | 766 m/z→521.3 m/z |
| CSH2_HUMAN | PK (SEQ ID NO: 22) | | |
| CSH1_HUMAN; | AHQLAIDTYQEFEETYI | | 766.04 m/z→357.25 m/z |
| CSH2_HUMAN | PK (SEQ ID NO: 22) | | |
| CSH1_HUMAN; | AHQLAIDTYQEFEETYI | | 766.04 m/z→244.17 m/z |
| CSH2_HUMAN | PK (SEQ ID NO: 22) | | |
| CSH1_HUMAN; | ISLLLIESWLEPVR (SEQ | 839.5 m/z→510.3 m/z | |
| CSH2_HUMAN | ID NO: 23) | | |
| CSH1_HUMAN; | ISLLLIESWLEPVR (SEQ | 839.5 m/z→381.2 m/z | |
| CSH2_HUMAN | ID NO: 23) | | |
| CSH1_HUMAN; | ISLLLIESWLEPVR (SEQ | | 834.5 m/z→500.3 m/z |
| CSH2_HUMAN | ID NO: 23) | | |
| CSH1_HUMAN; | ISLLLIESWLEPVR (SEQ | | 834.5 m/z→371.2 m/z |
| CSH2_HUMAN | ID NO: 23) | | |
| IBP3_HUMAN | FLNVLSPR (SEQ ID NO: | 478.3 m/z→695.4 m/z | |
| | 32) | | |
| IBP3_HUMAN | FLNVLSPR (SEQ ID NO: | 478.3 m/z→482.3 m/z | |
| | 32) | | |
| IBP3_HUMAN | FLNVLSPR (SEQ ID NO: | 478.28 m/z→369.21 m/z | |
| | 32) | | |
| IBP3_HUMAN | FLNVLSPR (SEQ ID NO: | | 473.3 m/z→685.4 m/z |
| | 32) | | |
| IBP3_HUMAN | FLNVLSPR (SEQ ID NO: | | 473.3 m/z→472.3 m/z |
| | 32) | | |
| IBP3_HUMAN | FLNVLSPR (SEQ ID NO: | | 473.28 m/z→359.2 m/z |
| | 32) | | |
| IBP3_HUMAN | YGQPLPGYTTK (SEQ | 616.8 m/z→884.5 m/z | |
| | ID NO: 33) | | |
| IBP3_HUMAN | YGQPLPGYTTK (SEQ | 616.8 m/z→674.4 m/z | |
| | ID NO: 33) | | |
| IBP3_HUMAN | YGQPLPGYTTK (SEQ | | 612.8 m/z→876.5 m/z |
| | ID NO: 33) | | |
| IBP3_HUMAN | YGQPLPGYTTK (SEQ | | 612.8 m/z→666.3 m/z |
| | ID NO: 33) | | |
| FA5_HUMAN | AEVDDVIQVR (SEQ ID | 577.31 m/z→739.43 m/z | |
| | NO: 60) | | |
| FA5_HUMAN | AEVDDVIQVR (SEQ ID | 577.31 m/z→854.46 m/z | |
| | NO: 60) | | |
| FA5_HUMAN | AEVDDVIQVR (SEQ ID | | 572.3 m/z→729.43 m/z |
| | NO: 60) | | |
| FA5_HUMAN | AEVDDVIQVR (SEQ ID | | 572.3 m/z→844.45 m/z |
| | NO: 60) | | |
| FA5_HUMAN | LSEGASYLDHTFPAEK | 591.62 m/z→722.35 m/z | |
| | (SEQ ID NO: 27) | | |
| FA5_HUMAN | LSEGASYLDHTFPAEK | 591.62 m/z→786.87 m/z | |
| | (SEQ ID NO: 27) | | |
| FA5_HUMAN | LSEGASYLDHTFPAEK | | 588.95 m/z→718.35 m/z |
| | (SEQ ID NO: 27) | | |
| FA5_HUMAN | LSEGASYLDHTFPAEK | | 588.95 m/z→782.87 m/z |
| | (SEQ ID NO: 27) | | |
| PAPP2_HUMAN | LLLRPEVLAEIPR (SEQ | 510.32 m/z→722.46 m/z | |
| | ID NO: 38) | | |
| PAPP2_HUMAN | LLLRPEVLAEIPR (SEQ | 510.32 m/z→821.52 m/z | |
| | ID NO: 38) | | |
| PAPP2_HUMAN | LLLRPEVLAEIPR (SEQ | | 506.98 m/z→722.46 m/z |
| | ID NO: 38) | | |
| PAPP2_HUMAN | LLLRPEVLAEIPR (SEQ | | 506.98 m/z→821.52 m/z |
| | ID NO: 38) | | |

While the enhanced IBP4/SHBG Classifier is defined from the natural log of the SIS normalized intensities of the IBP4, SHBG, EGLN, PRL and TETN peptides, the invention also comprises classifiers that include multiple reversals, e.g., score=ln(P1n/P2n)+ln(P3n+P4n)/P5n. Improved performance can be achieved by constructing predictors formed from more than one reversal and with one or more clinical variable In additional embodiments, the invention methods therefore comprise multiple reversals that have a strong predictive performance for example, for separate GABD windows, preterm premature rupture of membranes (PPROM) versus preterm labor in the absence of PPROM (PTL), fetal gender, primigravida versus multigravida.

The methods of the invention further include classifiers that contain an indicator variable that selects one or a subset of reversals based on known clinical factors, for example, blood draw period, fetal gender, gravidity as well as any other knowable patient features and/or risk factors described throughout this application. This embodiment is exemplified in Example 10, Tables 61 through 64 of PCT Publication WO2016/205723, which exemplify reversal performance (weeks 17-21) independently for two different phenotypes of sPTB, PPROM and PTL. This embodiment is similarly exemplified in Example 10, Tables 76 and 77 and FIGS. 108 and 109 of PCT Publication WO2016/205723, which exemplify reversal performance (weeks 19-21) independently for two different phenotypes of sPTB, preterm premature rupture of membranes (PPROM) and preterm labor in the absence of PPROM (PTL). The methods of the invention thus include selection of reversals to build independent predictors of PPROM and PTL, or to maximize performance overall with the combination of more than one reversal in a single predictor as described above. This embodiment is further exemplified in Example 10, Tables 65-68, PCT Publication WO2016/205723 which exemplify reversal performance (weeks 17-21) independently for two different types of sPTB, primigravida and multigravida. This embodiment is further exemplified in Example 10, Tables 69-72 and FIG. 106 PCT Publication WO2016/205723, which exemplify reversal performance (weeks 17-21) independently for two different types of sPTB based on fetal gender. While exemplified with regard to PPROM and PTL, gravidity and fetal gender, the methods of the invention include classifiers that contain an indicator variable that selects one or a subset of reversals based on GABD or any known clinical factors/risk factors described herein or otherwise known to those of skill in the art. As an alternative to having a classifier that includes an indicator variable, the invention further provides separate classifiers that are tailored to subsets of pregnant women based on GABD or any known clinical factors/risk factors described herein or otherwise known to those of skill in the art. For example, this embodiment encompasses separate classifiers for consecutive and/or overlapping time windows for GABD that are based on the best performing reversals for each time window.

As exemplified herein, the predictive performance of the claimed methods can be improved with a BMI stratification of greater than 22 and equal or less than 37 kg/m2. Accordingly, in some embodiments, the methods of the invention can be practiced with samples obtained from pregnant females with a specified BMI. Briefly, BMI is an individual's weight in kilograms divided by the square of height in meters. BMI does not measure body fat directly, but research has shown that BMI is correlated with more direct measures of body fat obtained from skinfold thickness measurements, bioelectrical impedance, densitometry (underwater weighing), dual energy x-ray absorptiometry (DXA) and other methods. Furthermore, BMI appears to be as strongly correlated with various metabolic and disease outcome as are these more direct measures of body fatness. Generally, an individual with a BMI below 18.5 is considered underweight, an individual with a BMI of equal or greater than 18.5 to 24.9 normal weight, while an individual with a BMI of equal or greater than 25.0 to 29.9 is considered overweight and an individual with a BMI of equal or greater than 30.0 is considered obese. In some embodiments, the predictive performance of the claimed methods can be improved with a BMI stratification of equal or greater than 18, equal or greater than 19, equal or greater than 20, equal or greater than 21, equal or greater than 22, equal or greater than 23, equal or greater than 24, equal or greater than 25, equal or greater than 26, equal or greater than 27, equal or greater than 28, equal or greater than 29 or equal or greater than 30. In other embodiments, the predictive performance of the claimed methods can be improved with a BMI stratification of equal or less than 18, equal or less than 19, equal or less than 20, equal or less than 21, equal or less than 22, equal or less than 23, equal or less than 24, equal or less than 25, equal or less than 26, equal or less than 27, equal or less than 28, equal or less than 29 or equal or less than 30.

In the context of the present invention, the term “biological sample,” encompasses any sample that is taken from pregnant female, e.g., containing one or more of the biomarkers disclosed herein.

As used herein, the term “preterm birth” refers to delivery or birth at a gestational age less than some specific number of completed weeks, e.g., less than 37 completed weeks. Other commonly used subcategories of preterm birth have been established and delineate moderately preterm (birth at 33 to 36 weeks of gestation), very preterm (birth at <33 weeks of gestation), and extremely preterm (birth at ≤28 weeks of gestation). With regard to the methods disclosed herein, those skilled in the art understand that the cut-offs that delineate preterm birth and term birth as well as the cut-offs that delineate subcategories of preterm birth can be adjusted in practicing the methods disclosed herein, for example, to maximize a particular health benefit. In various embodiments of the invention, cut-offs that delineate preterm birth include, for example, birth at ≤37 weeks of gestation, ≤36 weeks of gestation, ≤35 weeks of gestation, ≤34 weeks of gestation, ≤33 weeks of gestation, ≤32 weeks of gestation, ≤30 weeks of gestation, ≤29 weeks of gestation, ≤28 weeks of gestation, ≤27 weeks of gestation, ≤26 weeks of gestation, ≤25 weeks of gestation, ≤24 weeks of gestation, ≤23 weeks of gestation or ≤22 weeks of gestation. In some embodiments, the cut-off delineating preterm birth is ≤35 weeks of gestation. It is further understood that such adjustments are well within the skill set of individuals considered skilled in the art and encompassed within the scope of the inventions disclosed herein. Gestational age is a proxy for the extent of fetal development and the fetus's readiness for birth. Gestational age has typically been defined as the length of time from the date of the last normal menses to the date of birth. However, obstetric measures and ultrasound estimates also can aid in estimating gestational age. Preterm births have generally been classified into two separate subgroups: (1) spontaneous preterm births occurring subsequent to spontaneous onset of preterm labor or preterm premature rupture of membranes regardless of subsequent labor augmentation or cesarean delivery, or (2) medically indicated preterm births occurring following induction or cesarean section for one or more conditions that the woman's caregiver determines to threaten the health or life of the mother and/or fetus. In some embodiments, the methods disclosed herein are directed to determining the probability for spontaneous preterm birth or medically indicated preterm birth. In some embodiments, the methods disclosed herein are directed to determining the probability for spontaneous preterm birth. In additional embodiments, the methods disclosed herein are directed to medically indicated preterm birth. In additional embodiments, the methods disclosed herein are directed to predicting gestational age at birth.

As used herein, the term “estimated gestational age” or “estimated GA” refers to the GA determined based on the date of the last normal menses and additional obstetric measures, ultrasound estimates or other clinical parameters including, without limitation, those described in the preceding paragraph. In contrast the term “predicted gestational age at birth” or “predicted GAB” refers to the GAB determined based on the methods of the invention as disclosed herein. As used herein, “term birth” refers to birth at a gestational age equal or more than 37 completed weeks.

The term “amount” or “level” as used herein refers to a quantity of a biomarker that is detectable or measurable in a biological sample and/or control. The quantity of a biomarker can be, for example, a quantity of polypeptide, the quantity of nucleic acid, or the quantity of a fragment or surrogate. The term can alternatively include combinations thereof. The term “amount” or “level” of a biomarker is a measurable feature of that biomarker.

A “proteomics work-flow” generally encompasses one or more of the following steps: Serum samples are thawed and depleted of the 14 highest abundance proteins by immune-affinity chromatography. Depleted serum is digested with a protease, for example, trypsin, to yield peptides. The digest is subsequently fortified with a mixture of SIS peptides and then desalted and subjected to LC-MS/MS with a triple quadrapole instrument operated in MRM mode. Response ratios are formed from the area ratios of endogenous peptide peaks and the corresponding SIS peptide counterpart peaks. Those skilled in the art appreciate that other types of MS such as, for example, MALDI-TOF, or ESI-TOF, can be used in the methods of the invention. In addition, one skilled in the art can modify a proteomics work-flow, for example, by selecting particular reagents (such as proteases) or omitting or changing the order of certain steps, for example, it may not be necessary to immunodeplete, the SIS peptide could be added earlier or later and stable isotope labeled proteins could be used as standards instead of peptides.

As used herein, the term “mass spectrometer” refers to a device able to volatilize/ionize analytes to form gas-phase ions and determine their absolute or relative molecular masses. Suitable methods of volatilization/ionization are matrix-assisted laser desorption ionization (MALDI), electrospray, laser/light, thermal, electrical, atomized/sprayed and the like, or combinations thereof. Suitable forms of mass spectrometry include, but are not limited to, ion trap instruments, quadrupole instruments, electrostatic and magnetic sector instruments, time of flight instruments, time of flight tandem mass spectrometer (TOF MS/MS), Fourier-transform mass spectrometers, Orbitraps and hybrid instruments composed of various combinations of these types of mass analyzers. These instruments can, in turn, be interfaced with a variety of other instruments that fractionate the samples (for example, liquid chromatography or solid-phase adsorption techniques based on chemical, or biological properties) and that ionize the samples for introduction into the mass spectrometer, including matrix-assisted laser desorption (MALDI), electrospray, or nanospray ionization (ESI) or combinations thereof.

As used herein, the terms “multiple reaction monitoring (MRM)” or “selected reaction monitoring (SRM)” refer to an MS-based quantification method that is particularly useful for quantifying analytes that are in low abundance. In an SRM experiment, a predefined precursor ion and one or more of its fragments are selected by the two mass filters of a triple quadrupole instrument and monitored over time for precise quantification. Multiple SRM precursor and fragment ion pairs can be measured within the same experiment on the chromatographic time scale by rapidly toggling between the different precursor/fragment pairs to perform an MRM experiment. A series of transitions (precursor/fragment ion pairs) in combination with the retention time of the targeted analyte (e.g., peptide or small molecule such as chemical entity, steroid, hormone) can constitute a definitive assay. A large number of analytes can be quantified during a single LC-MS experiment. The term “scheduled,” or “dynamic” in reference to MRM or SRM, refers to a variation of the assay wherein the transitions for a particular analyte are only acquired in a time window around the expected retention time, significantly increasing the number of analytes that can be detected and quantified in a single LC-MS experiment and contributing to the selectivity of the test, as retention time is a property dependent on the physical nature of the analyte. A single analyte can also be monitored with more than one transition. Finally, included in the assay can be standards that correspond to the analytes of interest (e.g., same amino acid sequence), but differ by the inclusion of stable isotopes. Stable isotopic standards (SIS) can be incorporated into the assay at precise levels and used to quantify the corresponding unknown analyte. An additional level of specificity is contributed by the co-elution of the unknown analyte and its corresponding SIS and properties of their transitions (e.g., the similarity in the ratio of the level of two transitions of the unknown and the ratio of the two transitions of its corresponding SIS).

In the context of the invention, the term “capture agent” refers to a compound that can specifically bind to a target, in particular a biomarker. The term includes antibodies, antibody fragments, nucleic acid-based protein binding reagents (e.g. aptamers, Slow Off-rate Modified Aptamers (SOMAmer™)), protein-capture agents, natural ligands (i.e. a hormone for its receptor or vice versa), small molecules or engineered nanoparticles (e.g., SEER Proteograph™ product suite.

Included within the embodiments of the invention, are iterative methods of determining probability for preterm birth in a pregnant female, the method comprising measuring in a biological sample obtained from the pregnant female a reversal value or combined reversal value to determine the probability for preterm birth in said pregnant female, wherein the existence of a change in reversal value or combined reversal value between the pregnant female and a term control determines the probability for preterm birth in the pregnant female. Iterative performance of the methods described herein includes subsequent measurements obtained from a single sample as well as obtaining subsequent samples for measurement. For example, if it is determined that the probability for preterm birth in a pregnant female, which can be expressed as a risk score, is above a specified value, the method can be repeated using a distinct reversal pair, reversal triplet or reversal group from the same sample or the same or a distinct reversal pair, reversal triplet or reversal group from a subsequent sample to further stratify the risk for sPTB.

The present disclosure also includes methods for generating a result useful in determining probability for preterm birth in a pregnant female by obtaining a dataset associated with a sample, where the dataset at least includes quantitative data about the relative expression of biomarker pairs, triplets or groups that have been identified as exhibiting changes in reversal value or combined reversal value predictive of preterm birth, and inputting the dataset into an analytic process that uses the dataset to generate a result useful in determining probability for preterm birth in a pregnant female. As described further below, quantitative data can include amino acids, peptides, polypeptides, proteins, nucleotides, nucleic acids, nucleosides, sugars, fatty acids, steroids, metabolites, carbohydrates, lipids, hormones, antibodies, regions of interest that serve as surrogates for biological macromolecules and combinations thereof.

The disclosure further provides compositions, panels and kits for determining the probability for preterm birth in a pregnant female. In some embodiments, such compositions, panels and kits determine the probability for perterm birth in only nultips. In some embodiments, such compositions, panels and kits determine the probability for preterm birth in only multips. In some embodiments, such compositions, panels, and kits determine the probability for perterm birth in both nullips and multips. One major advantage of the present disclosure is that risk of developing preterm birth can be assessed early during pregnancy so that appropriate monitoring and clinical management to prevent preterm delivery can be initiated in a timely fashion. The present invention is of particular benefit to females lacking any risk factors for preterm birth and who would not otherwise be identified and treated.

### Composition

In one aspect, the invention provides a composition comprising at least three pairs of biomarkers wherein said biomarkers exhibit a change in reversal value between pregnant females at risk for preterm birth and term controls. In some embodiments, the at least three pairs of biomarkers can include any of the biomarkers set forth in Table 3. In some embodiments, the at least three pairs of biomarkers can include other biomarkers not set forth in Table 3. In some embodiments, the biomarkers are similar to the biomarkers in Table 3. In other embodiments, the at least three pairs of biomarkers can include any of the biomarkers set forth in the reversal groups in Tables 5-18, and 21-22. In some embodiments, the at least three pairs of biomarkers can include IBP4/SHBG, IBP4/TETN, EGLN/SHBG, EGLN/TETN, PRL/SHBG, or PRL/TETN. In particular embodiments, the at least three pairs of biomarkers consist of IBP4/SHBG, EGLN/TETN, and PRL/TETN.

In one aspect, the invention provides a composition comprising at least two pairs of biomarkers, wherein said biomarkers exhibit a change in reversal value between pregnant females at risk for preterm birth and term controls. In some embodiments, the at least two pairs of biomarkers comprise a first pair and a second pair of biomarkers. In some embodiments, the at least two pairs of biomarkers comprise a first reversal pair of IBP4/SHBG, and a second reversal pair selected from Table 22. In some embodiments, the biomarkers can include any of the biomarkers set forth in Table 3. In some embodiments, the biomarker pairs can include any of the biomarker pairs set forth in Table 22. In some embodiments, the at least two pairs of biomarkers can include other biomarkers not set forth in Table 3. In some embodiments, the biomarkers are similar to the biomarkers in Table 3. In some embodiments, the at least two pairs of biomarkers can include other biomarker pairs not set forth in Table 22. In some embodiments, the biomarker pairs are similar to the biomarkers in Table 22.

TABLE 3
Peptides for use in the compositions, panels, methods and kits
disclosed herein.

| Protein Description | Uniprot ID (name) | Peptide |
|---|---|---|
| Adam 12 | ADA12_HUMAN | FGFGGSTDSGPIR (SEQ ID NO: 9) |
| Apolipoprotein C-III | APOC3_HUMAN | GWVTDGFSSLK (SEQ ID NO: 10) |
| ADAM | ATS13_HUMAN | YGSQLAPETFYR (SEQ ID NO: 11) |
| Metallopeptidase with | | |
| Thrombospondin Type | | |
| 1 Motif 13 | | |
| Complement Factor B | CFAB_HUMAN | YGLVTYATYPK (SEQ ID NO: 12) |
| Neural Cell Adhesion | CHL1_HUMAN | VIAVNEVGR (SEQ ID NO: 13) |
| Molecule L1-like | | |
| protein | | |
| Endoglin | EGLN_HUMAN | TQILEWAAER (SEQ ID NO: 4) |
| Insulin Like Growth | IBP1_HUMAN | VVESLAK (SEQ ID NO: 14) |
| Factor Binding Protein | | |
| 1 | | |
| Insulin like growth | IBP4_HUMAN | QCHPALDGQR (SEQ ID NO: 2) |
| factor binding protein | | |
| Peptidoglycan | PGRP2_HUMAN | AGLLRPDYALLGHR (SEQ ID NO: |
| Recognition Protein 2 | | 15) |
| Prolactin | PRL_HUMAN | LSAYYNLLHCLR (SEQ ID NO: 5) |
| Pregnancy Specific | PSG3_HUMAN | VSAPSGTGHLPGLNPL (SEQ ID |
| Beta-1-Glycoprotein 3 | | NO: 16) |
| Sex Hormone Binding | SHBG_HUMAN | IALGGLLFPASNLR (SEQ ID NO: |
| Globulin | | 1) |
| Tetranectin | TETN_HUMAN | CFLAFTQTK (SEQ ID NO: 7) |
| Tissue Inhibitor of | TIMP1_HUMAN | HLACLPR (SEQ ID NO: 17) |
| Metalloproteinases 1 | | |
| Leucine-rich alpha-2- | A2GL_HUMAN | DLLLPQPDLR (SEQ ID NO: 18) |
| glyocoprotein precursor | | |
| Insulin-like growth | ALS_HUMAN | IRPHTFTGLSGLR (SEQ ID NO: 19) |
| factor-binding protein | | |
| complex acid labile | | |
| subunit | | |
| Angiotensinogen | ANGT_HUMAN | DPTFIPAPIQAK (SEQ ID NO: 20) |
| Clusterin Preproprotein | CLUS_HUMAN | ASSIIDELFQDR (SEQ ID NO: 21) |
| Chorionic | CSH1_HUMAN | AHQLAIDTYQEFEETYIPK (SEQ |
| Somatomammotropin | CSH2_HUMAN | ID NO: 22) |
| Hormone 1 | | |
| Chorionic | | |
| Somatomammotropin | | |
| Hormone 2 | | |
| Chorionic | CSH1_HUMAN | ISLLLIESWLEPVR (SEQ ID NO: |
| Somatomammotropin | CSH2_HUMAN | 23) |
| Hormone 1 | | |
| Chorionic | | |
| Somatomammotropin | | |
| Hormone 2 | | |
| Defensin, Alpha 1 | DEF1_HUMAN | YGTCIYQGR (SEQ ID NO: 24) |
| Dipeptidase 2 | DPEP2_HUMAN | ALEVSQAPVIFSHSAAR (SEQ ID |
| | | NO: 25) |
| Coagulation Factor | F13B_HUMAN | GDTYPAELYITGSILR (SEQ ID |
| XIII B Chain | | NO: 26) |
| Coagulation Factor V | FA5_HUMAN | LSEGASYLDHTFPAEK (SEQ ID |
| | | NO: 27) |
| Fibulin 3 | FBLN3_HUMAN | IPSNPSHR (SEQ ID NO: 28) |
| Alpha-2-HS | FETUA_HUMAN | HTLNQIDEVK (SEQ ID NO: 29) |
| glycoprotein | | |
| preproprotein | | |
| Hemopexin Precursor | HEMO_HUMAN | NFPSPVDAAFR (SEQ ID NO: 30) |
| Insulin Like Growth | IBP2_HUMAN | LIQGAPTIR (SEQ ID NO: 31) |
| Factor Binding Protein | | |
| 2 | | |
| Insulin Like Growth | IBP3_HUMAN | FLNVLSPR (SEQ ID NO: 32) |
| Factor Binding Protein | | |
| 3 | | |
| Insulin Like Growth | IBP3_HUMAN | YGQPLPGYTTK (SEQ ID NO: 33) |
| Factor Binding Protein | | |
| 3 | | |
| Insulin Like Growth | IGF1_HUMAN | GFYFNKPTGYGSSSR (SEQ ID NO: |
| Factor 1 | | 34) |
| Insulin-Like Growth | IGF2_HUMAN | GIVEECCFR (SEQ ID NO: 35) |
| Factor 2 | | |
| Lymphocyte adhesion | LYAM1_HUMAN | SYYWIGIR (SEQ ID NO: 36) |
| molecule 1 | | |
| Pappalysin-1 | PAPP1_HUMAN | DIPHWLNPTR (SEQ ID NO: 37) |
| preproprotein | | |
| Pappalysin-2 | PAPP2_HUMAN | LLLRPEVLAEIPR (SEQ ID NO: 38) |
| preproprotein | | |
| Pigment epithelium- | PEDF_HUMAN | TVQAVLTVPK (SEQ ID NO: 39) |
| derived | | |
| factor | | |
| Prolactin | PRL_HUMAN | SWNEPLYHLVTEVR (SEQ ID NO: |
| | | 6) |
| Pregnancy Specific | PSG2_HUMAN | IHPSYTNYR (SEQ ID NO: 40) |
| Beta-1-Glycoprotein 2 | | |
| Prostaglandin D2 | PTGDS_HUMAN | AQGFTEDTIVFLPQTDK (SEQ ID |
| Synthase | | NO: 41) |
| Tenascin XB | TENX_HUMAN | LSQLSVTDVTTSSLR (SEQ ID NO: |
| | | 42) |
| Tetranectin | TETN_HUMAN | CFLAFTQTK (SEQ ID NO: 7) |
| Tetranectin | TETN_HUMAN | LDTLAQEVALLK (SEQ ID NO: 8) |
| Vascular endothelial | VGFR1_HUMAN | YLAVPTSK (SEQ ID NO: 43) |
| growth factor receptor | | |
| 1 | | |
| Lipopolysaccharide- | LBP_HUMAN | ITGFLKPGK (SEQ ID NO: 61) |
| binding protein (LBP) | | ITLPDFTGDLR (SEQ ID NO: 44) |
| Inter-alpha-trypsin | ITIH4_HUMAN | ILDDLSPR (SEQ ID NO: 45) |
| inhibitor heavy chain | | |
| H4 | | |
| Pregnancy-specific | PSG1_HUMAN | FQLPGQK (SEQ ID NO: 46) |
| beta-1-glycoprotein 1 | | |
| Complement Clq | CIQB_HUMAN | IAFSATR (SEQ ID NO: 62) |
| subcomponent subunit | | LEQGENVFLQATDK (SEQ ID NO: |
| B | | 47) |
| Beta-2-glycoprotein 1 | APOH_HUMAN | ATVVYQGER (SEQ ID NO: 48) |
| Complement Clq | CIQC_HUMAN | FNAVLTNPQGDYDTSTGK (SEQ |
| subcomponent subunit | | ID NO: 63) |
| C | | TNQVNSGGVLLR (SEQ ID NO: 49) |
| Extracellular matrix | ECM1_HUMAN | ELLALIQLER (SEQ ID NO: 64) |
| protein 1 (Secretory | | LLPAQLPAEK (SEQ ID NO: 50) |
| component p85) | | |
| Sushi, von Willebrand | SVEP1_HUMAN | LLSDFPVVPTATR (SEQ ID NO: |
| factor type A, EGF and | | 51) |
| pentraxin domain- | | |
| containing protein 1 | | |
| Protein AMBP | AMBP_HUMAN | ETLLQDFR (SEQ ID NO: 52) |
| Cathelicidin | CAMP_HUMAN | AIDGINQR (SEQ ID NO: 65) |
| antimicrobial peptide | | SSDANLYR (SEQ ID NO: 53) |
| Complement | CO8A_HUMAN | SLLQPNK (SEQ ID NO: 54) |
| component C8 alpha | | |
| chain | | |
| Gelsolin | GELS_HUMAN | AQPVQVAEGSEPDGFWEALGGK |
| | | (SEQ ID NO: 66) |
| | | TASDFITK (SEQ ID NO: 55) |
| Somatotropin, growth | SOM2_HUMAN | NYGLLYCFR (SEQ ID NO: 56) |
| hormone variant | CSH1_HUMAN | |
| Chorionic | CSH2_HUMAN | |
| Somatomammotropin | | |
| Hormone 1 | | |
| Chorionic | | |
| Somatomammotropin | | |
| Hormone 2 | | |
| Cathepsin D | CATD_HUMAN | VGFAEAAR (SEQ ID NO: 57) |
| Afamin | AFAM_HUMAN | HFQNLGK (SEQ ID NO: 58) |
| Coagulation factor IX | FA9_HUMAN | FGSGYVSGWGR (SEQ ID NO: 67) |
| | | SALVLQYLR (SEQ ID NO: 59) |

Note that, in some cases, one fragment is assayed for two different proteins because there exists overlap in peptide sequence. In some cases, there are two or more protein names assigned to one peptide.

The invention provides a composition comprising a reversal group of isolated biomarkers, comprising a reversal pair and a reversal triplet, wherein the reversal group of biomarkers exhibits a change in combined reversal value between pregnant females at risk for pre-term birth and term controls. In some embodiments, the reversal pair of the reversal group can include IBP4/SHBG. In other embodiments, the reversal pair can include any of the isolated biomarkers set forth in Table 3. In some embodiments, the reversal triplet can include EGLN, PRL and TETN. In other embodiments, the reversal triplet can include any of the isolated biomarkers set forth in Table 3. In some embodiments, the reversal group of isolated biomarkers can include any of the reversal groups set forth in Tables 6-19. In some embodiments, the reversal group of isolated biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In particular embodiments, the reversal group can be (EGLN+PRL)/TETN+IBP4/SHBG.

The invention provides a composition comprising a reversal group of isolated biomarkers, comprising a reversal triplet of isolated biomarkers, wherein said reversal group of biomarkers exhibits a change in combined reversal value between pregnant females at risk for pre-term birth and term controls. In some embodiments, the reversal triplet can include (EGLN+IBP4)/SHBG or (PAPP2+IBP4)/SHBG. In some embodiments, the reversal triplet can include any of the isolated biomarkers set forth in Table 3 or Table 20.

The invention provides a composition comprising a reversal group of isolated biomarkers comprising at least two pairs of biomarkers, wherein the at least two pairs of biomarkers comprise a first reversal pair and a second reversal pair, wherein said pairs of biomarkers exhibit a change in reversal value between pregnant females at risk for pre-term birth and term controls. In some embodiments, the first reversal pair of the reversal group can include IBP4/SHBG. In some embodiments, the second reversal pair of the reversal group can include any of the reversal pair of biomarkers set forth in Table 22. In some embodiments, the composition comprises a third reversal pair of biomarkers. In some embodiments, the third reversal pair can include any of the biomarkers set forth in Table 22, except the third reversal pair and second reversal pair are not the same.

In another aspect, the invention provides a composition comprising a reversal group of surrogate peptides of the isolated biomarkers comprising a reversal pair and a reversal triplet, wherein the reversal group exhibits a change in combined reversal value between pregnant females at risk for preterm birth and term controls. In some embodiments, the reversal pair of surrogate peptides can include IBP4/SHBG. In other embodiments, the reversal pair of surrogate peptides can include any of the isolated biomarkers set forth in Table 3. In some embodiments, the reversal triplet of surrogate peptides can include EGLN, PRL and TETN. In other embodiments, the reversal triplet of surrogate peptides can include any of the isolated biomarkers set forth in Table 3. In some embodiments, the reversal group of surrogate peptides can include any of the reversal groups set forth in Tables 6-19. In some embodiments, the reversal group of isolated biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In particular embodiments, the reversal group of surrogate peptides can be (EGLN+PRL)/TETN+IBP4/SHBG.

In another aspect, the invention provides a composition comprising a reversal group of surrogate peptides of the isolated biomarkers comprising a reversal triplet of isolated biomarkers, wherein said reversal group exhibits a change in combined reversal value between pregnant females at risk for pre-term birth and term controls. In some embodiments, the reversal triplet of surrogate peptides can include EGLN, IBP4, and SHBG. In some embodiments, the reversal triplet of surrogate peptides can include PAPP2, IBP4, and SHBG. In other embodiments, the reversal triplet of surrogate peptides can include any of the isolated biomarkers set forth in Tables 3 or 20. In some embodiments, the reversal group of surrogate peptides can be (EGLN+IBP4)/SHBG or (PAPP2+IBP4)/SHBG.

In another aspect, the invention provides a composition comprising a reversal group of surrogate peptides of the isolated biomarkers comprising at least two reversal pairs of isolated biomarkers. In some embodiments, the at least two reversal pairs of isolated biomarkers can include a first reversal pair and a second reversal pair. In some embodiments, the first reversal pair of biomarkers can be IBP4/SHBG. In some embodiments, the second reversal pair of biomarkers can include any of the biomarker pairs set forth in Table 22. In some embodiments, the reversal group of surrogate peptides comprises a third reversal pair of biomarkers. In some embodiments, the third reversal pair can be any reversal pair of biomarkers set forth in Table 22, except the third reversal pair and the second reversal pair are not the same.

In further embodiments, the composition can comprise stable isotope labeled standard peptides (SIS peptides) corresponding the surrogate peptides disclosed herein.

### Panel

In a first aspect, the invention provides a panel for determining biomarker expression in a biological sample obtained from a pregnant female. Levels of biomarkers in a biological sample from a pregnant female can be determined by any known techniques in the art, e.g., HPLC, mass spectrometry or an assay using a capture agent such as antibodies specific to selected proteins (e.g., IHC, ELISA, etc.). In some embodiments, the panel can be used to determine expression of hundreds or thousands of peptides. In a preferred embodiment, only a subset the hundreds or thousands of peptides are analyzed. In alternative embodiments, a panel for determining biomarker expression can include gene expression at the RNA level (i.e. mRNA or noncoding RNA (ncRNA)) (e.g., miRNA, tRNA, rRNA, snoRNA, siRNA and piRNA).

In one aspect, the invention provides a panel comprising at least three pairs of biomarkers wherein said pairs of biomarkers exhibit a change in reversal value between pregnant females at risk for preterm birth and term controls. In some embodiments, the at least three pairs of biomarkers can include any of the biomarkers set forth in Table 3. In some embodiments, the at least three pairs of biomarkers can include other biomarkers not set forth in Table 3. In some embodiments, the biomarkers are similar to the biomarkers in Table 3. In other embodiments, the at least three pairs of biomarkers can include any of the biomarkers set forth in the reversal groups in Tables 5-19, and 21-22. In some embodiments, the at least three pairs of biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In some embodiments, the at least three pairs of biomarkers can include IBP4/SHBG, IBP4/TETN, EGLN/SHBG, EGLN/TETN, PRL/SHBG, or PRL/TETN. In particular embodiments, the at least three pairs of biomarkers consist of IBP4/SHBG, EGLN/TETN, and PRL/TETN.

The invention provides a panel comprising a reversal group of isolated biomarkers, comprising a reversal pair and a reversal triplet, wherein the reversal group of biomarkers exhibits a change in combined reversal value between pregnant females at risk for pre-term birth and term controls. In some embodiments, the reversal pair of the reversal group can include IBP4/SHBG. In other embodiments, the reversal pair can include any of the isolated biomarkers set forth in Table 3. In some embodiments, the reversal triplet can include EGLN, PRL and TETN. In other embodiments, the reversal triplet can include any of the isolated biomarkers set forth in Table 3. In some embodiments, the reversal group of isolated biomarkers can include any of the reversal groups set forth in Tables 6-19, and 21-22. In some embodiments, the reversal group of isolated biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In particular embodiments, the reversal group can be (EGLN+PRL)/TETN+IBP4/SHBG.

In one aspect, the invention provides a panel of biomarkers comprising a reversal group of biomarkers comprising a reversal triplet of isolated biomarkers, wherein said reversal group of isolated biomarkers exhibits a change in combined reversal value between pregnant females at risk for pre-term birth and term controls. In some embodiments, the reversal triplet can include EGLN, IBP4, and SHBG. In some embodiments, the reversal triplet can include PAPP2, IBP4, and SHBG. In other embodiments, the reversal triplet can include any of the isolated biomarkers set forth in Table 3. In some embodiments, the reversal triplet can include (EGLN+IBP4)/SHBG or (PAPP2+IBP4)/SHBG.

In one aspect, the invention provides a panel comprising at least two pairs of biomarkers, wherein the at least two pairs of biomarkers comprise a first reversal pair and a second reversal pair, wherein said pairs of biomarkers exhibit a change in reversal value between pregnant females at risk for pre-term birth and term controls. In some embodiments, the first reversal pair can include IBP4/SHBG. In some embodiments, the second reversal pair can include any of the isolated biomarker pairs set forth in Table 22. In some embodiments, the panel comprises a third pair of biomarkers, wherein the third pair of biomarkers can include any of the reversal pair of isolated biomarkers set forth in Table 22, except the third reversal pair and the second reversal pair are not the same.

In one aspect, the invention provides a panel of biomarkers comprising a reversal group of biomarkers comprising a first reversal pair of isolated biomarkers, and second reversal pair of isolated biomarkers, wherein said reversal group of biomarkers exhibits a change in combined reversal value between pregnant females at risk for pre-term birth and term controls. In some embodiments, the first reversal pair of isolated biomarkers can include IBP4/SHBG. In some embodiments, the second reversal pair can include any of the isolated biomarker pairs set forth in Table 22. In some embodiments, the panel comprising the reversal group further comprises a third reversal pair. In some embodiments, the third reversal pair can include any of the reversal pairs of isolated biomarkers set forth in Table 22, except the third reversal pair and the second reversal pair are not the same.

In another aspect, the invention provides a panel comprising a reversal group of surrogate peptides of the isolated biomarkers comprising a reversal pair and a reversal triplet, wherein the reversal group exhibits a change in combined reversal value between pregnant females at risk for preterm birth and term controls. In some embodiments, the reversal pair of surrogate peptides can include IBP4/SHBG. In other embodiments, the reversal pair of surrogate peptides can include any of the isolated biomarkers set forth in Table 3. In some embodiments, the reversal triplet of surrogate peptides can include EGLN, PRL and TETN. In other embodiments, the reversal triplet of surrogate peptides can include any of the isolated biomarkers set forth in Table 3. In some embodiments, the reversal group of surrogate peptides can include any of the reversal groups set forth in Tables 6-19, and 21-22. In some embodiments, the reversal group of isolated biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In particular embodiments, the reversal group of surrogate peptides can be (EGLN+PRL)/TETN+IBP4/SHBG.

In one aspect, the invention provides a panel comprising a reversal group of surrogate peptides of the isolated biomarkers comprising a reversal triplet, wherein the reversal group exhibits a change in combined reversal value between pregnant females at risk for pre-term birth and term controls. In some embodiments, the reversal triplet of surrogate peptides can include EGLN, IBP4, and SHBG. In some embodiments, the reversal triplet of surrogate peptides can include PAPP2, IBP4, and SHBG. In other embodiments, the reversal triplet of surrogate peptides can include any of the isolated biomarkers set forth in Table 3. In particular embodiments, the reversal group of surrogate peptides can include (EGLN+IBP4)/SHBG or (PAPP2+IBP4)/SHBG.

In one aspect, the invention provides a panel of surrogate peptides comprising a reversal group of surrogate peptides for a reversal group of biomarkers comprising at least two pairs of biomarkers, wherein the at least two pairs of biomarkers comprise a first reversal pair of isolated biomarkers and a second isolated pair of biomarkers, wherein said reversal group of biomarkers exhibits a change in combined reversal value between pregnant females at risk for pre-term birth and term controls. In some embodiments, the first reversal pair of isolated biomarkers can include IBP4/SHBG. In some embodiments, the second reversal pair of biomarkers can include any of the biomarker pairs set forth in Table 22. In some embodiments, the panel of surrogate peptides comprising the reversal group comprises a third reversal pair. In some embodiments, the third reversal pair can be any of the reversal pair of isolated biomarkers set forth in Table 22, except the third reversal pair and the second reversal pair are not the same.

In further embodiments, the panel can comprise stable isotope labeled standard peptides (SIS peptides) corresponding the surrogate peptides disclosed herein.

### Methods

The present disclosure provides methods for predicting probability of preterm birth comprising measuring a change in reversal value, or combined reversal value, of a reversal group or pair of biomarkers. Such biomarkers can be proteins (or peptides), nucleic acids, or metabolites. In some embodiments the sample to be analyzed, either the sample obtained from the patient (e.g., blood) or a derivative thereof (e.g., serum), is enriched for the biomarker(s) of interest. For example, a biological sample can be contacted with one or more binding agents (e.g., antibodies against the protein or peptide of interest, polynucleotide probes or primers) to enrich the sample for the biomarker(s) or interest. The level of one or more biomarkers in the sample can be evaluated according to the methods disclosed below, e.g., with or without the use of enrichment techniques. Skilled practitioners appreciate that in the methods described herein, a measurement of peptide levels can be automated. For example, a system that can carry out multiplexed measurement of peptide levels can be used, e.g., providing measurements of the relative abundance of hundreds of peptides simultaneously.

Some embodiments of the invention involve techniques to detect and measure the levels of proteins or peptides in a sample. Examples of such techniques include, but are not limited to, mass spectrometry and immunoassays. For example, the level of proteins or peptides of interest (e.g., biomarkers or biomarker pairs of the present invention) can be measured in a sample from a patient (or a sample derived therefrom) using mass spectrometry as described in the art (e.g., Sambrook et al., Molecular Cloning, A laboratory Manual, pp. 7.37-7.57 (2nd ed., 1989); Lin et al., in Diagnostic Molecular Microbiology, Principles and Applications, pp. 605-16 (Persing et al., eds. (1993); Ausubel et al., Current Protocols in Molecular Biology (2001 and subsequent updates); Liebler, D. C Introduction to proteomics: Tools for the new biology. Totowa, NJ: Humana Press (2002)); Sechi, Salvatore. Quantitative proteomics by mass spectrometry. Ed. Salvatore Sechi. Vol. 359. Totowa, NJ: Humana Press, (2007); A. L. Burlingame and S. A. Carr, Mass Spectrometry in the Biological Sciences. Eds. (1996), M. R. Wilkins, K. L. Williams, R. D. Apple, D. F. Hochstrasser, Eds. Proteome Research: New Frontiers in Functional Genomics Totowa, NJ: Humana Press (1997); Heidelberg B Methods in Molecular Biology, v. 146. Mass Spectrometry of Proteins and Peptides. John R. Chapman Ed. (2000), Totowa, NJ: Humana Press.

Immunoassays described in the art (Wild, D. & Kodak, E. (2013). The Immunoassay Handbook. 10.1016/C2010-0-66244-4; Crowther J R. The ELISA guidebook. Methods Mol Biol. 2000; 149:III-IV, 1-413. doi: 10.1385/1592590497. PMID: 11028258) can be used to enrich a sample for proteins or peptides of interest (e.g., biomarkers and biomarker pairs of the present invention) and/or to measure those proteins or peptides. A non-limiting example of enrichment includes the use of affinity-capture immunoassays to capture proteins and peptides of interest out of a complex mixture of molecules in a sample, after which the presence and level of such proteins and peptides can be measured by the same or a different technique (e.g., an immunoassay or mass spectrometry). In some embodiments, the use of a capture agent can be combined with mass spectrometry as the detection step. Combining a capture agent for affinity enrichment can greatly reduce the sample complexity, which can allow shorter chromatographic separations that increase throughput (e.g., a 15-minute LC step for LC-MS can be shortened to <5 min per sample).

In some embodiments, measuring nucleic acids (e.g., mRNA) in a biological sample can be used as a surrogate for detection of the level of the corresponding protein biomarker in a biological sample. Thus, any of the biomarkers, biomarker pairs or biomarker reversal panels described herein can also be detected by detecting the appropriate nucleic acid. Levels of nucleic acid (e.g., mRNA) can measured by reverse transcription quantitative polymerase chain reaction (RT-PCR followed with qPCR). RT-PCR is used to create a cDNA from the mRNA. The cDNA can be measured in a number of ways, including quantitative sequencing (e.g., sequencing-by-synthesis such as the Illumina and Solexa systems) or a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure levels of mRNA in a sample. See Gene Expression Profiling: Methods and Protocols, Richard A. Shimkets, editor, Humana Press, 2004.

In some embodiments, nucleic acid amplification methods can be used to detect a polynucleotide biomarker. For example, the oligonucleotide primers and probes of the present invention can be used in amplification and detection methods that use nucleic acid substrates isolated by any of a variety of well-known and established methodologies (e.g., Sambrook et al., Molecular Cloning, A laboratory Manual, pp. 7.37-7.57 (2nd ed., 1989); Lin et al., in Diagnostic Molecular Microbiology, Principles and Applications, pp. 605-16 (Persing et al., eds. (1993); Ausubel et al., Current Protocols in Molecular Biology (2001 and subsequent updates)). Methods for amplifying nucleic acids include, but are not limited to, for example the polymerase chain reaction (PCR) and reverse transcription PCR (RT-PCR) (see e.g., U.S. Pat. Nos. 4,683,195; 4,683,202; 4,800,159; 4,965,188), ligase chain reaction (LCR) (see, e.g., Weiss, Science 254:1292-93 (1991)), strand displacement amplification (SDA) (see e.g., Walker et al., Proc. Natl. Acad. Sci. USA 89:392-396 (1992); U.S. Pat. Nos. 5,270,184 and 5,455,166), Thermophilic SDA (tSDA) (see e.g., European Pat. No. 0 684 315) and methods described in U.S. Pat. No. 5,130,238; Lizardi et al., BioTechnol. 6:1197-1202 (1988); Kwoh et al., Proc. Natl. Acad. Sci. USA 86:1173-77 (1989); Guatelli et al., Proc. Natl. Acad. Sci. USA 87:1874-78 (1990); U.S. Pat. Nos. 5,480,784; 5,399,491; US Publication No. 2006/46265.

In some embodiments, measuring mRNA in a biological sample can be used as a surrogate for detection of the level of the corresponding protein biomarker in a biological sample. Thus, any of the biomarkers, biomarker pairs or biomarker reversal panels described herein can also be detected by detecting the appropriate RNA. Levels of mRNA can measured by reverse transcription quantitative polymerase chain reaction (RT-PCR followed with qPCR). RT-PCR is used to create a cDNA from the mRNA. The cDNA can be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

Some embodiments disclosed herein relate to diagnostic and prognostic methods of determining the probability for preterm birth in a pregnant female. The detection of the level of one or more biomarkers and/or the determination of a ratio of biomarkers can be used to determine the probability for preterm birth in a pregnant female. Such detection methods can be used, for example, for early diagnosis of the condition, to determine whether a subject is predisposed to preterm birth, to monitor the progress of preterm birth or the progress of treatment protocols, to assess the severity of preterm birth, to forecast the outcome of preterm birth and/or prospects of recovery or birth at full term, or to aid in the determination of a suitable treatment for preterm birth.

The quantitation of biomarkers in a biological sample can be determined, without limitation, by the methods described above as well as any other method known in the art. The quantitative data thus obtained can then be subjected to an analytic classification process. In such a process, the raw data can be manipulated according to an algorithm, where the algorithm has been pre-defined by a training set of data, for example as described in the examples provided herein. An algorithm can utilize the training set of data provided herein, or can utilize the guidelines provided herein to generate an algorithm with a different set of data.

In some embodiments, analyzing a measurable feature to determine the probability for preterm birth in a pregnant female encompasses the use of a predictive model. In further embodiments, analyzing a measurable feature to determine the probability for preterm birth in a pregnant female encompasses comparing said measurable feature with a reference feature. As those skilled in the art can appreciate, such comparison can be a direct comparison to the reference feature or an indirect comparison where the reference feature has been incorporated into the predictive model, which may include other measurable features. In further embodiments, analyzing a measurable feature to determine the probability for preterm birth in a pregnant female encompasses one or more of a linear discriminant analysis model, a support vector machine classification algorithm, a recursive feature elimination model, a prediction analysis of microarray model, a logistic regression model, a CART algorithm, a flex tree algorithm, a LART algorithm, a random forest algorithm, a MART algorithm, a machine learning algorithm, a penalized regression method, or a combination thereof. In particular embodiments, the analysis comprises logistic regression.

An analytic classification process can use any one of a variety of statistical analytic methods to manipulate the quantitative data and provide for classification of the sample. Examples of useful methods include linear discriminant analysis, recursive feature elimination, a prediction analysis of microarray, a logistic regression, a CART algorithm, a FlexTree algorithm, a LART algorithm, a random forest algorithm, a MART algorithm, machine learning algorithms; etc.

For creation of a random forest for prediction of GAB one skilled in the art can consider a set of k subjects (pregnant women) for whom the gestational age at birth (GAB) is known, and for whom N analytes (transitions) have been measured in a blood specimen taken several weeks prior to birth. A regression tree begins with a root node that contains all the subjects. The average GAB for all subjects can be calculated in the root node. The variance of the GAB within the root node will be high, because there is a mixture of women with different GAB's. The root node is then divided (partitioned) into two branches, so that each branch contains women with a similar GAB. The average GAB for subjects in each branch is again calculated. The variance of the GAB within each branch will be lower than in the root node, because the subset of women within each branch has relatively more similar GAB's than those in the root node. The two branches are created by selecting an analyte and a threshold value for the analyte that creates branches with similar GAB. The analyte and threshold value are chosen from among the set of all analytes and threshold values, usually with a random subset of the analytes at each node. The procedure continues recursively producing branches to create leaves (terminal nodes) in which the subjects have very similar GAB's. The predicted GAB in each terminal node is the average GAB for subjects in that terminal node. This procedure creates a single regression tree. A random forest can consist of several hundred or several thousand such trees.

Classification can be made according to predictive modeling methods that set a threshold for determining the probability that a sample belongs to a given class. The probability preferably is at least 50%, or at least 60%, or at least 70%, or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUROC (area under the ROC curve) or accuracy, of a particular value, or range of values. Area under the curve measures are useful for comparing the accuracy of a classifier across the complete data range. Classifiers with a greater AUC have a greater capacity to classify unknowns correctly between two groups of interest. In some embodiments, a desired quality threshold is a predictive model that will classify a sample with an accuracy of at least about 0.5, at least about 0.55, at least about 0.6, at least about 0.7, at least about 0.75, at least about 0.8, at least about 0.85, at least about 0.9, at least about 0.95, or higher. As an alternative measure, a desired quality threshold can refer to a predictive model that will classify a sample with an AUC of at least about 0.7, at least about 0.75, at least about 0.8, at least about 0.85, at least about 0.9, or higher.

As is known in the art, the relative sensitivity and specificity of a predictive model can be adjusted to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about 0.7, at least about 0.75, at least about 0.8, at least about 0.85, at least about 0.9, or higher.

The raw data can be initially analyzed by measuring the values for each biomarker, usually in triplicate or in multiple triplicates. The data can be manipulated, for example, raw data can be transformed using standard curves, and the average of triplicate measurements used to calculate the average and standard deviation for each patient. These values can be transformed before being used in the models, e.g. log-transformed, Box-Cox transformed (Box and Cox, Royal Stat. Soc., Series B, 26:211-246(1964). The data are then input into a predictive model, which will classify the sample according to the state. The resulting information can be communicated to a patient or health care provider.

To generate a predictive model for preterm birth, a robust data set, comprising known control samples and samples corresponding to the preterm birth classification of interest is used in a training set. A sample size can be selected using generally accepted criteria. As discussed above, different statistical methods can be used to obtain a highly accurate predictive model. Examples of such analysis are provided in Example 2.

In one embodiment, hierarchical clustering is performed in the derivation of a predictive model, where the Pearson correlation is employed as the clustering metric. One approach is to consider a preterm birth dataset as a “learning sample” in a problem of “supervised learning.” CART is a standard in applications to medicine (Singer, Recursive Partitioning in the Health Sciences, Springer (1999)) and can be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

This approach led to what is termed FlexTree (Huang, Proc. Nat. Acad. Sci. U.S.A. 101:10529-10534(2004)). FlexTree performs very well in simulations and when applied to multiple forms of data and is useful for practicing the claimed methods. Software automating FlexTree has been developed. Alternatively, LARTree or LART can be used (Turnbull (2005) Classification Trees with Subset Analysis Selection by the Lasso, Stanford University). The name reflects binary trees, as in CART and FlexTree; the lasso, as has been noted; and the implementation of the lasso through what is termed LARS by Efron et al. (2004) Annals of Statistics 32:407-451 (2004). See, also, Huang et al., Proc. Natl. Acad. Sci. U.S.A. 101(29):10529-34 (2004). Other methods of analysis that can be used include logic regression. One method of logic regression Ruczinski, Journal of Computational and Graphical Statistics 12:475-512 (2003). Logic regression resembles CART in that its classifier can be displayed as a binary tree. It is different in that each node has Boolean statements about features that are more general than the simple “and” statements produced by CART.

Another approach is that of nearest shrunken centroids (Tibshirani, Proc. Natl. Acad. Sci. U.S.A. 99:6567-72 (2002)). The technology is k-means-like, but has the advantage that by shrinking cluster centers, one automatically selects features, as is the case in the lasso, to focus attention on small numbers of those that are informative. The approach is available as PAM software and is widely used. Two further sets of algorithms that can be used are random forests (Breiman, Machine Learning 45:5-32 (2001)) and MART (Hastie, The Elements of Statistical Learning, Springer (2001)). These two methods are known in the art as “committee methods,” that involve predictors that “vote” on outcome.

To provide significance ordering, the false discovery rate (FDR) can be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (Tusher et al., Proc. Natl. Acad. Sci. U.S.A 98, 5116-21 (2001)). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value can be applied to the correlations between experimental profiles. Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pair wise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

In an alternative analytical approach, variables chosen in the cross-sectional analysis are separately employed as predictors in a time-to-event analysis (survival analysis), where the event is the occurrence of preterm birth, and subjects with no event are considered censored at the time of giving birth. Given the specific pregnancy outcome (preterm birth event or no event), the random lengths of time each patient will be observed, and selection of proteomic and other features, a parametric approach to analyzing survival can be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and corresponding functions are available with this model.

In addition the Cox models can be used, especially since reductions of numbers of covariates to manageable size with the lasso will significantly simplify the analysis, allowing the possibility of a nonparametric or semi-parametric approach to prediction of time to preterm birth. These statistical tools are known in the art and applicable to all manner of proteomic data. A set of biomarker, clinical and genetic data that can be easily determined, and that is highly informative regarding the probability for preterm birth and predicted time to a preterm birth event in said pregnant female is provided. Also, algorithms provide information regarding the probability for preterm birth in the pregnant female.

Accordingly, one skilled in the art understands that the probability for preterm birth according to the invention can be determined using either a quantitative or a categorical variable. For example, in practicing the methods of the invention the measurable feature of each of N biomarkers can be subjected to categorical data analysis to determine the probability for preterm birth as a binary categorical outcome. Alternatively, the methods of the invention may analyze the measurable feature of each of N biomarkers by initially calculating quantitative variables, in particular, predicted gestational age at birth. The predicted gestational age at birth can subsequently be used as a basis to predict risk of preterm birth. By initially using a quantitative variable and subsequently converting the quantitative variable into a categorical variable the methods of the invention take into account the continuum of measurements detected for the measurable features. For example, by predicting the gestational age at birth rather than making a binary prediction of preterm birth versus term birth, it is possible to tailor the treatment for the pregnant female. For example, an earlier predicted gestational age at birth will result in more intensive prenatal intervention, i.e. monitoring and treatment, than a predicted gestational age that approaches full term.

Among women with a predicted GAB of j days plus or minus k days, p(PTB) can estimated as the proportion of women in the PAPR clinical trial (see Example 1) with a predicted GAB of j days plus or minus k days who actually deliver before 37 weeks gestational age. More generally, for women with a predicted GAB of j days plus or minus k days, the probability that the actual gestational age at birth will be less than a specified gestational age, p(actual GAB<specified GAB), was estimated as the proportion of women in the PAPR clinical trial with a predicted GAB of j days plus or minus k days who actually deliver before the specified gestational age.

In the development of a predictive model, it can be desirable to select a subset of markers, i.e. at least 3, at least 4, at least 5, at least 6, up to the complete set of markers. Usually a subset of markers will be chosen that provides for the needs of the quantitative sample analysis, e.g. availability of reagents, convenience of quantitation, etc., while maintaining a highly accurate predictive model. The selection of a number of informative markers for building classification models requires the definition of a performance metric and a user-defined threshold for producing a model with useful predictive ability based on this metric. For example, the performance metric can be the AUC, the sensitivity and/or specificity of the prediction as well as the overall accuracy of the prediction model.

As will be understood by those skilled in the art, an analytic classification process can use any one of a variety of statistical analytic methods to manipulate the quantitative data and provide for classification of the sample. Examples of useful methods include, without limitation, linear discriminant analysis, recursive feature elimination, a prediction analysis of microarray, a logistic regression, a CART algorithm, a FlexTree algorithm, a LART algorithm, a random forest algorithm, a MART algorithm, and machine learning algorithms. Various methods are used in a training model. The selection of a subset of markers can be for a forward selection or a backward selection of a marker subset. The number of markers can be selected that will optimize the performance of a model without the use of all the markers. One way to define the optimum number of terms is to choose the number of terms that produce a model with desired predictive ability (e.g. an AUC>0.75, or equivalent measures of sensitivity/specificity) that lies no more than one standard error from the maximum value obtained for this metric using any combination and number of terms used for the given algorithm.

In one aspect, the invention provides a method of determining probability for preterm birth in a pregnant female comprising measuring at least three pairs of biomarkers, wherein the pairs of biomarkers exhibit a change in reversal value between pregnant females at risk for preterm birth and term controls. In some embodiments, the at least three pairs of biomarkers can include any of the biomarkers set forth in Table 3. In some embodiments, the at least three pairs of biomarkers can include other biomarkers not set forth in Table 3. In some embodiments, the biomarkers are similar to the biomarkers in Table 3. In other embodiments, the at least three pairs of biomarkers can include any of the biomarkers set forth in the reversal groups in Tables 5-18, and 21-22. In some embodiments, the at least three pairs of biomarkers can include IBP4/SHBG, IBP4/TETN, EGLN/SHBG, EGLN/TETN, PRL/SHBG, or PRL/TETN. In particular embodiments, the at least three pairs of biomarkers consist of IBP4/SHBG, EGLN/TETN, and PRL/TETN.

In another aspect, the invention provides a method of determining probability for preterm birth in a pregnant female, the method comprising measuring a reversal group of biomarkers in a biological sample comprising a reversal pair and a reversal triplet, wherein the reversal group exhibits a change between pregnant females at risk for preterm birth and term controls. In some embodiments, the reversal pair of the reversal group can include IBP4/SHBG. In other embodiments, the reversal pair can include any of the isolated biomarkers set forth in Table 3. In some embodiments, the reversal triplet can include EGLN, PRL and TETN. In other embodiments, the reversal triplet can include any of the isolated biomarkers set forth in Table 3. In some embodiments, the reversal group of isolated biomarkers can include any of the reversal groups set forth in Tables 6-19, and 21-22. In some embodiments, the reversal group of isolated biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In particular embodiments, the reversal group can be (EGLN+PRL)/TETN+IBP4/SHBG.

In another aspect, the invention provides a method of determining probability for preterm birth in a pregnant female comprising measuring a reversal group of surrogate peptides in a biological sample, wherein the reversal group comprises a reversal pair and a reversal triplet, and wherein the reversal group exhibits a change between pregnant females at risk for preterm birth and term controls. In some embodiments, the reversal pair of surrogate peptides can include IBP4/SHBG. In other embodiments, the reversal pair of surrogate peptides can include any of the isolated biomarkers set forth in Table 3. In some embodiments, the reversal triplet of surrogate peptides can include EGLN, PRL and TETN. In other embodiments, the reversal triplet of surrogate peptides can include any of the isolated biomarkers set forth in Table 3. In some embodiments, the reversal group of surrogate peptides can include any of the reversal groups set forth in Tables 6-19, and 21-22. In some embodiments, the reversal group of isolated biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In particular embodiments, the reversal group of surrogate peptides can be (EGLN+PRL)/TETN+IBP4/SHBG.

In another aspect, the invention provides a method of determining probability for preterm birth in a pregnant female comprising obtaining a biological sample from the pregnant female, measuring a reversal group of biomarkers comprising a reversal pair and a reversal triplet and determining the combined reversal value of said reversal group, wherein the reversal group exhibits a change between pregnant females at risk for preterm birth and term controls. In some embodiments, the reversal pair of the reversal group can include IBP4/SHBG. In other embodiments, the reversal pair can include any of the isolated biomarkers set forth in Table 3. In some embodiments, the reversal triplet can include EGLN, PRL and TETN. In other embodiments, the reversal triplet can include any of the isolated biomarkers set forth in Table 3. In some embodiments, the reversal group of isolated biomarkers can include any of the reversal groups set forth in Tables 6-19, and 21-22. In some embodiments, the reversal group of isolated biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In particular embodiments, the reversal group can be (EGLN+PRL)/TETN+IBP4/SHBG

In another aspect, the invention provides a method of determining probability for preterm birth in a pregnant female comprising obtaining a biological sample from the pregnant female, measuring a reversal pair of biomarkers, determining the reversal value of said reversal pair, measuring a reversal triplet of biomarkers, determining the combined reversal value of said reversal triplet, combining the reversal value of the reversal pair and reversal triplet into a combined final reversal value, wherein said combined final reversal value exhibits a change between pregnant females at risk for preterm birth and term controls. In some embodiments, the reversal value of the reversal pair is measured first. In some embodiments, the combined reversal value of the reversal triplet is measured second. In some embodiments, the reversal value of the reversal pair is measure second. In some embodiments, the combined reversal value of the reversal triplet is measured first. In some embodiments, the reversal pair of the reversal group can include IBP4/SHBG. In other embodiments, the reversal pair can include any of the isolated biomarkers set forth in Table 3. In some embodiments, the reversal triplet can include EGLN, PRL and TETN. In other embodiments, the reversal triplet can include any of the isolated biomarkers set forth in Table 3. In some embodiments, the reversal group of isolated biomarkers can include any of the reversal groups set forth in Tables 6-19, and 21-22. In some embodiments, the reversal group of isolated biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In particular embodiments, the reversal group can be (EGLN+PRL)/TETN+IBP4/SHBG.

In one aspect, the invention provides a method for determining probability for perterm birth in a pregnant female comprising measuring a reversal group of biomarkers in a biological sample comprising a reversal triplet, wherein the reversal group exhibits a change between pregnant females at risk for preterm birth and term controls. In some embodiments, the reversal triplet can include EGLN, IBP4, and SHBG. In some embodiments, the reversal triplet can include PAPP2, IBP4, and SHBG. In other embodiments, the reversal triplet can include any of the isolated biomarkers set forth in Table 3. In particular embodiments, the reversal group can be (EGLN+IBP4)/SHBG or (PAPP2+IBP4)/SHBG.

In one aspect, the invention provides a method of determining probability for preterm birth in a pregnant female comprising measuring in a biological sample obtained from said pregnant female, a reversal group of surrogate peptides comprising a reversal triplet of isolated biomarkers, wherein said reversal group exhibits a change between pregnant females at risk for pre-term birth and term controls. In some embodiments, the reversal triplet can include EGLN, IBP4, and SHBG. In some embodiments, the reversal triplet can include PAPP2, IBP4, and SHBG. In other embodiments, the reversal triplet can include any of the isolated biomarkers set forth in Table 3. In particular embodiments, the reversal group of surrogate peptides can include (EGLN+IBP4)/SHBG or (PAPP2+IBP4)/SHBG.

In one aspect, the invention provides a method of determining probability for preterm birth in a pregnant female, the method comprising obtaining a biological sample from the pregnant female, measuring a reversal group of biomarkers comprising a reversal triplet of isolated biomarkers, and determining a combined reversal value of said reversal group, wherein said combined reversal value of said reversal group of biomarkers exhibits a change between pregnant females at risk for pre-term birth and term controls. In some embodiments, the reversal triplet can include EGLN, IBP4, and SHBG. In some embodiments, the reversal triplet can include PAPP2, IBP4, and SHBG. In other embodiments, the reversal triplet can include any of the isolated biomarkers set forth in Table 3. In particular embodiments, the reversal group can include (EGLN+IBP4)/SHBG or (PAPP2+IBP4)/SHBG.

In one aspect, the invention provides a method of determining probability for preterm birth in a pregnant female, the method comprising obtaining a biological sample from the pregnant female, measuring a reversal triplet of isolated biomarkers, and determining a combined reversal value of said reversal triplet, wherein said combined reversal value exhibits a change between pregnant females at risk for pre-term birth and term controls. In some embodiments, the reversal triplet can include EGLN, IBP4, and SHBG. In some embodiments, the reversal triplet can include PAPP2, IBP4, and SHBG. In other embodiments, the reversal triplet can include any of the isolated biomarkers set forth in Table 3. In particular embodiments, the reversal triplet can include (EGLN+IBP4)/SHBG or (PAPP2+IBP4)/SHBG.

In one aspect, the invention provides a method for determining a probability for preterm birth in a pregnant female comprising measuring at least two pairs of biomarkers, wherein the at least two pairs of biomarkers comprise a first reversal pair and a second reversal pair of biomarkers, wherein the pairs of biomarkers exhibit a change in reversal value between pregnant females at risk for preterm birth and term controls. In some embodiments, the at least two pairs of biomarkers can include any of the biomarkers set forth in Table 3. In some embodiments, the first reversal pair can include IBP4/SHBG. In some embodiments, the second reversal pair can include any of the reversal pairs of biomarkers set forth in Table 22. In some embodiments, the method comprises measuring a third pair of biomarkers. In some embodiments, the third pair of biomarkers can include any of the reversal pairs of biomarkers set forth in Table 22, except the third reversal pair and the second reversal pair are not the same.

In one aspect, the invention provides a method of determining probability for preterm birth in a pregnant female, the method comprising measuring a reversal group of biomarkers in a biological sample comprising a first reversal pair and a second reversal pair of biomarkers, wherein said reversal group of biomarkers exhibits a change between pregnant females at risk for pre-term birth and term controls. In some embodiments, the first reversal pair can include IBP4/SHBG. In some embodiments, the second reversal pair can include any of the reversal pairs of biomarkers set forth in Table 22. In some embodiments, the method comprises measuring a third reversal pair of biomarkers. In some embodiments, the third reversal pair can include any of the reversal pairs of biomarkers set forth in Table 22, except the third reversal pair and the second reversal pair are not the same.

In another aspect, the invention provides a method of determining probability for preterm birth in a pregnant female comprising obtaining a biological sample from the pregnant female, measuring a reversal group of biomarkers comprising a first reversal pair and second reversal pair, and determining the combined reversal value of said reversal group, wherein the reversal group exhibits a change between pregnant females at risk for preterm birth and term controls. In some embodiments, the first reversal pair of the reversal group can include IBP4/SHBG. In other embodiments, the first reversal pair can include any of the isolated biomarkers set forth in Table 3. In some embodiments, the second reversal pair can include any of the reversal pairs of biomarkers set forth in Table 22. In other embodiments, the second reversal pair can include any of the isolated biomarkers set forth in Table 3. In some embodiments, the reversal group of isolated biomarkers can include any of the reversal groups set forth in Tables 6-19, and 21-22.

In another aspect, the invention provides a method of determining probability for preterm birth in a pregnant female comprising obtaining a biological sample from the pregnant female, measuring a first reversal pair of biomarkers, determining the reversal value of said first reversal pair, measuring a second reversal pair of biomarkers, determining the reversal value of said second reversal pair of biomarkers, combining the reversal value of the first reversal pair and second reversal pair into a final reversal value, wherein said final reversal value exhibits a change between pregnant females at risk for preterm birth and term controls. In some embodiments, the reversal value of the first reversal pair is measured first. In some embodiments, the reversal value of the second reversal pair is measured second. In some embodiments, the reversal value of the first reversal pair is measure second. In some embodiments, the reversal value of the second reversal pair is measured first. In some embodiments, the first reversal pair of the reversal group can include IBP4/SHBG. In other embodiments, the first reversal pair can include any of the isolated biomarkers set forth in Table 3. In some embodiments, the second reversal pair can include any of the reversal pairs of biomarkers set forth in Table 22. In other embodiments, the second reversal pair can include any of the isolated biomarkers set forth in Table 3. In some embodiments, the reversal group of isolated biomarkers can include any of the reversal groups set forth in Tables 6-19, and 21-22. In some embodiments, the method comprises measuring a third reversal pair of biomarkers. In some embodiments, the third reversal pair can include any of the reversal pairs of biomarkers set forth in Table 22, except the third reversal pair and the second reversal pair are not the same.

In one aspect, the invention provides a method of determining probability for preterm birth in a pregnant female comprising measuring a reversal group of surrogate peptides in a biological sample comprising a first reversal pair and a second reversal pair, wherein the reversal group exhibits a change between pregnant females at risk for preterm birth and term controls. In some embodiments, the first reversal pair of surrogate peptides can include IBP4/SHBG. In some embodiments, the second reversal pair of surrogate peptides can include any of the reversal pairs of biomarkers set forth in Table 22. In some embodiments, the first and second reversal pair of surrogate peptides can include any of the isolated biomarkers set forth in Table 3. In some embodiments, the reversal group of surrogate peptides comprise a third reversal pair of biomarkers. In some embodiments, the third reversal pair can include any of the reversal pairs of biomarkers set forth in Table 22, except the third reversal pair and the second reversal pair are not the same

In one aspect, the invention provides a method of detecting at least three pairs of biomarkers in a pregnant female comprising obtaining a biological sample from the pregnant female and detecting whether the at least three pairs of biomarkers are present in the biological sample, wherein said pairs of biomarkers exhibit a change in reversal value between pregnant females at risk for pre-term birth and term controls. In some embodiments, the at least three pairs of biomarkers can include any of the biomarkers set forth in Table 3. In other embodiments, the at least three pairs of biomarkers can include any of the biomarkers set forth in the reversal groups in Tables 5-19, and 21-22. In some embodiments, the at least three pairs of isolated biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In some embodiments, the at least three pairs of biomarkers can include IBP4/SHBG, IBP4/TETN, EGLN/SHBG, EGLN/TETN, PRL/SHBG, or PRL/TETN. In particular embodiments, the at least three pairs of biomarkers consist of IBP4/SHBG, EGLN/TETN, and PRL/TETN.

In another aspect, the invention provides a method of detecting a reversal group of biomarkers comprising a reversal pair of biomarkers and a reversal triplet of biomarkers in a pregnant female comprising obtaining a biological sample from said pregnant female and detecting whether the reversal group of biomarkers is present in the biological sample, wherein said detecting comprises subjecting said sample to a proteomics work-flow comprised of mass spectrometry (MS) quantification. In some embodiments, the reversal pair of the reversal group can include IBP4/SHBG. In other embodiments, the reversal pair can include any of the isolated biomarkers set forth in Table 3. In some embodiments, the reversal triplet can include EGLN, PRL and TETN. In other embodiments, the reversal triplet can include any of the isolated biomarkers set forth in Table 3. In some embodiments, the reversal group of isolated biomarkers can include any of the reversal groups set forth in Tables 6-19, and 21-22. In some embodiments, the reversal group of isolated biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In particular embodiments, the reversal group can be (EGLN+PRL)/TETN+IBP4/SHBG.

In another aspect, the invention provides a method of detecting a reversal group of biomarkers comprising a reversal pair and a reversal triplet of biomarkers in a pregnant female comprising obtaining a biological sample from said pregnant female, first detecting whether the reversal pair of biomarkers is present in the biological sample, second detecting whether the reversal triplet of biomarkers is present in the biological sample, wherein said first detecting step and/or said second detecting step comprises subjecting said sample to a proteomics work-flow comprised of mass spectrometry (MS) quantification. In some embodiments, the reversal pair of the reversal group can include IBP4/SHBG. In other embodiments, the reversal pair can include any of the isolated biomarkers set forth in Table 3. In some embodiments, the reversal triplet can include EGLN, PRL and TETN. In other embodiments, the reversal triplet can include any of the isolated biomarkers set forth in Table 3. In some embodiments, the reversal group of isolated biomarkers can include any of the reversal groups set forth in Tables 6-19, and 21-22. In some embodiments, the reversal group of isolated biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In particular embodiments, the reversal group can be (EGLN+PRL)/TETN+IBP4/SHBG.

In one aspect, the invention provides a method of detecting at least two pairs of biomarkers in a pregnant female comprising obtaining a biological sample from the pregnant female and detecting whether the at least two pairs of biomarkers are present in the biological sample, wherein said pairs of biomarkers exhibit a change in reversal value between pregnant females at risk for pre-term birth and term controls. In some embodiments, the at least two pairs of biomarkers comprise a first reversal pair and a second reversal pair of biomarkers. In some embodiments, the at least two pairs of biomarkers can include any of the biomarkers set forth in Table 3. In some embodiments, the first reversal pair of biomarkers can include IBP4/SHBG. In some embodiments, the second reversal pair can include any of the reversal pairs of biomarkers set forth in Table 22. In other embodiments, the at least two pairs of biomarkers can include any of the biomarkers set forth in the reversal groups in Tables 5-19, and 21-22. In some embodiments, the method comprises detecting a third reversal pair of biomarkers. In some embodiments, the third reversal pair can include any of the reversal pairs of biomarkers set forth in Table 22, except the third biomarker pair and the second biomarker pair are not the same.

In another aspect, the invention provides a method of detecting a reversal group of biomarkers comprising a first reversal pair of biomarkers and a second reversal pair of biomarkers in a pregnant female comprising obtaining a biological sample from said pregnant female and detecting whether the reversal group of biomarkers is present in the biological sample, wherein said detecting comprises subjecting said sample to a proteomics work-flow comprised of mass spectrometry (MS) quantification. In some embodiments, the first reversal pair of the reversal group can include IBP4/SHBG. In other embodiments, the first reversal pair can include any of the isolated biomarkers set forth in Table 3. In some embodiments, the second reversal pair can include any of the reversal pairs of biomarkers set forth in Table 22. In other embodiments, the second reversal pair can include any of the isolated biomarkers set forth in Table 3. In some embodiments, the reversal group of isolated biomarkers can include any of the reversal groups set forth in Tables 6-19, and 21-22. In some embodiments, the method comprises detecting a third reversal pair of biomarkers. In some embodiments, the third reversal pair of biomarkers can include any of the reversal pairs of biomarkers set forth in Table 22, except the third reversal pair and the second reversal pair are not the same.

In another aspect, the invention provides a method of detecting a reversal group of biomarkers comprising a first reversal pair of biomarkers and a second reversal pair of biomarkers in a pregnant female comprising obtaining a biological sample from said pregnant female, first detecting whether the first reversal pair of biomarkers is present in the biological sample, second detecting whether the second reversal pair of biomarkers is present in the biological sample, wherein said first detecting step and/or said second detecting step comprises subjecting said sample to a proteomics work-flow comprised of mass spectrometry (MS) quantification. In some embodiments, the first reversal pair of the reversal group can include IBP4/SHBG. In other embodiments, the first reversal pair can include any of the isolated biomarkers set forth in Table 3. In some embodiments, the second reversal pair can include any of the reversal pairs of biomarkers set forth in Table 22. In other embodiments, the second reversal pair can include any of the isolated biomarkers set forth in Table 3. In some embodiments, the reversal group of isolated biomarkers can include any of the reversal groups set forth in Tables 6-19, and 21-22. In some embodiments, the method comprises detecting a reversal group of biomarkers comprising a third reversal pair. In some embodiments, the third reversal pair can include any of the reversal pairs of biomarkers set forth in Table 22, except the third reversal pair and the second reversal pair are not the same.

In another aspect, the invention provides a method of detecting a reversal group of biomarkers comprising a reversal triplet of biomarkers in a pregnant female comprising obtaining a biological sample from said pregnant female and detecting whether the reversal group of biomarkers is present in the biological sample, wherein said detecting comprises subjecting said sample to a proteomics work-flow comprised of mass spectrometry (MS) quantification. In some embodiments, the reversal triplet can include EGLN, IBP4, and SHBG. In some embodiments, the reversal triplet can include PAPP2, IBP4, and SHBG. In other embodiments, the reversal triplet can include any of the isolated biomarkers set forth in Table 3. In particular embodiments, the reversal triplet can be ((EGLN+IBP4)/SHBG or (PAPP2+IBP4)/SHBG.

In another aspect, the invention provides a method of detecting a reversal group of biomarkers comprising a reversal triplet of biomarkers in a pregnant female comprising obtaining a biological sample from said pregnant female, first detecting whether the reversal triplet of biomarkers is present in the biological sample, wherein said detecting step comprises subjecting said sample to a proteomics work-flow comprised of mass spectrometry (MS) quantification. In some embodiments, the reversal triplet can include EGLN, IBP4, and SHBG. In some embodiments, the reversal triplet can include PAPP2, IBP4, and SHBG. In other embodiments, the reversal triplet can include any of the isolated biomarkers set forth in Table 3. In particular embodiments, the reversal triplet can be ((EGLN+IBP4)/SHBG or (PAPP2+IBP4)/SHBG.

In another aspect, the invention provides methods of treating or preventing preterm birth in a pregnant female comprising:
(a) obtaining a biological sample from said pregnant female;
(b) detecting a reversal group of biomarkers in said sample;
(c) providing a risk score for said pregnant female;
(d) prognosing said pregnant female as having an increased risk of preterm birth; and
(e) administering one or more therapies to said pregnant female to prevent preterm birth.

In some embodiments, the providing a risk score comprises receiving a report communicating the relevant status (e.g., risk of preterm birth). In some embodiments this report communicates such status in a qualitative manner (e.g., “high” or “increased” risk). In some embodiments, this report communicates such status indirectly by communicating a score, such as a risk score, that incorporates status.

In some embodiments, the method comprises a reversal group comprising a reversal pair and a reversal triplet of biomarkers. In some embodiments the reversal pair comprises IBP4/SHBG and the reversal triplet comprises (EGLN+PRL)/TETN.

In some embodiments, the method comprises a reversal group comprising a reversal triplet of biomarkers. In some embodiments, the reversal triplet comprises (EGLN+IBP4)/SHBG or (PAPP2+IBP4)/SHBG.

In some embodiments, the method comprises a reversal group comprising at least two reversal pairs of biomarkers. In some embodiments, the two reversal pairs of biomarkers comprise a first reversal pair and a second reversal pair of biomarkers. In some embodiments, the first reversal pair comprises IBP4. In some embodiments, the second reversal pair comprises any reversal pair of biomarkers set forth in Table 22. In some embodiments, the reversal group comprises third reversal pair. In other embodiments, the third reversal pair comprises any reversal pair of biomarkers set forth in Table 22, except the third reversal pair and the second reversal pair are not the same.

In some embodiments, the therapy includes a surgical procedure (e.g., cervical cerclage). In other embodiments, the therapy comprises administering 17-α hydroxyprogesterone caproate, vaginal progesterone gel, antenatal corticosteroids or cervical pessaries or cerclage. In other embodiments, the therapy includes elevated care (interchangeably referred to as care coordination, care management, case management or high intensity care/case management) that may involve more frequent examination and/or counseling. Elevated care many enable more timely and effective administration of the procedures mentioned above.

In one aspect, the invention provides a method of detecting at least three pairs of biomarkers in a pregnant female, said method comprising, obtaining a biological sample from said pregnant female and detecting whether the pair of isolated biomarkers is present in the biological sample by contacting the biological sample with a first capture agent that specifically binds a first member of said pair and a second capture agent that specifically binds a second member of said pair and detecting binding between the first biomarker of said pair and the first capture agent and between the second member of said pair and the second capture agent, wherein said detecting comprises an assay that utilizes the capture agent. In some embodiments, the at least three pairs of biomarkers can include any of the biomarkers set forth in Table 3. In other embodiments, the at least three pairs of biomarkers can include any of the biomarkers set forth in the reversal groups in Tables 5-19, and 21-22. In some embodiments, the at least three pairs of biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In some embodiments, the at least three pairs of biomarkers can include IBP4/SHBG, IBP4/TETN, EGLN/SHBG, EGLN/TETN, PRL/SHBG, or PRL/TETN. In particular embodiments, the at least three pairs of biomarkers consist of IBP4/SHBG, EGLN/TETN, and PRL/TETN.

In one aspect, the invention provides a method of detecting a reversal group of biomarkers comprising a reversal pair of biomarkers and a reversal triplet of biomarkers in a pregnant female, said method comprising obtaining a biological sample from the pregnant female, detecting whether the reversal group of biomarkers is present in the biological sample by contacting the biological sample with a capture agent that specifically binds an individual biomarker of the reversal group; and detecting binding between the capture agent and the individual biomarker of the reversal group wherein said detecting comprises an assay that utilizes the capture agent. In some embodiments, the reversal pair of the reversal group can include IBP4/SHBG. In other embodiments, the reversal pair can include any of the isolated biomarkers set forth in Table 3. In some embodiments, the reversal triplet can include EGLN, PRL and TETN. In other embodiments, the reversal triplet can include any of the isolated biomarkers set forth in Table 3. In some embodiments, the reversal group of isolated biomarkers can include any of the reversal groups set forth in Tables 6-19, and 21-22. In some embodiments, the reversal group of isolated biomarkers can include one or more biomarkers of ADA12, APOC3, ATS13, CFAB, CHL1, IBP1, EGLN, PGRP2, PRL, PSG3, TETN, or TIMP and/or one or more of CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, or CAMP. In particular embodiments, the reversal group can be (EGLN+PRL)/TETN+IBP4/SHBG.

In one aspect, the invention provides a method of detecting at least two pairs of biomarkers in a pregnant female, said method comprising, obtaining a biological sample from said pregnant female and detecting whether the pair of isolated biomarkers is present in the biological sample by contacting the biological sample with a first capture agent that specifically binds a first member of said pair and a second capture agent that specifically binds a second member of said pair and detecting binding between the first biomarker of said pair and the first capture agent and between the second member of said pair and the second capture agent, wherein said detecting comprises an assay that utilizes the capture agent. In some embodiments, the at least two pairs of biomarkers comprise a first reversal pair and a second reversal pair of biomarkers. In some embodiments, the first reversal pair can include IBP4/SHBG. In some embodiments, the second reversal pair can include any of the reversal pairs of biomarkers set forth in Table 22. In some embodiments, the at least two pairs of biomarkers can include any of the isolated biomarkers set forth in Table 3. In other embodiments, the at least two pairs of biomarkers can include any of the biomarkers set forth in the reversal groups in Tables 5-19, and 21-22. In some embodiments, the method comprises detecting a third pair of biomarkers. In some embodiments, the third pair of biomarkers can include any of the reversal pairs of biomarkers set forth in Table 22, except the third pair of biomarkers and second pair of biomarkers are not the same.

In one aspect, the invention provides a method of detecting a reversal group of biomarkers comprising a first reversal pair of biomarkers and a second reversal pair of biomarkers in a pregnant female, said method comprising obtaining a biological sample from the pregnant female, detecting whether the reversal group of biomarkers is present in the biological sample by contacting the biological sample with a capture agent that specifically binds an individual biomarker of the reversal group, and detecting binding between the capture agent and the individual biomarker of the reversal group wherein said detecting comprises an assay that utilizes the capture agent. In some embodiments, the first reversal pair of the reversal group can include IBP4/SHBG. In other embodiments, the reversal pair can include any of the isolated biomarkers set forth in Table 3. In some embodiments, the second reversal pair can include any of the reversal pairs of biomarkers set forth in Table 22. In other embodiments, the second reversal pair can include any of the isolated biomarkers set forth in Table 3. In some embodiments, the reversal group of isolated biomarkers can include any of the reversal groups set forth in Tables 6-19 and 21-22. In some embodiments, the reversal group of isolated biomarkers can include a third reversal pair of biomarkers. In some embodiments, the third reversal pair can include any of the isolated biomarkers set forth in Table 22, except the third reversal pair and the second reversal pair are not the same.

A method of detecting a reversal group of biomarkers comprising a reversal triplet of biomarkers in a pregnant female, said method comprising obtaining a biological sample from the pregnant female, detecting whether the reversal group of biomarkers is present in the biological sample by contacting the biological sample with a capture agent that specifically binds an individual biomarker of the reversal group; and detecting binding between the capture agent and the individual biomarker of the reversal group wherein said detecting comprises an assay that utilizes the capture agent. In some embodiments, the reversal triplet can include EGLN, IBP4 and SHBG. In other embodiments, the reversal triplet can include any of the isolated biomarkers set forth in Table 3. In particular embodiments, the reversal group can be (EGLN+IBP4)/SHBG or (PAPP2+IBP4)/SHBG.

Although described with reference to protein biomarkers, changes in reversal values or combined reversal values can be identified in protein or gene expression levels for pairs of biomarkers. In addition to the specific biomarkers disclosed herein, the invention further includes biomarker variants that are about 90%, about 95%, or about 97% identical to the exemplified sequences in Table 3 that are now known or later discovered and that have utility for the methods of the invention. Variants, as used herein, include polymorphisms, splice variants, mutations, and the like. Variants can also include post-translational modifications. In this regard, the instant specification discloses multiple art-known proteins in the context of the invention and provides exemplary accession numbers associated with one or more public databases as well as exemplary references to published journal articles relating to these art-known proteins. However, those skilled in the art appreciate that additional accession numbers and journal articles can easily be identified that can provide additional characteristics of the disclosed biomarkers and that the exemplified references are in no way limiting with regard to the disclosed biomarkers. As described herein, various techniques and reagents find use in the methods of the present invention. Suitable samples in the context of the present invention include, for example, blood, plasma, serum, amniotic fluid, vaginal secretions, saliva, and urine. In some embodiments, the biological sample is selected from the group consisting of whole blood, plasma, and serum. In a particular embodiment, the biological sample is serum. As described herein, biomarkers can be detected through a variety of assays and techniques known in the art. As further described herein, such assays include, without limitation, mass spectrometry (MS)-based assays, antibody-based assays as well as assays that combine aspects of the two.

As disclosed above, the methods of detecting or determining probability for preterm birth comprises an initial step of measuring biomarker pairs, reversal pairs or reversal triplets. Any existing, available or conventional separation, detection and quantification methods can be used herein to measure the presence or absence (e.g., readout being present vs. absent; or detectable amount vs. undetectable amount) and/or quantity (e.g., readout being an absolute or relative quantity, such as, for example, absolute or relative concentration) of biomarkers, peptides, polypeptides, proteins and/or fragments thereof and optionally of the one or more other biomarkers or fragments thereof in samples. In some embodiments, detection and/or quantification of one or more biomarkers comprises an assay that utilizes a capture agent. In further embodiments, the capture agent is an antibody, antibody fragment, nucleic acid-based protein binding reagent, small molecule or variant thereof. In additional embodiments, the assay is an enzyme immunoassay (EIA), enzyme-linked immunosorbent assay (ELISA), and radioimmunoassay (RIA). In some embodiments, detection and/or quantification of one or more biomarkers further comprises mass spectrometry (MS). In yet further embodiments, the mass spectrometry is co-immunoprecipitation-mass spectrometry (co-IP MS), where co-immunoprecipitation, a technique suitable for the isolation of whole protein complexes is followed by mass spectrometric analysis.

In some embodiments, measuring surrogate peptides that correspond to said biomarker pairs, reversal pairs or reversal triplets. Surrogate peptides can correspond to any of the biomarkers disclosed herein including Tables 2, 5-18, and 21-22. In other embodiments, measuring comprises measuring stable isotope labeled standard peptides for each of the surrogate peptides.

In some embodiments, measuring biomarkers, reversal pairs or reversal triplets for determining probability of preterm birth comprises mass spectrometry (MS). In some embodiments, MS comprises matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS), APPI-MS/MS; or APPI-(MS)n. In additional embodiments, MS comprises co-immunoprecipitation MS (co-IP MS), LC-MS, MRM or SRM.

Generally, any mass spectrometric (MS) technique that can provide precise information on the mass of peptides, and preferably also on fragmentation and/or (partial) amino acid sequence of selected peptides (e.g., in tandem mass spectrometry, MS/MS; or in post source decay, TOF MS), can be used in the methods disclosed herein. Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000; Biemann 1990. Methods Enzymol 193: 455-79; or Methods in Enzymology, vol. 402: “Biological Mass Spectrometry”, by Burlingame, ed., Academic Press 2005) and can be used in practicing the methods disclosed herein. Accordingly, in some embodiments, the disclosed methods comprise performing quantitative MS to measure one or more biomarkers. Such quantitative methods can be performed in an automated (Villanueva, et al., Nature Protocols (2006) 1(2):880-891) or semi-automated format. In particular embodiments, MS can be operably linked to a liquid chromatography device (LC-MS/MS or LC-MS) or gas chromatography device (GC-MS or GC-MS/MS). Other methods useful in this context include isotope-coded affinity tag (ICAT), tandem mass tags (TMT), or stable isotope labeling by amino acids in cell culture (SILAC), followed by chromatography and MS/MS.

Mass spectrometry assays, instruments and systems suitable for biomarker peptide analysis can include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)ₙ (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS); APPI-MS/MS; and APPI-(MS)n. Peptide ion fragmentation in tandem MS (MS/MS) arrangements can be achieved using manners established in the art, such as, e.g., collision induced dissociation (CID). As described herein, detection and quantification of biomarkers by mass spectrometry can involve multiple reaction monitoring (MRM), such as described among others by Kuhn et al. Proteomics 4: 1175-86 (2004). Scheduled multiple-reaction-monitoring (Scheduled MRM) mode acquisition during LC-MS/MS analysis enhances the sensitivity and accuracy of peptide quantitation. Anderson and Hunter, Molecular and Cellular Proteomics 5(4):573 (2006). As described herein, mass spectrometry-based assays can be advantageously combined with upstream peptide or protein separation or fractionation methods, such as for example with the chromatographic and other methods described herein below. As further described herein, shotgun quantitative proteomics can be combined with SRM/MRM-based assays for high-throughput identification and verification of prognostic biomarkers of preterm birth.

A person skilled in the art will appreciate that a number of methods can be used to determine the amount of a biomarker, including mass spectrometry approaches, such as MS/MS, LC-MS/MS, multiple reaction monitoring (MRM) or SRM and production monitoring (PIM) and also including antibody based methods such as immunoassays such as Western blots, enzyme-linked immunosorbant assay (ELISA), immunoprecipitation, immunohistochemistry, immunofluorescence, radioimmunoassay, dot blotting, and FACS. Accordingly, in some embodiments, determining the level of the at least one biomarker comprises using an immunoassay and/or mass spectrometric methods. In additional embodiments, the mass spectrometric methods are selected from MS, MS/MS, LC-MS/MS, SRM, PIM, and other such methods that are known in the art. In other embodiments, LC-MS/MS further comprises 1D LC-MS/MS, 2D LC-MS/MS or 3D LC-MS/MS. Immunoassay techniques and protocols are generally known to those skilled in the art (Price and Newman, Principles and Practice of Immunoassay, 2nd Edition, Grove's Dictionaries, 1997; and Gosling, Immunoassays: A Practical Approach, Oxford University Press, 2000.) A variety of immunoassay techniques, including competitive and non-competitive immunoassays, can be used (Self et al., Curr. Opin. Biotechnol., 7:60-65 (1996).

In further embodiments, the immunoassay is selected from Western blot, ELISA, immunoprecipitation, immunohistochemistry, immunofluorescence, radioimmunoassay (RIA), dot blotting, and FACS. In certain embodiments, the immunoassay is an ELISA. In yet a further embodiment, the ELISA is direct ELISA (enzyme-linked immunosorbent assay), indirect ELISA, sandwich ELISA, competitive ELISA, multiplex ELISA, ELISPOT technologies, and other similar techniques known in the art. Principles of these immunoassay methods are known in the art, for example John R. Crowther, The ELISA Guidebook, 1st ed., Humana Press 2000, ISBN 0896037282. Typically ELISAs are performed with antibodies but they can be performed with any capture agents that bind specifically to one or more biomarkers of the invention and that can be detected. Multiplex ELISA allows simultaneous detection of two or more analytes within a single compartment (e.g., microplate well) usually at a plurality of array addresses (Nielsen and Geierstanger 2004. J Immunol Methods 290: 107-20 (2004) and Ling et al. 2007. Expert Rev Mol Diagn 7: 87-98 (2007)).

In some embodiments, Radioimmunoassay (RIA) can be used to detect one or more biomarkers in the methods of the invention. RIA is a competition-based assay that is well known in the art and involves mixing known quantities of radioactively-labelled (e.g., ¹²⁵I or ¹³¹I-labelled) target analyte with antibody specific for the analyte, then adding non-labeled analyte from a sample and measuring the amount of labeled analyte that is displaced (see, e.g., An Introduction to Radioimmunoassay and Related Techniques, by Chard T, ed., Elsevier Science 1995, ISBN 0444821198 for guidance).

A detectable label can be used in the assays described herein for direct or indirect detection of the biomarkers in the methods of the invention. A wide variety of detectable labels can be used, with the choice of label depending on the sensitivity required, ease of conjugation with the antibody, stability requirements, and available instrumentation and disposal provisions. Those skilled in the art are familiar with selection of a suitable detectable label based on the assay detection of the biomarkers in the methods of the invention. Suitable detectable labels include, but are not limited to, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), enzymes (e.g., luciferase, horseradish peroxidase, alkaline phosphatase, etc.), nanoparticles, biotin, digoxigenin, metals, and the like.

For mass-spectrometry based analysis, differential tagging with isotopic reagents, e.g., isotope-coded affinity tags (ICAT) or the more recent variation that uses isobaric tagging reagents, iTRAQ (Applied Biosystems, Foster City, Calif), or tandem mass tags, TMT, (Thermo Scientific, Rockford, IL), followed by multidimensional liquid chromatography (LC) and tandem mass spectrometry (MS/MS) analysis can provide a further methodology in practicing the methods of the invention.

A chemiluminescence assay using a chemiluminescent antibody can be used for sensitive, non-radioactive detection of protein levels. An antibody labeled with fluorochrome also can be suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), beta-galactosidase, urease, and the like. Detection systems using suitable substrates for horseradish-peroxidase, alkaline phosphatase, and beta-galactosidase are well known in the art.

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif) in accordance with the manufacturer's instructions. If desired, assays used to practice the invention can be automated or performed robotically, and the signal from multiple samples can be detected simultaneously.

In some embodiments, the methods described herein encompass quantification of the biomarkers using mass spectrometry (MS). In further embodiments, the mass spectrometry can be liquid chromatography-mass spectrometry (LC-MS), multiple reaction monitoring (MRM) or selected reaction monitoring (SRM). In additional embodiments, the MRM or SRM can further encompass scheduled MRM or scheduled SRM.

As described above, chromatography can also be used in practicing the methods of the invention. Chromatography encompasses methods for separating chemical substances and generally involves a process in which a mixture of analytes is carried by a moving stream of liquid or gas (“mobile phase”) and separated into components as a result of differential distribution of the analytes as they flow around or over a stationary liquid or solid phase (“stationary phase”), between the mobile phase and said stationary phase. The stationary phase can be usually a finely divided solid, a sheet of filter material, or a thin film of a liquid on the surface of a solid, or the like. Chromatography is well understood by those skilled in the art as a technique applicable for the separation of chemical compounds of biological origin, such as, e.g., amino acids, proteins, fragments of proteins or peptides, etc.

Chromatography can be columnar (i.e., wherein the stationary phase is deposited or packed in a column), preferably liquid chromatography, and yet more preferably high-performance liquid chromatography (HPLC), or ultra high performance/pressure liquid chromatography (UHPLC). Particulars of chromatography are well known in the art (Bidlingmeyer, Practical HPLC Methodology and Applications, John Wiley & Sons Inc., 1993). Exemplary types of chromatography include, without limitation, high-performance liquid chromatography (HPLC), UHPLC, normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilized metal affinity chromatography, and the like. Chromatography, including single-, two- or more-dimensional chromatography, can be used as a peptide fractionation method in conjunction with a further peptide analysis method, such as for example, with a downstream mass spectrometry analysis as described elsewhere in this specification.

Further peptide or polypeptide separation, identification or quantification methods can be used, optionally in conjunction with any of the above described analysis methods, for measuring biomarkers in the present disclosure. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

In other embodiments, measuring biomarkers, reversal pairs or reversal triplets for determining probability of preterm birth comprises an assay that utilizes a capture agent. Capture agents can be configured to specifically bind to a target, in particular a biomarker. Capture agents can include but are not limited to organic molecules, such as polypeptides, polynucleotides and other non polymeric molecules that are identifiable to a skilled person. In the embodiments disclosed herein, capture agents include any agent that can be used to detect, purify, isolate, or enrich a target, in particular a biomarker. Any art-known affinity capture technologies can be used to selectively isolate and enrich or concentrate biomarkers that are components of complex mixtures of biological media for use in the disclosed methods. In some embodiments, the assay comprises EIA, ELISA or RIA. In some embodiments, the capture agent comprises an antibody, antibody fragment, nucleic acid-based protein binding reagent, small molecule or variant thereof.

Antibody capture agents that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986). Antibody capture agents can be any immunoglobulin or derivative thereof, whether natural or wholly or partially synthetically produced. All derivatives thereof which maintain specific binding ability are also included in the term. Antibody capture agents have a binding domain that is homologous or largely homologous to an immunoglobulin binding domain and can be derived from natural sources, or partly or wholly synthetically produced. Antibody capture agents can be monoclonal or polyclonal antibodies. In some embodiments, an antibody is a single chain antibody. Those of ordinary skill in the art will appreciate that antibodies can be provided in any of a variety of forms including, for example, humanized, partially humanized, chimeric, chimeric humanized, etc. Antibody capture agents can be antibody fragments including, but not limited to, Fab, Fab′, F(ab′)2, scFv, Fv, dsFv diabody, and Fd fragments. An antibody capture agent can be produced by any means. For example, an antibody capture agent can be enzymatically or chemically produced by fragmentation of an intact antibody and/or it can be recombinantly produced from a gene encoding the partial antibody sequence. An antibody capture agent can comprise a single chain antibody fragment. Alternatively or additionally, antibody capture agent can comprise multiple chains which are linked together, for example, by disulfide linkages; and, any functional fragments obtained from such molecules, wherein such fragments retain specific-binding properties of the parent antibody molecule. Because of their smaller size as functional components of the whole molecule, antibody fragments can offer advantages over intact antibodies for use in certain immunochemical techniques and experimental applications.

Suitable capture agents useful for practicing the invention also include aptamers. Aptamers are oligonucleotide sequences that can bind to their targets specifically via unique three dimensional (3-D) structures. An aptamer can include any suitable number of nucleotides and different aptamers can have either the same or different numbers of nucleotides. Aptamers can be DNA or RNA or chemically modified nucleic acids and can be single stranded, double stranded, or contain double stranded regions, and can include higher ordered structures. An aptamer can also be a photoaptamer, where a photoreactive or chemically reactive functional group is included in the aptamer to allow it to be covalently linked to its corresponding target. Use of an aptamer capture agent can include the use of two or more aptamers that specifically bind the same biomarker. An aptamer can include a tag. An aptamer can be identified using any known method, including the SELEX (systematic evolution of ligands by exponential enrichment), process. Once identified, an aptamer can be prepared or synthesized in accordance with any known method, including chemical synthetic methods and enzymatic synthetic methods and used in a variety of applications for biomarker detection. Liu et al., Curr Med Chem. 18(27):4117-25 (2011). Capture agents useful in practicing the methods of the invention also include SOMAmers (Slow Off-Rate Modified Aptamers) known in the art to have improved off-rate characteristics. Brody et al., J Mol Biol. 422(5):595-606 (2012). SOMAmers can be generated using any known method, including the SELEX method.

In some embodiments, the use of a capture agent can be combined with mass spectrometry as the detection step. In some embodiments, the combined detection step comprises affinity capture MS (AC-MS). Use of a capture agent prior to detection by MS greatly reduces sample complexity, allowing for shorter chromatographic separations that increase throughput. In yet further embodiments, the combined detection step comprises co-immunoprecipitation-mass spectrometry (co-IP MS), where co-immunoprecipitation, a technique suitable for the isolation of whole protein complexes, is followed by mass spectrometric analysis.

It is understood by those skilled in the art that biomarkers can be modified prior to analysis to improve their resolution or to determine their identity. For example, the biomarkers can be subject to proteolytic digestion before analysis. Any protease can be used. Proteases, such as trypsin, that are likely to cleave the biomarkers into a discrete number of fragments are particularly useful. The fragments that result from digestion function as a fingerprint for the biomarkers, thereby enabling their detection indirectly. This is particularly useful where there are biomarkers with similar molecular masses that might be confused for the biomarker in question. Also, proteolytic fragmentation is useful for high molecular weight biomarkers because smaller biomarkers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the biomarkers can be modified by the attachment of a tag of particular molecular weight that specifically binds to molecular biomarkers, further distinguishing them. Optionally, after detecting such modified biomarkers, the identity of the biomarkers can be further determined by matching the physical and chemical characteristics of the modified biomarkers in a protein database (e.g., SwissProt).

It is further appreciated in the art that biomarkers in a sample can be captured on a substrate for detection. Traditional substrates include antibody-coated 96-well plates or nitrocellulose membranes that are subsequently probed for the presence of the proteins. Alternatively, protein-binding molecules attached to microspheres, microparticles, microbeads, beads, or other particles can be used for capture and detection of biomarkers. The protein-binding molecules can be antibodies, peptides, peptoids, aptamers, small molecule ligands or other protein-binding capture agents attached to the surface of particles. Each protein-binding molecule can include unique detectable label that is coded such that it can be distinguished from other detectable labels attached to other protein-binding molecules to allow detection of biomarkers in multiplex assays. Examples include, but are not limited to, color-coded microspheres with known fluorescent light intensities (see e.g., microspheres with xMAP technology produced by Luminex (Austin, Tex.); microspheres containing quantum dot nanocrystals, for example, having different ratios and combinations of quantum dot colors (e.g., Qdot nanocrystals produced by Life Technologies (Carlsbad, Calif); glass coated metal nanoparticles (see e.g., SERS nanotags produced by Nanoplex Technologies, Inc. (Mountain View, Calif); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.); chemiluminescent dyes, combinations of dye compounds; and beads of detectably different sizes.

In another aspect, biochips can be used for capture and detection of the biomarkers of the invention. Many protein biochips are known in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture agent bound there. The capture agent can be a biological molecule, such as a polypeptide or a nucleic acid, which captures other biomarkers in a specific manner. Alternatively, the capture agent can be a chromatographic material, such as an anion exchange material or a hydrophilic material. Examples of protein biochips are well known in the art.

In some embodiments, the methods disclosed herein further comprise steps of determining body mass index (BMI), gestational age at blood draw (GABD), or one or more risk indicia. In other embodiments, the methods disclosed herein further comprise an initial step of measuring BMI, GABD and one more risk indicia.

As exemplified herein, the predictive performance of the claimed methods may include BMI of the pregnant female. The methods of the invention can be practiced with samples obtained from pregnant females with any BMI. Briefly, BMI is an individual's weight in kilograms divided by the square of height in meters. BMI does not measure body fat directly, but research has shown that BMI is correlated with more direct measures of body fat obtained from skinfold thickness measurements, bioelectrical impedance, densitometry (underwater weighing), dual energy x-ray absorptiometry (DXA) and other methods. Furthermore, BMI appears to be as strongly correlated with various metabolic and disease outcome as are these more direct measures of body fatness. Generally, an individual with a BMI below 18.5 is considered underweight, an individual with a BMI of equal or greater than 18.5 to 24.9 normal weight, while an individual with a BMI of equal or greater than 25.0 to 29.9 is considered overweight and an individual with a BMI of equal or greater than 30.0 is considered obese. In some embodiments, the predictive performance of the claimed methods can be improved with a BMI stratification of equal or greater than 18, equal or greater than 19, equal or greater than 20, equal or greater than 21, equal or greater than 22, equal or greater than 23, equal or greater than 24, equal or greater than 25, equal or greater than 26, equal or greater than 27, equal or greater than 28, equal or greater than 29 or equal or greater than 30. In other embodiments, the predictive performance of the claimed methods can be improved with a BMI stratification of equal or less than 18, equal or less than 19, equal or less than 20, equal or less than 21, equal or less than 22, equal or less than 23, equal or less than 24, equal or less than 25, equal or less than 26, equal or less than 27, equal or less than 28, equal or less than 29 or equal or less than 30.

In some embodiments, the pregnant female is between 17 and 28 weeks of gestation at the time the biological sample is collected, also referred to as GABD (Gestational Age at Blood Draw). In other embodiments, the pregnant female is between 16 and 29 weeks, between 17 and 28 weeks, between 18 and 27 weeks, between 19 and 26 weeks, between 20 and 25 weeks, between 21 and 24 weeks, or between 22 and 23 weeks of gestation at the time the biological sample is collected. In further embodiments, the pregnant female is between about 17 and 22 weeks, between about 16 and 22 weeks between about 22 and 25 weeks, between about 13 and 25 weeks, between about 26 and 28, or between about 26 and 29 weeks of gestation at the time the biological sample is collected. Accordingly, the gestational age of a pregnant female at the time the biological sample is collected can be 15, 16, 17, 18, 19, 20, 21, 22, 23, 24, 25, 26, 27, 28, 29 or 30 weeks. In particular embodiments, the biological sample is collected between 19 and 21 weeks of gestational age. In particular embodiments, the biological sample is collected between 19 and 22 weeks of gestational age. In particular embodiments, the biological sample is collected between 19 and 21 weeks of gestational age. In particular embodiments, the biological sample is collected between 19 and 22 weeks of gestational age. In particular embodiments, the biological sample is collected at 18 weeks of gestational age. While specific periods of GABD may be optimal, in some embodiments, any blood draw period can be used in the practice of the present disclosure. In further embodiments, the highest performing reversals for consecutive or overlapping time windows can be combined in a single classifier to predict the probability of sPTB over a wider window of gestational age at blood draw.

In some embodiments, one or more risk indicia can include prior preterm birth, short cervical length, pregnancy loss (e.g., prior miscarriage, ectopic or molar pregnancy or therapeutic termination), prior stillbirth, Body Mass Index (BMI), maternal age, parity, gravidity, fetal gender, height and weight separately from BMI, race, pre-existing diabetes, use of antidiabetic medications, pre-existing hypertension, and use of antihypertensive medications, and socioeconomic status. Additional markers can be selected from one or more risk indicia, including but not limited to, maternal characteristics, medical history, past pregnancy history, and obstetrical history. Additional markers, or particular subsets of the recited markers, can include, for example, previous low birth weight or preterm delivery, multiple 2nd trimester spontaneous abortions, prior first trimester induced abortion, familial and intergenerational factors, history of infertility, nulliparity, gravidity, primigravida, multigravida, placental abnormalities, cervical and uterine anomalies, short cervical length measurements, gestational bleeding, intrauterine growth restriction, in utero diethylstilbestrol exposure, multiple gestations, infant sex, short stature, low prepregnancy weight, low or high body mass index, diabetes, hypertension, urogenital infections (i.e. urinary tract infection), asthma, anxiety and depression, asthma, hypertension, hypothyroidism, low educational attainment, drug use and alcohol consumption. Demographic risk indicia for preterm birth can include, for example, maternal age, race/ethnicity, single marital status, low socioeconomic status, maternal age, employment-related physical activity, occupational exposures and environment exposures and stress. Further risk indicia can include, inadequate prenatal care, cigarette smoking, use of marijuana and other illicit drugs, cocaine use, alcohol consumption, caffeine intake, maternal weight gain, selenium (e.g., SEPP1), dietary intake, sexual activity during late pregnancy and leisure-time physical activities. (Preterm Birth: Causes, Consequences, and Prevention, Institute of Medicine (US) Committee on Understanding Premature Birth and Assuring Healthy Outcomes; Behrman R E, Butler A S, editors. Washington (DC): National Academies Press (US); 2007). Additional risk indicia useful for as markers can be identified using learning algorithms known in the art, such as linear discriminant analysis, support vector machine classification, recursive feature elimination, prediction analysis of microarray, logistic regression, CART, FlexTree, LART, random forest, MART, and/or survival analysis regression, which are known to those of skill in the art and are further described herein.

In some embodiments, one or more of the disclosed risk indica are used to calculate a probability of preterm birth, wherein the one or more risk indica are assigned a score and that score is input into a formula, as described above, for calculating the combined score. For example, the combined score can be calculated formula:

$$Combined score = ( A \times Biomarker level ( s ) ) + ( B \times GABD ) + ( C \times BMI ) + G \times GABD \times BMI ) + [ \text{} one or more of ( D \times priori history of preterm birth [ 0 or 1 ] ) . ( E \times current progesterone treatment [ 0 or 1 ] ) , ( F \times shortened cervix [ 0 or 1 ] ) ] \qquad ( 1 )$$

In some cases, a risk indica may not have a specified coefficient or have a value of 1 or 0. In some embodiments, risk indica (also referred herein as a risk factor) can be continuous, and in other embodiments the risk indica can be a categorical value. Moreover, risk indica can be measured at or before the time of risk prediction. For example, weight gain or smoking as risk factors may only include observations occurring before the risk calculation algorithm is employed.

Although described and exemplified with reference to methods of determining probability for preterm birth in a pregnant female, the present disclosure is similarly applicable to methods of predicting gestational age at birth (GAB), methods for predicting term birth, methods for determining the probability of term birth in a pregnant female as well methods of predicating time to birth (TTB) in a pregnant female. In some embodiments, the methods disclosed herein further comprise predicting gestational age at birth before determining probability of preterm birth. It will be apparent to one skilled in the art that each of the aforementioned methods has specific and substantial utilities and benefits with regard maternal-fetal health considerations. For methods directed to predicating time to birth, it is understood that “birth” means birth following spontaneous onset of labor, with or without rupture of membranes.

Furthermore, although described and exemplified with reference to methods of determining probability for preterm birth in a pregnant female, the present disclosure is similarly applicable to methods of predicting an abnormal glucose test, gestational diabetes, hypertension, preeclampsia, intrauterine growth restriction, stillbirth, fetal growth restriction, HELLP syndrome, oligohyramnios, chorioamnionitis, chorioamnionitis, placental previa, placental acreta, abruption, abruptio placenta, placental hemorrhage, preterm premature rupture of membranes, preterm labor, unfavorable cervix, postterm pregnancy, cholelithiasis, uterine over distention, stress. As described in more detail below, the classifier described herein is sensitive to a component of medically indicated PTB based on conditions such as, for example, preeclampsia or gestational diabetes.

In some embodiments, existence of a change in reversal value (or combined reversal value), classifier, or score (e.g., molecular score, clinical score, combined score) between the pregnant female and a term control indicates probability for preterm birth in a pregnant female. In some embodiments, the change in such value, classifier or score is significant. In other embodiments, the change is at least 0.1, 0.2, 0.5, 0.75, 1.0, 1.5 or 2.0 standard deviations or more greater than the mean value, classifier or score in a population of term controls. In other embodiments, the change in such a value, classifier, or score indicates the likelihood of preterm birth in the pregnant female is at least 0.1, 0.2, 0.5, 0.75, 1.0, 1.5 or 2.0 standard deviations or more greater than the likelihood in the term control.

One skilled in the art will appreciate that any algorithm can be used to determine the regression value. In a particular embodiment, the algorithm can include clinical and demographic variables. In some embodiments, clinical & demographic variables allow for calculation of a prior probability (Bayesian) of preterm birth based on clinical risks alone, such as may arise from underweight or deprivation. In this view, reversals in a regression model with clinical & demographic variables provide likelihood ratios that increase predictive accuracy over clinical & demographic models alone. In other words, the reversal is adjusting the clinical & demographic variables to be more aligned with the outcome. In other embodiments, the clinical & demographic variables adjust for variation in reversal scores that is not related to the outcome. Protein expression may be simultaneously anticipating future preterm birth and also be influenced by current, less relevant factors like gestational age progression or diet. In this view, clinical & demographic variables cancel out biological noise in reversal scores to provide a clearer signal.

In some embodiments, the regression value can be associated with a continuous risk of PTB and that the risk of PTB at some regression value may be statistically significant above the background risk of PTB or may otherwise correspond to some actionable threshold of risk.

In some embodiments, probability of preterm birth is expressed as a risk score. In some embodiments, the risk score is expressed as the log of the reversal value or combined reversal value, i.e. the ratio of the relative intensities of the individual biomarkers. One skilled in the art will appreciate that a risk score can be expressed based on a various data transformations as well as being expressed as the ratio itself. Furthermore, with particular regard to reversal pairs, one skilled in the art will appreciate the any ratio is equally informative if the biomarkers in the numerator and denominator are switched or that related data transformations (e.g. subtraction) are applied. Because the level of a biomarker may not be static throughout pregnancy, a standard or reference score has to have been obtained for the gestational time point that corresponds to that of the pregnant female at the time the sample was taken. The standard or reference score can be predetermined and built into a predictor model such that the comparison is indirect rather than actually performed every time the probability is determined for a subject. A risk score can be a standard (e.g., a number) or a threshold (e.g., a line on a graph). The value of the risk score correlates to the deviation, upwards or downwards, from the average amount of the one or more biomarkers calculated from biological samples obtained from a random pool of pregnant females. In certain embodiments, if a risk score is greater than a standard or reference risk score, the pregnant female can have an increased likelihood of preterm birth. In some embodiments, the magnitude of a pregnant female's risk score, or the amount by which it exceeds a reference risk score, can be indicative of or correlated to that pregnant female's level of risk.

Suitable biological samples that can be used in the methods of determining probability of preterm birth as disclosed above include, for example, blood, plasma, serum, amniotic fluid, vaginal secretions, saliva, and urine. In some embodiments, the biological sample is selected from the group consisting of whole blood, plasma, serum, saliva, urine, amniotic fluid, cervical vaginal fluid. As will be appreciated by those skilled in the art, a biological sample can include any fraction or component of blood, without limitation, T cells, monocytes, neutrophils, erythrocytes, platelets and microvesicles such as exosomes and exosome-like vesicles. In a particular embodiment, the biological sample is serum.

### Kits

In one aspect, the invention provides kits for determining probability of preterm birth. The kit can include one or more agents for detection of biomarkers, a container for holding a biological sample isolated from a pregnant female; and printed instructions for reacting agents with the biological sample or a portion of the biological sample to detect the presence or amount of the isolated biomarkers in the biological sample. The agents can be packaged in separate containers. The kit can further comprise one or more control reference samples and reagents for performing an immunoassay.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of determining probability of preterm birth.

Techniques for analyzing the biomarkers detected by the kit include hardware, software or a combination thereof in one or more computer systems or other processing systems capable of effectuating such analysis. Thus in one aspect, the present disclosure provides systems related to the above methods of the disclosure involving mass spectrometer analysis. In some embodiments, the system comprises an inlet, an ion source, a mass analyzer, an ion detector and a data system. In further embodiments, the system comprises a display module displaying the comparison between the test value and one or more reference values or displaying a result of the comparing step or displaying the patient's prognosis. In another embodiment provides a system for determining biomarker expression in a sample from a pregnant female.

From the foregoing description, it will be apparent that variations and modifications can be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

The following examples are provided by way of illustration, not limitation.

### EXAMPLES

### Example 1. Clinical Characteristics of the AMANHI Cohort

### Details of Ethics Approval

The study protocol was approved by the following ethics committees: WHO Institutional Review Board (IRB) (RPC532; 22 Jul. 2014), the ethics committees of the International Centre for Diarrheal Disease Research Bangladesh (icddr,b) in Bangladesh (PR 12073; 23 Mar. 2014), Aga Khan University in Pakistan (AKU Ethics Review Committee (OMB No. 0990-0279), 4359-Ped-ERC-16 and 2790-Ped-ERC-13); and Zanzibar Medical Research and Ethics Committee in Tanzania (ZAMREC/0002/October/013; 25 Mar. 2014). The Bangladesh protocol was also reviewed and approved by the IRB of John Hopkins Bloomberg School of Public Health, USA (IRB No: 00004508; October 2017, 2013) and the WHO (RPC532; 22 Jul. 2014).

### Study Design, Settings and Participants

Between 2014 and 2018, the Alliance for Maternal and Newborn Health Improvement (AMANHI) study prospectively enrolled 10,000 pregnant women that were identified through population-based surveillance in three countries of South Asia and sub-Saharan Africa: Bangladesh (Sylhet), Pakistan (Karachi) and Tanzania (Pemba Island). Mother-infant dyads were followed until 42 days postpartum to collect detailed epidemiological data and biological specimens during pregnancy, delivery, and the postpartum period. Baqui et al. Understanding biological mechanisms underlying adverse birth outcomes in developing countries: protocol for a prospective cohort (AMANHI bio-banking) study. J Glob Health. 2017; 7(2):021202.

### Biospecimen Collection, Processing and Storage

Each biorepository involved collection, processing, and storage of biological samples across three countries. Trained community health workers visited all women of child-bearing age in the study areas every two to three months to identify pregnancies and obtain informed consent. After women provided consent, pregnancies were confirmed via a strip-based pregnancy test and gestational age was determined using ultrasound conducted by trained ultrasonologists before 20 weeks of gestation. For fetuses less than 14 weeks, crown-rump length (CRL) was measured, whereas biparietal diameter (BPD) and femur length (FL) were the biometric measures for fetuses greater than 14 weeks gestation. Baqui et al. Understanding biological mechanisms underlying adverse birth outcomes in developing countries: protocol for a prospective cohort (AMANHI bio-banking) study. J Glob Health. 2017; 7(2):021202. A random sample of the images were reviewed internally and externally to ensure good quality control. Baqui et al. Understanding biological mechanisms underlying adverse birth outcomes in developing countries: protocol for a prospective cohort (AMANHI bio-banking) study. J Glob Health. 2017; 7(2):021202.

Maternal blood was collected during three intervals: at enrollment (8-19 weeks of gestation), either at 24-28 weeks or at 32-36 weeks of gestation, and 42-60 days postpartum, in pre-labelled tubes and processed to prepare aliquots of serum. All samples were stored at −80° C. Samples for the current analysis were shipped to the US via courier in a liquid nitrogen dry shipper. Samples did not have case/control assignment or clinical data on either the label or the paperwork received with the samples.

### Phenotype Data

Community health workers made four antepartum and two postpartum home visits to collect detailed phenotypic and epidemiological data including background characteristics, previous medical history, risk factors, exposures, and outcomes and morbidity for the index pregnancy. Maternal height was measured at the baseline visit immediately after enrollment and weight was measured at each antenatal and postnatal visit.

### Criteria for Selection of Cases and Controls

The current case-control study (100 sPTB before 37 weeks of gestation (cases) and 200 term deliveries at or after 37 weeks (controls) combined demographic data with proteomic results from maternal serum samples collected during 2014-2016, prior to completion of enrollment. Inclusion criteria included ability to consent, singleton pregnancies and blood collection performed within 17 weeks 0 days and 19 weeks 6 days. Exclusion criteria included signs/symptoms of preterm labor at time of specimen collection, known or suspected fetal anomaly, blood transfusion during current pregnancy, clinical diagnosis of jaundice, use of progesterone after 12 weeks 6 days gestation, use of heparin, or serum hemolysis greater than or equal to 100 mg/dl. Two controls per case matched by gestational week of blood draw and site were selected randomly from qualifying and available samples at the three sites: Bangladesh (36 sPTB/72 term), Pakistan (23 sPTB/46 term), and Tanzania (40 sPTB/80 term). One case and one control from Bangladesh were excluded from further analyses; the case sample did not show pregnancy specific proteins and the control sample was drawn in week 16, outside the study window.

### Laboratory Methods

At the time of laboratory analysis, Sera employees were blinded to and did not have access to case/control assignments or any clinical data on the AMANHI samples. The total set of samples were randomized (i.e., not separated by geography) to batches and batch positions using the R Statistical program (R 3.0.2). Team RC. R: A language and environment for statistical computing. Vienna, Austria: R Foundation for Statistical Computing; 2017. Serum was processed and analyzed in a CLIA-certified laboratory according to a standard operating procedure, essentially as described. Saade et al. Development and validation of a spontaneous preterm delivery predictor in asymptomatic women. Am J Obstet Gynecol. 2016; 214(5):633.e1-633.e24; Bradford et al. Analytical validation of protein biomarkers for risk of spontaneous preterm birth. Clinical Mass Spectrometry. 2017; 3:25-38. Briefly, serum samples were depleted of high abundance proteins and proteolyzed with trypsin. The resulting peptide mixture was fortified with stable isotope labeled versions of the proteotypic peptides of the target biomarkers and desalted. The desalted peptide mixture was then analyzed by coupled liquid chromatography-multiple reaction monitoring mass spectrometry (LC-MRM) measuring 83 peptides from 61 proteins associated with pregnancy or pregnancy complications (Saade et al. Development and validation of a spontaneous preterm delivery predictor in asymptomatic women. Am J Obstet Gynecol. 2016; 214(5):633.e1-633.e24) and in a second MRM assay measuring 48 additional proteins, enriched for placentally-derived proteins. Detected peptides were quantified by the response ratio between endogenous and isotope labeled peak area counts. Quality was assessed for each batch (Saade et al. Development and validation of a spontaneous preterm delivery predictor in asymptomatic women. Am J Obstet Gynecol. 2016; 214(5):633.e1-633.e24; Bradford et al. Analytical validation of protein biomarkers for risk of spontaneous preterm birth. Clinical Mass Spectrometry. 2017; 3:25-38) and after all runs were completed.

### Modeling

Peptides were evaluated in three windows of gestational age at blood draw (GABD), which included early GABD (days 119-152), late GABD (days 156-196) and full GABD (days 119-202) of gestation. A variation of a stacking algorithm in which four modeling approaches were combined and a brute force approach to inverse rank sum each peptide in the dataset within each GABD window. The four algorithms included approaches for penalized selection, causal inference, conditional inference and a gradient boosting tool. To help identify reversals we added a fifth brute-force ranking based on significance of enrichment of individual peptides amongst the top performing reversals. Top reversals per GABD window were determined by first ranking all reversals by inverse rank sum, and then by association of the first principal component with the outcome. Each of the different models ranked the peptides from most important to least important and the final score was determined by taking a weighted inverse rank sum across all the different models plus the brute force approach.

### Statistical Analyses

Significant differences (p<0.05) in demographics and clinical variables between the U.S. validation cohort and the AMANHI cohort were determined using a t-test (means) or a Wilcoxon test (medians) for continuous clinical variables and the Fisher-exact test for categorical variables, with missing values excluded from analyses. Rich B. table1: Tables of Descriptive Statistics in HTML. R package version 1.2. 2020; Robinson L. demoGraphic: Providing Demographic Table with the P-Value, Standardized Mean Difference Value. R package version 0.1.0. 2019. IBP4/SHBG predictor scores were calculated as the natural logarithm of the response ratios of IBP4 and SHBG. Saade et al. Development and validation of a spontaneous preterm delivery predictor in asymptomatic women. Am J Obstet Gynecol. 2016; 214(5):633.e1-633.e24; Markenson et al. Performance of a proteomic preterm delivery predictor in a large independent prospective cohort. Am J Obstet Gynecol MFM. 2020 August; 2(3):100140.

To assess performance of the IBP4/SHBG predictor in the absence of clinical variables, all samples were used. Logistic regression models were tested when clinical variables were added, and subjects with missing values (see Table 4) were omitted. Imputation of missing BMI values using Multivariate Imputation by Chained Equations (Buuren et al.: Multivariate Imputation by Chained Equations in R. J Stat Softw. 2011; 45(3):1-67), yielded similar results. For discovery analyses causal inference network analysis (Badsha et al.: An R package for accurate inference of causal graphs; Badsha et al. Learning Causal Biological Networks With the Principle of Mendelian Randomization. Front Genet. 2019; 10:460) was used to rank additional proteins (log transformed ratios) and clinical variables important for predicting preterm birth. The IBP4/SHBG ratio, GABD, BMI, twelve additional proteins and one additional clinical variable (prior sPTB) were identified with a mean bootstrap score ≥0.05 as direct parents of sPTB. These additional biomarkers (1, 2 or 3 at a time) were combined in the parent predictor (IBP4/SHBG predictor score, gestational age at blood draw and BMI) in logistic regression models. Overall performance for all models was reported by AUC with prespecified direction (cases>controls), with 95% confidence intervals calculated by DeLong's method. DeLong et al. Comparing the areas under two or more correlated receiver operating characteristic curves: a nonparametric approach. Biometrics. 1988 September; 44(3):837-45. A Wilcoxon one-sided test was used to calculate p-values.

TABLE 4
Comparison of AMANHI and US Validation Cohorts

| | | | US Va [symbol] dation | | |
|---|---|---|---|---|---|
| | AMANHI | | Cohort [11] | | |
| | (N = 298) | | (N = 54) | | p-value |
| Maternal Age (years) | | | | | |
| Mean (SD) | 26.1 | (5.97) | 26.2 | (6.25) | 0.90 |
| Median [Q1, Q3] | 25.0 [21.0, | | 24.5 [21.3, | | 0.94 |
| | 30.0) | | 30.0] | | |
| Body Mass Index ( [symbol] g/m¹) | | | | | |
| Mean (SD) | 21.8 | (4.19) | 27.7 | (6.22) | <0.001 |
| Median [Q1, Q3] | 20.8 (18.6, | | 26.5 (22.3, | | <0.001 |
| | 24.0) | | 31.3] | | |
| Missing | 39 | (13.1%) | 1 | (1.9%) | |
| Gravida | | | | | |
| Multigravida | 235 | (78.9%) | 41 | (75.9%) | 0.60 |
| Primigravida | 63 | (21.1%) | 13 | (24.1%) | |
| Prior PTB | | | | | |
| No | 288 | (96.6%) | 47 | (87.0%) | 0.008 |
| Yes | 10 | (3.4%) | 7 | (13.0%) | |
| Gestational Age at Blood | | | | | |
| Draw (days) | | | | | |
| Mean (SD) | 128 | (6.39) | 140 | (4.20) | <0.001 |
| Median (Q1, Q3] | 127 [122, | | 139 [135, | | <0.001 |
| | 134] | | 144] | | |
| Gestational Age at Birth | | | | | |
| (days) | | | | | |
| Mean (SD) | 267 | (18.4) | 265 | (24.2) | 0.44 |

| | | | |
|---|---|---|---|
| Median [Q1, Q3] | 271 (256, | 273 (256, | 0.90 |
| | 281) | 281] | |

Note:
Prior PTB [symbol] restricted to [symbol] spontaneous pre [symbol] births in the US validation cohort. Prior PTB is not limited by timing of initiation of delivery in the AMANHI cohort. [symbol]
indicates data missing or illegible when filed

[symbol]

To assess prediction of early sPTB, all gestational ages at birth were represented at their prevalence in the source population, without exclusion of patients at the definition boundary. Boniface et al. Effects of Selective Exclusion of Patients on Preterm Birth Test Performance. Obstetrics and gynecology. 2019; 134(6):1333-1338. For sPTB<34 weeks, subjects delivering ≥34 weeks (preterm and term births) were defined as controls. To maintain a representative distribution of gestational age amongst controls, subjects with later (i.e. between 34 and 37 weeks) preterm birth were down sampled to their prevalence in the source (i.e. intended use) population. To avoid bias, down-sampling was repeated 100 times and median AUCs were reported. Importantly, the coefficients within the predictor learned for sPTB<37 were retained for prediction of sPTB<34 without retraining. For Kaplan-Meier analysis subjects were divided into low- and high-risk groups based on screen positive rates from 5 to 95%, in 5% increments. Time of gestation was measured from the estimated last menstrual period to the event of delivery, and significance was assessed by the log-rank statistic.

### Comparison of the AMANHI Cohort to the US Validation Cohort

Clinical characteristics of the AMANHI cohort (99 sPTB+199 term controls) were compared to the cohort used in the original US validation study (Saade G R et al. Development and validation of a spontaneous preterm delivery predictor in asymptomatic women. Am J Obstet Gynecol. 2016 May 2016; 214(5):633.e1-633.e24) (Table 4). The mean gestational age at blood draw was significantly different between the two studies (128 vs. 140 days, p<0.001). This is due to the study design: the optimal gestational age at blood draw for the IBP4/SHBG predictor was within weeks 191/6-206/7 gestation, while the AMANHI samples spanned weeks 170/6-196/7 (the three weeks that overlapped with the entire Proteomic Assessment of Preterm Risk (PAPR, NCT01371019) study (170/6-286/7 weeks)).

The mean BMI of the AMANHI cohort was significantly lower than in the US cohort (21.8 kg/m2 vs. 27.7 kg/m2, p<0.001), and of the 259 subjects with a recorded BMI, 155 fell below the reported optimal US BMI- (22< to ≤37 kg/m2) (Saade G R et al. Development and validation of a spontaneous preterm delivery predictor in asymptomatic women. Am J Obstet Gynecol. 2016 May 2016; 214(5):633.e1-633.e24).

The proportion of subjects with a prior PTB was lower in AMANHI than in the US despite inclusion of both spontaneous and indicated PTB in the AMANHI data definition, likely due to the fact that US subjects were recruited, in part, from high-risk centers whereas in the AMANHI study all resident women identified by routine surveillance with pregnancies earlier than 20 weeks were eligible for enrollment and women may not have had access to ultrasound dating in prior pregnancies thus reducing the sensitivity of detection of prior PTB. There were no significant differences in maternal age or in the proportion of gravidity between the cohorts. The mean gestational age at delivery was also similar between geographies.

### Example 2. Discovery of Novel Predictors for the AMANHI Cohort

IBP4/SHBG demonstrates dependence on gestational age at blood draw and BMI (Saade et al. Development and validation of a spontaneous preterm delivery predictor in asymptomatic women. Am J Obstet Gynecol. 2016; 214(5):633.e1-633.e24) and SHBG blood levels are reported to be associated with BMI. Xargay-Torrent et al. Circulating sex hormone binding globulin: An integrating biomarker for an adverse cardio-metabolic profile in obese pregnant women. PLOS ONE. 2018; 13(10):e0205592. Without adjustment for BMI or blood draw window the IBP4/SHBG predictor score did not reach statistical significance (p=0.069, Table 5). However, with adjustment for GABD and BMI the IBP4/SHBG predictor score significantly classified sPTB subjects vs. term (AUC=0.64, 95% CI: 0.57-0.71, p=<0.001). (Table 5). Importantly, this adjusted IBP4/SHBG predictor was also able to classify early (<34 weeks) preterm births (AUC=0.66, 95% CI: 0.51-0.82, p=0.0.013)).

TABLE 5
Performance of IBP4/SHBG and IBP4/SHBG + (EGLN + PRL)/TETN in predicting sPTB

| | sPTB < 37 | | | | | sPTB < 34 | | | | |
|---|---|---|---|---|---|---|---|---|---|---|
| | | 95% | p- | Cases | Controls | | 95% | p- | Cases | Controls |
| Model | AUC | CI | value | (n) | (n) | AUC | CI | value | (n) | (n) |
| IBP4/SHBG | 0.55 | 0.48- | 0.069 | 99 | 199 | 0.62 | 0.49:0.75 | 0.044 | 19 | 214 |
| | | 0.62 | | | | | | | | |
| IBP4/SHBG + | 0.64 | 0.57- | <0.001 | 88 | 171 | 0.66 | 0.51:0.82 | 0.013 | 17 | 184 |
| GABD*BMI | | 0.71 | | | | | | | | |
| IBP4/SHBG + | 0.73 | 0.67- | <0.001 | 88 | 171 | 0.78 | 0.66:0.90 | <0.001 | 17 | 184 |
| GABD*BMI + | | 0.80 | | | | | | | | |
| (EGLN + | | | | | | | | | | |
| PRL)/TETN | | | | | | | | | | |

To see if the predictor could be optimized to the three AMANHI geographies, we used AI network techniques to select new features. Complexity was reduced by feature selection prior to model building. Specifically, 105 proteins and 107 individual peptide measurements available in the entire dataset were reduced using a conditional correlation network to identify biomarkers that are inferred to be upstream of the event of sPTB.

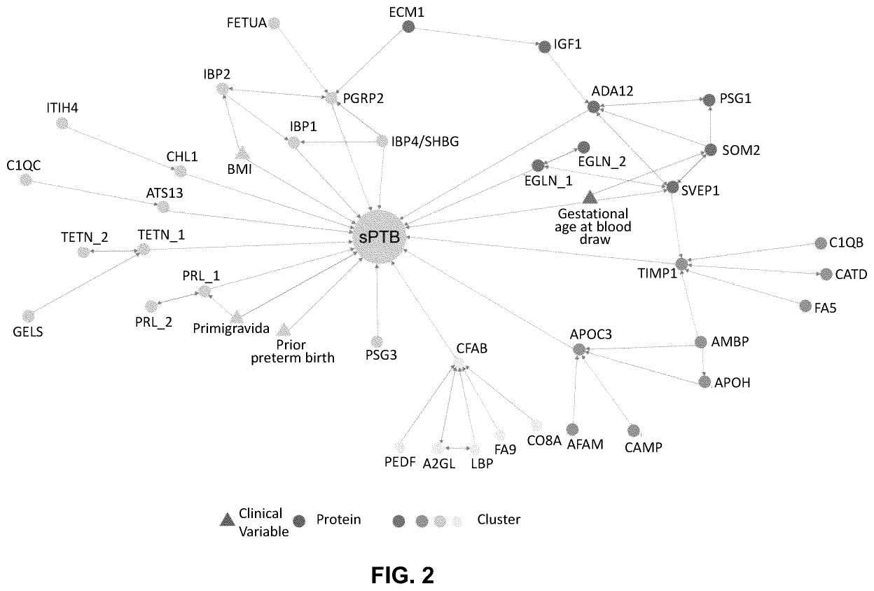

FIG. 2 shows data taken from a causal network analysis showing relationships of proteins measured by MRM-MS and clinical variables to preterm birth (sPTB, center circle). For model building proteins directly connected to sPTB in the causal network analysis (ADA12, APOC3, ATS13, CFAB, CHL1, IBP1, EGLN, PGRP2, PRL, PSG3, TETN, and TIMP) were tested individually, in pairs, or in triplets with the adjusted predictor (IBP4/SHBG+GA at blood draw*BMI). Each model was also tested with and without prior PTB as a variable. Prior PTB did not significantly improve performance, likely because this variable was obtained from recall and may not be accurate.

The analysis causal network analysis identified thirteen features (twelve proteins and one clinical variable), in addition to IBP4/SHBG, gestational age at blood draw, and BMI, as direct antecedents of sPTB. These top features were then added to the adjusted IBP4/SHBG predictor individually, in pairs, or in triplets. AUC was significantly improved over the adjusted predictor with the addition of three proteins, i.e., EGLN, prolactin (PRL), and tetranetin (TETN). Extra clinical veriables did not significantly improve performance.

In addition, the causal network analysis identified twenty-two features as indirect antecendents of sPTB. The indirect antecendents of sPTB can be identified in the causal network analysis (see FIG. 2) as those features that are indirectly connected to sPTB via one of the direct antecndents (i.e., ADA12, APOC3, ATS13, CFAB, CHL1, IBP1, EGLN, PGRP2, PRL, PSG3, TETN, and TIMP). The indirect antecendents of sPTB include proteins CO8A, LBP, A2GL, PEDF, GELS, ITIH4, IBP2, FETUA, PSG1, SOM2, C1QB, CATD, APOH, AFAM, C1QC, ECM1, IGF1, FA9, SVEP1, FA5, AMBP, and CAMP. In some instances, relationships between direct antecendents and indirect antecedents may offer valuable information on sPTB prediction. For example, ratios between one or more indirect antecendents and a corresponding direct antecendent can provide a more complete representation of biomarker activity for sPTB prediction. In other instances, indirect antecendents may be added, either alone or in combination with its corresponding direct antecendent, to an IBP4/SHBG predictor, individually, in pairs, or in triplets, to enhance the performance of the predictor.

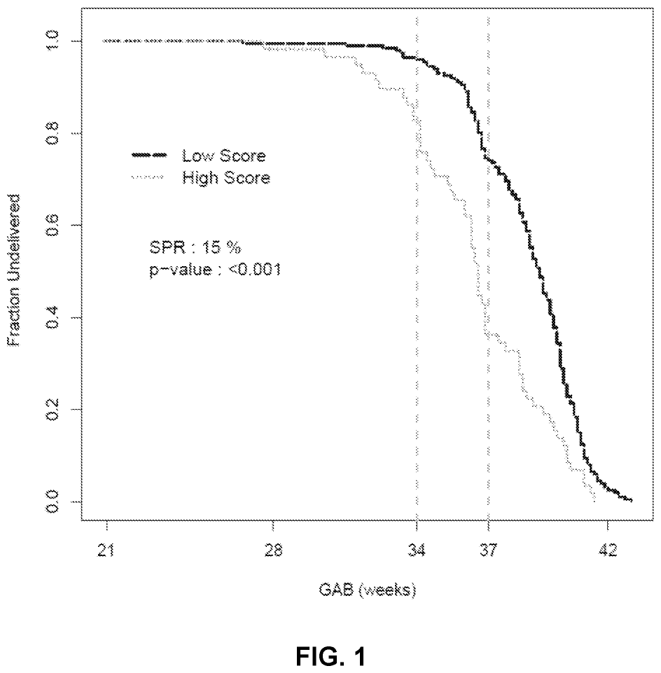

The top predictor ranked by AUC included three new analytes: EGLN (EGLN), prolactin (PRL), and tetranectin (TETN), forming a new ratio as (EGLN+PRL)/TETN. Each gave similar performance when added alone (AUC=0.66-68 for sPTB<37 and AUC=0.68-0.70 for sPTB<34) or in various pairwise combinations (AUC=0.69-0.71 for sPTB<37 and AUC=0.72-0.76), but AUCs were not significantly different from adjusted IBP4/SHBG. However, significant improvement in prediction of sPTB<37 was seen for the adjusted IBP4/SHBG predictor encompassing the three proteins with an AUC=0.73 (95% CI: 0.67-0.80, p-value=<0.001) and an AUC of 0.78 (95% CI: 0.66-0.90, p<0.001) for prediction of early sPTB<34 (Table 5). Using this predictor, subjects were stratified into low- and high-risk groups. At a 15% screen positive rate, Kaplan-Meier analysis indicated that subjects in the high-risk group (the top 15%) delivered earlier than those in the lower-risk group, (FIG. 1). The separation of gestational age at birth is highly significant (p=<0.001). The adjusted predictor is strongly associated with gestational age at birth, giving significant separation at thresholds corresponding screen positive rates (from 5 to 85% (p<0.001-0.047).

Additional reversal groups tested in the methods disclosed herein are included in Tables 6-19. While all of these models significantly predicted sPTB, only a small number showed statistically significant improvement over the IBP4/SHBG predictor adjusted for gestational age at birth (GABD) and BMI (Tables 18-19). Improvement over the adjusted predictor was considered significant when the AUC of the predictor containing additional analytes was larger than the upper 95% confidence interval (CI) of the adjusted predictor.

TABLE 6
Reversal Group Classification Performance at Predicting sPTB <34
Weeks Gestation in women. Reversal group comprises IBP4/SHBG, two
additional clinical variables and one additional protein biomarker.

| Reversal Group | AUC | CI low | CI high | Sum Rank |
|---|---|---|---|---|
| ATD_~CFAB + IBP4/SHBG + GABD*BMI | 0.737 | 0.725 | 0.744 | 22.63 |
| ATD_~1/APOC3 + IBP4/SHBG + GABD*BMI | 0.675 | 0.665 | 0.684 | 11.52 |
| ATD_~EGLN + IBP4/SHBG + GABD*BMI | 0.704 | 0.692 | 0.716 | 7.31 |
| ATD_~1/PGRP2 + IBP4/SHBG + GABD*BMI | 0.669 | 0.658 | 0.679 | 7.21 |
| ATD_~TIMP1 + IBP4/SHBG + GABD*BMI | 0.679 | 0.668 | 0.688 | 6.13 |
| ATD_~PRL + IBP4/SHBG + GABD*BMI | 0.697 | 0.686 | 0.710 | 6.13 |
| ATD_~PSG3 + IBP4/SHBG + GABD*BMI | 0.680 | 0.670 | 0.689 | 5.78 |
| ATD_~CHL1 + IBP4/SHBG + GABD*BMI | 0.670 | 0.661 | 0.678 | 5.60 |
| ATD_~ADA12 + IBP4/SHBG + GABD*BMI | 0.673 | 0.663 | 0.683 | 4.57 |
| ATD_~IBP1 + IBP4/SHBG + GABD*BMI | 0.663 | 0.655 | 0.672 | 4.49 |
| ATD_~1/TETN + IBP4/SHBG + GABD*BMI | 0.677 | 0.669 | 0.685 | 4.32 |
| ATD_~1/ATS13 + IBP4/SHBG + GABD*BMI | 0.644 | 0.635 | 0.654 | 4.31 |

TABLE 7
Reversal Group Classification Performance at Predicting sPTB <34 Weeks
Gestation. Reversal group comprises IBP4/SHBG, two additional clinical variables
and an additional reversal pair or two additional protein biomarkers.

| Reversal Group | AUC | CI low | CI high | Sum Rank |
|---|---|---|---|---|
| ATD_~EGLN + CFAB + IBP4/SHBG + BMI*GABD | 0.764 | 0.755 | 0.772 | 9.65 |
| ATD_~EGLN/TETN + IBP4/SHBG + BMI*GABD | 0.757 | 0.746 | 0.769 | 3.07 |
| ATD_~PSG3 + CFAB + IBP4/SHBG + BMI*GABD | 0.748 | 0.741 | 0.755 | 3.32 |
| ATD_~PRL + CFAB + IBP4/SHBG + BMI*GABD | 0.747 | 0.736 | 0.758 | 4.82 |
| ATD_~CFAB + TIMP1 + IBP4/SHBG + BMI*GABD | 0.745 | 0.735 | 0.753 | 6.60 |
| ATD_~CFAB/APOC3 + IBP4/SHBG + BMI*GABD | 0.742 | 0.730 | 0.750 | 12.23 |
| ATD_~PRL/TETN + IBP4/SHBG + BMI*GABD | 0.741 | 0.731 | 0.749 | 1.80 |
| ATD_~CFAB/PGRP2 + IBP4/SHBG + BMI*GABD | 0.738 | 0.728 | 0.745 | 8.76 |
| ATD_~CFAB + CHL1 + IBP4/SHBG + BMI*GABD | 0.735 | 0.725 | 0.742 | 5.49 |
| ATD_~ADA12 + CFAB + IBP4/SHBG + BMI*GABD | 0.731 | 0.720 | 0.741 | 2.31 |
| ATD_~CFAB + IBP1 + IBP4/SHBG + BMI*GABD | 0.731 | 0.722 | 0.738 | 4.77 |
| ATD_~CFAB/TETN + IBP4/SHBG + BMI*GABD | 0.730 | 0.722 | 0.737 | 3.59 |
| ATD_~PRL + EGLN + IBP4/SHBG + BMI*GABD | 0.724 | 0.715 | 0.734 | 3.22 |
| ATD_~PRL + PSG3 + IBP4/SHBG + BMI*GABD | 0.719 | 0.709 | 0.730 | 1.28 |
| ATD_~EGLN/PGRP2 + IBP4/SHBG + BMI*GABD | 0.719 | 0.707 | 0.729 | 1.43 |
| ATD_~CFAB/ATS13 + IBP4/SHBG + BMI*GABD | 0.718 | 0.707 | 0.725 | 2.68 |
| ATD_~EGLN + TIMP1 + IBP4/SHBG + BMI*GABD | 0.717 | 0.706 | 0.727 | 1.57 |
| ATD_~EGLN + PSG3 + IBP4/SHBG + BMI*GABD | 0.717 | 0.706 | 0.727 | 1.85 |
| ATD_~TIMP1/TETN + IBP4/SHBG + BMI*GABD | 0.714 | 0.706 | 0.722 | 1.27 |
| ATD_~EGLN + IBP1 + IBP4/SHBG + BMI*GABD | 0.713 | 0.702 | 0.725 | 1.41 |
| ATD_~PRL + ADA12 + IBP4/SHBG + BMI*GABD | 0.710 | 0.699 | 0.723 | 1.12 |
| ATD_~EGLN + CHL1 + IBP4/SHBG + BMI*GABD | 0.710 | 0.698 | 0.722 | 2.90 |
| ATD_~EGLN/ATS13 + IBP4/SHBG + BMI*GABD | 0.708 | 0.695 | 0.720 | 1.21 |
| ATD_~PRL + IBP1 + IBP4/SHBG + BMI*GABD | 0.707 | 0.694 | 0.719 | 0.77 |
| ATD_~ADA12 + EGLN + IBP4/SHBG + BMI*GABD | 0.706 | 0.695 | 0.718 | 0.93 |
| ATD_~ADA12/TETN + IBP4/SHBG + BMI*GABD | 0.706 | 0.697 | 0.713 | 1.04 |
| ATD_~PRL/APOC3 + IBP4/SHBG + BMI*GABD | 0.705 | 0.693 | 0.718 | 0.89 |
| ATD_~EGLN/APOC3 + IBP4/SHBG + BMI*GABD | 0.702 | 0.691 | 0.713 | 0.93 |
| ATD_~PRL + CHL1 + IBP4/SHBG + BMI*GABD | 0.699 | 0.689 | 0.710 | 1.02 |
| ATD_~PRL/ATS13 + IBP4/SHBG + BMI*GABD | 0.699 | 0.687 | 0.710 | 1.64 |
| ATD_~PSG3 + TIMP1 + IBP4/SHBG + BMI*GABD | 0.699 | 0.690 | 0.708 | 1.07 |
| ATD_~PRL/PGRP2 + IBP4/SHBG + BMI*GABD | 0.699 | 0.686 | 0.710 | 1.10 |
| ATD_~PRL + TIMP1 + IBP4/SHBG + BMI*GABD | 0.698 | 0.688 | 0.709 | 0.94 |
| ATD_~PSG3/TETN + IBP4/SHBG + BMI*GABD | 0.698 | 0.691 | 0.707 | 0.82 |
| ATD_~ADA12 + PSG3 + IBP4/SHBG + BMI*GABD | 0.696 | 0.686 | 0.705 | 1.02 |
| ATD_~ADA12/APOC3 + IBP4/SHBG + BMI*GABD | 0.694 | 0.683 | 0.704 | 1.50 |
| ATD_~1/(TETN + APOC3) + IBP4/SHBG + BMI*GABD | 0.693 | 0.684 | 0.702 | 0.97 |
| ATD_~PSG3/APOC3 + IBP4/SHBG + BMI*GABD | 0.692 | 0.684 | 0.701 | 1.09 |
| ATD_~PSG3/PGRP2 + IBP4/SHBG + BMI*GABD | 0.692 | 0.683 | 0.701 | 1.13 |
| ATD_~TIMP1/PGRP2 + IBP4/SHBG + BMI*GABD | 0.689 | 0.678 | 0.698 | 0.80 |
| ATD_~PSG3 + IBP1 + IBP4/SHBG + BMI*GABD | 0.688 | 0.679 | 0.698 | 1.66 |
| ATD_~CHL1 + TIMP1 + IBP4/SHBG + BMI*GABD | 0.688 | 0.677 | 0.697 | 0.67 |
| ATD_~TIMP1/APOC3 + IBP4/SHBG + BMI*GABD | 0.686 | 0.677 | 0.694 | 0.98 |
| ATD_~ADA12/PGRP2 + IBP4/SHBG + BMI*GABD | 0.686 | 0.677 | 0.695 | 0.94 |
| ATD_~IBP1/TETN + IBP4/SHBG + BMI*GABD | 0.685 | 0.676 | 0.693 | 1.45 |
| ATD_~CHL1/TETN + IBP4/SHBG + BMI*GABD | 0.684 | 0.676 | 0.693 | 0.71 |
| ATD_~IBP1 + TIMP1 + IBP4/SHBG + BMI*GABD | 0.683 | 0.673 | 0.691 | 1.84 |
| ATD_~PSG3 + CHL1 + IBP4/SHBG + BMI*GABD | 0.680 | 0.672 | 0.689 | 0.88 |
| ATD_~CHL1/APOC3 + IBP4/SHBG + BMI*GABD | 0.680 | 0.670 | 0.689 | 2.35 |
| ATD_~ADA12 + IBP1 + IBP4/SHBG + BMI*GABD | 0.680 | 0.670 | 0.689 | 0.99 |
| ATD_~ADA12 + CHL1 + IBP4/SHBG + BMI*GABD | 0.680 | 0.670 | 0.690 | 0.89 |
| ATD_~CHL1/PGRP2 + IBP4/SHBG + BMI*GABD | 0.679 | 0.671 | 0.688 | 3.74 |
| ATD_~ADA12 + TIMP1 + IBP4/SHBG + BMI*GABD | 0.679 | 0.668 | 0.689 | 0.60 |
| ATD_~1/(TETN + PGRP2) + IBP4/SHBG + BMI*GABD | 0.678 | 0.671 | 0.685 | 0.90 |
| ATD_~ADA12/ATS13 + IBP4/SHBG + BMI*GABD | 0.677 | 0.667 | 0.687 | 1.95 |
| ATD_~1/(PGRP2 + APOC3) + IBP4/SHBG + BMI*GABD | 0.674 | 0.665 | 0.683 | 1.71 |
| ATD_~TIMP1/ATS13 + IBP4/SHBG + BMI*GABD | 0.672 | 0.662 | 0.681 | 0.54 |
| ATD_~IBP1/APOC3 + IBP4/SHBG + BMI*GABD | 0.671 | 0.662 | 0.680 | 1.50 |
| ATD_~IBP1/PGRP2 + IBP4/SHBG + BMI*GABD | 0.671 | 0.663 | 0.679 | 1.83 |
| ATD_~PSG3/ATS13 + IBP4/SHBG + BMI*GABD | 0.667 | 0.658 | 0.677 | 1.03 |
| ATD_~IBP1 + CHL1 + IBP4/SHBG + BMI*GABD | 0.667 | 0.659 | 0.673 | 0.80 |
| ATD_~1/(TETN + ATS13) + IBP4/SHBG + BMI*GABD | 0.667 | 0.659 | 0.674 | 1.37 |
| ATD_~1/(ATS13 + APOC3) + IBP4/SHBG + BMI*GABD | 0.664 | 0.652 | 0.674 | 1.36 |
| ATD_~1/(PGRP2 + ATS13) + IBP4/SHBG + BMI*GABD | 0.658 | 0.648 | 0.668 | 0.67 |
| ATD_~CHL1/ATS13 + IBP4/SHBG + BMI*GABD | 0.653 | 0.645 | 0.662 | 1.20 |
| ATD_~IBP1/ATS13 + IBP4/SHBG + BMI*GABD | 0.649 | 0.639 | 0.657 | 1.89 |

TABLE 8
Reversal Group Classification Performance at Predicting sPTB <34 weeks
gestation. Reversal group comprises IBP4/SHBG, two additional clinical
variables and a reversal triplet or three additional protein biomarkers.

| Reversal Group | AUC | CI low | CI high | Sum Rank |
|---|---|---|---|---|
| ATD_~(EGLN + CFAB)/TETN + IBP4/SHBG + BMI*GABD | 0.786 | 0.778 | 0.794 | 5.25 |
| ATD_~(PRL + EGLN)/TETN + IBP4/SHBG + BMI*GABD | 0.783 | 0.773 | 0.793 | 2.12 |
| ATD_~EGLN + PSG3 + CFAB + IBP4/SHBG + BMI*GABD | 0.780 | 0.772 | 0.789 | 1.64 |
| ATD_~PRL + EGLN + CFAB + IBP4/SHBG + BMI*GABD | 0.773 | 0.762 | 0.781 | 3.51 |
| ATD_~(EGLN + TIMP1)/TETN + IBP4/SHBG + BMI*GABD | 0.772 | 0.762 | 0.783 | 0.96 |
| ATD_~(EGLN + PSG3)/TETN + IBP4/SHBG + BMI*GABD | 0.772 | 0.762 | 0.782 | 1.93 |
| ATD_~(EGLN + CFAB)/PGRP2 + IBP4/SHBG + BMI*GABD | 0.771 | 0.762 | 0.780 | 2.40 |
| ATD_~(EGLN + CFAB)/APOC3 + IBP4/SHBG + BMI*GABD | 0.770 | 0.761 | 0.778 | 4.18 |
| ATD_~PRL + PSG3 + CFAB + IBP4/SHBG + BMI*GABD | 0.768 | 0.758 | 0.779 | 1.18 |
| ATD_~EGLN + CFAB + TIMP1 + IBP4/SHBG + BMI*GABD | 0.767 | 0.756 | 0.776 | 1.43 |
| ATD_~(EGLN + PGRP2)/TETN + IBP4/SHBG + BMI*GABD | 0.767 | 0.756 | 0.777 | 0.73 |
| ATD_~EGLN + CFAB + IBP1 + IBP4/SHBG + BMI*GABD | 0.767 | 0.759 | 0.775 | 3.21 |
| ATD_~(PRL + CFAB)/TETN + IBP4/SHBG + BMI*GABD | 0.765 | 0.756 | 0.772 | 1.03 |
| ATD_~(EGLN + IBP1)/TETN + IBP4/SHBG + BMI*GABD | 0.763 | 0.753 | 0.773 | 0.74 |
| ATD_~EGLN + CFAB + CHL1 + IBP4/SHBG + BMI*GABD | 0.763 | 0.754 | 0.770 | 1.37 |
| ATD_~(EGLN + CHL1)/TETN + IBP4/SHBG + BMI*GABD | 0.762 | 0.752 | 0.772 | 0.63 |
| ATD_~PSG3 + CFAB + TIMP1 + IBP4/SHBG + BMI*GABD | 0.762 | 0.752 | 0.770 | 1.48 |
| ATD_~(PRL + PSG3)/TETN + IBP4/SHBG + BMI*GABD | 0.762 | 0.752 | 0.772 | 0.70 |
| ATD_~ADA12 + EGLN + CFAB + IBP4/SHBG + BMI*GABD | 0.761 | 0.752 | 0.769 | 0.75 |
| ATD_~(PRL + CFAB)/APOC3 + IBP4/SHBG + BMI*GABD | 0.760 | 0.749 | 0.769 | 1.58 |
| ATD_~(PRL + ADA12)/TETN + IBP4/SHBG + BMI*GABD | 0.758 | 0.749 | 0.770 | 0.49 |
| ATD_~(ADA12 + EGLN)/TETN + IBP4/SHBG + BMI*GABD | 0.758 | 0.748 | 0.768 | 0.77 |
| ATD_~(PRL + TIMP1)/TETN + IBP4/SHBG + BMI*GABD | 0.758 | 0.747 | 0.768 | 0.73 |
| ATD_~EGLN/(TETN + APOC3) + IBP4/SHBG + BMI*GABD | 0.757 | 0.748 | 0.768 | 0.49 |
| ATD_~PRL + CFAB + IBP1 + IBP4/SHBG + BMI*GABD | 0.757 | 0.744 | 0.766 | 0.72 |
| ATD_~(CFAB + TIMP1)/APOC3 + IBP4/SHBG + BMI*GABD | 0.757 | 0.748 | 0.765 | 11.73 |
| ATD_~(EGLN + CFAB)/ATS13 + IBP4/SHBG + BMI*GABD | 0.756 | 0.747 | 0.764 | 0.65 |
| ATD_~(PSG3 + CFAB)/APOC3 + IBP4/SHBG + BMI*GABD | 0.756 | 0.745 | 0.765 | 1.50 |
| ATD_~(PRL + IBP1)/TETN + IBP4/SHBG + BMI*GABD | 0.754 | 0.744 | 0.764 | 0.44 |
| ATD_~ADA12 + PSG3 + CFAB + IBP4/SHBG + BMI*GABD | 0.753 | 0.743 | 0.763 | 0.54 |
| ATD_~(PSG3 + CFAB)/PGRP2 + IBP4/SHBG + BMI*GABD | 0.753 | 0.743 | 0.761 | 3.44 |
| ATD_~PRL/(TETN + APOC3) + IBP4/SHBG + BMI*GABD | 0.753 | 0.742 | 0.765 | 0.54 |
| ATD_~(PRL + CFAB)/PGRP2 + IBP4/SHBG + BMI*GABD | 0.751 | 0.740 | 0.759 | 1.97 |
| ATD_~(CFAB + TIMP1)/TETN + IBP4/SHBG + BMI*GABD | 0.750 | 0.742 | 0.756 | 0.86 |
| ATD_~PRL + ADA12 + CFAB + IBP4/SHBG + BMI*GABD | 0.749 | 0.739 | 0.759 | 0.58 |
| ATD_~(CFAB + TIMP1)/PGRP2 + IBP4/SHBG + BMI*GABD | 0.749 | 0.740 | 0.757 | 2.13 |
| ATD_~PRL + CFAB + TIMP1 + IBP4/SHBG + BMI*GABD | 0.748 | 0.739 | 0.757 | 0.52 |
| ATD_~PSG3 + CFAB + CHL1 + IBP4/SHBG + BMI*GABD | 0.747 | 0.738 | 0.754 | 0.54 |
| ATD_~PRL + CFAB + CHL1 + IBP4/SHBG + BMI*GABD | 0.746 | 0.736 | 0.755 | 1.17 |
| ATD_~(PSG3 + CFAB)/TETN + IBP4/SHBG + BMI*GABD | 0.746 | 0.739 | 0.752 | 0.83 |
| ATD_~PRL/(TETN + PGRP2) + IBP4/SHBG + BMI*GABD | 0.746 | 0.736 | 0.755 | 0.35 |
| ATD_~CFAB/(TETN + APOC3) + IBP4/SHBG + BMI*GABD | 0.745 | 0.736 | 0.752 | 5.40 |
| ATD_~PSG3 + CFAB + IBP1 + IBP4/SHBG + BMI*GABD | 0.745 | 0.737 | 0.751 | 0.60 |
| ATD_~(CFAB + IBP1)/APOC3 + IBP4/SHBG + BMI*GABD | 0.744 | 0.734 | 0.754 | 1.99 |
| ATD_~CFAB/(PGRP2 + APOC3) + IBP4/SHBG + BMI*GABD | 0.744 | 0.734 | 0.753 | 2.29 |
| ATD_~CFAB + CHL1 + TIMP1 + IBP4/SHBG + BMI*GABD | 0.743 | 0.733 | 0.752 | 0.85 |
| ATD_~(PRL + CHL1)/TETN + IBP4/SHBG + BMI*GABD | 0.742 | 0.732 | 0.751 | 0.35 |
| ATD_~(ADA12 + CFAB)/PGRP2 + IBP4/SHBG + BMI*GABD | 0.742 | 0.732 | 0.750 | 0.69 |
| ATD_~CFAB + IBP1 + TIMP1 + IBP4/SHBG + BMI*GABD | 0.742 | 0.732 | 0.749 | 2.07 |
| ATD_~(CFAB + IBP1)/PGRP2 + IBP4/SHBG + BMI*GABD | 0.742 | 0.732 | 0.749 | 2.25 |
| ATD_~(CFAB + CHL1)/APOC3 + IBP4/SHBG + BMI*GABD | 0.742 | 0.730 | 0.750 | 1.62 |
| ATD_~(ADA12 + CFAB)/TETN + IBP4/SHBG + BMI*GABD | 0.741 | 0.734 | 0.749 | 0.56 |
| ATD_~EGLN/(TETN + ATS13) + IBP4/SHBG + BMI*GABD | 0.741 | 0.732 | 0.751 | 0.38 |
| ATD_~(ADA12 + CFAB)/APOC3 + IBP4/SHBG + BMI*GABD | 0.741 | 0.730 | 0.751 | 1.85 |
| ATD_~(CFAB + CHL1)/PGRP2 + IBP4/SHBG + BMI*GABD | 0.740 | 0.731 | 0.747 | 3.55 |
| ATD_~CFAB/(TETN + PGRP2) + IBP4/SHBG + BMI*GABD | 0.739 | 0.732 | 0.746 | 1.33 |
| ATD_~(CFAB + IBP1)/TETN + IBP4/SHBG + BMI*GABD | 0.739 | 0.730 | 0.746 | 1.00 |
| ATD_~(PRL + CFAB)/ATS13 + IBP4/SHBG + BMI*GABD | 0.739 | 0.727 | 0.750 | 2.51 |
| ATD_~(PRL + EGLN)/ATS13 + IBP4/SHBG + BMI*GABD | 0.737 | 0.725 | 0.748 | 0.93 |
| ATD_~PRL + EGLN + PSG3 + IBP4/SHBG + BMI*GABD | 0.737 | 0.723 | 0.750 | 3.24 |
| ATD_~(PSG3 + TIMP1)/TETN + IBP4/SHBG + BMI*GABD | 0.735 | 0.726 | 0.744 | 0.34 |
| ATD_~PRL + EGLN + IBP1 + IBP4/SHBG + BMI*GABD | 0.735 | 0.724 | 0.746 | 0.56 |
| ATD_~CFAB + IBP1 + CHL1 + IBP4/SHBG + BMI*GABD | 0.734 | 0.726 | 0.741 | 0.84 |
| ATD_~ADA12 + CFAB + TIMP1 + IBP4/SHBG + BMI*GABD | 0.734 | 0.723 | 0.743 | 0.48 |
| ATD_~ADA12 + CFAB + IBP1 + IBP4/SHBG + BMI*GABD | 0.734 | 0.724 | 0.741 | 4.94 |
| ATD_~(EGLN + PSG3)/PGRP2 + IBP4/SHBG + BMI*GABD | 0.732 | 0.722 | 0.742 | 0.29 |
| ATD_~PRL + PSG3 + IBP1 + IBP4/SHBG + BMI*GABD | 0.732 | 0.721 | 0.741 | 0.34 |
| ATD_~CFAB/(ATS13 + APOC3) + IBP4/SHBG + BMI*GABD | 0.731 | 0.719 | 0.741 | 1.02 |
| ATD_~ADA12 + CFAB + CHL1 + IBP4/SHBG + BMI*GABD | 0.731 | 0.721 | 0.739 | 0.52 |
| ATD_~PRL + ADA12 + PSG3 + IBP4/SHBG + BMI*GABD | 0.731 | 0.719 | 0.743 | 0.34 |
| ATD_~(ADA12 + TIMP1)/TETN + IBP4/SHBG + BMI*GABD | 0.730 | 0.721 | 0.738 | 0.43 |
| ATD_~(PRL + PSG3)/APOC3 + IBP4/SHBG + BMI*GABD | 0.729 | 0.718 | 0.740 | 0.48 |
| ATD_~(CFAB + CHL1)/TETN + IBP4/SHBG + BMI*GABD | 0.729 | 0.724 | 0.736 | 0.77 |
| ATD_~TIMP1/(TETN + APOC3) + IBP4/SHBG + BMI*GABD | 0.729 | 0.721 | 0.737 | 0.44 |
| ATD_~(PSG3 + CFAB)/ATS13 + IBP4/SHBG + BMI*GABD | 0.729 | 0.720 | 0.738 | 0.58 |
| ATD_~CFAB/(PGRP2 + ATS13) + IBP4/SHBG + BMI*GABD | 0.729 | 0.718 | 0.737 | 1.94 |
| ATD_~PRL + EGLN + CHL1 + IBP4/SHBG + BMI*GABD | 0.729 | 0.717 | 0.741 | 0.45 |
| ATD_~(PRL + PSG3)/PGRP2 + IBP4/SHBG + BMI*GABD | 0.728 | 0.716 | 0.742 | 0.29 |
| ATD_~PRL + EGLN + TIMP1 + IBP4/SHBG + BMI*GABD | 0.728 | 0.717 | 0.740 | 0.30 |
| ATD_~(ADA12 + PSG3)/TETN + IBP4/SHBG + BMI*GABD | 0.728 | 0.720 | 0.737 | 0.27 |
| ATD_~PRL/(TETN + ATS13) + IBP4/SHBG + BMI*GABD | 0.727 | 0.718 | 0.736 | 0.37 |
| ATD_~(PRL + EGLN)/PGRP2 + IBP4/SHBG + BMI*GABD | 0.727 | 0.715 | 0.737 | 0.97 |
| ATD_~PRL + ADA12 + EGLN + IBP4/SHBG + BMI*GABD | 0.727 | 0.716 | 0.737 | 1.71 |
| ATD_~(PRL + EGLN)/APOC3 + IBP4/SHBG + BMI*GABD | 0.725 | 0.716 | 0.735 | 0.30 |
| ATD_~(CFAB + TIMP1)/ATS13 + IBP4/SHBG + BMI*GABD | 0.725 | 0.716 | 0.733 | 0.68 |
| ATD_~(EGLN + CHL1)/PGRP2 + IBP4/SHBG + BMI*GABD | 0.724 | 0.714 | 0.735 | 0.67 |
| ATD_~EGLN + PSG3 + IBP1 + IBP4/SHBG + BMI*GABD | 0.724 | 0.715 | 0.734 | 1.07 |
| ATD_~(IBP1 + TIMP1)/TETN + IBP4/SHBG + BMI*GABD | 0.724 | 0.715 | 0.731 | 0.37 |
| ATD_~ADA12/(TETN + APOC3) + IBP4/SHBG + BMI*GABD | 0.722 | 0.713 | 0.730 | 0.45 |
| ATD_~(EGLN + TIMP1)/ATS13 + IBP4/SHBG + BMI*GABD | 0.722 | 0.711 | 0.733 | 0.26 |
| ATD_~EGLN + PSG3 + TIMP1 + IBP4/SHBG + BMI*GABD | 0.722 | 0.712 | 0.732 | 0.48 |
| ATD_~EGLN + IBP1 + TIMP1 + IBP4/SHBG + BMI*GABD | 0.721 | 0.710 | 0.732 | 0.31 |
| ATD_~(EGLN + IBP1)/PGRP2 + IBP4/SHBG + BMI*GABD | 0.720 | 0.712 | 0.729 | 0.27 |
| ATD_~EGLN + PSG3 + CHL1 + IBP4/SHBG + BMI*GABD | 0.720 | 0.707 | 0.731 | 0.58 |
| ATD_~(PRL + PSG3)/ATS13 + IBP4/SHBG + BMI*GABD | 0.719 | 0.708 | 0.729 | 0.34 |
| ATD_~PRL + PSG3 + TIMP1 + IBP4/SHBG + BMI*GABD | 0.719 | 0.707 | 0.732 | 0.22 |
| ATD_~(ADA12 + EGLN)/ATS13 + IBP4/SHBG + BMI*GABD | 0.719 | 0.707 | 0.728 | 0.57 |
| ATD_~PRL + PSG3 + CHL1 + IBP4/SHBG + BMI*GABD | 0.718 | 0.708 | 0.728 | 0.24 |
| ATD_~(ADA12 + CFAB)/ATS13 + IBP4/SHBG + BMI*GABD | 0.718 | 0.708 | 0.728 | 1.07 |
| ATD_~(EGLN + TIMP1)/PGRP2 + IBP4/SHBG + BMI*GABD | 0.718 | 0.707 | 0.730 | 0.38 |
| ATD_~EGLN/(PGRP2 + ATS13) + IBP4/SHBG + BMI*GABD | 0.718 | 0.707 | 0.729 | 0.37 |
| ATD_~EGLN + IBP1 + CHL1 + IBP4/SHBG + BMI*GABD | 0.718 | 0.707 | 0.728 | 0.31 |
| ATD_~(EGLN + PSG3)/APOC3 + IBP4/SHBG + BMI*GABD | 0.718 | 0.707 | 0.728 | 0.38 |
| ATD_~EGLN/(ATS13 + APOC3) + IBP4/SHBG + BMI*GABD | 0.718 | 0.706 | 0.729 | 0.40 |
| ATD_~PRL + ADA12 + IBP1 + IBP4/SHBG + BMI*GABD | 0.718 | 0.707 | 0.728 | 0.25 |
| ATD_~(PRL + ADA12)/ATS13 + IBP4/SHBG + BMI*GABD | 0.717 | 0.704 | 0.732 | 1.90 |
| ATD_~(ADA12 + IBP1)TETN + IBP4/SHBG + BMI*GABD | 0.717 | 0.709 | 0.726 | 0.62 |
| ATD_~CFAB/(TETN + ATS13) + IBP4/SHBG + BMI*GABD | 0.717 | 0.707 | 0.724 | 0.86 |
| ATD_~PRL + IBP1 + TIMP1 + IBP4/SHBG + BMI*GABD | 0.717 | 0.705 | 0.727 | 0.20 |
| ATD_~(EGLN + PSG3)/ATS13 + IBP4/SHBG + BMI*GABD | 0.717 | 0.706 | 0.728 | 0.24 |
| ATD_~(CFAB + IBP1)/ATS13 + IBP4/SHBG + BMI*GABD | 0.716 | 0.707 | 0.723 | 0.66 |
| ATD_~(CHL1 + TIMP1)/TETN + IBP4/SHBG + BMI*GABD | 0.716 | 0.709 | 0.723 | 0.32 |
| ATD_~ADA12 + EGLN + IBP1 + IBP4/SHBG + BMI*GABD | 0.716 | 0.707 | 0.727 | 0.67 |
| ATD_~TIMP1/(TETN + PGRP2) + IBP4/SHBG + BMI*GABD | 0.716 | 0.707 | 0.724 | 0.34 |
| ATD_~(CFAB + CHL1)/ATS13 + IBP4/SHBG + BMI*GABD | 0.715 | 0.707 | 0.723 | 0.71 |
| ATD_~EGLN + CHL1 + TIMP1 + IBP4/SHBG + BMI*GABD | 0.715 | 0.701 | 0.727 | 0.36 |
| ATD_~(EGLN + IBP1)/ATS13 + IBP4/SHBG + BMI*GABD | 0.715 | 0.705 | 0.724 | 0.24 |
| ATD_~(PRL + IBP1)/APOC3 + IBP4/SHBG + BMI*GABD | 0.715 | 0.703 | 0.726 | 0.19 |
| ATD_~(ADA12 + EGLN)/APOC3 + IBP4/SHBG + BMI*GABD | 0.714 | 0.703 | 0.725 | 0.59 |
| ATD_~(PRL + IBP1)/PGRP2 + IBP4/SHBG + BMI*GABD | 0.713 | 0.702 | 0.724 | 0.31 |
| ATD_~(ADA12 + EGLN)/PGRP2 + IBP4/SHBG + BMI*GABD | 0.713 | 0.701 | 0.725 | 0.27 |
| ATD_~(ADA12 + PSG3)/PGRP2 + IBP4/SHBG + BMI*GABD | 0.712 | 0.703 | 0.721 | 0.81 |
| ATD_~ADA12 + EGLN + PSG3 + IBP4/SHBG + BMI*GABD | 0.712 | 0.703 | 0.722 | 0.53 |
| ATD_~(EGLN + CHL1)/ATS13 + IBP4/SHBG + BMI*GABD | 0.711 | 0.700 | 0.722 | 0.28 |
| ATD_~(ADA12 + PSG3)/APOC3 + IBP4/SHBG + BMI*GABD | 0.711 | 0.699 | 0.720 | 0.52 |
| ATD_~PRL + IBP1 + CHL1 + IBP4/SHBG + BMI*GABD | 0.711 | 0.700 | 0.722 | 0.25 |
| ATD_~PSG3 + IBP1 + TIMP1 + IBP4/SHBG + BMI*GABD | 0.710 | 0.701 | 0.719 | 0.23 |
| ATD_~PSG3/(TETN + APOC3) + IBP4/SHBG + BMI*GABD | 0.710 | 0.703 | 0.719 | 0.21 |
| ATD_~PRL + ADA12 + CHL1 + IBP4/SHBG + BMI*GABD | 0.710 | 0.698 | 0.721 | 0.21 |
| ATD_~(PRL + TIMP1)/APOC3 + IBP4/SHBG + BMI*GABD | 0.710 | 0.698 | 0.721 | 0.37 |
| ATD_~PRL + ADA12 + TIMP1 + IBP4/SHBG + BMI*GABD | 0.710 | 0.698 | 0.724 | 0.19 |
| ATD_~ADA12/(TETN + PGRP2) + IBP4/SHBG + BMI*GABD | 0.709 | 0.701 | 0.718 | 1.25 |
| ATD_~(PSG3 + TIMP1)/PGRP2 + IBP4/SHBG + BMI*GABD | 0.709 | 0.699 | 0.719 | 0.39 |
| ATD_~PRL/(ATS13 + APOC3) + IBP4/SHBG + BMI*GABD | 0.709 | 0.693 | 0.722 | 0.28 |
| ATD_~(PRL + ADA12)/APOC3 + IBP4/SHBG + BMI*GABD | 0.709 | 0.699 | 0.719 | 0.25 |
| ATD_~(PRL + IBP1)/ATS13 + IBP4/SHBG + BMI*GABD | 0.709 | 0.699 | 0.718 | 0.27 |
| ATD_~ADA12 + EGLN + TIMP1 + IBP4/SHBG + BMI*GABD | 0.709 | 0.699 | 0.718 | 0.23 |
| ATD_~(PSG3 + TIMP1)/APOC3 + IBP4/SHBG + BMI*GABD | 0.708 | 0.699 | 0.717 | 0.36 |
| ATD_~ADA12 + EGLN + CHL1 + IBP4/SHBG + BMI*GABD | 0.708 | 0.697 | 0.719 | 0.25 |
| ATD_~(ADA12 + CHL1)/TETN + IBP4/SHBG + BMI*GABD | 0.708 | 0.700 | 0.716 | 0.31 |
| ATD_~(EGLN + CHL1)/APOC3 + IBP4/SHBG + BMI*GABD | 0.708 | 0.696 | 0.718 | 0.29 |
| ATD_~ADA12 + PSG3 + IBP1 + IBP4/SHBG + BMI*GABD | 0.707 | 0.698 | 0.715 | 0.22 |
| ATD_~ADA12 + PSG3 + TIMP1 + IBP4/SHBG + BMI*GABD | 0.706 | 0.696 | 0.716 | 0.42 |
| ATD_~(EGLN + IBP1)/APOC3 + IBP4/SHBG + BMI*GABD | 0.706 | 0.696 | 0.715 | 0.24 |
| ATD_~EGLN/(PGRP2 + APOC3) + IBP4/SHBG + BMI*GABD | 0.705 | 0.693 | 0.716 | 0.31 |
| ATD_~PSG3/(TETN + PGRP2) + IBP4/SHBG + BMI*GABD | 0.705 | 0.697 | 0.711 | 0.22 |
| ATD_~(EGLN + TIMP1)/APOC3 + IBP4/SHBG + BMI*GABD | 0.705 | 0.695 | 0.714 | 0.22 |
| ATD_~(PRL + CHL1)/APOC3 + IBP4/SHBG + BMI*GABD | 0.704 | 0.695 | 0.715 | 0.30 |
| ATD_~(PRL + ADA12)/PGRP2 + IBP4/SHBG + BMI*GABD | 0.704 | 0.692 | 0.714 | 0.36 |
| ATD_~(PSG3 + IBP1)/PGRP2 + IBP4/SHBG + BMI*GABD | 0.704 | 0.697 | 0.711 | 0.32 |
| ATD_~PRL + CHL1 + TIMP1 + IBP4/SHBG + BMI*GABD | 0.703 | 0.694 | 0.714 | 0.21 |
| ATD_~(PRL + CHL1)/PGRP2 + IBP4/SHBG + BMI*GABD | 0.703 | 0.694 | 0.713 | 0.31 |
| ATD_~(PSG3 + IBP1)/TETN + IBP4/SHBG + BMI*GABD | 0.703 | 0.695 | 0.710 | 0.33 |
| ATD_~(PRL + CHL1)/ATS13 + IBP4/SHBG + BMI*GABD | 0.701 | 0.690 | 0.713 | 0.53 |
| ATD_~PSG3 + CHL1 + TIMP1 + IBP4/SHBG + BMI*GABD | 0.700 | 0.689 | 0.708 | 0.29 |
| ATD_~CHL1/(TETN + APOC3) + IBP4/SHBG + BMI*GABD | 0.700 | 0.690 | 0.708 | 0.31 |
| ATD_~PRL/(PGRP2 + ATS13) + IBP4/SHBG + BMI*GABD | 0.699 | 0.685 | 0.710 | 0.59 |
| ATD_~(ADA12 + IBP1)/PGRP2 + IBP4/SHBG + BMI*GABD | 0.699 | 0.689 | 0.707 | 0.24 |
| ATD_~(PSG3 + CHL1)/TETN + IBP4/SHBG + BMI*GABD | 0.698 | 0.690 | 0.707 | 0.24 |
| ATD_~PRL/(PGRP2 + APOC3) + IBP4/SHBG + BMI*GABD | 0.698 | 0.687 | 0.708 | 0.22 |
| ATD_~(ADA12 + CHL1)/PGRP2 + IBP4/SHBG + BMI*GABD | 0.698 | 0.687 | 0.707 | 0.25 |
| ATD_~(PRL + TIMP1)/PGRP2 + IBP4/SHBG + BMI*GABD | 0.697 | 0.687 | 0.705 | 0.23 |
| ATD_~(CHL1 + TIMP1)/PGRP2 + IBP4/SHBG + BMI*GABD | 0.697 | 0.688 | 0.706 | 0.30 |
| ATD_~ADA12/(TETN + ATS13) + IBP4/SHBG + BMI*GABD | 0.697 | 0.689 | 0.704 | 0.27 |
| ATD_~ADA12/(PGRP2 + APOC3) + IBP4/SHBG + BMI*GABD | 0.696 | 0.685 | 0.707 | 0.27 |
| ATD_~TIMP1/(TETN + ATS13) + IBP4/SHBG + BMI*GABD | 0.696 | 0.686 | 0.705 | 0.22 |
| ATD_~ADA12 + IBP1 + TIMP1 + IBP4/SHBG + BMI*GABD | 0.696 | 0.685 | 0.704 | 0.33 |
| ATD_~ADA12 + PSG3 + CHL1 + IBP4/SHBG + BMI*GABD | 0.696 | 0.685 | 0.704 | 0.61 |
| ATD_~(PRL + TIMP1)/ATS13 + IBP4/SHBG + BMI*GABD | 0.695 | 0.685 | 0.707 | 0.22 |
| ATD_~(ADA12 + PSG3)/ATS13 + IBP4/SHBG + BMI*GABD | 0.695 | 0.686 | 0.705 | 0.51 |
| ATD_~IBP1/(TETN + PGRP2) + IBP4/SHBG + BMI*GABD | 0.695 | 0.687 | 0.703 | 0.36 |
| ATD_~(ADA12 + TIMP1)/APOC3/+ IBP4/SHBG + BMI*GABD | 0.694 | 0.686 | 0.703 | 0.25 |
| ATD_~(ADA12 + CHL1)/APOC3 + IBP4/SHBG + BMI*GABD | 0.694 | 0.682 | 0.704 | 0.35 |
| ATD_ ~1/(TETN + PGRP2 + APOC3) + IBP4/SHBG + BMI*GABD | 0.694 | 0.686 | 0.702 | 0.31 |
| ATD_~(IBP1 + TIMP1)/PGPR2 + IBP4/SHBG + BMI*GABD | 0.694 | 0.683 | 0.704 | 0.27 |
| ATD_~PSG3/(PGRP2 + APOC3) + IBP4/SHBG + BMI*GABD | 0.694 | 0.681 | 0.703 | 0.24 |
| ATD_~(PSG3 + IBP1)/APOC3 + IBP4/SHBG + BMI*GABD | 0.694 | 0.685 | 0.701 | 0.21 |
| ATD_~(ADA12 + IBP1)/APOC3 + IBP4/SHBG + BMI*GABD | 0.694 | 0.682 | 0.703 | 0.28 |
| ATD_~(PSG3 + TIMP1)/ATS13 + IBP4/SHBG + BMI*GABD | 0.693 | 0.683 | 0.703 | 0.44 |
| ATD_~(IBP1 + CHL1)/TETN + IBP4/SHBG + BMI*GABD | 0.693 | 0.686 | 0.701 | 0.23 |
| ATD_~IBP1/(TETN + APOC3) + IBP4/SHBG + BMI*GABD | 0.693 | 0.684 | 0.702 | 0.36 |
| ATD_~(IBP1 + TIMP1)/APOC3 + IBP4/SHBG + BMI*GABD | 0.692 | 0.683 | 0.700 | 0.61 |
| ATD_~(PSG3 + CHL1)/PGRP2 + IBP4/SHBG + BMI*GABD | 0.691 | 0.682 | 0.699 | 1.52 |
| ATD_~(ADA12 + TIMP1)/PGRP2 + IBP4/SHBG + BMI*GABD | 0.690 | 0.681 | 0.699 | 0.84 |
| ATD_~(PSG3 + CHL1)/APOC3 + IBP4/SHBG + BMI*GABD | 0.690 | 0.681 | 0.697 | 0.28 |
| ATD_~TIMP1/(ATS13 + APOC3) + IBP4/SHBG + BMI*GABD | 0.689 | 0.678 | 0.702 | 0.20 |
| ATD_~TIMP1/(PGRP2 + APOC3) + IBP4/SHBG + BMI*GABD | 0.689 | 0.681 | 0.697 | 0.25 |
| ATD_~(CHL1 + TIMP1)/APOC3 + IBP4/SHBG + BMI*GABD | 0.689 | 0.680 | 0.699 | 0.22 |
| ATD_~CHL1/(TETN + PGRP2) + IBP4/SHBG + BMI*GABD | 0.689 | 0.680 | 0.696 | 0.20 |
| ATD_~PSG3 + IBP1 + CHL1 + IBP4/SHBG + BMI*GABD | 0.689 | 0.681 | 0.696 | 0.28 |
| ATD_~(ADA12 + TIMP1)/ATS13 + IBP4/SHBG + BMI*GABD | 0.688 | 0.675 | 0.698 | 0.27 |
| ATD_~IBP1 + CHL1 + TIMP1 + IBP4/SHBG + BMI*GABD | 0.687 | 0.678 | 0.696 | 0.22 |
| ATD_~(ADA12 + IBP1)/ATS13 + IBP4/SHBG + BMI*GABD | 0.685 | 0.675 | 0.694 | 0.28 |
| ATD_~1/(TETN + ATS13 + APOC3) + IBP4/SHBG + BMI*GABD | 0.685 | 0.677 | 0.692 | 0.19 |
| ATD_~PSG3/(TETN + ATS13) + IBP4/SHBG + BMI*GABD | 0.684 | 0.675 | 0.692 | 0.28 |
| ATD_~PSG3/(ATS13 + APOC3) + IBP4/SHBG + BMI*GABD | 0.684 | 0.674 | 0.692 | 0.44 |
| ATD_~ADA12 + CHL1 + TIMP1 + IBP4/SHBG + BMI*GABD | 0.682 | 0.672 | 0.693 | 0.17 |
| ATD_~ADA12 + IBP1 + CHL1 + IBP4/SHBG + BMI*GABD | 0.682 | 0.673 | 0.692 | 0.43 |
| ATD_~(IBP1 + CHL1)/PGRP2 + IBP4/SHBG + BMI*GABD | 0.681 | 0.673 | 0.690 | 0.30 |
| ATD_~(ADA12 + CHL1)/ATS13 + IBP4/SHBG + BMI*GABD | 0.681 | 0.672 | 0.689 | 1.45 |
| ATD_~ADA12/(PGRP2 + ATS13) + IBP4/SHBG + BMI*GABD | 0.681 | 0.671 | 0.690 | 0.46 |
| ATD_~(PSG3 + IBP1)/ATS13 + IBP4/SHBG + BMI*GABD | 0.680 | 0.671 | 0.689 | 0.30 |
| ATD_~ADA12/(ATS13 + APOC3) + IBP4/SHBG + BMI*GABD | 0.680 | 0.669 | 0.690 | 0.51 |
| ATD_~CHL1/(PGRP2 + APOC3) + IBP4/SHBG + BMI*GABD | 0.678 | 0.670 | 0.687 | 0.32 |
| ATD_~(IBP1 + TIMP1)/ATS13 + IBP4/SHBG + BMI*GABD | 0.678 | 0.669 | 0.688 | 0.41 |
| ATD_~(CHL1 + TIMP1)/ATS13 + IBP4/SHBG + BMI*GABD | 0.676 | 0.666 | 0.687 | 0.18 |
| ATD_~1/(TETN + PGRP2 + ATS13) + IBP4/SHBG + BMI*GABD | 0.676 | 0.667 | 0.684 | 0.30 |
| ATD_~IBP1/(PGRP2 + APOC3) + IBP4/SHBG + BMI*GABD | 0.676 | 0.667 | 0.683 | 0.21 |
| ATD_~IBP1/(TETN + ATS13) + IBP4/SHBG + BMI*GABD | 0.676 | 0.667 | 0.684 | 0.50 |
| ATD_~TIMP1/(PGRP2 + ATS13) + IBP4/SHBG + BMI*GABD | 0.675 | 0.664 | 0.685 | 0.21 |
| ATD_~PSG3/(PGRP2 + ATS13) + IBP4/SHBG + BMI*GABD | 0.674 | 0.665 | 0.684 | 0.17 |
| ATD_ ~1/(PGRP2 + ATS13 + APOC3) + IBP4/SHBG + BMI*GABD | 0.673 | 0.662 | 0.682 | 0.27 |
| ATD_~(IBP1 + CHL1)/APOC3 + IBP4/SHBG + BMI*GABD | 0.672 | 0.662 | 0.679 | 0.35 |
| ATD_~CHL1/(TETN + ATS13) + IBP4/SHBG + BMI*GABD | 0.672 | 0.663 | 0.680 | 0.19 |
| ATD_~(PSG3 + CHL1)/ATS13 + IBP4/SHBG + BMI*GABD | 0.670 | 0.660 | 0.680 | 0.73 |
| ATD_~CHL1/(PGRP2 + ATS13) + IBP4/SHBG + BMI*GABD | 0.669 | 0.659 | 0.678 | 0.25 |
| ATD_~CHL1/(ATS13 + APOC3) + IBP4/SHBG + BMI*GABD | 0.669 | 0.658 | 0.679 | 0.31 |
| ATD_~IBP1/(ATS13 + APOC3) + IBP4/SHBG + BMI*GABD | 0.662 | 0.653 | 0.671 | 0.25 |
| ATD_~IBP1/(PGRP2 + ATS13) + IBP4/SHBG + BMI*GABD | 0.657 | 0.647 | 0.665 | 0.18 |
| ATD_~(IBP1 + CHL1)/ATS13 + IBP4/SHBG + BMI*GABD | 0.656 | 0.648 | 0.662 | 0.40 |

TABLE 9
Reversal Group Classification Performance at Predicting sPTB <34 weeks gestation.
Reversal group comprises IBP4/SHBG, one additional protein biomarker, two
additional clinical variables and a demographic variable of prior sPTB.

| Reversal Group | AUC | CI low | CI high | Sum Rank |
|---|---|---|---|---|
| ATD_~CFAB + IBP4/SHBG + GABD*BMI + PriorPTB | 0.728 | 0.719 | 0.735 | 19.49 |
| ATD_~1/APOC3 + IBP4/SHBG + GABD*BMI + PriorPTB | 0.697 | 0.687 | 0.706 | 16.29 |
| ATD_~IBP1 + IBP4/SHBG + GABD*BMI + PriorPTB | 0.695 | 0.687 | 0.702 | 6.54 |
| ATD_~1/PGRP2 + IBP4/SHBG + GABD*BMI + PriorPTB | 0.689 | 0.678 | 0.698 | 6.51 |
| ATD_~EGLN + IBP4/SHBG + GABD*BMI + PriorPTB | 0.702 | 0.691 | 0.711 | 6.28 |
| ATD_~1/TETN + IBP4/SHBG + GABD*BMI + PriorPTB | 0.693 | 0.683 | 0.701 | 5.93 |
| ATD_~ADA12 + IBP4/SHBG + GABD*BMI + PriorPTB | 0.691 | 0.681 | 0.701 | 5.46 |
| ATD_~1/ATS13 + IBP4/SHBG + GABD*BMI + PriorPTB | 0.657 | 0.648 | 0.666 | 5.22 |
| ATD_~PSG3 + IBP4/SHBG + GABD*BMI + PriorPTB | 0.703 | 0.693 | 0.713 | 4.90 |
| ATD_~CHL1 + IBP4/SHBG + GABD*BMI + PriorPTB | 0.683 | 0.674 | 0.690 | 4.90 |
| ATD_~TIMP1 + IBP4/SHBG + GABD*BMI + PriorPTB | 0.691 | 0.681 | 0.700 | 4.55 |
| ATD_~PRL + IBP4/SHBG + GABD*BMI + PriorPTB | 0.699 | 0.688 | 0.708 | 3.93 |

TABLE 10
Reversal Group Classification Performance at Predicting sPTB <34 weeks gestation.
Reversal group comprises IBP4/SHBG, two additional protein biomarkers, two
additional clinical variables and a demographic variable of prior sPTB.

| Reversal Group | AUC | CI low | CI high | Sum Rank |
|---|---|---|---|---|
| ATD_~EGLN + CFAB + IBP4/SHBG + PriorPTB + BMI*GABD | 0.753 | 0.744 | 0.761 | 6.66 |
| ATD_~EGLN/TETN + IBP4/SHBG + PriorPTB + BMI*GABD | 0.752 | 0.742 | 0.762 | 3.13 |
| ATD_~PSG3 + CFAB + IBP4/SHBG + PriorPTB + BMI*GABD | 0.741 | 0.733 | 0.747 | 4.10 |
| ATD_~PRL + CFAB + IBP4/SHBG + PriorPTB + BMI*GABD | 0.738 | 0.728 | 0.748 | 7.28 |
| ATD_~CFAB/APOC3 + IBP4/SHBG + PriorPTB + BMI*GABD | 0.737 | 0.728 | 0.745 | 12.58 |
| ATD_~PRL/TETN + IBP4/SHBG + PriorPTB + BMI*GABD | 0.736 | 0.725 | 0.747 | 1.41 |
| ATD_~CFAB + TIMP1 + IBP4/SHBG + PriorPTB + BMI*GABD | 0.735 | 0.725 | 0.743 | 5.55 |
| ATD_~CFAB/PGRP2 + IBP4/SHBG + PriorPTB + BMI*GABD | 0.735 | 0.727 | 0.742 | 10.58 |
| ATD_~CFAB + IBP1 + IBP4/SHBG + PriorPTB + BMI*GABD | 0.731 | 0.722 | 0.738 | 2.42 |
| ATD_~CFAB + CHL1 + IBP4/SHBG + PriorPTB + BMI*GABD | 0.731 | 0.722 | 0.737 | 2.96 |
| ATD_~EGLN + IBP1 + IBP4/SHBG + PriorPTB + BMI*GABD | 0.729 | 0.719 | 0.739 | 1.58 |
| ATD_~PRL + PSG3 + IBP4/SHBG + PriorPTB + BMI*GABD | 0.727 | 0.715 | 0.738 | 0.97 |
| ATD_~PRL + IBP1 + IBP4/SHBG + PriorPTB + BMI*GABD | 0.726 | 0.714 | 0.736 | 0.94 |
| ATD_~EGLN + PSG3 + IBP4/SHBG + PriorPTB + BMI*GABD | 0.726 | 0.717 | 0.736 | 1.30 |
| ATD_~TIMP/TETN + IBP4/SHBG + PriorPTB + BMI*GABD | 0.725 | 0.716 | 0.733 | 1.25 |
| ATD_~CFAB/TETN + IBP4/SHBG + PriorPTB + BMI*GABD | 0.724 | 0.716 | 0.730 | 4.18 |
| ATD_~EGLN/PGRP2 + IBP4/SHBG + PriorPTB + BMI*GABD | 0.723 | 0.712 | 0.733 | 1.30 |
| ATD_~PSG3/PGRP2 + IBP4/SHBG + PriorPTB + BMI*GABD | 0.720 | 0.710 | 0.728 | 1.16 |
| ATD_~ADA12 + CFAB + IBP4/SHBG + PriorPTB + BMI*GABD | 0.719 | 0.711 | 0.727 | 4.73 |
| ATD_~PRL + EGLN + IBP4/SHBG + PriorPTB + BMI*GABD | 0.719 | 0.707 | 0.729 | 3.40 |
| ATD_~ADA12 + PSG3 + IBP4/SHBG + PriorPTB + BMI*GABD | 0.718 | 0.709 | 0.727 | 0.91 |
| ATD_~ADA12/TETN + IBP4/SHBG + PriorPTB + BMI*GABD | 0.717 | 0.710 | 0.727 | 1.04 |
| ATD_~PSG3/TETN + IBP4/SHBG + PriorPTB + BMI*GABD | 0.717 | 0.709 | 0.725 | 0.88 |
| ATD_~PSG3 + TIMP1 + IBP4/SHBG + PriorPTB + BMI*GABD | 0.717 | 0.706 | 0.727 | 0.77 |
| ATD_~PSG3 + IBP1 + IBP4/SHBG + PriorPTB + BMI*GABD | 0.717 | 0.707 | 0.725 | 1.52 |
| ATD_~PSG3/APOC3 + IBP4/SHBG + PriorPTB + BMI*GABD | 0.716 | 0.706 | 0.725 | 1.59 |
| ATD_~EGLN/APOC3 + IBP4/SHBG + PriorPTB + BMI*GABD | 0.713 | 0.703 | 0.722 | 1.36 |
| ATD_~EGLN + TIMP1 + IBP4/SHBG + PriorPTB + BMI*GABD | 0.712 | 0.701 | 0.722 | 1.42 |
| ATD_~IBP1 + TIMP1 + IBP4/SHBG + PriorPTB + BMI*GABD | 0.711 | 0.701 | 0.720 | 0.83 |
| ATD_~ADA12/APOC3 + IBP4/SHBG + PriorPTB + BMI*GABD | 0.711 | 0.699 | 0.721 | 1.22 |
| ATD_~PRL + ADA12 + IBP4/SHBG + PriorPTB + BMI*GABD | 0.711 | 0.700 | 0.723 | 0.62 |
| ATD_~IBP1/TETN + IBP4/SHBG + PriorPTB + BMI*GABD | 0.711 | 0.703 | 0.718 | 0.87 |
| ATD_~1/(TETN + APOC3) + IBP4/SHBG + PriorPTB + BMI*GABD | 0.711 | 0.701 | 0.719 | 1.80 |
| ATD_~ADA12 + EGLN + IBP4/SHBG + PriorPTB + BMI*GABD | 0.710 | 0.699 | 0.720 | 1.46 |
| ATD_~CFAB/ATS13 + IBP4/SHBG + PriorPTB + BMI*GABD | 0.709 | 0.699 | 0.718 | 3.25 |
| ATD_~IBP1/PGRP2 + IBP4/SHBG + PriorPTB + BMI*GABD | 0.709 | 0.700 | 0.717 | 1.38 |
| ATD_~TIMP1/APOC3 + IBP4/SHBG + PriorPTB + BMI*GABD | 0.709 | 0.699 | 0.717 | 1.24 |
| ATD_~EGLN + CHL1 + IBP4/SHBG + PriorPTB + BMI*GABD | 0.708 | 0.697 | 0.718 | 2.63 |
| ATD_~TIMP1/PGRP2 + IBP4/SHBG + PriorPTB + BMI*GABD | 0.707 | 0.697 | 0.716 | 0.98 |
| ATD_~IBP1/APOC3 + IBP4/SHBG + PriorPTB + BMI*GABD | 0.706 | 0.695 | 0.715 | 3.75 |
| ATD_~PRL/APOC3 + IBP4/SHBG + PriorPTB + BMI*GABD | 0.706 | 0.695 | 0.717 | 0.74 |
| ATD_~PRL/PGRP2 + IBP4/SHBG + PriorPTB + BMI*GABD | 0.705 | 0.694 | 0.715 | 0.92 |
| ATD_~ADA12 + IBP1 + IBP4/SHBG + PriorPTB + BMI*GABD | 0.704 | 0.695 | 0.714 | 1.29 |
| ATD_~PRL + TIMP1 + IBP4/SHBG + PriorPTB + BMI*GABD | 0.704 | 0.694 | 0.713 | 0.76 |
| ATD_~PSG3 + CHL1 + IBP4/SHBG + PriorPTB + BMI*GABD | 0.703 | 0.694 | 0.712 | 0.94 |
| ATD_~CHL1 + TIMP1 + IBP4/SHBG + PriorPTB + BMI*GABD | 0.700 | 0.692 | 0.708 | 0.66 |
| ATD_~1/(TETN + PGRP2) + IBP4/SHBG + PriorPTB + BMI*GABD | 0.699 | 0.690 | 0.705 | 1.08 |
| ATD_~ADA12/PGRP2 + IBP4/SHBG + PriorPTB + BMI*GABD | 0.699 | 0.688 | 0.708 | 1.65 |
| ATD_~PRL/ATS13 + IBP4/SHBG + PriorPTB + BMI*GABD | 0.698 | 0.686 | 0.709 | 1.03 |
| ATD_~CHL1/APOC3 + IBP4/SHBG + PriorPTB + BMI*GABD | 0.698 | 0.689 | 0.706 | 3.45 |
| ATD_~EGLN/ATS13 + IBP4/SHBG + PriorPTB + BMI*GABD | 0.698 | 0.689 | 0.708 | 1.17 |
| ATD_~1/(PGRP2 + APOC3) + IBP4/SHBG + PriorPTB + BMI*GABD | 0.698 | 0.688 | 0.706 | 1.13 |
| ATD_~ADA12 + TIMP1 + IBP4/SHBG + PriorPTB + BMI*GABD | 0.697 | 0.688 | 0.707 | 0.68 |
| ATD_~PRL + CHL1 + IBP4/SHBG + PriorPTB + BMI*GABD | 0.697 | 0.686 | 0.708 | 0.57 |
| ATD_~CHL1/PGRP2 + IBP4/SHBG + PriorPTB + BMI*GABD | 0.697 | 0.689 | 0.705 | 1.24 |
| ATD_~CHL1/TETN + IBP4/SHBG + PriorPTB + BMI*GABD | 0.696 | 0.689 | 0.701 | 0.89 |
| ATD_~IBP1 + CHL1 + IBP4/SHBG + PriorPTB + BMI*GABD | 0.695 | 0.687 | 0.703 | 1.04 |
| ATD_~ADA12 + CHL1 + IBP4/SHBG + PriorPTB + BMI*GABD | 0.692 | 0.683 | 0.701 | 1.23 |
| ATD_~PSG3/ATS13 + IBP4/SHBG + PriorPTB + BMI*GABD | 0.686 | 0.678 | 0.695 | 1.15 |
| ATD_~1/(ATS13 + APOC3) + IBP4/SHBG + PriorPTB + BMI*GABD | 0.685 | 0.675 | 0.694 | 1.48 |
| ATD_~ADA12/ATS13 + IBP4/SHBG + PriorPTB + BMI*GABD | 0.684 | 0.674 | 0.693 | 2.70 |
| ATD_~TIMP1/ATS13 + IBP4/SHBG + PriorPTB + BMI*GABD | 0.683 | 0.673 | 0.692 | 1.45 |
| ATD_~IBP1/ATS13 + IBP4/SHBG + PriorPTB + BMI*GABD | 0.680 | 0.672 | 0.688 | 1.12 |
| ATD_~1(TETN + ATS13) + IBP4/SHBG + PriorPTB + BMI*GABD | 0.678 | 0.669 | 0.685 | 0.82 |
| ATD_~1/(PGRP2 + ATS13) + IBP4/SHBG + PriorPTB + BMI*GABD | 0.673 | 0.663 | 0.681 | 0.69 |
| ATD_~CHL1/ATS13 + IBP4/SHBG + PriorPTB + BMI*GABD | 0.668 | 0.660 | 0.675 | 1.60 |

TABLE 11
Reversal Group Classification Performance at Predicting sPTB <34 weeks gestation.
Reversal group comprises IBP4/SHBG, three additional protein biomarkers, two
additional clinical variables and a demographic variable of prior sPTB.

| Reversal Group | AUC | CI low | CI high | Sum Rank |
|---|---|---|---|---|
| ATD_~(PRL + EGLN)/TETN + IBP4/SHBG + PriorPTB + BMI*GABD | 0.779 | 0.770 | 0.789 | 2.92 |
| ATD_~(EGLN + CFAB)/TETN + IBP4/SHBG + PriorPTB + BMI*GABD | 0.779 | 0.771 | 0.786 | 2.51 |
| ATD_~EGLN + PSG3 + TETN + IBP4/SHBG + PriorPTB + BMI*GABD | 0.773 | 0.763 | 0.785 | 2.75 |
| ATD_~(EGLN + TIMP1)/TETN + IBP4/SHBG + PriorPTB + BMI*GABD | 0.770 | 0.760 | 0.780 | 1.14 |
| ATD_~EGLN + PSG3 + CFAB + IBP4/SHBG + PriorPTB + BMI*GABD | 0.766 | 0.756 | 0.775 | 1.09 |
| ATD_~(EGLN + IBP1)/TETN + IBP4/SHBG + PriorPTB + BMI*GABD | 0.764 | 0.755 | 0.773 | 0.96 |
| ATD_~PRL + EGLN + CFAB + IBP4/SHBG + PriorPTB + BMI*GABD | 0.763 | 0.754 | 0.772 | 2.26 |
| ATD_~EGLN/(TETN + PGRP2) + IBP4/SHBG + PriorPTB + BMI*GABD | 0.763 | 0.755 | 0.772 | 0.83 |
| ATD_~(EGLN + CFAB)/PGRP2 + IBP4/SHBG + PriorPTB + BMI*GABD | 0.762 | 0.755 | 0.770 | 1.52 |
| ATD_~(EGLN + CFAB)/APOC3 + IBP4/SHBG + PriorPTB + BMI*GABD | 0.761 | 0.751 | 0.770 | 4.62 |
| ATD_~(PRL + PSG3)/TETN + IBP4/SHBG + PriorPTB + BMI*GABD | 0.761 | 0.751 | 0.770 | 0.74 |
| ATD_~EGLN/(TETN + APOC3) + IBP4/SHBG + PriorPTB + BMI*GABD | 0.760 | 0.751 | 0.769 | 1.66 |
| ATD_~(PRL + CFAB)/TETN + IBP4/SHBG + PriorPTB + BMI*GABD | 0.759 | 0.749 | 0.767 | 1.29 |
| ATD_~(PRL + IBP1)/TETN + IBP4/SHBG + PriorPTB + BMI*GABD | 0.758 | 0.748 | 0.768 | 0.53 |
| ATD_~PRL + PSG3 + CFAB + IBP4/SHBG + PriorPTB + BMI*GABD | 0.758 | 0.748 | 0.768 | 1.13 |
| ATD_~(EGLN + CHL1)/TETN + IBP4/SHBG + PriorPTB + BMI*GABD | 0.757 | 0.746 | 0.766 | 0.70 |
| ATD_~EGLN + CFAB + IBP1 + IBP4/SHBG + PriorPTB + BMI*GABD | 0.757 | 0.747 | 0.764 | 1.52 |
| ATD_~EGLN + CFAB + TIMP1 + IBP4/SHBG + PriorPTB + BMI*GABD | 0.756 | 0.747 | 0.764 | 1.02 |
| ATD_~(PRL + TIMP1)/TETN + IBP4/SHBG + PriorPTB + BMI*GABD | 0.755 | 0.747 | 0.765 | 0.42 |
| ATD_~(PSG3 + CFAB)/APOC3 + IBP4/SHBG + PriorPTB + BMI*GABD | 0.755 | 0.746 | 0.763 | 1.06 |
| ATD_~(ADA12 + EGLN)/TETN + IBP4/SHBG + PriorPTB + BMI*GABD | 0.755 | 0.746 | 0.762 | 0.54 |
| ATD_~(PRL + ADA12)/TETN + IBP4/SHBG + PriorPTB + BMI*GABD | 0.754 | 0.745 | 0.762 | 0.34 |
| ATD_~(PRL + CFAB)/APOC3 + IBP4/SHBG + PriorPTB + BMI*GABD | 0.753 | 0.741 | 0.765 | 6.49 |
| ATD_ ~(PSG3 + TIMP1)/TETN + IBP4/SHBG + PriorPTB + BMI*GABD | 0.753 | 0.745 | 0.761 | 0.50 |
| ATD_~PSG3 + CFAB + TIMP1 + IBP4/SHBG + PriorPTB + BMI*GABD | 0.753 | 0.744 | 0.761 | 0.60 |
| ATD_~PRL/(TETN + APOC3) + IBP4/SHBG + PriorPTB + BMI*GABD | 0.752 | 0.742 | 0.764 | 0.55 |
| ATD_~(PSG3 + CFAB)/PGRP2 + IBP4/SHBG + PriorPTB + BMI*GABD | 0.751 | 0.743 | 0.758 | 2.83 |
| ATD_~EGLN + CFAB + CHL1 + IBP4/SHBG + PriorPTB + BMI*GABD | 0.750 | 0.741 | 0.759 | 1.29 |
| ATD_~PRL + CFAB + IBP1 + IBP4/SHBG + PriorPTB + BMI*GABD | 0.750 | 0.740 | 0.759 | 0.99 |
| ATD_~(CFAB + TIMP1)/APOC3 + IBP4/SHBG + PriorPTB + BMI*GABD | 0.750 | 0.740 | 0.759 | 9.62 |
| ATD_~PRL + PSG3 + IBP1 + IBP4/SHBG + PriorPTB + BMI*GABD | 0.748 | 0.737 | 0.757 | 0.32 |
| ATD_~EGLN + PSG3 + IBP1 + IBP4/SHBG + PriorPTB + BMI*GABD | 0.747 | 0.735 | 0.759 | 1.15 |
| ATD_~(EGLN + CFAB)/ATS13 + IBP4/SHBG + PriorPTB + BMI*GABD | 0.747 | 0.736 | 0.755 | 0.80 |
| ATD_~(IBP1 + TIMP1)/TETN + IBP4/SHBG + PriorPTB + BMI*GABD | 0.746 | 0.738 | 0.753 | 0.88 |
| ATD_~ADA12 + PSG3 + CFAB + IBP4/SHBG + PriorPTB + BMI*GABD | 0.746 | 0.737 | 0.755 | 1.08 |
| ATD_~(CFAB + TIMP1)/PGRP2 + IBP4/SHBG + PriorPTB + BMI*GABD | 0.745 | 0.737 | 0.754 | 1.34 |
| ATD_~PSG3 + CFAB + IBP1 + IBP4/SHBG + PriorPTB + BMI*GABD | 0.745 | 0.737 | 0.753 | 0.49 |
| ATD_~(CFAB + IBP1)/PGRP2 + IBP4/SHBG + PriorPTB + BMI*GABD | 0.744 | 0.735 | 0.751 | 2.80 |
| ATD_~TIMP1/(TETN + APOC3) + IBP4/SHBG + PriorPTB + BMI*GABD | 0.744 | 0.734 | 0.753 | 2.19 |
| ATD_~(CFAB + TIMP1)/TETN + IBP4/SHBG + PriorPTB + BMI*GABD | 0.744 | 0.737 | 0.750 | 0.64 |
| ATD_~PRL + EGLN + IBP1 + IBP4/SHBG + PriorPTB + BMI*GABD | 0.744 | 0.733 | 0.755 | 0.61 |
| ATD_~ADA12 + EGLN + CFAB + IBP4/SHBG + PriorPTB + BMI*GABD | 0.743 | 0.734 | 0.752 | 0.34 |
| ATD_~(EGLN + PSG3)/PGRP2 + IBP4/SHBG + PriorPTB + BMI*GABD | 0.743 | 0.732 | 0.754 | 0.36 |
| ATD_~(ADA12 + PSG3)/TETN + IBP4/SHBG + PriorPTB + BMI*GABD | 0.743 | 0.736 | 0.751 | 0.45 |
| ATD_~(CFAB + IBP1)/APOC3 + IBP4/SHBG + PriorPTB + BMI*GABD | 0.742 | 0.732 | 0.750 | 1.07 |
| ATD_~(PSG3 + CFAB)/TETN + IBP4/SHBG + PriorPTB + BMI*GABD | 0.742 | 0.735 | 0.748 | 1.67 |
| ATD_~PRL/(TETN + PGRP2) + IBP4/SHBG + PriorPTB + BMI*GABD | 0.741 | 0.731 | 0.752 | 0.29 |
| ATD_~CFAB/(TETN + APOC3) + IBP4/SHBG + PriorPTB + BMI*GABD | 0.741 | 0.734 | 0.749 | 2.66 |
| ATD_~(PRL + CFAB)/PGRP2 + IBP4/SHBG + PriorPTB + BMI*GABD | 0.741 | 0.730 | 0.750 | 3.36 |
| ATD_~CFAB + IBP1 + TIMP1 + IBP4/SHBG + PriorPTB + BMI*GABD | 0.741 | 0.733 | 0.748 | 1.01 |
| ATD_~(PRL + PSG3)/APOC3 + IBP4/SHBG + PriorPTB + BMI*GABD | 0.740 | 0.729 | 0.750 | 0.34 |
| ATD_~CFAB/(PGRP2 + APOC3) + IBP4/SHBG + PriorPTB + BMI*GABD | 0.740 | 0.731 | 0.749 | 4.29 |
| ATD_~(PRL + PSG3)/PGRP2 + IBP4/SHBG + PriorPTB + BMI*GABD | 0.740 | 0.729 | 0.753 | 0.56 |
| ATD_~PRL + EGLN + PSG3 + IBP4/SHBG + PriorPTB + BMI*GABD | 0.740 | 0.729 | 0.750 | 3.23 |
| ATD_~(CFAB + IBP1)/TETN + IBP4/SHBG + PriorPTB + BMI*GABD | 0.739 | 0.731 | 0.746 | 0.80 |
| ATD_~PSG3 + IBP1 + TIMP1 + IBP4/SHBG + PriorPTB + BMI*GABD | 0.739 | 0.728 | 0.749 | 0.26 |
| ATD_~PRL + ADA12 + PSG3 + IBP4/SHBG + PriorPTB + BMI*GABD | 0.739 | 0.726 | 0.751 | 0.38 |
| ATD_~PRL + CFAB + CHL1 + IBP4/SHBG + PriorPTB + BMI*GABD | 0.739 | 0.727 | 0.748 | 1.10 |
| ATD_~ADA12/(TETN + APOC3) + IBP4/SHBG + PriorPTB + BMI*GABD | 0.739 | 0.728 | 0.747 | 0.77 |
| ATD_~PSG3 + CFAB + CHL1 + IBP4/SHBG + PriorPTB + BMI*GABD | 0.739 | 0.729 | 0.746 | 0.43 |
| ATD_~PRL + CFAB + TIMP1 + IBP4/SHBG + PriorPTB + BMI*GABD | 0.739 | 0.729 | 0.748 | 0.92 |
| ATD_~CFAB/(TETN + PGRP2) + IBP4/SHBG + PriorPTB + BMI*GABD | 0.738 | 0.731 | 0.745 | 1.40 |
| ATD_~(PSG3 + TIMP1)/APOC3 + IBP4/SHBG + PriorPTB + BMI*GABD | 0.738 | 0.725 | 0.748 | 0.95 |
| ATD_~(PRL + CHL1)/TETN + IBP4/SHBG + PriorPTB + BMI*GABD | 0.738 | 0.730 | 0.748 | 0.35 |
| ATD_~(EGLN + IBP1)/PGRP2 + IBP4/SHBG + PriorPTB + BMI*GABD | 0.738 | 0.727 | 0.748 | 0.32 |
| ATD_~(ADA12 + CFAB)/APOC3 + IBP4/SHBG + PriorPTB + BMI*GABD | 0.737 | 0.728 | 0.746 | 1.07 |
| ATD_~(CFAB + CHL1)/APOC3 + IBP4/SHBG + PriorPTB + BMI*GABD | 0.737 | 0.725 | 0.746 | 1.91 |
| ATD_~CFAB + CHL1 + TIMP1 + IBP4/SHBG + PriorPTB + BMI*GABD | 0.737 | 0.727 | 0.743 | 0.57 |
| ATD_~(CFAB + CHL1)/PGRP2 + IBP4/SHBG + PriorPTB + BMI*GABD | 0.736 | 0.729 | 0.743 | 3.73 |
| ATD_~(ADA12 + TIMP1)/TETN + IBP4/SHBG + PriorPTB + BMI*GABD | 0.736 | 0.727 | 0.745 | 0.31 |
| ATD_~PRL + ADA12 + CFAB + IBP4/SHBG + PriorPTB + BMI*GABD | 0.736 | 0.724 | 0.746 | 0.81 |
| ATD_~ADA12 + PSG3 + IBP1 + IBP4/SHBG + PriorPTB + BMI*GABD | 0.736 | 0.726 | 0.744 | 0.55 |
| ATD_~(ADA12 + PSG3)/PGRP2 + IBP4/SHBG + PriorPTB + BMI*GABD | 0.735 | 0.725 | 0.743 | 0.29 |
| ATD_~(ADA12 + PSG3)/APOC3 + IBP4/SHBG + PriorPTB + BMI*GABD | 0.735 | 0.724 | 0.745 | 0.50 |
| ATD_~(PSG3 + TIMP1)/PGRP2 + IBP4/SHBG + PriorPTB + BMI*GABD | 0.735 | 0.725 | 0.744 | 0.35 |
| ATD_~(ADA12 + IBP1)/TETN + IBP4/SHBG + PriorPTB + BMI*GABD | 0.734 | 0.725 | 0.743 | 0.39 |
| ATD_~(ADA12 + CFAB)/PGRP2 + IBP4/SHBG + PriorPTB + BMI*GABD | 0.734 | 0.725 | 0.742 | 0.87 |
| ATD_~EGLN/(TETN + ATS13) + IBP4/SHBG + PriorPTB + BMI*GABD | 0.734 | 0.723 | 0.745 | 0.56 |
| ATD_~EGLN + IBP1 + TIMP1 + IBP4/SHBG + PriorPTB + BMI*GABD | 0.734 | 0.724 | 0.745 | 0.35 |
| ATD_~(EGLN + PSG3)/APOC3 + IBP4/SHBG + PriorPTB + BMI*GABD | 0.734 | 0.725 | 0.742 | 0.46 |
| ATD_~PSG3/(TETN + APOC3) + IBP4/SHBG + PriorPTB + BMI*GABD | 0.733 | 0.724 | 0.741 | 0.27 |
| ATD_~(PSG3 + IBP1)/PGRP2 + IBP4/SHBG + PriorPTB + BMI*GABD | 0.733 | 0.724 | 0.742 | 0.25 |
| ATD_~(ADA12 + CFAB)/TETN + IBP4/SHBG + PriorPTB + BMI*GABD | 0.733 | 0.725 | 0.740 | 0.49 |
| ATD_~TIMP1/(TETN + PGRP2) + IBP4/SHBG + PriorPTB + BMI*GABD | 0.732 | 0.723 | 0.741 | 0.26 |
| ATD_~EGLN + PSG3 + TIMP1 + IBP4/SHBG + PriorPTB + BMI*GABD | 0.732 | 0.722 | 0.742 | 0.79 |
| ATD_~CFAB + IBP1 + CHL1 + IBP4/SHBG + PriorPTB + BMI*GABD | 0.731 | 0.723 | 0.738 | 0.52 |
| ATD_~(PRL + IBP1)/PGRP2 + IBP4/SHBG + PriorPTB + BMI*GABD | 0.730 | 0.718 | 0.740 | 0.41 |
| ATD_~(IBP1 + TIMP1)/PGRP2 + IBP4/SHBG + PriorPTB + BMI*GABD | 0.730 | 0.720 | 0.739 | 0.33 |
| ATD_~ADA12 + EGLN + IBP1 + IBP4/SHBG + PriorPTB + BMI*GABD | 0.730 | 0.720 | 0.741 | 1.48 |
| ATD_~(PRL + IBP1)/APOC3 + IBP4/SHBG + PriorPTB + BMI*GABD | 0.729 | 0.717 | 0.741 | 0.29 |
| ATD_~(EGLN + CHL1)/PGRP2 + IBP4/SHBG + PriorPTB + BMI*GABD | 0.729 | 0.718 | 0.738 | 0.32 |
| ATD_~(EGLN + TIMP1)/PGRP2 + IBP4/SHBG + PriorPTB + BMI*GABD | 0.728 | 0.718 | 0.739 | 0.40 |
| ATD_~(PSG3 + IBP1)/TETN + IBP4/SHBG + PriorPTB + BMI*GABD | 0.728 | 0.721 | 0.736 | 0.46 |
| ATD_~(PRL + CFAB)/ATS13 + IBP4/SHBG + PriorPTB + BMI*GABD | 0.728 | 0.717 | 0.738 | 2.30 |
| ATD_~EGLN + IBP1 + CHL1 + IBP4/SHBG + PriorPTB + BMI*GABD | 0.728 | 0.717 | 0.737 | 0.25 |
| ATD_~ADA12 + CFAB + IBP1 + IBP4/SHBG + PriorPTB + BMI*GABD | 0.728 | 0.717 | 0.736 | 0.59 |
| ATD_~PRL + IBP1 + TIMP1 + IBP4/SHBG + PriorPTB + BMI*GABD | 0.727 | 0.717 | 0.737 | 0.25 |
| ATD_~(PSG3 + IBP1)/APOC3 + IBP4/SHBG + PriorPTB + BMI*GABD | 0.727 | 0.717 | 0.736 | 0.28 |
| ATD_~PRL + ADA12 + IBP1 + IBP4/SHBG + PriorPTB + BMI*GABD | 0.727 | 0.714 | 0.738 | 0.31 |
| ATD_~(PRL + EGLN)/PGRP2 + IBP4/SHBG + PriorPTB + BMI*GABD | 0.727 | 0.718 | 0.737 | 0.70 |
| ATD_~(EGLN + IBP1)/APOC3 + IBP4/SHBG + PriorPTB + BMI*GABD | 0.727 | 0.718 | 0.737 | 0.40 |
| ATD_~PSG3/(TETN + PGRP2) + IBP4/SHBG + PriorPTB + BMI*GABD | 0.727 | 0.719 | 0.734 | 0.28 |
| ATD_~PRL + PSG3 + TIMP1 + IBP4/SHBG + PriorPTB + BMI*GABD | 0.727 | 0.717 | 0.736 | 0.23 |
| ATD_~(CHL1 + TIMP1)/TETN + IBP4/SHBG + PriorPTB + BMI*GABD | 0.726 | 0.718 | 0.734 | 0.31 |
| ATD_~EGLN + PSG3 + CHL1 + IBP4/SHBG + PriorPTB + BMI*GABD | 0.726 | 0.717 | 0.735 | 0.35 |
| ATD_~(ADA12 + IBP1)/PGRP2 + IBP4/SHBG + PriorPTB + BMI*GABD | 0.726 | 0.715 | 0.735 | 0.34 |
| ATD_~CFAB/(PGRP2 + ATS13) + IBP4/SHBG + PriorPTB + BMI*GABD | 0.726 | 0.717 | 0.734 | 1.73 |
| ATD_~IBP1/(TETN + APOC3) + IBP4/SHBG + PriorPTB + BMI*GABD | 0.726 | 0.716 | 0.734 | 0.39 |
| ATD_~ADA12 + PSG3 + TIMP1 + IBP4/SHBG + PriorPTB + BMI*GABD | 0.726 | 0.716 | 0.735 | 0.36 |
| ATD_~PRL + PSG3 + CHL1 + IBP4/SHBG + PriorPTB + BMI*GABD | 0.726 | 0.716 | 0.736 | 0.62 |
| ATD_~(CFAB + CHL1)/TETN + IBP4/SHBG + PriorPTB + BMI*GABD | 0.725 | 0.718 | 0.731 | 0.80 |
| ATD_~CFAB/(ATS13 + APOC3) + IBP4/SHBG + PriorPTB + BMI*GABD | 0.725 | 0.714 | 0.733 | 1.05 |
| ATD_~(PRL + EGLN)/ATS13 + IBP4/SHBG + PriorPTB + BMI*GABD | 0.724 | 0.714 | 0.735 | 0.20 |
| ATD_~PRL + IBP1 + CHL1 + IBP4/SHBG + PriorPTB + BMI*GABD | 0.724 | 0.713 | 0.734 | 0.41 |
| ATD_~PRL + ADA12 + EGLN + IBP4/SHBG + PriorPTB + BMI*GABD | 0.724 | 0.710 | 0.736 | 0.97 |
| ATD_~IBP1/(TETN + PGRP2) + IBP4/SHBG + PriorPTB + BMI*GABD | 0.723 | 0.713 | 0.732 | 0.34 |
| ATD_~PRL + EGLN + TIMP1 + IBP4/SHBG + PriorPTB + BMI*GABD | 0.723 | 0.712 | 0.735 | 0.30 |
| ATD_~ADA12 + EGLN + PSG3 + IBP4/SHBG + PriorPTB + BMI*GABD | 0.723 | 0.714 | 0.732 | 0.25 |
| ATD_~PSG3 + CHL1 + TIMP1 + IBP4/SHBG + PriorPTB + BMI*GABD | 0.723 | 0.712 | 0.731 | 1.10 |
| ATD_~(PRL + EGLN)/APOC3 + IBP4/SHBG + PriorPTB + BMI*GABD | 0.722 | 0.711 | 0.733 | 0.26 |
| ATD_~(PRL + PSG3)/ATS13 + IBP4/SHBG + PriorPTB + BMI*GABD | 0.722 | 0.710 | 0.732 | 0.73 |
| ATD_~PRL/(TETN + ATS13) + IBP4/SHBG + PriorPTB + BMI*GABD | 0.722 | 0.712 | 0.731 | 0.31 |
| ATD_~ADA12 + CFAB + TIMP1 + IBP4/SHBG + PriorPTB + BMI*GABD | 0.722 | 0.713 | 0.730 | 0.42 |
| ATD_~(ADA12 + EGLN)/PGRP2 + IBP4/SHBG + PriorPTB + BMI*GABD | 0.722 | 0.709 | 0.732 | 0.43 |
| ATD_~(ADA12 + EGLN)/APOC3 + IBP4/SHBG + PriorPTB + BMI*GABD | 0.722 | 0.711 | 0.732 | 2.59 |
| ATD_~PRL + EGLN + CHL1 + IBP4/SHBG + PriorPTB + BMI*GABD | 0.722 | 0.709 | 0.731 | 0.34 |
| ATD_~(PSG3 + CFAB)/ATS13 + IBP4/SHBG + PriorPTB + BMI*GABD | 0.721 | 0.713 | 0.728 | 0.48 |
| ATD_~PSG3/(PGRP2 + APOC3) + IBP4/SHBG + PriorPTB + BMI*GABD | 0.721 | 0.710 | 0.731 | 0.61 |
| ATD_~(IBP1 + TIMP1)/APOC3 + IBP4/SHBG + PriorPTB + BMI*GABD | 0.721 | 0.712 | 0.729 | 0.38 |
| ATD_~(EGLN + TIMP1)/APOC3 + IBP4/SHBG + PriorPTB + BMI*GABD | 0.721 | 0.712 | 0.730 | 0.25 |
| ATD_~(IBP1 + CHL1)/PGRP2 + IBP4/SHBG + PriorPTB + BMI*GABD | 0.721 | 0.711 | 0.729 | 0.37 |
| ATD_~ADA12 + CFAB + CHL1 + IBP4/SHBG + PriorPTB + BMI*GABD | 0.720 | 0.711 | 0.728 | 0.35 |
| ATD_~ADA12/(TETN + PGRP2) + IBP4/SHBG + PriorPTB + BMI*GABD | 0.720 | 0.710 | 0.729 | 0.75 |
| ATD_~PSG3 + IBP1 + CHL1 + IBP4/SHBG + PriorPTB + BMI*GABD | 0.720 | 0.708 | 0.728 | 0.35 |
| ATD_~EGLN/(PGRP2 + APOC3) + IBP4/SHBG + PriorPTB + BMI*GABD | 0.719 | 0.708 | 0.727 | 0.30 |
| ATD_~(ADA12 + CHL1)/TETN + IBP4/SHBG + PriorPTB + BMI*GABD | 0.718 | 0.710 | 0.727 | 0.30 |
| ATD_~(CFAB + IBP1)/ATS13 + IBP4/SHBG + PriorPTB + BMI*GABD | 0.718 | 0.710 | 0.725 | 1.23 |
| ATD_~(ADA12 + TIMP1)/APOC3 + IBP4/SHBG + PriorPTB + BMI*GABD | 0.718 | 0.707 | 0.728 | 0.32 |
| ATD_~(CFAB + TIMP1)/ATS13 + IBP4/SHBG + PriorPTB + BMI*GABD | 0.717 | 0.709 | 0.725 | 0.47 |
| ATD_~(ADA12 + IBP1)/APOC3 + IBP4/SHBG + PriorPTB + BMI*GABD | 0.717 | 0.707 | 0.725 | 0.26 |
| ATD_~ADA12 + IBP1 + TIMP1 + IBP4/SHBG + PriorPTB + BMI*GABD | 0.716 | 0.707 | 0.726 | 0.26 |
| ATD_~ADA12/(PGRP2 + APOC3) + IBP4/SHBG + PriorPTB + BMI*GABD | 0.716 | 0.706 | 0.729 | 0.24 |
| ATD_~(EGLN + CHL1)/APOC3 + IBP4/SHBG + PriorPTB + BMI*GABD | 0.716 | 0.706 | 0.728 | 0.30 |
| ATD_~(PRL + ADA12)/APOC3 + IBP4/SHBG + PriorPTB + BMI*GABD | 0.716 | 0.706 | 0.725 | 0.28 |
| ATD_~(PRL + ADA12)/ATS13 + IBP4/SHBG + PriorPTB + BMI*GABD | 0.716 | 0.705 | 0.728 | 2.73 |
| ATD_~ADA12 + PSG3 + CHL1 + IBP4/SHBG + PriorPTB + BMI*GABD | 0.716 | 0.706 | 0.725 | 0.23 |
| ATD_~EGLN/(ATS13 + APOC3) + IBP4/SHBG + PriorPTB + BMI*GABD | 0.716 | 0.704 | 0.725 | 0.28 |
| ATD_~(PSG3 + CHL1)/PGRP2 + IBP4/SHBG + PriorPTB + BMI*GABD | 0.716 | 0.707 | 0.723 | 0.40 |
| ATD_~CFAB/(TETN + ATS13) + IBP4/SHBG + PriorPTB + BMI*GABD | 0.715 | 0.707 | 0.722 | 0.86 |
| ATD_~TIMP1/(PGRP2 + APOC3) + IBP4/SHBG + PriorPTB + BMI*GABD | 0.715 | 0.705 | 0.723 | 0.61 |
| ATD_~EGLN/(PGRP2 + ATS13) + IBP4/SHBG + PriorPTB + BMI*GABD | 0.715 | 0.706 | 0.724 | 0.27 |
| ATD_~(PSG3 + CHL1)/APOC3 + IBP4/SHBG + PriorPTB + BMI*GABD | 0.715 | 0.706 | 0.724 | 0.31 |
| ATD_~(PSG3 + CHL1)/TETN + IBP4/SHBG + PriorPTB + BMI*GABD | 0.715 | 0.707 | 0.724 | 0.34 |
| ATD_~(EGLN + IBP1)/ATS13 + IBP4/SHBG + PriorPTB + BMI*GABD | 0.715 | 0.705 | 0.725 | 0.23 |
| ATD_~(IBP1 + CHL1)/TETN + IBP4/SHBG + PriorPTB + BMI*GABD | 0.715 | 0.707 | 0.722 | 0.26 |
| ATD_~CHL1/(TETN + APOC3) + IBP4/SHBG + PriorPTB + BMI*GABD | 0.714 | 0.706 | 0.721 | 0.58 |
| ATD_~1/(TETN + PGRP2 + APOC3) + IBP4/SHBG + PriorPTB + BMI*GABD | 0.714 | 0.706 | 0.722 | 0.35 |
| ATD_~(PRL + TIMP1)/APOC3 + IBP4/SHBG + PriorPTB + BMI*GABD | 0.714 | 0.704 | 0.723 | 1.67 |
| ATD_~(PRL + IBP1)/ATS13 + IBP4/SHBG + PriorPTB + BMI*GABD | 0.714 | 0.703 | 0.724 | 0.31 |
| ATD_~ADA12 + EGLN + CHL1 + IBP4/SHBG + PriorPTB + BMI*GABD | 0.713 | 0.703 | 0.722 | 0.23 |
| ATD_~(EGLN + TIMP1)/ATS13 + IBP4/SHBG + PriorPTB + BMI*GABD | 0.713 | 0.703 | 0.724 | 0.21 |
| ATD_~PRL + ADA12 + TIMP1 + IBP4/SHBG + PriorPTB + BMI*GABD | 0.713 | 0.701 | 0.723 | 0.17 |
| ATD_~(ADA12 + CHL1)/PGRP2 + IBP4/SHBG + PriorPTB + BMI*GABD | 0.712 | 0.704 | 0.721 | 0.35 |
| ATD_~(EGLN + PSG3)/ATS13 + IBP4/SHBG + PriorPTB + BMI*GABD | 0.712 | 0.703 | 0.723 | 0.20 |
| ATD_~(ADA12 + TIMP1)/PGRP2 + IBP4/SHBG + PriorPTB + BMI*GABD | 0.712 | 0.700 | 0.723 | 0.76 |
| ATD_~PRL/(ATS13 + APOC3) + IBP4/SHBG + PriorPTB + BMI*GABD | 0.712 | 0.702 | 0.723 | 0.24 |
| ATD_~(CHL1 + TIMP1)/PGRP2 + IBP4/SHBG + PriorPTB + BMI*GABD | 0.712 | 0.703 | 0.720 | 0.42 |
| ATD_~(ADA12 + PSG3)/ATS13 + IBP4/SHBG + PriorPTB + BMI*GABD | 0.712 | 0.702 | 0.721 | 0.31 |
| ATD_~(CFAB + CHL1)/ATS13 + IBP4/SHBG + PriorPTB + BMI*GABD | 0.711 | 0.704 | 0.718 | 2.33 |
| ATD_~IBP1 + CHL1 + TIMP1 + IBP4/SHBG + PriorPTB + BMI*GABD | 0.711 | 0.703 | 0.719 | 0.30 |
| ATD_~EGLN + CHL1 + TIMP1 + IBP4/SHBG + PriorPTB + BMI*GABD | 0.711 | 0.700 | 0.721 | 0.52 |
| ATD_~IBP1/(PGRP2 + APOC3) + IBP4/SHBG + PriorPTB + BMI*GABD | 0.710 | 0.701 | 0.719 | 0.29 |
| ATD_~ADA12 + EGLN + TIMP1 + IBP4/SHBG + PriorPTB + BMI*GABD | 0.710 | 0.699 | 0.720 | 0.22 |
| ATD_~(PRL + ADA12)/PGRP2 + IBP4/SHBG + PriorPTB + BMI*GABD | 0.710 | 0.699 | 0.721 | 0.20 |
| ATD_~(PSG3 + TIMP1)/ATS13 + IBP4/SHBG + PriorPTB + BMI*GABD | 0.709 | 0.700 | 0.719 | 0.37 |
| ATD_~PRL + ADA12 + CHL1 + IBP4/SHBG + PriorPTB + BMI*GABD | 0.709 | 0.695 | 0.720 | 0.16 |
| ATD_~CHL1/(TETN + PGRP2) + IBP4/SHBG + PriorPTB + BMI*GABD | 0.709 | 0.701 | 0.716 | 0.31 |
| ATD_~(ADA12 + CFAB)/ATS13 + IBP4/SHBG + PriorPTB + BMI*GABD | 0.709 | 0.699 | 0.717 | 1.04 |
| ATD_~(ADA12 + CHL1)/APOC3 + IBP4/SHBG + PriorPTB + BMI*GABD | 0.708 | 0.695 | 0.721 | 0.35 |
| ATD_~(PRL + CHL1)/PGRP2 + IBP4/SHBG + PriorPTB + BMI*GABD | 0.708 | 0.698 | 0.717 | 0.22 |
| ATD_~(PRL + CHL1)/APOC3 + IBP4/SHBG + PriorPTB + BMI*GABD | 0.708 | 0.695 | 0.718 | 0.45 |
| ATD_~(CHL1 + TIMP1)/APOC3 + IBP4/SHBG + PriorPTB + BMI*GABD | 0.708 | 0.698 | 0.715 | 0.34 |
| ATD_~TIMP1/(ATS13 + APOC3) + IBP4/SHBG + PriorPTB + BMI*GABD | 0.707 | 0.696 | 0.717 | 0.24 |
| ATD_~(ADA12 + EGLN)/ATS13 + IBP4/SHBG + PriorPTB + BMI*GABD | 0.707 | 0.697 | 0.718 | 0.48 |
| ATD_~(PSG3 + IBP1)/ATS13 + IBP4/SHBG + PriorPTB + BMI*GABD | 0.707 | 0.698 | 0.715 | 0.30 |
| ATD_~PRL + CHL1 + TIMP1 + IBP4/SHBG + PriorPTB + BMI*GABD | 0.707 | 0.698 | 0.717 | 0.25 |
| ATD_~ADA12/(TETN + ATS13) + IBP4/SHBG + PriorPTB + BMI*GABD | 0.705 | 0.697 | 0.713 | 0.24 |
| ATD_~TIMP1/(TETN + ATS13) + IBP4/SHBG + PriorPTB + BMI*GABD | 0.705 | 0.697 | 0.712 | 0.22 |
| ATD_~(EGLN + CHL1)/ATS13 + IBP4/SHBG + PriorPTB + BMI*GABD | 0.705 | 0.696 | 0.715 | 0.21 |
| ATD_~PRL/(PGRP2 + APOC3) + IBP4/SHBG + PriorPTB + BMI*GABD | 0.704 | 0.693 | 0.715 | 0.29 |
| ATD_~ADA12 + IBP1 + CHL1 + IBP4/SHBG + PriorPTB + BMI*GABD | 0.704 | 0.695 | 0.713 | 0.33 |
| ATD_~(IBP1 + TIMP1)/ATS13 + IBP4/SHBG + PriorPTB + BMI*GABD | 0.704 | 0.696 | 0.713 | 0.40 |
| ATD_~PSG3/(APOC3 + ATS13) + IBP4/SHBG + PriorPTB + BMI*GABD | 0.703 | 0.694 | 0.712 | 0.25 |
| ATD_~(ADA12 + IBP1)/ATS13 + IBP4/SHBG + PriorPTB + BMI*GABD | 0.703 | 0.693 | 0.712 | 0.46 |
| ATD_~1/(TETN + ATS13 + APOC3) + IBP4/SHBG + PriorPTB + BMI*GABD | 0.702 | 0.695 | 0.710 | 0.19 |
| ATD_~CHL1/(PGRP2 + APOC3) + IBP4/SHBG + PriorPTB + BMI*GABD | 0.702 | 0.693 | 0.711 | 0.32 |
| ATD_~(IBP1 + CHL1)/APOC3 + IBP4/SHBG + PriorPTB + BMI*GABD | 0.702 | 0.693 | 0.710 | 0.54 |
| ATD_~(PRL + TIMP1)/PGRP2 + IBP4/SHBG + PriorPTB + BMI*GABD | 0.701 | 0.692 | 0.711 | 0.20 |
| ATD_~PRL/(PGRP2 + ATS13) + IBP4/SHBG + PriorPTB + BMI*GABD | 0.701 | 0.690 | 0.712 | 0.52 |
| ATD_~(PRL + CHL1)/ATS13 + IBP4/SHBG + PriorPTB + BMI*GABD | 0.700 | 0.691 | 0.710 | 0.80 |
| ATD_~(ADA12 + TIMP1)/ATS13 + IBP4/SHBG + PriorPTB + BMI*GABD | 0.700 | 0.691 | 0.709 | 0.28 |
| ATD_~PSG3/(TETN + ATS13) + IBP4/SHBG + PriorPTB + BMI*GABD | 0.700 | 0.692 | 0.708 | 0.23 |
| ATD_~(PRL + TIMP1)/ATS13 + IBP4/SHBG + PriorPTB + BMI*GABD | 0.698 | 0.687 | 0.710 | 0.54 |
| ATD_~PSG3/(PGRP2 + ATS13) + IBP4/SHBG + PriorPTB + BMI*GABD | 0.698 | 0.688 | 0.706 | 0.22 |
| ATD_~IBP1/(TETN + ATS13) + IBP4/SHBG + PriorPTB + BMI*GABD | 0.696 | 0.689 | 0.704 | 0.28 |
| ATD_~IBP1/(PGRP2 + ATS13) + IBP4/SHBG + PriorPTB + BMI*GABD | 0.695 | 0.686 | 0.704 | 0.20 |
| ATD_~ADA12 + CHL1 + TIMP1 + IBP4/SHBG + PriorPTB + BMI*GABD | 0.695 | 0.685 | 0.704 | 0.22 |
| ATD_~1/(PGRP2 + ATS13 + APOC3) + IBP4/SHBG + PriorPTB + BMI*GABD | 0.693 | 0.683 | 0.702 | 0.70 |
| ATD_~IBP1/(ATS13 + APOC3) + IBP4/SHBG + PriorPTB + BMI*GABD | 0.693 | 0.684 | 0.701 | 0.44 |
| ATD_~ADA12/(PGRP2 + ATS13) + IBP4/SHBG + PriorPTB + BMI*GABD | 0.692 | 0.683 | 0.701 | 0.23 |
| ATD_~TIMP1/(PGRP2 + ATS13) + IBP4/SHBG + PriorPTB + BMI*GABD | 0.692 | 0.680 | 0.701 | 0.18 |
| ATD_~ADA12/(ATS13 + APOC3) + IBP4/SHBG + PriorPTB + BMI*GABD | 0.690 | 0.678 | 0.701 | 0.51 |
| ATD_~(ADA12 + CHL1)/ATS13 + IBP4/SHBG + PriorPTB + BMI*GABD | 0.689 | 0.679 | 0.697 | 0.35 |
| ATD_~(PSG3 + CHL1)/ATS13 + IBP4/SHBG + PriorPTB + BMI*GABD | 0.688 | 0.678 | 0.697 | 0.46 |
| ATD_~1/(TETN + PGRP2 + ATS13) + IBP4/SHBG + PriorPTB + BMI*GABD | 0.687 | 0.679 | 0.695 | 0.27 |
| ATD_~(CHL1 + TIMP1)/ATS13 + IBP4/SHBG + PriorPTB + BMI*GABD | 0.687 | 0.678 | 0.696 | 0.46 |
| ATD_~(IBP1 + CHL1)/ATS13 + IBP4/SHBG + PriorPTB + BMI*GABD | 0.684 | 0.675 | 0.691 | 0.40 |
| ATD_~CHL1/(PGRP2 + ATS13) + IBP4/SHBG + PriorPTB + BMI*GABD | 0.684 | 0.675 | 0.693 | 0.25 |
| ATD_~CHL1/(TETN + ATS13) + IBP4/SHBG + PriorPTB + BMI*GABD | 0.683 | 0.677 | 0.690 | 0.27 |
| ATD_~CHL1/(ATS13 + APOC3) + IBP4/SHBG + PriorPTB + BMI*GABD | 0.682 | 0.672 | 0.691 | 0.34 |

TABLE 12
Reversal Group Classification Performance at Predicting sPTB <37
weeks gestation. Reversal group comprises IBP4/SHBG and one additional
protein biomarker, and two additional clinical variables.

| Reversal Group | AUC | CI low | CI high | Sum Rank |
|---|---|---|---|---|
| ATD_~EGLN + IBP4/SHBG + BMI*GABD | 0.663 | 0.595 | 0.732 | 8.31 |
| ATD_~PRL + IBP4/SHBG + BMI*GABD | 0.679 | 0.61 | 0.748 | 7.59 |
| ATD_~IBP1 + IBP4/SHBG + BMI*GABD | 0.657 | 0.587 | 0.727 | 7.39 |
| ATD_~1/APOC3 + IBP4/SHBG + BMI*GABD | 0.668 | 0.597 | 0.739 | 5.69 |
| ATD_~1/PGRP2 + IBP4/SHBG + BMI*GABD | 0.653 | 0.584 | 0.722 | 4.65 |
| ATD_~TIMP1 + IBP4/SHBG + BMI*GABD | 0.647 | 0.578 | 0.716 | 4.15 |
| ATD_~CFAB + IBP4/SHBG + BMI*GABD | 0.669 | 0.6 | 0.739 | 4.03 |
| ATD_~1/TETN + IBP4/SHBG + BMI*GABD | 0.665 | 0.596 | 0.735 | 3.15 |
| ATD_~ADA12 + IBP4/SHBG + BMI*GABD | 0.657 | 0.589 | 0.726 | 2.68 |
| ATD_~1/ATS13 + IBP4/SHBG + BMI*GABD | 0.65 | 0.577 | 0.722 | 2.47 |
| ATD_~CHL1 + IBP4/SHBG + BMI*GABD | 0.649 | 0.579 | 0.719 | 2.46 |
| ATD_~PSG3 + IBP4/SHBG + BMI*GABD | 0.656 | 0.588 | 0.724 | 2.34 |

TABLE 13
Reversal Group Classification Performance at Predicting sPTB <37 weeks gestation. Reversal group
comprises IBP4/SHBG, two additional protein biomarkers, and two additional clinical variables.

| | Reversal Group | AUC | CI low | CI high | Sum Rank |
|---|---|---|---|---|---|
| 1. | ATD_~PRL/TETN + IBP4/SHBG + BMI*GABD | 0.707 | 0.638 | 0.776 | 3.06 |
| 2. | ATD_~EGLN/TETN + IBP4/SHBG + BMI*GABD | 0.705 | 0.639 | 0.77 | 2.36 |
| 3. | ATD_~PRL + CFAB + IBP4/SHBG + BMI*GABD | 0.695 | 0.626 | 0.764 | 1.86 |
| 4. | ATD_~ADA12/TETN + IBP4/SHBG + BMI*GABD | 0.695 | 0.628 | 0.762 | 1.27 |
| 5. | ATD_~PRL/APOC3 + IBP4/SHBG + BMI*GABD | 0.693 | 0.626 | 0.761 | 1.87 |
| 6. | ATD_~PRL + EGLN + IBP4/SHBG + BMI*GABD | 0.692 | 0.625 | 0.76 | 0.88 |
| 7. | ATD_~EGLN + CFAB + IBP4/SHBG + BMI*GABD | 0.691 | 0.624 | 0.757 | 1.11 |
| 8. | ATD_~CFAB/APOC3 + IBP4/SHBG + BMI*GABD | 0.691 | 0.622 | 0.76 | 0.76 |
| 9 | ATD_~PRL + PSG3 + IBP4/SHBG + BMI*GABD | 0.691 | 0.624 | 0.758 | 0.73 |
| 10. | ATD_~PRL/ATS13 + IBP4/SHBG + BMI*GABD | 0.689 | 0.62 | 0.759 | 1.04 |
| 11. | ATD_~EGLN/ATS13 + IBP4/SHBG + BMI*GABD | 0.687 | 0.617 | 0.756 | 0.73 |
| 12. | ATD_~PRL/PGRP2 + IBP4/SHBG + BMI*GABD | 0.686 | 0.618 | 0.754 | 1.29 |
| 13. | ATD_~1/(TETN + APOC3) + IBP4/SHBG + BMI*GABD | 0.686 | 0.617 | 0.756 | 0.77 |
| 14. | ATD_~PRL + ADA12 + IBP4/SHBG + BMI*GABD | 0.684 | 0.617 | 0.752 | 2.01 |
| 15. | ATD_~CFAB/TETN + IBP4/SHBG + BMI*GABD | 0.684 | 0.615 | 0.753 | 0.90 |
| 16. | ATD_~PRL + IBP1 + IBP4/SHBG + BMI*GABD | 0.684 | 0.615 | 0.754 | 0.84 |
| 17. | ATD_~PSG3/APOC3 + IBP4/SHBG + BMI*GABD | 0.682 | 0.614 | 0.75 | 0.92 |
| 18. | ATD_~ADA12 + CFAB + IBP4/SHBG + BMI*GABD | 0.682 | 0.614 | 0.75 | 0.74 |
| 19. | ATD_~PSG3 + CFAB + IBP4/SHBG + BMI*GABD | 0.682 | 0.615 | 0.75 | 0.61 |
| 20. | ATD_~PRL + CHL1 + IBP4/SHBG + BMI*GABD | 0.681 | 0.612 | 0.751 | 0.95 |
| 21. | ATD_~EGLN/APOC3 + IBP4/SHBG + BMI*GABD | 0.679 | 0.61 | 0.749 | 1.75 |
| 22. | ATD_~ADA12/ATS13 + IBP4/SHBG + BMI*GABD | 0.678 | 0.609 | 0.748 | 0.57 |
| 23. | ATD_~TIMP1/TETN + IBP4/SHBG + BMI*GABD | 0.678 | 0.61 | 0.746 | 0.54 |
| 24. | ATD_~ADA12/APOC3 + IBP4/SHBG + BMI*GABD | 0.678 | 0.61 | 0.746 | 0.53 |
| 25. | ATD_~PRL + TIMP1 + IBP4/SHBG + BMI*GABD | 0.677 | 0.608 | 0.746 | 0.54 |
| 26. | ATD_~IBP1/TETN + IBP4/SHBG + BMI*GABD | 0.676 | 0.606 | 0.746 | 0.83 |
| 27. | ATD_~PSG3/TETN + IBP4/SHBG + BMI*GABD | 0.676 | 0.609 | 0.743 | 0.54 |
| 28. | ATD_~CFAB + TIMP1 + IBP4/SHBG + BMI*GABD | 0.676 | 0.608 | 0.744 | 0.50 |
| 29. | ATD_~EGLN/PGRP2 + IBP4/SHBG + BMI*GABD | 0.675 | 0.607 | 0.742 | 2.91 |
| 30. | ATD_~CFAB + IBP1 + IBP4/SHBG + BMI*GABD | 0.675 | 0.605 | 0.744 | 0.60 |
| 31. | ATD_~EGLN + IBP1 + IBP4/SHBG + BMI*GABD | 0.674 | 0.606 | 0.742 | 3.79 |
| 32. | ATD_~ADA12 + EGLN + IBP4/SHBG + BMI*GABD | 0.674 | 0.607 | 0.742 | 0.45 |
| 33. | ATD_~ADA12 + IBP1 + IBP4/SHBG + BMI*GABD | 0.673 | 0.605 | 0.741 | 0.48 |
| 34. | ATD_~TIMP1/APOC3 + IBP4/SHBG + BMI*GABD | 0.672 | 0.602 | 0.742 | 6.02 |
| 35. | ATD_~EGLN + PSG3 + IBP4/SHBG + BMI*GABD | 0.672 | 0.605 | 0.739 | 1.26 |
| 36. | ATD_~1/(PGRP2 + APOC3) + IBP4/SHBG + BMI*GABD | 0.67 | 0.6 | 0.739 | 3.12 |
| 37. | ATD_~IBP1/APOC3 + IBP4/SHBG + BMI*GABD | 0.67 | 0.6 | 0.741 | 0.95 |
| 38. | ATD_~CFAB + CHL1 + IBP4/SHBG + BMI*GABD | 0.67 | 0.601 | 0.739 | 0.73 |
| 39. | ATD_~CFAB/PGRP2 + IBP4/SHBG + BMI*GABD | 0.67 | 0.601 | 0.739 | 0.66 |
| 40. | ATD_~ADA12/PGRP2 + IBP4/SHBG + BMI*GABD | 0.67 | 0.603 | 0.738 | 0.49 |
| 41. | ATD_~ADA12 + PSG3 + IBP4/SHBG + BMI*GABD | 0.67 | 0.603 | 0.738 | 0.41 |
| 42. | ATD_~CHL1/TETN + IBP4/SHBG + BMI*GABD | 0.67 | 0.6 | 0.739 | 0.38 |
| 43. | ATD_~CFAB/ATS13 + IBP4/SHBG + BMI*GABD | 0.669 | 0.598 | 0.74 | 1.39 |
| 44. | ATD_~1/(ATS13 + APOC3) + IBP4/SHBG + BMI*GABD | 0.669 | 0.596 | 0.742 | 0.72 |
| 45. | ATD_~EGLN + CHL1 + IBP4/SHBG + BMI*GABD | 0.668 | 0.599 | 0.737 | 1.42 |
| 46. | ATD_~1/(TETN + PGRP2) + IBP4/SHBG + BMI*GABD | 0.668 | 0.599 | 0.738 | 0.48 |
| 47. | ATD_~CHL1/APOC3 + IBP4/SHBG + BMI*GABD | 0.667 | 0.596 | 0.738 | 0.74 |
| 48. | ATD_~PSG3/PGRP2 + IBP4/SHBG + BMI*GABD | 0.666 | 0.598 | 0.734 | 0.94 |
| 49. | ATD_~PSG3 + IBP1 + IBP4/SHBG + BMI*GABD | 0.666 | 0.598 | 0.734 | 0.91 |
| 50. | ATD_~1/(TETN + ATS13) + IBP4/SHBG + BMI*GABD | 0.665 | 0.595 | 0.736 | 0.41 |
| 51. | ATD_~EGLN + TIMP1 + IBP4/SHBG + BMI*GABD | 0.663 | 0.595 | 0.732 | 5.35 |
| 52. | ATD_~PSG3/ATS13 + IBP4/SHBG + BMI*GABD | 0.662 | 0.593 | 0.732 | 0.70 |
| 53. | ATD_~ADA12 + CHL1 + IBP4/SHBG + BMI*GABD | 0.662 | 0.593 | 0.731 | 0.53 |
| 54. | ATD_~IBP1/ATS13 + IBP4/SHBG + BMI*GABD | 0.661 | 0.589 | 0.734 | 1.23 |
| 55. | ATD_~IBP1/PGRP2 + IBP4/SHBG + BMI*GABD | 0.661 | 0.592 | 0.73 | 1.14 |
| 56. | ATD_~TIMP1/ATS13 + IBP4/SHBG + BMI*GABD | 0.66 | 0.589 | 0.732 | 0.62 |
| 57. | ATD_~PSG3 + TIMP1 + IBP4/SHBG + BMI*GABD | 0.66 | 0.592 | 0.728 | 0.48 |
| 58. | ATD_~TIMP1/PGRP2 + IBP4/SHBG + BMI*GABD | 0.659 | 0.591 | 0.727 | 4.15 |
| 59. | ATD_~IBP1 + CHL1 + IBP4/SHBG + BMI*GABD | 0.659 | 0.588 | 0.729 | 2.80 |
| 60. | ATD_~PSG3 + CHL1 + IBP4/SHBG + BMI*GABD | 0.657 | 0.589 | 0.725 | 0.91 |
| 61. | ATD_~1/(PGRP2 + ATS13) + IBP4/SHBG + BMI*GABD | 0.657 | 0.585 | 0.728 | 0.78 |
| 62. | ATD_~ADA12 + TIMP1 + IBP4/SHBG + BMI*GABD | 0.657 | 0.588 | 0.727 | 0.40 |
| 63. | ATD_~IBP1 + TIMP1 + IBP4/SHBG + BMI*GABD | 0.656 | 0.586 | 0.725 | 1.45 |
| 64. | ATD_~CHL1/PGRP2 + IBP4/SHBG + BMI*GABD | 0.656 | 0.587 | 0.726 | 0.88 |
| 65. | ATD_~CHL1/ATS13 + IBP4/SHBG + BMI*GABD | 0.653 | 0.58 | 0.725 | 0.67 |
| 66. | ATD_~CHL1 + TIMP1 + IBP4/SHBG + BMI*GABD | 0.651 | 0.581 | 0.721 | 1.16 |

TABLE 14
Reversal Group Classification Performance at Predicting sPTB <37
weeks gestation. Reversal group comprises IBP4/SHBG, three additional
protein biomarkers and two additional clinical variables.

| | | CI | CI | Sum |
|---|---|---|---|---|
| Reversal Group | AUC | low | high | Rank |
| ATD_~PRL + EGLN/TETN + IBP4/SHBG + BMI*GABD | 0.728 | 0.663 | 0.793 | 3.12 |
| ATD_~PRL/(TETN + APOC3) + IBP4/SHBG + BMI*GABD | 0.722 | 0.656 | 0.788 | 1.64 |
| ATD_~(PRL + ADA12)/TETN + IBP4/SHBG + BMI*GABD | 0.719 | 0.654 | 0.785 | 1.11 |
| ATD_~(PRL + PSG3)/TETN + IBP4/SHBG + BMI*GABD | 0.718 | 0.651 | 0.784 | 0.79 |
| ATD_~EGLN/(TETN + APOC3) + IBP4/SHBG + BMI*GABD | 0.716 | 0.652 | 0.781 | 0.80 |
| ATD_~(PRL + EGLN)/ATS13 + IBP4/SHBG + BMI*GABD | 0.715 | 0.648 | 0.781 | 0.61 |
| ATD_~ADA12/(TETN + APOC3) + IBP4/SHBG + BMI*GABD | 0.715 | 0.651 | 0.78 | 0.60 |
| ATD_~(ADA12 + EGLN)/TETN + IBP4/SHBG + BMI*GABD | 0.714 | 0.65 | 0.778 | 0.51 |
| ATD_~(EGLN + CFAB)/TETN + IBP4/SHBG + BMI*GABD | 0.714 | 0.649 | 0.778 | 0.46 |
| ATD_~(PRL + CFAB)/APOC3 + IBP4/SHBG + BMI*GABD | 0.714 | 0.647 | 0.781 | 0.44 |
| ATD_~(PRL + CFAB)/TETN + IBP4/SHBG + BMI*GABD | 0.713 | 0.644 | 0.781 | 0.49 |
| ATD_~PRL/(TETN + PGRP2) + IBP4/SHBG + BMI*GABD | 0.712 | 0.644 | 0.779 | 0.71 |
| ATD_~PRL + EGLN + CFAB + IBP4/SHBG + BMI*GABD | 0.712 | 0.644 | 0.779 | 0.33 |
| ATD_~(PRL + IBP1)/TETN + IBP4/SHBG + BMI*GABD | 0.711 | 0.642 | 0.781 | 1.31 |
| ATD_~(EGLN + PSG3)/TETN + IBP4/SHBG + BMI*GABD | 0.711 | 0.646 | 0.775 | 0.33 |
| ATD_~EGLN/(TETN + ATS13) + IBP4/SHBG + BMI*GABD | 0.711 | 0.644 | 0.777 | 0.31 |
| ATD_~(EGLN + IBP1)/TETN + IBP4/SHBG + BMI*GABD | 0.71 | 0.644 | 0.775 | 0.56 |
| ATD_~(PRL + PSG3)/APOC3 + IBP4/SHBG + BMI*GABD | 0.71 | 0.645 | 0.776 | 0.38 |
| ATD_~(PRL + CHL1)/TETN + IBP4/SHBG + BMI*GABD | 0.709 | 0.64 | 0.778 | 0.36 |
| ATD_~(PSG3 + CFAB)/APOC3 + IBP4/SHBG + BMI*GABD | 0.709 | 0.642 | 0.776 | 0.32 |
| ATD_~(PRL + TIMP1)/TETN + IBP4/SHBG + BMI*GABD | 0.708 | 0.64 | 0.775 | 0.90 |
| ATD_~(ADA12 + CFAB)/TETN + IBP4/SHBG + BMI*GABD | 0.707 | 0.64 | 0.773 | 0.28 |
| ATD_~(EGLN + PGRP2)/TETN + IBP4/SHBG + BMI*GABD | 0.707 | 0.641 | 0.772 | 0.25 |
| ATD_~CFAB/(TETN + APOC3) + IBP4/SHBG + BMI*GABD | 0.706 | 0.638 | 0.774 | 0.35 |
| ATD_~(PRL + EGLN)/APOC3 + IBP4/SHBG + BMI*GABD | 0.705 | 0.638 | 0.771 | 0.60 |
| ATD_~TIMP1/(TETN + APOC3) + IBP4/SHBG + BMI*GABD | 0.705 | 0.639 | 0.771 | 0.32 |
| ATD_~(EGLN + CHL1)/TETN + IBP4/SHBG + BMI*GABD | 0.705 | 0.639 | 0.771 | 0.27 |
| ATD_~PRL/(TETN + ATS13) + IBP4/SHBG + BMI*GABD | 0.705 | 0.636 | 0.774 | 0.24 |
| ATD_~PRL + PSG3 + CFAB + IBP4/SHBG + BMI*GABD | 0.705 | 0.638 | 0.773 | 0.22 |
| ATD_~(EGLN + CFAB)/APOC3 + IBP4/SHBG + BMI*GABD | 0.704 | 0.637 | 0.771 | 0.29 |
| ATD_~(PRL + CFAB)/PGRP2 + IBP4/SHBG + BMI*GABD | 0.704 | 0.636 | 0.772 | 0.27 |
| ATD_~(ADA12 + PSG3)/TETN + IBP4/SHBG + BMI*GABD | 0.704 | 0.639 | 0.77 | 0.25 |
| ATD_~(PRL + ADA12)/ATS13 + IBP4/SHBG + BMI*GABD | 0.703 | 0.634 | 0.771 | 0.52 |
| ATD_~(PRL + PSG3)/PGRP2 + IBP4/SHBG + BMI*GABD | 0.702 | 0.636 | 0.768 | 0.44 |
| ATD_~PSG3/(TETN + APOC3) + IBP4/SHBG + BMI*GABD | 0.702 | 0.637 | 0.768 | 0.36 |
| ATD_~(EGLN + TIMP1)/TETN + IBP4/SHBG + BMI*GABD | 0.702 | 0.637 | 0.768 | 0.22 |
| ATD_~(PRL + EGLN)/PGRP2 + IBP4/SHBG + BMI*GABD | 0.701 | 0.634 | 0.767 | 0.47 |
| ATD_~(PRL + PSG3)/ATS13 + IBP4/SHBG + BMI*GABD | 0.701 | 0.633 | 0.769 | 0.19 |
| ATD_~(ADA12 + CFAB)/APOC3 + IBP4/SHBG + BMI*GABD | 0.701 | 0.634 | 0.767 | 0.19 |
| ATD_~(CFAB + TIMP1)/APOC3 + IBP4/SHBG + BMI*GABD | 0.7 | 0.633 | 0.768 | 0.24 |
| ATD_~ADA12/(TETN + PGRP2) + IBP4/SHBG + BMI*GABD | 0.7 | 0.634 | 0.766 | 0.23 |
| ATD_~(EGLN + CFAB)/ATS13 + IBP4/SHBG + BMI*GABD | 0.7 | 0.632 | 0.768 | 0.20 |
| ATD_~ADA12/(TETN + ATS13) + IBP4/SHBG + BMI*GABD | 0.7 | 0.633 | 0.768 | 0.19 |
| ATD_~(ADA12 + IBP1)TETN + IBP4/SHBG + BMI*GABD | 0.7 | 0.633 | 0.767 | 0.17 |
| ATD_~PRL + EGLN + PSG3 + IBP4/SHBG + BMI*GABD | 0.699 | 0.632 | 0.765 | 0.16 |
| ATD_~PRL + CFAB + IBP1 + IBP4/SHBG + BMI*GABD | 0.698 | 0.629 | 0.768 | 0.24 |
| ATD_~(ADA12 + EGLN)/ATS13 + IBP4/SHBG + BMI*GABD | 0.698 | 0.631 | 0.765 | 0.23 |
| ATD_~(ADA12 + TIMP1)/TETN + IBP4/SHBG + BMI*GABD | 0.698 | 0.631 | 0.765 | 0.18 |
| ATD_~PRL/(ATS13 + APOC3) + IBP4/SHBG + BMI*GABD | 0.698 | 0.629 | 0.767 | 0.18 |
| ATD_~PRL + ADA12 + CFAB + IBP4/SHBG + BMI*GABD | 0.697 | 0.629 | 0.765 | 0.32 |
| ATD_~EGLN/(ATS13 + APOC3) + IBP4/SHBG + BMI*GABD | 0.697 | 0.627 | 0.767 | 0.20 |
| ATD_~EGLN + PSG3 + CFAB + IBP4/SHBG + BMI*GABD | 0.696 | 0.63 | 0.762 | 0.92 |
| ATD_~PRL/(PGRP2 + APOC3) + IBP4/SHBG + BMI*GABD | 0.696 | 0.629 | 0.763 | 0.80 |
| ATD_~(PRL + TIMP1)/APOC3 + IBP4/SHBG + BMI*GABD | 0.696 | 0.629 | 0.763 | 0.42 |
| ATD_~(PSG3 + CFAB)/TETN + IBP4/SHBG + BMI*GABD | 0.696 | 0.629 | 0.763 | 0.23 |
| ATD_~(PRL + ADA12)/APOC3 + IBP4/SHBG + BMI*GABD | 0.696 | 0.63 | 0.762 | 0.22 |
| ATD_~PRL + EGLN + IBP1 + IBP4/SHBG + BMI*GABD | 0.696 | 0.628 | 0.764 | 0.21 |
| ATD_~(CFAB + TIMP1)/TETN + IBP4/SHBG + BMI*GABD | 0.696 | 0.63 | 0.763 | 0.19 |
| ATD_~(ADA12 + PSG3)/APOC3 + IBP4/SHBG + BMI*GABD | 0.696 | 0.629 | 0.762 | 0.18 |
| ATD_~(CFAB + IBP1)/APOC3 + IBP4/SHBG + BMI*GABD | 0.696 | 0.626 | 0.765 | 0.17 |
| ATD_~PRL + PSG3 + IBP1 + IBP4/SHBG + BMI*GABD | 0.696 | 0.628 | 0.763 | 0.16 |
| ATD_~PRL + CFAB + CHL1 + IBP4/SHBG + BMI*GABD | 0.695 | 0.626 | 0.764 | 0.58 |
| ATD_~(PRL + IBP1)/APOC3 + IBP4/SHBG + BMI*GABD | 0.695 | 0.627 | 0.762 | 0.32 |
| ATD_~(PRL + CHL1)/APOC3 + IBP4/SHBG + BMI*GABD | 0.695 | 0.627 | 0.762 | 0.27 |
| ATD_~PRL/(PGRP2 + ATS13) + IBP4/SHBG + BMI*GABD | 0.695 | 0.626 | 0.763 | 0.26 |
| ATD_~CFAB/(ATS13 + APOC3) + IBP4/SHBG + BMI*GABD | 0.695 | 0.625 | 0.765 | 0.19 |
| ATD_~PRL + ADA12 + PSG3 + IBP4/SHBG + BMI*GABD | 0.695 | 0.628 | 0.761 | 0.17 |
| ATD_~(PRL + CFAB)/ATS13 + IBP4/SHBG + BMI*GABD | 0.695 | 0.625 | 0.765 | 0.17 |
| ATD_~ADA12 + PSG3 + CFAB + IBP4/SHBG + BMI*GABD | 0.695 | 0.628 | 0.761 | 0.16 |
| ATD_~(ADA12 + CHL1)/TETN + IBP4/SHBG + BMI*GABD | 0.695 | 0.628 | 0.763 | 0.14 |
| ATD_~PRL + ADA12 + EGLN + IBP4/SHBG + BMI*GABD | 0.694 | 0.627 | 0.761 | 0.45 |
| ATD_~(EGLN + CFAB)/PGRP2 + IBP4/SHBG + BMI*GABD | 0.694 | 0.628 | 0.761 | 0.38 |
| ATD_~PRL + EGLN + CHL1 + IBP4/SHBG + BMI*GABD | 0.694 | 0.626 | 0.762 | 0.16 |
| ATD_~ADA12 + EGLN + CFAB + IBP4/SHBG + BMI*GABD | 0.694 | 0.627 | 0.761 | 0.16 |
| ATD_~(PRL + IBP1)/ATS13 + IBP4/SHBG + BMI*GABD | 0.693 | 0.624 | 0.763 | 0.40 |
| ATD_~(PRL + IBP1)/PGRP2 + IBP4/SHBG + BMI*GABD | 0.693 | 0.625 | 0.761 | 0.39 |
| ATD_~EGLN + CFAB + IBP1 + IBP4/SHBG + BMI*GABD | 0.693 | 0.626 | 0.761 | 0.35 |
| ATD_~EGLN/(PGRP2 + ATS13) + IBP4/SHBG + BMI*GABD | 0.692 | 0.624 | 0.76 | 0.44 |
| ATD_~(EGLN + IBP1)/ATS13 + IBP4/SHBG + BMI*GABD | 0.692 | 0.624 | 0.761 | 0.34 |
| ATD_~(ADA12 + CFAB)/PGRP2 + IBP4/SHBG + BMI*GABD | 0.692 | 0.625 | 0.76 | 0.26 |
| ATD_~(ADA12 + CFAB)/ATS13 + IBP4/SHBG + BMI*GABD | 0.692 | 0.624 | 0.761 | 0.25 |
| ATD_~(CFAB + CHL1)/APOC3 + IBP4/SHBG + BMI*GABD | 0.692 | 0.622 | 0.761 | 0.22 |
| ATD_~(PRL + TIMP1)/ATS13 + IBP4/SHBG + BMI*GABD | 0.692 | 0.623 | 0.761 | 0.15 |
| ATD_~(PRL + CHL1)/ATS13 + IBP4/SHBG + BMI*GABD | 0.692 | 0.622 | 0.762 | 0.15 |
| ATD_~PRL + CFAB + TIMP1 + IBP4/SHBG + BMI*GABD | 0.691 | 0.622 | 0.761 | 0.58 |
| ATD_~IBP1/(TETN + APOC3) + IBP4/SHBG + BMI*GABD | 0.691 | 0.621 | 0.76 | 0.43 |
| ATD_~(PRL + ADA12)/PGRP2 + IBP4/SHBG + BMI*GABD | 0.691 | 0.625 | 0.757 | 0.20 |
| ATD_~(EGLN + PSG3)/ATS13 + IBP4/SHBG + BMI*GABD | 0.691 | 0.623 | 0.758 | 0.17 |
| ATD_~(EGLN + PSG3)/APOC3 + IBP4/SHBG + BMI*GABD | 0.69 | 0.623 | 0.757 | 0.67 |
| ATD_~EGLN + CFAB + TIMP1 + IBP4/SHBG + BMI*GABD | 0.69 | 0.623 | 0.757 | 0.34 |
| ATD_~CFAB/(PGRP2 + APOC3) + IBP4/SHBG + BMI*GABD | 0.69 | 0.621 | 0.759 | 0.32 |
| ATD_~(ADA12 + EGLN)/APOC3 + IBP4/SHBG + BMI*GABD | 0.69 | 0.622 | 0.757 | 0.21 |
| ATD_~PRL + PSG3 + CHL1 + IBP4/SHBG + BMI*GABD | 0.69 | 0.623 | 0.758 | 0.15 |
| ATD_~ADA12 + CFAB + IBP1 + IBP4/SHBG + BMI*GABD | 0.69 | 0.622 | 0.758 | 0.14 |
| ATD_~(PRL + CHL1)/PGRP2 + IBP4/SHBG + BMI*GABD | 0.689 | 0.621 | 0.757 | 0.46 |
| ATD_~(PRL + TIMP1)/PGRP2 + IBP4/SHBG + BMI*GABD | 0.689 | 0.623 | 0.756 | 0.37 |
| ATD_~EGLN + CFAB + CHL1 + IBP4/SHBG + BMI*GABD | 0.689 | 0.621 | 0.756 | 0.36 |
| ATD_~(PSG3 + TIMP1)/APOC3 + IBP4/SHBG + BMI*GABD | 0.689 | 0.621 | 0.756 | 0.27 |
| ATD_~(EGLN + CHL1)/ATS13 + IBP4/SHBG + BMI*GABD | 0.689 | 0.62 | 0.759 | 0.23 |
| ATD_~PRL + EGLN + TIMP1 + IBP4/SHBG + BMI*GABD | 0.689 | 0.621 | 0.757 | 0.23 |
| ATD_~(ADA12 + IBP1)/ATS13 + IBP4/SHBG + BMI*GABD | 0.689 | 0.621 | 0.758 | 0.22 |
| ATD_~CHL1/(TETN + APOC3) + IBP4/SHBG + BMI*GABD | 0.689 | 0.62 | 0.759 | 0.22 |
| ATD_~(PSG3 + TIMP1)/TETN + IBP4/SHBG + BMI*GABD | 0.689 | 0.623 | 0.756 | 0.17 |
| ATD_~1/(TETN + PGRP2 + APOC3) + IBP4/SHBG + BMI*GABD | 0.688 | 0.618 | 0.757 | 0.28 |
| ATD_~(CFAB + IBP1)/TETN + IBP4/SHBG + BMI*GABD | 0.688 | 0.619 | 0.757 | 0.16 |
| ATD_~PSG3/(PGRP2 + APOC3) + IBP4/SHBG + BMI*GABD | 0.687 | 0.62 | 0.755 | 0.97 |
| ATD_~PRL + ADA12 + IBP1 + IBP4/SHBG + BMI*GABD | 0.687 | 0.619 | 0.756 | 0.80 |
| ATD_~PSG3/(ATS13 + APOC3) + IBP4/SHBG + BMI*GABD | 0.687 | 0.618 | 0.756 | 0.31 |
| ATD_~(EGLN + TIMP1)/ATS13 + IBP4/SHBG + BMI*GABD | 0.687 | 0.618 | 0.756 | 0.19 |
| ATD_~(PSG3 + IBP1)/APOC3 + IBP4/SHBG + BMI*GABD | 0.686 | 0.619 | 0.754 | 0.67 |
| ATD_~(PSG3 + CFAB)/PGRP2 + IBP4/SHBG + BMI*GABD | 0.686 | 0.619 | 0.754 | 0.48 |
| ATD_~PSG3 + CFAB + IBP1 + IBP4/SHBG + BMI*GABD | 0.686 | 0.618 | 0.753 | 0.27 |
| ATD_~ADA12/(ATS13 + APOC3) + IBP4/SHBG + BMI*GABD | 0.686 | 0.617 | 0.754 | 0.23 |
| ATD_~CFAB/(TETN + PGRP2) + IBP4/SHBG + BMI*GABD | 0.686 | 0.617 | 0.755 | 0.22 |
| ATD_~(ADA12 + IBP1)/APOC3 + IBP4/SHBG + BMI*GABD | 0.686 | 0.618 | 0.753 | 0.20 |
| ATD_~PRL + IBP1 + CHL1 + IBP4/SHBG + BMI*GABD | 0.686 | 0.616 | 0.755 | 0.18 |
| ATD_~(IBP1 + TIMP1)/TETN + IBP4/SHBG + BMI*GABD | 0.686 | 0.618 | 0.754 | 0.18 |
| ATD_~PSG3 + CFAB + TIMP1 + IBP4/SHBG + BMI*GABD | 0.686 | 0.619 | 0.753 | 0.16 |
| ATD_~PRL + PSG3 + TIMP1 + IBP4/SHBG + BMI*GABD | 0.686 | 0.618 | 0.754 | 0.15 |
| ATD_~(ADA12 + PSG3)/ATS13 + IBP4/SHBG + BMI*GABD | 0.686 | 0.618 | 0.754 | 0.13 |
| ATD_~PRL + ADA12 + CHL1 + IBP4/SHBG + BMI*GABD | 0.685 | 0.616 | 0.753 | 0.34 |
| ATD_~1/(TETN + ATS13 + APOC3) + IBP4/SHBG + BMI*GABD | 0.685 | 0.615 | 0.755 | 0.30 |
| ATD_~ADA12/(PGRP2 + APOC3) + IBP4/SHBG + BMI*GABD | 0.685 | 0.618 | 0.752 | 0.27 |
| ATD_~(ADA12 + EGLN)/PGRP2 + IBP4/SHBG + BMI*GABD | 0.685 | 0.618 | 0.751 | 0.23 |
| ATD_~(CFAB + CHL1)/TETN + IBP4/SHBG + BMI*GABD | 0.685 | 0.616 | 0.754 | 0.19 |
| ATD_~ADA12/(PGRP2 + ATS13) + IBP4/SHBG + BMI*GABD | 0.685 | 0.616 | 0.754 | 0.14 |
| ATD_~(EGLN + PSG3)/PGRP2 + IBP4/SHBG + BMI*GABD | 0.684 | 0.618 | 0.751 | 1.22 |
| ATD_~EGLN/(PGRP2 + APOC3) + IBP4/SHBG + BMI*GABD | 0.684 | 0.616 | 0.752 | 1.19 |
| ATD_~(PSG3 + IBP1)/TETN + IBP4/SHBG + BMI*GABD | 0.684 | 0.617 | 0.751 | 0.30 |
| ATD_~ADA12 + PSG3 + IBP1 + IBP4/SHBG + BMI*GABD | 0.684 | 0.617 | 0.75 | 0.16 |
| ATD_~(ADA12 + PSG3)/PGRP2 + IBP4/SHBG + BMI*GABD | 0.684 | 0.618 | 0.75 | 0.15 |
| ATD_~TIMP1/(ATS13 + APOC3) + IBP4/SHBG + BMI*GABD | 0.683 | 0.613 | 0.754 | 0.68 |
| ATD_~(EGLN + IBP1)/APOC3 + IBP4/SHBG + BMI*GABD | 0.683 | 0.615 | 0.752 | 0.52 |
| ATD_~ADA12 + EGLN + IBP1 + IBP4/SHBG + BMI*GABD | 0.683 | 0.616 | 0.75 | 0.23 |
| ATD_~PRL + IBP1 + TIMP1 + IBP4/SHBG + BMI*GABD | 0.683 | 0.614 | 0.753 | 0.19 |
| ATD_~(ADA12 + IBP1)/PGRP2 + IBP4/SHBG + BMI*GABD | 0.683 | 0.616 | 0.75 | 0.17 |
| ATD_~TIMP1/(TETN + PGRP2) + IBP4/SHBG + BMI*GABD | 0.683 | 0.616 | 0.751 | 0.16 |
| ATD_~(EGLN + IBP1)/PGRP2 + IBP4/SHBG + BMI*GABD | 0.682 | 0.615 | 0.749 | 4.54 |
| ATD_~(EGLN + CHL1)/APOC3 + IBP4/SHBG + BMI*GABD | 0.682 | 0.612 | 0.751 | 0.71 |
| ATD_~PRL + ADA12 + TIMP1 + IBP4/SHBG + BMI*GABD | 0.682 | 0.614 | 0.75 | 0.33 |
| ATD_~(PSG3 + CFAB)/ATS13 + IBP4/SHBG + BMI*GABD | 0.682 | 0.613 | 0.751 | 0.31 |
| ATD_~ADA12 + CFAB + CHL1 + IBP4/SHBG + BMI*GABD | 0.682 | 0.613 | 0.75 | 0.19 |
| ATD_~PSG3/(TETN + PGRP2) + IBP4/SHBG + BMI*GABD | 0.682 | 0.615 | 0.749 | 0.17 |
| ATD_~PSG3 + CFAB + CHL1 + IBP4/SHBG + BMI*GABD | 0.682 | 0.614 | 0.749 | 0.16 |
| ATD_~ADA12 + CFAB + TIMP1 + IBP4/SHBG + BMI*GABD | 0.682 | 0.613 | 0.75 | 0.15 |
| ATD_~IBP1/(TETN + PGRP2) + IBP4/SHBG + BMI*GABD | 0.681 | 0.612 | 0.75 | 0.37 |
| ATD_~(PSG3 + CHL1)/APOC3 + IBP4/SHBG + BMI*GABD | 0.681 | 0.613 | 0.749 | 0.30 |
| ATD_~CFAB/(TETN + ATS13) + IBP4/SHBG + BMI*GABD | 0.681 | 0.611 | 0.751 | 0.16 |
| ATD_~(EGLN + TIMP1)/APOC3 + IBP4/SHBG + BMI*GABD | 0.68 | 0.61 | 0.749 | 1.54 |
| ATD_~(IBP1 + TIMP1)/APOC3 + IBP4/SHBG + BMI*GABD | 0.68 | 0.611 | 0.749 | 0.62 |
| ATD_~TIMP1/(TETN + ATS13) + IBP4/SHBG + BMI*GABD | 0.68 | 0.611 | 0.748 | 0.38 |
| ATD_~(CFAB + TIMP1)/ATS13 + IBP4/SHBG + BMI*GABD | 0.68 | 0.611 | 0.749 | 0.27 |
| ATD_~(CFAB + TIMP1)/PGRP2 + IBP4/SHBG + BMI*GABD | 0.68 | 0.613 | 0.747 | 0.21 |
| ATD_~EGLN + PSG3 + IBP1 + IBP4/SHBG + BMI*GABD | 0.679 | 0.612 | 0.746 | 0.67 |
| ATD_~(CFAB + IBP1)/PGRP2 + IBP4/SHBG + BMI*GABD | 0.679 | 0.61 | 0.748 | 0.26 |
| ATD_~PRL + CHL1 + TIMP1 + IBP4/SHBG + BMI*GABD | 0.679 | 0.61 | 0.748 | 0.19 |
| ATD_~(ADA12 + CHL1)/ATS13 + IBP4/SHBG + BMI*GABD | 0.679 | 0.609 | 0.749 | 0.17 |
| ATD_~(CHL1 + TIMP1)/TETN + IBP4/SHBG + BMI*GABD | 0.679 | 0.611 | 0.747 | 0.15 |
| ATD_~CFAB + IBP1 + TIMP1 + IBP4/SHBG + BMI*GABD | 0.679 | 0.611 | 0.748 | 0.12 |
| ATD_~TIMP1/(PGRP2 + APOC3) + IBP4/SHBG + BMI*GABD | 0.678 | 0.61 | 0.747 | 3.75 |
| ATD_~(ADA12 + TIMP1)/APOC3/+ IBP4/SHBG + BMI*GABD | 0.678 | 0.609 | 0.747 | 0.18 |
| ATD_~PSG3/(TETN + ATS13) + IBP4/SHBG + BMI*GABD | 0.678 | 0.61 | 0.745 | 0.15 |
| ATD_~(ADA12 + CHL1)/APOC3 + IBP4/SHBG + BMI*GABD | 0.678 | 0.61 | 0.746 | 0.13 |
| ATD_~EGLN + IBP1 + CHL1 + IBP4/SHBG + BMI*GABD | 0.677 | 0.608 | 0.745 | 2.08 |
| ATD_~(PSG3 + CHL1)/TETN + IBP4/SHBG + BMI*GABD | 0.677 | 0.61 | 0.744 | 0.25 |
| ATD_~(IBP1 + CHL1)/TETN + IBP4/SHBG + BMI*GABD | 0.677 | 0.607 | 0.747 | 0.16 |
| ATD_~(ADA12 + TIMP1)/ATS13 + IBP4/SHBG + BMI*GABD | 0.677 | 0.608 | 0.747 | 0.12 |
| ATD_~(EGLN + CHL1)/PGRP2 + IBP4/SHBG + BMI*GABD | 0.676 | 0.608 | 0.744 | 0.90 |
| ATD_~(PSG3 + IBP1)/PGRP2 + IBP4/SHBG + BMI*GABD | 0.676 | 0.609 | 0.743 | 0.67 |
| ATD_~(CFAB + IBP1)/ATS13 + IBP4/SHBG + BMI*GABD | 0.676 | 0.605 | 0.747 | 0.17 |
| ATD_~ADA12 + IBP1 + CHL1 + IBP4/SHBG + BMI*GABD | 0.676 | 0.608 | 0.744 | 0.17 |
| ATD_~ADA12 + EGLN + PSG3 + IBP4/SHBG + BMI*GABD | 0.676 | 0.609 | 0.742 | 0.14 |
| ATD_~CFAB + CHL1 + TIMP1 + IBP4/SHBG + BMI*GABD | 0.676 | 0.607 | 0.744 | 0.14 |
| ATD_~1/(PGRP2 + ATS13 + APOC3) + IBP4/SHBG + BMI*GABD | 0.675 | 0.604 | 0.747 | 1.41 |
| ATD_~(PSG3 + IBP1)/ATS13 + IBP4/SHBG + BMI*GABD | 0.675 | 0.606 | 0.743 | 0.36 |
| ATD_~CFAB + IBP1 + CHL1 + IBP4/SHBG + BMI*GABD | 0.675 | 0.605 | 0.745 | 0.17 |
| ATD_~IBP1/(ATS13 + APOC3) + IBP4/SHBG + BMI*GABD | 0.674 | 0.601 | 0.747 | 0.98 |
| ATD_~IBP1/(PGRP2 + APOC3) + IBP4/SHBG + BMI*GABD | 0.674 | 0.604 | 0.743 | 0.95 |
| ATD_~IBP1/(TETN + ATS13) + IBP4/SHBG + BMI*GABD | 0.674 | 0.603 | 0.745 | 0.23 |
| ATD_~ADA12 + EGLN + CHL1 + IBP4/SHBG + BMI*GABD | 0.674 | 0.606 | 0.742 | 0.16 |
| ATD_~(ADA12 + CHL1)/PGRP2 + IBP4/SHBG + BMI*GABD | 0.673 | 0.606 | 0.741 | 0.13 |
| ATD_~(CHL1 + TIMP1)/APOC3 + IBP4/SHBG + BMI*GABD | 0.672 | 0.602 | 0.742 | 3.11 |
| ATD_~CFAB/(PGRP2 + ATS13) + IBP4/SHBG + BMI*GABD | 0.672 | 0.601 | 0.743 | 0.66 |
| ATD_~(IBP1 + CHL1)/APOC3 + IBP4/SHBG + BMI*GABD | 0.672 | 0.601 | 0.743 | 0.53 |
| ATD_~EGLN + PSG3 + CHL1 + IBP4/SHBG + BMI*GABD | 0.672 | 0.605 | 0.74 | 0.32 |
| ATD_~PSG3/(PGRP2 + ATS13) + IBP4/SHBG + BMI*GABD | 0.672 | 0.604 | 0.741 | 0.24 |
| ATD_~(PSG3 + TIMP1)/ATS13 + IBP4/SHBG + BMI*GABD | 0.672 | 0.602 | 0.741 | 0.18 |
| ATD_~ADA12 + IBP1 + TIMP1 + IBP4/SHBG + BMI*GABD | 0.672 | 0.604 | 0.741 | 0.15 |
| ATD_~(EGLN + TIMP1)/PGRP2 + IBP4/SHBG + BMI*GABD | 0.671 | 0.603 | 0.738 | 5.40 |
| ATD_~CHL1/(PGRP2 + APOC3) + IBP4/SHBG + BMI*GABD | 0.671 | 0.601 | 0.741 | 0.44 |
| ATD_~(PSG3 + TIMP1)/PGRP2 + IBP4/SHBG + BMI*GABD | 0.671 | 0.603 | 0.739 | 0.26 |
| ATD_~1/(TETN + PGRP2 + ATS13) + IBP4/SHBG + BMI*GABD | 0.671 | 0.601 | 0.741 | 0.17 |
| ATD_~CHL1/(TETN + PGRP2) + IBP4/SHBG + BMI*GABD | 0.671 | 0.602 | 0.741 | 0.12 |
| ATD_~EGLN + IBP1 + TIMP1 + IBP4/SHBG + BMI*GABD | 0.67 | 0.602 | 0.738 | 5.24 |
| ATD_~PSG3 + IBP1 + TIMP1 + IBP4/SHBG + BMI*GABD | 0.67 | 0.602 | 0.738 | 0.20 |
| ATD_~(CFAB + CHL1)/PGRP2 + IBP4/SHBG + BMI*GABD | 0.67 | 0.601 | 0.739 | 0.19 |
| ATD_~ADA12 + EGLN + TIMP1 + IBP4/SHBG + BMI*GABD | 0.67 | 0.602 | 0.738 | 0.14 |
| ATD_~ADA12 + PSG3 + CHL1 + IBP4/SHBG + BMI*GABD | 0.67 | 0.602 | 0.737 | 0.11 |
| ATD_~ADA12 + PSG3 + TIMP1 + IBP4/SHBG + BMI*GABD | 0.67 | 0.603 | 0.738 | 0.11 |
| ATD_~EGLN + PSG3 + TIMP1 + IBP4/SHBG + BMI*GABD | 0.669 | 0.602 | 0.737 | 0.54 |
| ATD_~(IBP1 + TIMP1)/ATS13 + IBP4/SHBG + BMI*GABD | 0.669 | 0.599 | 0.74 | 0.34 |
| ATD_~(CFAB + CHL1)/ATS13 + IBP4/SHBG + BMI*GABD | 0.669 | 0.597 | 0.74 | 0.20 |
| ATD_~CHL1/(ATS13 + APOC3) + IBP4/SHBG + BMI*GABD | 0.668 | 0.595 | 0.741 | 0.28 |
| ATD_~CHL1/(TETN + ATS13) + IBP4/SHBG + BMI*GABD | 0.668 | 0.597 | 0.739 | 0.13 |
| ATD_~IBP1/(PGRP2 + ATS13) + IBP4/SHBG + BMI*GABD | 0.667 | 0.595 | 0.738 | 0.41 |
| ATD_~(ADA12 + TIMP1)/PGRP2 + IBP4/SHBG + BMI*GABD | 0.667 | 0.599 | 0.735 | 0.12 |
| ATD_~TIMP1/(PGRP2 + ATS13) + IBP4/SHBG + BMI*GABD | 0.666 | 0.597 | 0.736 | 0.27 |
| ATD_~EGLN + CHL1 + TIMP1 + IBP4/SHBG + BMI*GABD | 0.665 | 0.596 | 0.734 | 1.21 |
| ATD_~(IBP1 + TIMP1)/PGPR2 + IBP4/SHBG + BMI*GABD | 0.665 | 0.596 | 0.733 | 0.65 |
| ATD_~(IBP1 + CHL1)/PGRP2 + IBP4/SHBG + BMI*GABD | 0.665 | 0.595 | 0.735 | 0.41 |
| ATD_~(PSG3 + CHL1)/PGRP2 + IBP4/SHBG + BMI*GABD | 0.665 | 0.597 | 0.733 | 0.28 |
| ATD_~PSG3 + IBP1 + CHL1 + IBP4/SHBG + BMI*GABD | 0.664 | 0.595 | 0.732 | 0.31 |
| ATD_~(PSG3 + CHL1)/ATS13 + IBP4/SHBG + BMI*GABD | 0.663 | 0.593 | 0.733 | 0.20 |
| ATD_~(IBP1 + CHL1)/ATS13 + IBP4/SHBG + BMI*GABD | 0.662 | 0.588 | 0.735 | 0.26 |
| ATD_~CHL1/(PGRP2 + ATS13) + IBP4/SHBG + BMI*GABD | 0.661 | 0.589 | 0.733 | 0.29 |
| ATD_~(CHL1 + TIMP1)/ATS13 + IBP4/SHBG + BMI*GABD | 0.661 | 0.589 | 0.733 | 0.28 |
| ATD_~(CHL1 + TIMP1)/PGRP2 + IBP4/SHBG + BMI*GABD | 0.66 | 0.592 | 0.729 | 3.44 |
| ATD_~IBP1 + CHL1 + TIMP1 + IBP4/SHBG + BMI*GABD | 0.659 | 0.589 | 0.729 | 0.66 |
| ATD_~PSG3 + CHL1 + TIMP1 + IBP4/SHBG + BMI*GABD | 0.659 | 0.591 | 0.728 | 0.14 |
| ATD_~ADA12 + CHL1 + TIMP1 + IBP4/SHBG + BMI*GABD | 0.658 | 0.589 | 0.728 | 0.13 |

TABLE 15
Reversal Group Classification Performance at Predicting sPTB <37 weeks gestation.
Reversal group comprises IBP4/SHBG, three additional protein biomarkers, two
additional clinical variables and a demographic variable of prior sPTB.

| | | CI | CI | Sum |
|---|---|---|---|---|
| Reversal Group | AUC | low | high | Rank |
| ATD_~PRL + IBP4/SHBG + PriorPTB + BMI*GABD | 0.691 | 0.623 | 0.759 | 9.58 |
| ATD_~1/PGRP2 + IBP4/SHBG + PriorPTB + BMI*GABD | 0.665 | 0.597 | 0.732 | 6.93 |
| ATD_~EGLN + IBP4/SHBG + PriorPTB + BMI*GABD | 0.679 | 0.612 | 0.746 | 6.03 |
| ATD_~IBP1 + IBP4/SHBG + PriorPTB + BMI*GABD | 0.675 | 0.606 | 0.743 | 5.52 |
| ATD_~TIMP1 + IBP4/SHBG + PriorPTB + BMI*GABD | 0.665 | 0.597 | 0.733 | 4.87 |
| ATD_~CFAB + IBP4/SHBG + PriorPTB + BMI*GABD | 0.682 | 0.614 | 0.75 | 4.22 |
| ATD_~1/APOC3 + IBP4/SHBG + PriorPTB + BMI*GABD | 0.682 | 0.613 | 0.751 | 4.14 |
| ATD_~PSG3 + IBP4/SHBG + PriorPTB + BMI*GABD | 0.676 | 0.609 | 0.743 | 3.06 |
| ATD_~CHL1 + IBP4/SHBG + PriorPTB + BMI*GABD | 0.665 | 0.596 | 0.734 | 2.77 |
| ATD_~1/ATS13 + IBP4/SHBG + PriorPTB + BMI*GABD | 0.664 | 0.592 | 0.735 | 2.64 |
| ATD_~ADA12 + IBP4/SHBG + PriorPTB + BMI*GABD | 0.677 | 0.61 | 0.744 | 2.46 |
| ATD_~1/TETN + IBP4/SHBG + PriorPTB + BMI*GABD | 0.674 | 0.605 | 0.743 | 2.17 |

TABLE 16
Reversal Group Classification Performance at Predicting sPTB <37 weeks gestation.
Reversal group comprises IBP4/SHBG, two additional protein biomarkers, two
additional clinical variables and a demographic variable of prior sPTB.

| | | CI | CI | Sum |
|---|---|---|---|---|
| Reversal Group | AUC | low | high | Rank |
| ATD_~EGLN/TETN + IBP4/SHBG + PriorPTB + BMI*GABD | 0.710 | 0.646 | 0.775 | 2.82 |
| ATD_~PRL/TETN + IBP4/SHBG + PriorPTB + BMI*GABD | 0.710 | 0.643 | 0.778 | 2.76 |
| ATD_~PRL + PSG3 + IBP4/SHBG + PriorPTB + BMI*GABD | 0.707 | 0.642 | 0.773 | 1.33 |
| ATD_~EGLN + CFAB + IBP4/SHBG + PriorPTB + BMI*GABD | 0.706 | 0.642 | 0.771 | 2.08 |
| ATD_~PRL + CFAB + IBP4/SHBG + PriorPTB + BMI*GABD | 0.706 | 0.638 | 0.773 | 1.87 |
| ATD_~PRL + EGLN + IBP4/SHBG + PriorPTB + BMI*GABD | 0.706 | 0.64 | 0.772 | 1.12 |
| ATD_~PRL/APOC3 + IBP4/SHBG + PriorPTB + BMI*GABD | 0.703 | 0.637 | 0.77 | 1.15 |
| ATD_~CFAB/APOC3 + IBP4/SHBG + PriorPTB + BMI*GABD | 0.702 | 0.634 | 0.77 | 0.84 |
| ATD_~ADA12/TETN + IBP4/SHBG + PriorPTB + BMI*GABD | 0.702 | 0.635 | 0.768 | 0.79 |
| ATD_~PSG3/APOC3 + IBP4/SHBG + PriorPTB + BMI*GABD | 0.7 | 0.634 | 0.766 | 0.92 |
| ATD_~PRL + IBP1 + IBP4/SHBG + PriorPTB + BMI*GABD | 0.698 | 0.63 | 0.765 | 1.03 |
| ATD_~PRL/ATS13 + IBP4/SHBG + PriorPTB + BMI*GABD | 0.698 | 0.63 | 0.767 | 0.83 |
| ATD_~PRL/PGRP2 + IBP4/SHBG + PriorPTB + BMI*GABD | 0.697 | 0.631 | 0.763 | 2.22 |
| ATD_~EGLN/ATS13 + IBP4/SHBG + PriorPTB + BMI*GABD | 0.697 | 0.629 | 0.764 | 0.71 |
| ATD_~PRL + ADA12 + IBP4/SHBG + PriorPTB + BMI*GABD | 0.696 | 0.629 | 0.762 | 1.71 |
| ATD_~1/(TETN + APOC3) + IBP4/SHBG + PriorPTB + BMI*GABD | 0.696 | 0.628 | 0.765 | 0.77 |
| ATD_~ADA12 + CFAB + IBP4/SHBG + PriorPTB + BMI*GABD | 0.696 | 0.63 | 0.763 | 0.66 |
| ATD_~EGLN/APOC3 + IBP4/SHBG + PriorPTB + BMI*GABD | 0.695 | 0.628 | 0.763 | 2.95 |
| ATD_~PSG3 + CFAB + IBP4/SHBG + PriorPTB + BMI*GABD | 0.695 | 0.629 | 0.761 | 0.76 |
| ATD_~ADA12/ATS13 + IBP4/SHBG + PriorPTB + BMI*GABD | 0.695 | 0.627 | 0.764 | 0.47 |
| ATD_~PRL + CHL1 + IBP4/SHBG + PriorPTB + BMI*GABD | 0.693 | 0.625 | 0.761 | 2.44 |
| ATD_~PRL + TIMP1 + IBP4/SHBG + PriorPTB + BMI*GABD | 0.693 | 0.625 | 0.76 | 1.41 |
| ATD_~ADA12/APOC3 + IBP4/SHBG + PriorPTB + BMI*GABD | 0.693 | 0.627 | 0.759 | 0.52 |
| ATD_~ADA12 + PSG3 + IBP4/SHBG + PriorPTB + BMI*GABD | 0.693 | 0.628 | 0.759 | 0.49 |
| ATD_~EGLN + PSG3 + IBP4/SHBG + PriorPTB + BMI*GABD | 0.692 | 0.626 | 0.757 | 1.13 |
| ATD_~ADA12 + IBP1 + IBP4/SHBG + PriorPTB + BMI*GABD | 0.692 | 0.625 | 0.758 | 0.50 |
| ATD_~EGLN + IBP1 + IBP4/SHBG + PriorPTB + BMI*GABD | 0.69 | 0.624 | 0.756 | 3.18 |
| ATD_~TIMP/TETN + IBP4/SHBG + PriorPTB + BMI*GABD | 0.69 | 0.624 | 0.757 | 0.50 |
| ATD_~CFAB + TIMP1 + IBP4/SHBG + PriorPTB + BMI*GABD | 0.69 | 0.623 | 0.757 | 0.50 |
| ATD_~ADA12 + EGLN + IBP4/SHBG + PriorPTB + BMI*GABD | 0.69 | 0.624 | 0.756 | 0.43 |
| ATD_~TIMP1/APOC3 + IBP4/SHBG + PriorPTB + BMI*GABD | 0.689 | 0.621 | 0.758 | 8.65 |
| ATD_~CFAB + IBP1 + IBP4/SHBG + PriorPTB + BMI*GABD | 0.689 | 0.621 | 0.757 | 0.87 |
| ATD_~CFAB/TETN + IBP4/SHBG + PriorPTB + BMI*GABD | 0.689 | 0.621 | 0.757 | 0.61 |
| ATD_~PSG3/TETN + IBP4/SHBG + PriorPTB + BMI*GABD | 0.689 | 0.623 | 0.755 | 0.44 |
| ATD_~IBP1/APOC3 + IBP4/SHBG + PriorPTB + BMI*GABD | 0.688 | 0.62 | 0.757 | 0.85 |
| ATD_~EGLN/PGRP2 + IBP4/SHBG + PriorPTB + BMI*GABD | 0.687 | 0.621 | 0.753 | 2.38 |
| ATD_~IBP1/TETN + IBP4/SHBG + PriorPTB + BMI*GABD | 0.686 | 0.617 | 0.754 | 0.94 |
| ATD_~PSG3 + IBP1 + IBP4/SHBG + PriorPTB + BMI*GABD | 0.686 | 0.62 | 0.752 | 0.65 |
| ATD_~PSG3/PGRP2 + IBP4/SHBG + PriorPTB + BMI*GABD | 0.684 | 0.618 | 0.75 | 0.82 |
| ATD_~EGLN + CHL1 + IBP4/SHBG + PriorPTB + BMI*GABD | 0.683 | 0.616 | 0.75 | 1.09 |
| ATD_~PSG3 + TIMP1 + IBP4/SHBG + PriorPTB + BMI*GABD | 0.683 | 0.616 | 0.749 | 0.97 |
| ATD_~CHL1/APOC3 + IBP4/SHBG + PriorPTB + BMI*GABD | 0.683 | 0.614 | 0.752 | 0.68 |
| ATD_~1/(ATS13 + APOC3) + IBP4/SHBG + PriorPTB + BMI*GABD | 0.683 | 0.611 | 0.754 | 0.53 |
| ATD_~CFAB + CHL1 + IBP4/SHBG + PriorPTB + BMI*GABD | 0.683 | 0.615 | 0.752 | 0.49 |
| ATD_~1/(PGRP2 + APOC3) + IBP4/SHBG + PriorPTB + BMI*GABD | 0.682 | 0.614 | 0.75 | 4.55 |
| ATD_~PSG3/ATS13 + IBP4/SHBG + PriorPTB + BMI*GABD | 0.681 | 0.612 | 0.749 | 0.60 |
| ATD_~ADA12/PGRP2 + IBP4/SHBG + PriorPTB + BMI*GABD | 0.681 | 0.615 | 0.748 | 0.53 |
| ATD_~ADA12 + CHL1 + IBP4/SHBG + PriorPTB + BMI*GABD | 0.681 | 0.613 | 0.748 | 0.47 |
| ATD_~EGLN + TIMP1 + IBP4/SHBG + PriorPTB + BMI*GABD | 0.68 | 0.613 | 0.747 | 3.80 |
| ATD_~IBP1 + TIMP1 + IBP4/SHBG + PriorPTB + BMI*GABD | 0.679 | 0.611 | 0.746 | 0.81 |
| ATD_~CFAB/PGRP2 + IBP4/SHBG + PriorPTB + BMI*GABD | 0.679 | 0.611 | 0.747 | 0.69 |
| ATD_~ADA12 + TIMP1 + IBP4/SHBG + PriorPTB + BMI*GABD | 0.679 | 0.611 | 0.746 | 0.38 |
| ATD_~CFAB/ATS13 + IBP4/SHBG + PriorPTB + BMI*GABD | 0.678 | 0.607 | 0.748 | 0.87 |
| ATD_~PSG3 + CHL1 + IBP4/SHBG + PriorPTB + BMI*GABD | 0.677 | 0.61 | 0.744 | 0.71 |
| ATD_~IBP1/ATS13 + IBP4/SHBG + PriorPTB + BMI*GABD | 0.677 | 0.607 | 0.748 | 0.70 |
| ATD_~TIMP1/ATS13 + IBP4/SHBG + PriorPTB + BMI*GABD | 0.677 | 0.607 | 0.747 | 0.58 |
| ATD_~CHL1/TETN + IBP4/SHBG + PriorPTB + BMI*GABD | 0.677 | 0.608 | 0.746 | 0.43 |
| ATD_~IBP1/PGRP2 + IBP4/SHBG + PriorPTB + BMI*GABD | 0.676 | 0.609 | 0.744 | 1.51 |
| ATD_~TIMP1/PGRP2 + IBP4/SHBG + PriorPTB + BMI*GABD | 0.675 | 0.608 | 0.741 | 2.11 |
| ATD_~IBP1 + CHL1 + IBP4/SHBG + PriorPTB + BMI*GABD | 0.675 | 0.606 | 0.744 | 1.20 |
| ATD_~1/(TETN + PGRP2) + IBP4/SHBG + PriorPTB + BMI*GABD | 0.675 | 0.607 | 0.743 | 0.69 |
| ATD_~1(TETN + ATS13) + IBP4/SHBG + PriorPTB + BMI*GABD | 0.674 | 0.604 | 0.744 | 0.41 |
| ATD_~CHL1 + TIMP1 + IBP4/SHBG + PriorPTB + BMI*GABD | 0.671 | 0.603 | 0.739 | 0.63 |
| ATD_~CHL1/PGRP2 + IBP4/SHBG + PriorPTB + BMI*GABD | 0.668 | 0.6 | 0.736 | 1.10 |
| ATD_~CHL1/ATS13 + IBP4/SHBG + PriorPTB + BMI*GABD | 0.668 | 0.596 | 0.739 | 0.45 |
| ATD_~1/(PGRP2 + ATS13) + IBP4/SHBG + PriorPTB + BMI*GABD | 0.667 | 0.597 | 0.738 | 1.83 |

TABLE 17
Reversal Group Classification Performance at Predicting sPTB <37 weeks gestation.
Reversal group comprises IBP4/SHBG, three additional protein biomarkers, two
additional clinical variables and a demographic variable of prior sPTB.

| | | CI | CI | Sum |
|---|---|---|---|---|
| Reversal Group | AUC | low | high | Rank |
| ATD_~(PRL + EGLN)/TETN + IBP4/SHBG + PriorPTB + BMI*GABD | 0.733 | 0.669 | 0.797 | 3.12 |
| ATD_~(PRL + ADA12)/TETN + IBP4/SHBG + PriorPTB + BMI*GABD | 0.726 | 0.661 | 0.79 | 1.37 |
| ATD_~(PRL + PSG3)/APOC3 + IBP4/SHBG + PriorPTB + BMI*GABD | 0.725 | 0.662 | 0.789 | 1.05 |
| ATD_~EGLN/(TETN + APOC3) + IBP4/SHBG + PriorPTB + BMI*GABD | 0.725 | 0.662 | 0.789 | 0.98 |
| ATD_~(PRL + EGLN)/ATS13 + IBP4/SHBG + PriorPTB + BMI*GABD | 0.725 | 0.66 | 0.79 | 0.71 |
| ATD_~PRL/(TETN + APOC3) + IBP4/SHBG + PriorPTB + BMI*GABD | 0.725 | 0.66 | 0.79 | 0.65 |
| ATD_~(PRL + PSG3)/TETN + IBP4/SHBG + PriorPTB + BMI*GABD | 0.725 | 0.66 | 0.79 | 0.60 |
| ATD_~PRL + EGLN + CFAB + IBP4/SHBG + PriorPTB + BMI*GABD | 0.724 | 0.659 | 0.789 | 0.47 |
| ATD_~ADA12/(TETN + APOC3) + IBP4/SHBG + PriorPTB + BMI*GABD | 0.723 | 0.66 | 0.787 | 0.48 |
| ATD_~(EGLN + CFAB)/TETN + IBP4/SHBG + PriorPTB + BMI*GABD | 0.722 | 0.659 | 0.785 | 0.48 |
| ATD_~(ADA12 + EGLN)/TETN + IBP4/SHBG + PriorPTB + BMI*GABD | 0.721 | 0.658 | 0.784 | 0.45 |
| ATD_~(PRL + CFAB)/APOC3 + IBP4/SHBG + PriorPTB + BMI*GABD | 0.721 | 0.655 | 0.787 | 0.36 |
| ATD_~(PSG3 + CFAB)/APOC3 + IBP4/SHBG + PriorPTB + BMI*GABD | 0.719 | 0.653 | 0.785 | 0.36 |
| ATD_~EGLN + PSG3 + TETN + IBP4/SHBG + PriorPTB + BMI*GABD | 0.719 | 0.655 | 0.782 | 0.33 |
| ATD_~(PRL + CFAB)/TETN + IBP4/SHBG + PriorPTB + BMI*GABD | 0.718 | 0.651 | 0.785 | 0.46 |
| ATD_~PRL + PSG3 + CFAB + IBP4/SHBG + PriorPTB + BMI*GABD | 0.718 | 0.652 | 0.783 | 0.33 |
| ATD_~(ADA12 + PSG3)/TETN + IBP4/SHBG + PriorPTB + BMI*GABD | 0.718 | 0.654 | 0.782 | 0.32 |
| ATD_~(PRL + EGLN)/APOC3 + IBP4/SHBG + PriorPTB + BMI*GABD | 0.717 | 0.652 | 0.782 | 0.87 |
| ATD_~(EGLN + CFAB)/APOC3 + IBP4/SHBG + PriorPTB + BMI*GABD | 0.717 | 0.651 | 0.782 | 0.36 |
| ATD_~EGLN/(TETN + ATS13) + IBP4/SHBG + PriorPTB + BMI*GABD | 0.717 | 0.652 | 0.781 | 0.29 |
| ATD_~TIMP1/(TETN + APOC3) + IBP4/SHBG + PriorPTB + BMI*GABD | 0.717 | 0.652 | 0.782 | 0.29 |
| ATD_~(PRL + PSG3)/PGRP2 + IBP4/SHBG + PriorPTB + BMI*GABD | 0.716 | 0.652 | 0.78 | 0.38 |
| ATD_~(EGLN + IBP1)/TETN + IBP4/SHBG + PriorPTB + BMI*GABD | 0.716 | 0.652 | 0.781 | 0.33 |
| ATD_~(PRL + IBP1)/TETN + IBP4/SHBG + PriorPTB + BMI*GABD | 0.716 | 0.648 | 0.784 | 0.31 |
| ATD_~PSG3/(TETN + APOC3) + IBP4/SHBG + PriorPTB + BMI*GABD | 0.716 | 0.652 | 0.78 | 0.28 |
| ATD_~(PRL + TIMP1)/TETN + IBP4/SHBG + PriorPTB + BMI*GABD | 0.715 | 0.649 | 0.782 | 0.87 |
| ATD_~(PRL + ADA12)/ATS13 + IBP4/SHBG + PriorPTB + BMI*GABD | 0.715 | 0.647 | 0.782 | 0.50 |
| ATD_~(EGLN + CFAB)/ATS13 + IBP4/SHBG + PriorPTB + BMI*GABD | 0.715 | 0.649 | 0.781 | 0.28 |
| ATD_~(ADA12 + CFAB)/TETN + IBP4/SHBG + PriorPTB + BMI*GABD | 0.715 | 0.65 | 0.781 | 0.26 |
| ATD_~(PRL + PSG3)/ATS13 + IBP4/SHBG + PriorPTB + BMI*GABD | 0.715 | 0.648 | 0.781 | 0.22 |
| ATD_~(ADA12 + PSG3)/APOC3 + IBP4/SHBG + PriorPTB + BMI*GABD | 0.715 | 0.651 | 0.78 | 0.22 |
| ATD_~PRL/(TETN + PGRP2) + IBP4/SHBG + PriorPTB + BMI*GABD | 0.714 | 0.648 | 0.781 | 1.30 |
| ATD_~(ADA12 + CFAB)/APOC3 + IBP4/SHBG + PriorPTB + BMI*GABD | 0.714 | 0.648 | 0.779 | 0.21 |
| ATD_~PRL + EGLN + PSG3 + IBP4/SHBG + PriorPTB + BMI*GABD | 0.714 | 0.649 | 0.779 | 0.20 |
| ATD_~PRL + PSG3 + IBP1 + IBP4/SHBG + PriorPTB + BMI*GABD | 0.714 | 0.648 | 0.779 | 0.19 |
| ATD_~PRL + ADA12 + PSG3 + IBP4/SHBG + PriorPTB + BMI*GABD | 0.713 | 0.648 | 0.778 | 0.22 |
| ATD_~(PRL + CHL1)/TETN + IBP4/SHBG + PriorPTB + BMI*GABD | 0.712 | 0.644 | 0.78 | 0.86 |
| ATD_~(EGLN + TIMP1)/TETN + IBP4/SHBG + PriorPTB + BMI*GABD | 0.712 | 0.648 | 0.776 | 0.21 |
| ATD_~(CFAB + TIMP1)/APOC3 + IBP4/SHBG + PriorPTB + BMI*GABD | 0.712 | 0.644 | 0.779 | 0.20 |
| ATD_~(PRL + EGLN)/PGRP2 + IBP4/SHBG + PriorPTB + BMI*GABD | 0.711 | 0.646 | 0.775 | 0.68 |
| ATD_~(PRL + TIMP1)/APOC3 + IBP4/SHBG + PriorPTB + BMI*GABD | 0.711 | 0.645 | 0.777 | 0.38 |
| ATD_~(PRL + CFAB)/PGRP2 + IBP4/SHBG + PriorPTB + BMI*GABD | 0.711 | 0.645 | 0.778 | 0.37 |
| ATD_~(EGLN + CHL1)/TETN + IBP4/SHBG + PriorPTB + BMI*GABD | 0.711 | 0.646 | 0.776 | 0.23 |
| ATD_~(ADA12 + EGLN)/ATS13 + IBP4/SHBG + PriorPTB + BMI*GABD | 0.711 | 0.646 | 0.777 | 0.22 |
| ATD_~EGLN/(TETN + PGRP2) + IBP4/SHBG + PriorPTB + BMI*GABD | 0.711 | 0.647 | 0.776 | 0.20 |
| ATD_~CFAB/(TETN + APOC3) + IBP4/SHBG + PriorPTB + BMI*GABD | 0.71 | 0.643 | 0.777 | 0.36 |
| ATD_~PRL + EGLN + IBP1 + IBP4/SHBG + PriorPTB + BMI*GABD | 0.71 | 0.644 | 0.776 | 0.23 |
| ATD_~EGLN/(ATS13 + APOC3) + IBP4/SHBG + PriorPTB + BMI*GABD | 0.71 | 0.642 | 0.778 | 0.20 |
| ATD_~(ADA12 + IBP1)/TETN + IBP4/SHBG + PriorPTB + BMI*GABD | 0.71 | 0.644 | 0.776 | 0.17 |
| ATD_~EGLN + PSG3 + CFAB + IBP4/SHBG + PriorPTB + BMI*GABD | 0.709 | 0.645 | 0.773 | 0.88 |
| ATD_~(PRL + ADA12)/APOC3 + IBP4/SHBG + PriorPTB + BMI*GABD | 0.709 | 0.644 | 0.773 | 0.43 |
| ATD_~PRL/(ATS13 + APOC3) + IBP4/SHBG + PriorPTB + BMI*GABD | 0.709 | 0.641 | 0.776 | 0.43 |
| ATD_~PRL + CFAB + IBP1 + IBP4/SHBG + PriorPTB + BMI*GABD | 0.709 | 0.642 | 0.777 | 0.28 |
| ATD_~(PSG3 + TIMP1)/APOC3 + IBP4/SHBG + PriorPTB + BMI*GABD | 0.709 | 0.643 | 0.776 | 0.28 |
| ATD_~PRL/(TETN + ATS13) + IBP4/SHBG + PriorPTB + BMI*GABD | 0.709 | 0.641 | 0.777 | 0.22 |
| ATD_~ADA12 + PSG3 + CFAB + IBP4/SHBG + PriorPTB + BMI*GABD | 0.709 | 0.645 | 0.774 | 0.21 |
| ATD_~ADA12 + EGLN + CFAB + IBP4/SHBG + PriorPTB + BMI*GABD | 0.709 | 0.644 | 0.773 | 0.18 |
| ATD_~PRL + ADA12 + CFAB + IBP4/SHBG + PriorPTB + BMI*GABD | 0.708 | 0.641 | 0.774 | 1.17 |
| ATD_~EGLN + CFAB + IBP1 + IBP4/SHBG + PriorPTB + BMI*GABD | 0.708 | 0.643 | 0.773 | 0.53 |
| ATD_~PRL + EGLN + CHL1 + IBP4/SHBG + PriorPTB + BMI*GABD | 0.708 | 0.642 | 0.775 | 0.21 |
| ATD_~ADA12/(TETN + ATS13) + IBP4/SHBG + PriorPTB + BMI*GABD | 0.708 | 0.641 | 0.775 | 0.17 |
| ATD_~(EGLN + PSG3)/APOC3 + IBP4/SHBG + PriorPTB + BMI*GABD | 0.707 | 0.641 | 0.772 | 0.61 |
| ATD_~EGLN + CFAB + TIMP1 + IBP4/SHBG + PriorPTB + BMI*GABD | 0.707 | 0.643 | 0.772 | 0.44 |
| ATD_~(PRL + IBP1)/APOC3 + IBP4/SHBG + PriorPTB + BMI*GABD | 0.707 | 0.641 | 0.773 | 0.25 |
| ATD_~(CFAB + TIMP1)/TETN + IBP4/SHBG + PriorPTB + BMI*GABD | 0.707 | 0.642 | 0.773 | 0.19 |
| ATD_~(ADA12 + TIMP1)/TETN + IBP4/SHBG + PriorPTB + BMI*GABD | 0.707 | 0.642 | 0.773 | 0.17 |
| ATD_~PRL + PSG3 + CHL1 + IBP4/SHBG + PriorPTB + BMI*GABD | 0.707 | 0.641 | 0.773 | 0.17 |
| ATD_~PRL + PSG3 + TIMP1 + IBP4/SHBG + PriorPTB + BMI*GABD | 0.707 | 0.641 | 0.773 | 0.15 |
| ATD_~(ADA12 + PSG3)/ATS13 + IBP4/SHBG + PriorPTB + BMI*GABD | 0.707 | 0.64 | 0.773 | 0.15 |
| ATD_~(ADA12 + CFAB)/ATS13 + IBP4/SHBG + PriorPTB + BMI*GABD | 0.706 | 0.639 | 0.774 | 0.59 |
| ATD_~(EGLN + CFAB)/PGRP2 + IBP4/SHBG + PriorPTB + BMI*GABD | 0.706 | 0.641 | 0.771 | 0.54 |
| ATD_~EGLN + CFAB + CHL1 + IBP4/SHBG + PriorPTB + BMI*GABD | 0.706 | 0.641 | 0.771 | 0.52 |
| ATD_~PRL + ADA12 + EGLN + IBP4/SHBG + PriorPTB + BMI*GABD | 0.706 | 0.641 | 0.772 | 0.29 |
| ATD_~(PSG3 + TIMP1)/TETN + IBP4/SHBG + PriorPTB + BMI*GABD | 0.706 | 0.64 | 0.771 | 0.22 |
| ATD_~PRL + EGLN + TIMP1 + IBP4/SHBG + PriorPTB + BMI*GABD | 0.706 | 0.64 | 0.772 | 0.22 |
| ATD_~(PRL + CFAB)/ATS13 + IBP4/SHBG + PriorPTB + BMI*GABD | 0.706 | 0.637 | 0.776 | 0.17 |
| ATD_~(ADA12 + IBP1)/ATS13 + IBP4/SHBG + PriorPTB + BMI*GABD | 0.706 | 0.639 | 0.773 | 0.16 |
| ATD_~(PRL + TIMP1)/ATS13 + IBP4/SHBG + PriorPTB + BMI*GABD | 0.706 | 0.638 | 0.775 | 0.16 |
| ATD_~PRL + CFAB + CHL1 + IBP4/SHBG + PriorPTB + BMI*GABD | 0.705 | 0.637 | 0.773 | 0.48 |
| ATD_~PRL + CFAB + TIMP1 + IBP4/SHBG + PriorPTB + BMI*GABD | 0.705 | 0.637 | 0.773 | 0.42 |
| ATD_~(PRL + CHL1)/APOC3 + IBP4/SHBG + PriorPTB + BMI*GABD | 0.705 | 0.639 | 0.772 | 0.33 |
| ATD_~(PSG3 + IBP1)/APOC3 + IBP4/SHBG + PriorPTB + BMI*GABD | 0.705 | 0.64 | 0.77 | 0.31 |
| ATD_~(CFAB + IBP1)/APOC3 + IBP4/SHBG + PriorPTB + BMI*GABD | 0.705 | 0.638 | 0.773 | 0.22 |
| ATD_~(EGLN + PSG3)/ATS13 + IBP4/SHBG + PriorPTB + BMI*GABD | 0.705 | 0.639 | 0.771 | 0.20 |
| ATD_~(ADA12 + EGLN)/APOC3 + IBP4/SHBG + PriorPTB + BMI*GABD | 0.705 | 0.639 | 0.771 | 0.16 |
| ATD_~ADA12 + CFAB + IBP1 + IBP4/SHBG + PriorPTB + BMI*GABD | 0.705 | 0.639 | 0.77 | 0.15 |
| ATD_~ADA12 + PSG3 + IBP1 + IBP4/SHBG + PriorPTB + BMI*GABD | 0.705 | 0.641 | 0.769 | 0.13 |
| ATD_~PRL/(PGRP2 + APOC3) + IBP4/SHBG + PriorPTB + BMI*GABD | 0.704 | 0.639 | 0.77 | 1.45 |
| ATD_~(EGLN + IBP1)/ATS13 + IBP4/SHBG + PriorPTB + BMI*GABD | 0.704 | 0.637 | 0.771 | 0.40 |
| ATD_~(PSG3 + CFAB)/TETN + IBP4/SHBG + PriorPTB + BMI*GABD | 0.704 | 0.638 | 0.77 | 0.23 |
| ATD_~PSG3/(APOC3 + ATS13) + IBP4/SHBG + PriorPTB + BMI*GABD | 0.704 | 0.637 | 0.772 | 0.20 |
| ATD_~IBP1/(TETN + APOC3) + IBP4/SHBG + PriorPTB + BMI*GABD | 0.704 | 0.636 | 0.772 | 0.17 |
| ATD_~(PRL + CHL1)/ATS13 + IBP4/SHBG + PriorPTB + BMI*GABD | 0.704 | 0.635 | 0.772 | 0.17 |
| ATD_~ADA12/(TETN + PGRP2) + IBP4/SHBG + PriorPTB + BMI*GABD | 0.704 | 0.638 | 0.77 | 0.14 |
| ATD_~PRL + ADA12 + IBP1 + IBP4/SHBG + PriorPTB + BMI*GABD | 0.703 | 0.636 | 0.769 | 0.65 |
| ATD_~(PRL + IBP1)/ATS13 + IBP4/SHBG + PriorPTB + BMI*GABD | 0.703 | 0.635 | 0.771 | 0.42 |
| ATD_~(PRL + TIMP1)/PGRP2 + IBP4/SHBG + PriorPTB + BMI*GABD | 0.703 | 0.637 | 0.768 | 0.42 |
| ATD_~PSG3/(PGRP2 + APOC3) + IBP4/SHBG + PriorPTB + BMI*GABD | 0.703 | 0.638 | 0.769 | 0.37 |
| ATD_~PRL/(PGRP2 + ATS13) + IBP4/SHBG + PriorPTB + BMI*GABD | 0.703 | 0.636 | 0.77 | 0.27 |
| ATD_~(EGLN + IBP1)/APOC3 + IBP4/SHBG + PriorPTB + BMI*GABD | 0.702 | 0.636 | 0.768 | 1.06 |
| ATD_~(PRL + IBP1)/PGRP2 + IBP4/SHBG + PriorPTB + BMI*GABD | 0.702 | 0.635 | 0.768 | 0.35 |
| ATD_~EGLN/(PGRP2 + ATS13) + IBP4/SHBG + PriorPTB + BMI*GABD | 0.702 | 0.635 | 0.768 | 0.30 |
| ATD_~(EGLN + TIMP1)/ATS13 + IBP4/SHBG + PriorPTB + BMI*GABD | 0.702 | 0.635 | 0.769 | 0.26 |
| ATD_~CFAB/(ATS13 + APOC3) + IBP4/SHBG + PriorPTB + BMI*GABD | 0.702 | 0.633 | 0.771 | 0.25 |
| ATD_~(CFAB + CHL1)/APOC3 + IBP4/SHBG + PriorPTB + BMI*GABD | 0.702 | 0.635 | 0.77 | 0.24 |
| ATD_~(PRL + ADA12)/PGRP2 + IBP4/SHBG + PriorPTB + BMI*GABD | 0.702 | 0.637 | 0.766 | 0.23 |
| ATD_~(ADA12 + CFAB)/PGRP2 + IBP4/SHBG + PriorPTB + BMI*GABD | 0.702 | 0.635 | 0.768 | 0.21 |
| ATD_~ADA12 + EGLN + IBP1 + IBP4/SHBG + PriorPTB + BMI*GABD | 0.702 | 0.637 | 0.766 | 0.19 |
| ATD_~(ADA12 + IBP1)/APOC3 + IBP4/SHBG + PriorPTB + BMI*GABD | 0.702 | 0.637 | 0.767 | 0.19 |
| ATD_~(ADA12 + CHL1)/TETN + IBP4/SHBG + PriorPTB + BMI*GABD | 0.702 | 0.635 | 0.769 | 0.18 |
| ATD_~(ADA12 + PSG3)/PGRP2 + IBP4/SHBG + PriorPTB + BMI*GABD | 0.702 | 0.637 | 0.766 | 0.16 |
| ATD_~PSG3 + CFAB + TIMP1 + IBP4/SHBG + PriorPTB + BMI*GABD | 0.702 | 0.637 | 0.768 | 0.15 |
| ATD_~(EGLN + CHL1)/ATS13 + IBP4/SHBG + PriorPTB + BMI*GABD | 0.701 | 0.634 | 0.769 | 0.58 |
| ATD_~PRL + IBP1 + TIMP1 + IBP4/SHBG + PriorPTB + BMI*GABD | 0.701 | 0.634 | 0.769 | 0.20 |
| ATD_~ADA12/(ATS13 + APOC3) + IBP4/SHBG + PriorPTB + BMI*GABD | 0.701 | 0.634 | 0.769 | 0.15 |
| ATD_~(EGLN + TIMP1)/APOC3 + IBP4/SHBG + PriorPTB + BMI*GABD | 0.7 | 0.633 | 0.767 | 2.87 |
| ATD_~(PRL + CHL1)/PGRP2 + IBP4/SHBG + PriorPTB + BMI*GABD | 0.7 | 0.633 | 0.766 | 0.58 |
| ATD_~TIMP1/(ATS13 + APOC3) + IBP4/SHBG + PriorPTB + BMI*GABD | 0.7 | 0.63 | 0.769 | 0.39 |
| ATD_~PSG3 + CFAB + IBP1 + IBP4/SHBG + PriorPTB + BMI*GABD | 0.7 | 0.634 | 0.766 | 0.28 |
| ATD_~PRL + IBP1 + CHL1 + IBP4/SHBG + PriorPTB + BMI*GABD | 0.7 | 0.632 | 0.768 | 0.20 |
| ATD_~(ADA12 + TIMP1)/APOC3 + IBP4/SHBG + PriorPTB + BMI*GABD | 0.7 | 0.633 | 0.767 | 0.17 |
| ATD_~EGLN + PSG3 + IBP1 + IBP4/SHBG + PriorPTB + BMI*GABD | 0.699 | 0.634 | 0.764 | 0.55 |
| ATD_~(PSG3 + CHL1)/APOC3 + IBP4/SHBG + PriorPTB + BMI*GABD | 0.699 | 0.633 | 0.765 | 0.19 |
| ATD_~(IBP1 + TIMP1)/TETN + IBP4/SHBG + PriorPTB + BMI*GABD | 0.699 | 0.632 | 0.766 | 0.18 |
| ATD_~CHL1/(TETN + APOC3) + IBP4/SHBG + PriorPTB + BMI*GABD | 0.699 | 0.631 | 0.767 | 0.16 |
| ATD_~(EGLN + CHL1)/APOC3 + IBP4/SHBG + PriorPTB + BMI*GABD | 0.698 | 0.63 | 0.765 | 1.87 |
| ATD_~PRL + ADA12 + CHL1 + IBP4/SHBG + PriorPTB + BMI*GABD | 0.698 | 0.631 | 0.765 | 0.32 |
| ATD_~(IBP1 + TIMP1)/APOC3 + IBP4/SHBG + PriorPTB + BMI*GABD | 0.698 | 0.631 | 0.765 | 0.31 |
| ATD_~(CFAB + IBP1)/TETN + IBP4/SHBG + PriorPTB + BMI*GABD | 0.698 | 0.63 | 0.765 | 0.19 |
| ATD_~(PSG3 + IBP1)/TETN + IBP4/SHBG + PriorPTB + BMI*GABD | 0.698 | 0.632 | 0.763 | 0.17 |
| ATD_~ADA12 + EGLN + PSG3 + IBP4/SHBG + PriorPTB + BMI*GABD | 0.698 | 0.634 | 0.763 | 0.14 |
| ATD_~(ADA12 + TIMP1)/ATS13 + IBP4/SHBG + PriorPTB + BMI*GABD | 0.698 | 0.629 | 0.766 | 0.13 |
| ATD_~EGLN/(PGRP2 + APOC3) + IBP4/SHBG + PriorPTB + BMI*GABD | 0.697 | 0.631 | 0.763 | 2.09 |
| ATD_~(EGLN + PSG3)/PGRP2 + IBP4/SHBG + PriorPTB + BMI*GABD | 0.697 | 0.632 | 0.762 | 1.34 |
| ATD_~CFAB/(PGRP2 + APOC3) + IBP4/SHBG + PriorPTB + BMI*GABD | 0.697 | 0.629 | 0.765 | 0.42 |
| ATD_~1/(TETN + PGRP2 + APOC3) + IBP4/SHBG + PriorPTB + BMI*GABD | 0.697 | 0.629 | 0.765 | 0.31 |
| ATD_~(PSG3 + CFAB)/PGRP2 + IBP4/SHBG + PriorPTB + BMI*GABD | 0.697 | 0.631 | 0.763 | 0.31 |
| ATD_~(ADA12 + EGLN)/PGRP2 + IBP4/SHBG + PriorPTB + BMI*GABD | 0.697 | 0.632 | 0.762 | 0.30 |
| ATD_~ADA12/(PGRP2 + APOC3) + IBP4/SHBG + PriorPTB + BMI*GABD | 0.697 | 0.632 | 0.762 | 0.30 |
| ATD_~(ADA12 + CHL1)/ATS13 + IBP4/SHBG + PriorPTB + BMI*GABD | 0.697 | 0.628 | 0.765 | 0.14 |
| ATD_~PRL + ADA12 + TIMP1 + IBP4/SHBG + PriorPTB + BMI*GABD | 0.696 | 0.629 | 0.763 | 0.29 |
| ATD_~1/(TETN + ATS13 + APOC3) + IBP4/SHBG + PriorPTB + BMI*GABD | 0.696 | 0.627 | 0.765 | 0.28 |
| ATD_~PRL + CHL1 + TIMP1 + IBP4/SHBG + PriorPTB + BMI*GABD | 0.696 | 0.629 | 0.764 | 0.28 |
| ATD_~CFAB + IBP1 + TIMP1 + IBP4/SHBG + PriorPTB + BMI*GABD | 0.696 | 0.629 | 0.762 | 0.26 |
| ATD_~ADA12/(PGRP2 + ATS13) + IBP4/SHBG + PriorPTB + BMI*GABD | 0.696 | 0.628 | 0.765 | 0.16 |
| ATD_~(ADA12 + IBP1)/PGRP2 + IBP4/SHBG + PriorPTB + BMI*GABD | 0.696 | 0.63 | 0.762 | 0.15 |
| ATD_~ADA12 + CFAB + CHL1 + IBP4/SHBG + PriorPTB + BMI*GABD | 0.696 | 0.629 | 0.763 | 0.15 |
| ATD_~ADA12 + PSG3 + TIMP1 + IBP4/SHBG + PriorPTB + BMI*GABD | 0.696 | 0.63 | 0.761 | 0.13 |
| ATD_~ADA12 + CFAB + TIMP1 + IBP4/SHBG + PriorPTB + BMI*GABD | 0.696 | 0.63 | 0.763 | 0.12 |
| ATD_~TIMP1/(PGRP2 + APOC3) + IBP4/SHBG + PriorPTB + BMI*GABD | 0.695 | 0.628 | 0.762 | 3.56 |
| ATD_~PSG3 + IBP1 + TIMP1 + IBP4/SHBG + PriorPTB + BMI*GABD | 0.695 | 0.629 | 0.76 | 0.14 |
| ATD_~ADA12 + IBP1 + CHL1 + IBP4/SHBG + PriorPTB + BMI*GABD | 0.695 | 0.628 | 0.761 | 0.14 |
| ATD_~ADA12 + IBP1 + TIMP1 + IBP4/SHBG + PriorPTB + BMI*GABD | 0.695 | 0.629 | 0.761 | 0.12 |
| ATD_~EGLN + IBP1 + CHL1 + IBP4/SHBG + PriorPTB + BMI*GABD | 0.694 | 0.628 | 0.761 | 4.00 |
| ATD_~(PSG3 + IBP1)/ATS13 + IBP4/SHBG + PriorPTB + BMI*GABD | 0.694 | 0.627 | 0.761 | 0.44 |
| ATD_~(PSG3 + CFAB)/ATS13 + IBP4/SHBG + PriorPTB + BMI*GABD | 0.694 | 0.625 | 0.763 | 0.38 |
| ATD_~(CFAB + TIMP1)/ATS13 + IBP4/SHBG + PriorPTB + BMI*GABD | 0.694 | 0.625 | 0.762 | 0.22 |
| ATD_~(ADA12 + CHL1)/APOC3 + IBP4/SHBG + PriorPTB + BMI*GABD | 0.694 | 0.627 | 0.76 | 0.13 |
| ATD_~ADA12 + PSG3 + CHL1 + IBP4/SHBG + PriorPTB + BMI*GABD | 0.694 | 0.628 | 0.76 | 0.12 |
| ATD_~(EGLN + IBP1)/PGRP2 + IBP4/SHBG + PriorPTB + BMI*GABD | 0.693 | 0.628 | 0.759 | 5.92 |
| ATD_~(PSG3 + IBP1)/PGRP2 + IBP4/SHBG + PriorPTB + BMI*GABD | 0.693 | 0.628 | 0.759 | 0.40 |
| ATD_~PSG3 + CFAB + CHL1 + IBP4/SHBG + PriorPTB + BMI*GABD | 0.693 | 0.626 | 0.759 | 0.21 |
| ATD_~(PSG3 + TIMP1)/ATS13 + IBP4/SHBG + PriorPTB + BMI*GABD | 0.693 | 0.624 | 0.761 | 0.20 |
| ATD_~ADA12 + EGLN + CHL1 + IBP4/SHBG + PriorPTB + BMI*GABD | 0.693 | 0.627 | 0.759 | 0.12 |
| ATD_~EGLN + PSG3 + CHL1 + IBP4/SHBG + PriorPTB + BMI*GABD | 0.692 | 0.626 | 0.757 | 0.70 |
| ATD_~TIMP1/(TETN + PGRP2) + IBP4/SHBG + PriorPTB + BMI*GABD | 0.692 | 0.626 | 0.759 | 0.27 |
| ATD_~(CFAB + CHL1)/TETN + IBP4/SHBG + PriorPTB + BMI*GABD | 0.692 | 0.624 | 0.76 | 0.21 |
| ATD_~PSG3/(TETN + PGRP2) + IBP4/SHBG + PriorPTB + BMI*GABD | 0.692 | 0.627 | 0.758 | 0.14 |
| ATD_~(CHL1 + TIMP1)/APOC3 + IBP4/SHBG + PriorPTB + BMI*GABD | 0.691 | 0.623 | 0.759 | 3.11 |
| ATD_~EGLN + IBP1 + TIMP1 + IBP4/SHBG + PriorPTB + BMI*GABD | 0.691 | 0.625 | 0.757 | 2.06 |
| ATD_~TIMP1/(TETN + ATS13) + IBP4/SHBG + PriorPTB + BMI*GABD | 0.691 | 0.623 | 0.759 | 0.27 |
| ATD_~(IBP1 + TIMP1)/ATS13 + IBP4/SHBG + PriorPTB + BMI*GABD | 0.691 | 0.622 | 0.76 | 0.21 |
| ATD_~(CHL1 + TIMP1)/TETN + IBP4/SHBG + PriorPTB + BMI*GABD | 0.691 | 0.624 | 0.758 | 0.17 |
| ATD_~(EGLN + CHL1)/PGRP2 + IBP4/SHBG + PriorPTB + BMI*GABD | 0.69 | 0.624 | 0.756 | 1.41 |
| ATD_~EGLN + PSG3 + TIMP1 + IBP4/SHBG + PriorPTB + BMI*GABD | 0.69 | 0.624 | 0.756 | 0.42 |
| ATD_~(IBP1 + CHL1)/APOC3 + IBP4/SHBG + PriorPTB + BMI*GABD | 0.69 | 0.622 | 0.759 | 0.36 |
| ATD_~(PSG3 + TIMP1)/PGRP2 + IBP4/SHBG + PriorPTB + BMI*GABD | 0.69 | 0.624 | 0.756 | 0.16 |
| ATD_~ADA12 + EGLN + TIMP1 + IBP4/SHBG + PriorPTB + BMI*GABD | 0.69 | 0.624 | 0.756 | 0.13 |
| ATD_~(CFAB + IBP1)/PGRP2 + IBP4/SHBG + PriorPTB + BMI*GABD | 0.689 | 0.621 | 0.757 | 0.38 |
| ATD_~CFAB/(TETN + PGRP2) + IBP4/SHBG + PriorPTB + BMI*GABD | 0.689 | 0.621 | 0.757 | 0.36 |
| ATD_~CFAB + IBP1 + CHL1 + IBP4/SHBG + PriorPTB + BMI*GABD | 0.689 | 0.621 | 0.757 | 0.28 |
| ATD_~(CFAB + TIMP1)/PGRP2 + IBP4/SHBG + PriorPTB + BMI*GABD | 0.689 | 0.623 | 0.756 | 0.22 |
| ATD_~(PSG3 + CHL1)/TETN + IBP4/SHBG + PriorPTB + BMI*GABD | 0.689 | 0.623 | 0.755 | 0.19 |
| ATD_~CFAB + CHL1 + TIMP1 + IBP4/SHBG + PriorPTB + BMI*GABD | 0.689 | 0.621 | 0.756 | 0.15 |
| ATD_~PSG3/(TETN + ATS13) + IBP4/SHBG + PriorPTB + BMI*GABD | 0.689 | 0.622 | 0.756 | 0.14 |
| ATD_~IBP1/(PGRP2 + APOC3) + IBP4/SHBG + PriorPTB + BMI*GABD | 0.688 | 0.62 | 0.755 | 0.99 |
| ATD_~IBP1/(ATS13 + APOC3) + IBP4/SHBG + PriorPTB + BMI*GABD | 0.688 | 0.617 | 0.759 | 0.69 |
| ATD_~IBP1/(TETN + PGRP2) + IBP4/SHBG + PriorPTB + BMI*GABD | 0.688 | 0.62 | 0.756 | 0.33 |
| ATD_~PSG3/(PGRP2 + ATS13) + IBP4/SHBG + PriorPTB + BMI*GABD | 0.688 | 0.62 | 0.755 | 0.22 |
| ATD_~(IBP1 + CHL1)/TETN + IBP4/SHBG + PriorPTB + BMI*GABD | 0.688 | 0.619 | 0.757 | 0.22 |
| ATD_~(CFAB + IBP1)/ATS13 + IBP4/SHBG + PriorPTB + BMI*GABD | 0.688 | 0.618 | 0.758 | 0.20 |
| ATD_~(EGLN + TIMP1)/PGRP2 + IBP4/SHBG + PriorPTB + BMI*GABD | 0.687 | 0.621 | 0.753 | 2.43 |
| ATD_~1/(PGRP2 + ATS13 + APOC3) + IBP4/SHBG + PriorPTB + BMI*GABD | 0.686 | 0.616 | 0.756 | 0.57 |
| ATD_~PSG3 + IBP1 + CHL1 + IBP4/SHBG + PriorPTB + BMI*GABD | 0.686 | 0.62 | 0.752 | 0.22 |
| ATD_~(IBP1 + TIMP1)/PGRP2 + IBP4/SHBG + PriorPTB + BMI*GABD | 0.685 | 0.618 | 0.751 | 0.48 |
| ATD_~(ADA12 + CHL1)/PGRP2 + IBP4/SHBG + PriorPTB + BMI*GABD | 0.685 | 0.619 | 0.752 | 0.14 |
| ATD_~(ADA12 + TIMP1)/PGRP2 + IBP4/SHBG + PriorPTB + BMI*GABD | 0.685 | 0.618 | 0.752 | 0.13 |
| ATD_~CHL1/(PGRP2 + APOC3) + IBP4/SHBG + PriorPTB + BMI*GABD | 0.684 | 0.616 | 0.752 | 0.40 |
| ATD_~(PSG3 + CHL1)/PGRP2 + IBP4/SHBG + PriorPTB + BMI*GABD | 0.684 | 0.618 | 0.75 | 0.20 |
| ATD_~PSG3 + CHL1 + TIMP1 + IBP4/SHBG + PriorPTB + BMI*GABD | 0.684 | 0.617 | 0.751 | 0.17 |
| ATD_~CFAB/(TETN + ATS13) + IBP4/SHBG + PriorPTB + BMI*GABD | 0.684 | 0.614 | 0.754 | 0.15 |
| ATD_~EGLN + CHL1 + TIMP1 + IBP4/SHBG + PriorPTB + BMI*GABD | 0.683 | 0.616 | 0.75 | 0.64 |
| ATD_~IBP1/(TETN + ATS13) + IBP4/SHBG + PriorPTB + BMI*GABD | 0.683 | 0.613 | 0.753 | 0.19 |
| ATD_~CHL1/(ATS13 + APOC3) + IBP4/SHBG + PriorPTB + BMI*GABD | 0.683 | 0.612 | 0.755 | 0.16 |
| ATD_~ADA12 + CHL1 + TIMP1 + IBP4/SHBG + PriorPTB + BMI*GABD | 0.683 | 0.615 | 0.751 | 0.13 |
| ATD_~CFAB/(PGRP2 + ATS13) + IBP4/SHBG + PriorPTB + BMI*GABD | 0.682 | 0.612 | 0.752 | 0.67 |
| ATD_~TIMP1/(PGRP2 + ATS13) + IBP4/SHBG + PriorPTB + BMI*GABD | 0.682 | 0.613 | 0.75 | 0.38 |
| ATD_~IBP1 + CHL1 + TIMP1 + IBP4/SHBG + PriorPTB + BMI*GABD | 0.681 | 0.613 | 0.749 | 0.42 |
| ATD_~(CFAB + CHL1)/PGRP2 + IBP4/SHBG + PriorPTB + BMI*GABD | 0.681 | 0.613 | 0.75 | 0.19 |
| ATD_~(IBP1 + CHL1)/PGRP2 + IBP4/SHBG + PriorPTB + BMI*GABD | 0.68 | 0.612 | 0.748 | 0.52 |
| ATD_~(CFAB + CHL1)/ATS13 + IBP4/SHBG + PriorPTB + BMI*GABD | 0.68 | 0.609 | 0.751 | 0.44 |
| ATD_~(PSG3 + CHL1)/ATS13 + IBP4/SHBG + PriorPTB + BMI*GABD | 0.68 | 0.612 | 0.749 | 0.18 |
| ATD_~IBP1/(PGRP2 + ATS13) + IBP4/SHBG + PriorPTB + BMI*GABD | 0.679 | 0.61 | 0.749 | 0.23 |
| ATD_~(IBP1 + CHL1)/ATS13 + IBP4/SHBG + PriorPTB + BMI*GABD | 0.679 | 0.607 | 0.75 | 0.16 |
| ATD_~CHL1/(TETN + PGRP2) + IBP4/SHBG + PriorPTB + BMI*GABD | 0.678 | 0.61 | 0.747 | 0.28 |
| ATD_~(CHL1 + TIMP1)/ATS13 + IBP4/SHBG + PriorPTB + BMI*GABD | 0.677 | 0.607 | 0.748 | 0.27 |
| ATD_~1/(TETN + PGRP2 + ATS13) + IBP4/SHBG + PriorPTB + BMI*GABD | 0.676 | 0.606 | 0.745 | 0.22 |
| ATD_~CHL1/(TETN + ATS13) + IBP4/SHBG + PriorPTB + BMI*GABD | 0.676 | 0.606 | 0.746 | 0.14 |
| ATD_~(CHL1 + TIMP1)/PGRP2 + IBP4/SHBG + PriorPTB + BMI*GABD | 0.675 | 0.608 | 0.742 | 0.63 |
| ATD_~CHL1/(PGRP2 + ATS13) + IBP4/SHBG + PriorPTB + BMI*GABD | 0.672 | 0.602 | 0.743 | 1.25 |

TABLE 18
Reversal Groups Showing Improvement over the Adjusted Predictor
for Predicting sPTB <37 Weeks. Reversal group comprises IBP4/SHBG
and a reversal triplet or three additional biomarkers.

| Formula | AUC | 95% CI |
|---|---|---|
| ATD_~(PRL + EGLN)/TETN + IBP4/SHBG + BMI*GABD | 0.728 | 0.663:0.793 |
| ATD_~PRL/(APOC3 + TETN) + IBP4/SHBG + BMI*GABD | 0.722 | 0.656:0.788 |
| ATD_~(PRL + ADA12)/TETN + IBP4/SHBG + BMI*GABD | 0.719 | 0.654:0.785 |
| ATD_~(PRL + PSG3)/TETN + IBP4/SHBG + BMI*GABD | 0.718 | 0.651:0.784 |
| ATD_~EGLN/(APOC3 + TETN) + IBP4/SHBG + BMI*GABD | 0.716 | 0.652:0.781 |
| ATD_~(PRL + EGLN)/ATS13 + IBP4/SHBG + BMI*GABD | 0.715 | 0.648:0.781 |
| ATD_~ADA12/(APOC3 + TETN) + IBP4/SHBG + BMI*GABD | 0.715 | 0.651:0.78 |

# Adjusted predictor: IBP4/SHBG + GABD*BMI - 95% CI high = 0.714. Improvement over the adjusted predictor's AUC was considered significant for AUCs above its 95% confidence interval.

TABLE 19
Reversal Groups Showing Improvement over the Adjusted Predictor for Predicting
sPTB<37 Weeks. Reversal group comprises IBP4/SHBG, and additional clinical
variable, and a reversal triplet or three additional biomarkers.

| Formula | AUC | 95% CI |
|---|---|---|
| ATD_~(PRL + EGLN)/TETN + IBP4/SHBG + PriorPTB + BMI*GABD | 0.733 | 0.669:0.797 |
| ATD_~(PRL + ADA12)/TETN + IBP4/SHBG + PriorPTB + BMI*GABD | 0.726 | 0.661:0.79 |
| ATD_~(PRL + PSG3)/APOC3 + IBP4/SHBG + PriorPTB + BMI*GABD | 0.725 | 0.662:0.789 |
| ATD_~EGLN/(APOC3 + TETN) + IBP4/SHBG + PriorPTB + BMI*GABD | 0.725 | 0.662:0.789 |
| ATD_~(PRL + EGLN)/ATS13 + IBP4/SHBG + PriorPTB + BMI*GABD | 0.725 | 0.66:0.79 |
| ATD_~PRL/(APOC3 + TETN) + IBP4/SHBG + PriorPTB + BMI*GABD | 0.725 | 0.66:0.79 |
| ATD_~(PRL + PSG3)/TETN + IBP4/SHBG + PriorPTB + BMI*GABD | 0.725 | 0.66:0.79 |
| ATD_~PRL + EGLN + CFAB + IBP4/SHBG + PriorPTB + BMI*GABD | 0.724 | 0.659:0.789 |
| ATD_~ADA12/(APOC3 + TETN) + IBP4/SHBG + PriorPTB + BMI*GABD | 0.723 | 0.66:0.787 |
| ATD_~(CFAB + EGLN)/TETN + IBP4/SHBG + PriorPTB + BMI*GABD | 0.722 | 0.659:0.785 |
| ATD_~(EGLN + ADA12)/TETN + IBP4/SHBG + PriorPTB + BMI*GABD | 0.721 | 0.658:0.784 |
| ATD_~(PRL + CFAB)/APOC + IBP4/SHBG + PriorPTB + BMI*GABD | 0.721 | 0.655:0.787 |
| ATD_~(PSG3 + CFAB)/APOC + IBP4/SHBG + PriorPTB + BMI*GABD | 0.719 | 0.653:0.785 |
| ATD_~(EGLN + PSG3)/TETN + IBP4/SHBG + PriorPTB + BMI*GABD | 0.719 | 0.655:0.782 |
| ATD_~(PRL + CFAB)/TETN + IBP4/SHBG + PriorPTB + BMI*GABD | 0.718 | 0.651:0.785 |
| ATD_~PRL + PSG3 + CFAB + IBP4/SHBG + PriorPTB + BMI*GABD | 0.718 | 0.652:0.783 |
| ATD_~(ADA12 + PSG3)/TETN + IBP4/SHBG + PriorPTB + BMI*GABD | 0.718 | 0.654:0.782 |
| ATD_~(PRL + EGLN)/APOC3 + IBP4/SHBG + PriorPTB + BMI*GABD | 0.717 | 0.652:0.782 |
| ATD_~(CFAB + EGLN)/APOC3 + IBP4/SHBG + PriorPTB + BMI*GABD | 0.717 | 0.651:0.782 |
| ATD_~EGLN/(TETN + ATS13) + IBP4/SHBG + PriorPTB + BMI*GABD | 0.717 | 0.652:0.781 |
| ATD_~TIMP1/(APOC3 + TETN) + IBP4/SHBG + PriorPTB + BMI*GABD | 0.717 | 0.652:0.782 |
| ATD_~(PRL + PSG3)/PGRP2 + IBP4/SHBG + PriorPTB + BMI*GABD | 0.716 | 0.652:0.78 |
| ATD_~(EGLN + IBP1)/TETN + IBP4/SHBG + PriorPTB + BMI*GABD | 0.716 | 0.652:0.781 |
| ATD_~(PRL + IBP1)/TETN + IBP4/SHBG + PriorPTB + BMI*GABD | 0.716 | 0.648:0.784 |
| ATD_~PSG3/(APOC3 + TETN) + IBP4/SHBG + PriorPTB + BMI*GABD | 0.716 | 0.652:0.78 |
| ATD_~(PRL + TIMP1)/TETN + IBP4/SHBG + PriorPTB + BMI*GABD | 0.715 | 0.649:0.782 |
| ATD_~(PRL + ADA12)/ATS13 + IBP4/SHBG + PriorPTB + BMI*GABD | 0.715 | 0.647:0.782 |
| ATD_~(CFAB + EGLN)/ATS13 + IBP4/SHBG + PriorPTB + BMI*GABD | 0.715 | 0.649:0.781 |
| ATD_~(ADA12 + CFAB)/TETN + IBP4/SHBG + PriorPTB + BMI*GABD | 0.715 | 0.65:0.781 |
| ATD_~(PRL + PSG3)/ATS13 + IBP4/SHBG + PriorPTB + BMI*GABD | 0.715 | 0.648:0.781 |
| ATD_~(ADA12 + PSG3)/APOC3 + IBP4/SHBG + PriorPTB + BMI*GABD | 0.715 | 0.651:0.78 |

# Adjusted predictor: IBP4/SHBG + GABD*BMI - 95% CI high = 0.714. Improvement over the adjusted predictor's AUC was considered significant for AUCs above its 95% confidence interval.

### Example 3. Vietnam Preterm Birth Biomarker Study (PBB)

This study was designed to identify a series of biomarkers and biomarker-containing predictive models for the prediction of preterm birth by any cause (PTB) or spontaneous preterm birth (sPTB) in a Vietnamese population.

### Study Design

The Vietnam Preterm Birth Biomarker study (PBB) was a prospective observational cohort study comprised of women seeking antenatal care and planning to deliver at Tu Du hospital in Ho Chi Minh City. Biomarker levels were compared between all women delivering preterm with a randomly selected control group from the entire cohort (case cohort design). In this verification study, 176 subjects delivered spontaneously prior to 37 weeks GA, 44 delivered prior to 37 weeks GA due to medical indications, and 312 delivered term.

In a CLIA- and CAP-certified laboratory, serum from all subjects was analyzed by liquid chromatography-multiple reaction monitoring mass spectrometry (LC-MRM/MS) following depletion of the top fourteen most abundant proteins (MARS14, Agilent Technologies), digestion of serum proteins into peptides using trypsin (Trypsin Gold, Promega), and addition of stable isotopic standards (SIS, New England Peptide) as previously described (Saade, Bradford).

### Analyte Selection and Model Building

Clinical variables were surveyed for robustness of assessment, clinical relevance, and complementarity to IBP4/SHBG. One clinical variable was selected: ObRisk, comprising prior miscarriage for Nullips (women who had not had a previous birth) and prior preterm birth for Multips (women with one or more births). This established a base model on which further development proceeded: IBP4/SHBG+ObRisk.

In order to determine whether additional analytes improve the performance of the base model (IBP4/SHBG+ObRisk), the effects of adding one, two and three additional protein biomarkers (henceforth referred to as one-, two- and three-analyte models) were assessed.

In this population, the association between protein biomarkers and PTB was strongly modulated by parity. For this reason, one-, two- and three-analyte models were developed for nullips and multips separately.

Two- and three-analyte models were constructed by identifying analyte pairs per parity that performed better than the single analyte model. This resulted in 31 two-analyte models (sharing the same two analytes for nullips and multips in addition to IBP4/SHBG and ObRisk) with predictive performance for PTB<37 better than the best single-analyte model. A total 261 three-analyte models for PTB<37 were identified. The process was repeated for prediction of sPTB<37 which yielded a total of 416 additional models.

Models were then tested on two independent case-control datasets internal to Sera Prognostics. This testing process resulted in 50 models (37 three-analyte, 12 two-analyte and 1 one-analyte models) that passed in both independent datasets as well as in the PBB training set with a significant AUC. The individual proteins in these models and the peptides that were measured are shown in Table 20. Performance of the models is summarized in Table 21 based on their component predictors (reversals and clinical variable(s)). All models include a reversal formed by the ratio of IBP4/SHBG protein biomarker response ratios plus the OBRisk clinical variable. A simplified list of all the biomarkers and biomarker pairs measured with IBP4/SHBG plus the OBRisk clinical variable in the study are shown in Table 22.

TABLE 20
Proteins and peptides measured in Vietnam
Preterm Birth Biomarker study (PBB)

| Uniprot ID | Peptide |
|---|---|
| A2GL_HUMAN | DLLLPQPDLR (SEQ ID NO: 18) |
| ALS_HUMAN | IRPHTFTGLSGLR (SEQ ID NO: 19) |
| ANGT_HUMAN | DPTFIPAPIQAK (SEQ ID NO: 20) |
| CLUS_HUMAN | ASSIIDELFQDR (SEQ ID NO: 21) |
| CSH1_HUMAN | AHQLAIDTYQEFEETYIPK (SEQ ID NO: 22) |
| CSH2_HUMAN | |
| CSH1_HUMAN | ISLLLIESWLEPVR (SEQ ID NO: 23) |
| CSH2_HUMAN | |
| DEFI_HUMAN | YGTCIYQGR (SEQ ID NO: 24) |
| DPEP2_HUMAN | ALEVSQAPVIFSHSAAR (SEQ ID NO: 25) |
| EGLN_HUMAN | TQILEWAAER (SEQ ID NO: 4) |
| F13B_HUMAN | GDTYPAELYITGSILR (SEQ ID NO: 26) |
| FA5_HUMAN | LSEGASYLDHTFPAEK (SEQ ID NO: 27) |
| FBLN3_HUMAN | IPSNPSHR (SEQ ID NO: 28) |
| FETUA_HUMAN | HTLNQIDEVK (SEQ ID NO: 29) |
| HEMO_HUMAN | NFPSPVDAAFR (SEQ ID NO: 30) |
| IBP2_HUMAN | LIQGAPTIR (SEQ ID NO: 31) |
| IBP3_HUMAN | FLNVLSPR (SEQ ID NO: 32) |
| IBP3_HUMAN | YGQPLPGYTTK (SEQ ID NO: 33) |
| IGF1_HUMAN | GFYFNKPTGYGSSSR (SEQ ID NO: 34) |
| IGF2_HUMAN | GIVEECCFR (SEQ ID NO: 35) |
| LYAM1_HUMAN | SYYWIGIR (SEQ ID NO: 36) |
| PAPP1_HUMAN | DIPHWLNPTR (SEQ ID NO: 37) |
| PAPP2_HUMAN | LLLRPEVLAEIPR (SEQ ID NO: 38) |
| PEDF_HUMAN | TVQAVLTVPK (SEQ ID NO: 39) |
| PRL_HUMAN | SWNEPLYHLVTEVR (SEQ ID NO: 6) |
| PSG2_HUMAN | IHPSYTNYR (SEQ ID NO: 40) |
| PTGDS_HUMAN | AQGFTEDTIVFLPQTDK (SEQ ID NO: 41) |
| TENX_HUMAN | LSQLSVTDVTTSSLR (SEQ ID NO: 42) |
| TETN_HUMAN | CFLAFTQTK (SEQ ID NO: 7) |
| TETN_HUMAN | LDTLAQEVALLK (SEQ ID NO: 8) |
| VGFR1_HUMAN | YLAVPTSK (SEQ ID NO: 43) |

Note that, in some cases, one fragment is assayed for two different proteins because there exists overlap in peptide sequence. In some cases, there are two or more protein names assigned to one peptide.

TABLE 21
Summary of models and their performance. All models include a reversal formed by the ratio
of IBP4/SHBG protein biomarker response ratios plus the OBRisk clinical variable.

| | Nullip Reversal | | Multip Reversal | | | | | |
|---|---|---|---|---|---|---|---|---|
| | (Analyte1/Analyte2) | | (Analyte3/Analyte4) | | | AUC Per Outcome | | |
| Base Model+ | Analyte 1 | Analyte 2 | Analyte 3 | Analyte 4 | Model | SPTB | PTB | PTB34 |
| IBP4/SHBG + | EGLN | CSH | EGLN | CSH | IBP4/SHBG + ObRisk + | 0.672 | 0.679 | 0.766 |
| ObRisk+ | | | | | EGLN/CSH(nullip), EGLN/CSH(multip) | | | |
| IBP4/SHBG + | EGLN | CSH | EGLN | CSH | IBP4/SHBG + ObRisk + | 0.673 | 0.678 | 0.762 |
| ObRisk+ | | | | | EGLN/CSH(nullip), EGLN/CSH(multip) | | | |
| IBP4/SHBG + | EGLN | DPEP2 | EGLN | DPEP2 | IBP4/SHBG + ObRisk + | 0.669 | 0.679 | 0.759 |
| ObRisk+ | | | | | EGLN/DPEP2(nullip), EGLN/DPEP2(multip) | | | |
| IBP4/SHBG + | EGLN | FA5 | EGLN | A2GL | IBP4/SHBG + ObRisk + | 0.697 | 0.694 | 0.739 |
| ObRisk+ | | | | | EGLN/FA5(nullip), EGLN/A2GL(multip) | | | |
| IBP4/SHBG + | EGLN | FA5 | EGLN | ALS | IBP4/SHBG + ObRisk + | 0.696 | 0.696 | 0.746 |
| ObRisk+ | | | | | EGLN/FA5(nullip), EGLN/ALS(multip) | | | |
| IBP4/SHBG + | EGLN | FA5 | EGLN | ANGT | IBP4/SHBG + ObRisk + | 0.688 | 0.689 | 0.723 |
| ObRisk+ | | | | | EGLN/FA5(nullip), EGLN/ANGT(multip) | | | |
| IBP4/SHBG + | EGLN | FA5 | EGLN | CLUS | IBP4/SHBG + ObRisk + | 0.696 | 0.693 | 0.744 |
| ObRisk+ | | | | | EGLN/FA5(nullip), EGLN/CLUS(multip) | | | |
| IBP4/SHBG + | EGLN | FA5 | EGLN | CSH | IBP4/SHBG + ObRisk + | 0.691 | 0.692 | 0.741 |
| ObRisk+ | | | | | EGLN/FA5(nullip), EGLN/CSH(multip) | | | |
| IBP4/SHBG + | EGLN | FA5 | EGLN | DEF1 | IBP4/SHBG + ObRisk + | 0.696 | 0.696 | 0.725 |
| ObRisk+ | | | | | EGLN/FA5(nullip), EGLN/DEF1(multip) | | | |
| IBP4/SHBG + | EGLN | FA5 | EGLN | F13B | IBP4/SHBG + ObRisk + | 0.693 | 0.693 | 0.741 |
| ObRisk+ | | | | | EGLN/FA5(nullip), EGLN/F13B(multip) | | | |
| IBP4/SHBG + | EGLN | FA5 | EGLN | FA5 | IBP4/SHBG + ObRisk + | 0.69 | 0.689 | 0.743 |
| ObRisk+ | | | | | EGLN/FA5(nullip), EGLN/FA5(multip) | | | |
| IBP4/SHBG + | EGLN | FA5 | EGLN | FBLN3 | IBP4/SHBG + ObRisk + | 0.693 | 0.696 | 0.743 |
| ObRisk+ | | | | | EGLN/FA5(nullip), EGLN/FBLN3(multip) | | | |
| IBP4/SHBG + | EGLN | FA5 | EGLN | FETUA | IBP4/SHBG + ObRisk + | 0.693 | 0.692 | 0.74 |
| ObRisk+ | | | | | EGLN/FA5(nullip), EGLN/FETUA(multip) | | | |
| IBP4/SHBG + | EGLN | FA5 | EGLN | HEMO | IBP4/SHBG + ObRisk + | 0.695 | 0.692 | 0.742 |
| ObRisk+ | | | | | EGLN/FA5(nullip), EGLN/HEMO(multip) | | | |
| IBP4/SHBG + | EGLN | FA5 | EGLN | IBP3 | IBP4/SHBG + ObRisk + | 0.696 | 0.694 | 0.747 |
| ObRisk+ | | | | | EGLN/FA5(nullip), EGLN/IBP3(multip) | | | |
| IBP4/SHBG + | EGLN | FA5 | EGLN | IBP3 | IBP4/SHBG + ObRisk + | 0.694 | 0.693 | 0.749 |
| ObRisk+ | | | | | EGLN/FA5(nullip), EGLN/IBP3(multip) | | | |
| IBP4/SHBG + | EGLN | FA5 | EGLN | IGF1 | IBP4/SHBG + ObRisk + | 0.695 | 0.695 | 0.736 |
| ObRisk+ | | | | | EGLN/FA5(nullip), EGLN/IGF1(multip) | | | |
| IBP4/SHBG + | EGLN | FA5 | EGLN | IGF2 | IBP4/SHBG + ObRisk + | 0.69 | 0.692 | 0.746 |
| ObRisk+ | | | | | EGLN/FA5(nullip), EGLN/IGF2(multip) | | | |
| IBP4/SHBG + | EGLN | FA5 | EGLN | PEDF | IBP4/SHBG + ObRisk + | 0.694 | 0.694 | 0.742 |
| ObRisk+ | | | | | EGLN/FA5(nullip), EGLN/PEDF(multip) | | | |
| IBP4/SHBG + | EGLN | FA5 | EGLN | PTGDS | IBP4/SHBG + ObRisk + | 0.689 | 0.694 | 0.721 |
| ObRisk+ | | | | | EGLN/FA5(nullip), EGLN/PTGDS(multip) | | | |
| IBP4/SHBG + | EGLN | FA5 | EGLN | TENX | IBP4/SHBG + ObRisk + | 0.703 | 0.697 | 0.735 |
| ObRisk+ | | | | | EGLN/FA5(nullip), EGLN/TENX(multip) | | | |
| IBP4/SHBG + | EGLN | FA5 | FA5 | TENX | IBP4/SHBG + ObRisk + | 0.704 | 0.693 | 0.706 |
| ObRisk+ | | | | | EGLN/FA5(nullip), FA5/TENX(multip) | | | |
| IBP4/SHBG + | EGLN | FBLN3 | EGLN | FBLN3 | IBP4/SHBG + ObRisk + | 0.677 | 0.681 | 0.759 |
| ObRisk+ | | | | | EGLN/FBLN3(nullip), EGLN/FBLN3(multip) | | | |
| IBP4/SHBG + | EGLN | IGF1 | EGLN | IGF1 | IBP4/SHBG + ObRisk + | 0.677 | 0.682 | 0.756 |
| ObRisk+ | | | | | EGLN/IGF1(nullip), EGLN/IGF1(multip) | | | |
| IBP4/SHBG + | EGLN | | EGLN | | IBP4/SHBG + ObRisk + | 0.672 | 0.676 | 0.767 |
| ObRisk+ | | | | | EGLN/(nullip), EGLN/(multip) | | | |
| IBP4/SHBG + | EGLN | PAPP1 | EGLN | PAPP1 | IBP4/SHBG + ObRisk + | 0.671 | 0.681 | 0.764 |
| ObRisk+ | | | | | EGLN/PAPP 1(nullip), EGLN/PAPP 1(multip) | | | |
| IBP4/SHBG + | EGLN | PSG2 | EGLN | PSG2 | IBP4/SHBG + ObRisk + | 0.676 | 0.68 | 0.768 |
| ObRisk+ | | | | | EGLN/PSG2(nullip), EGLN/PSG2(multip) | | | |
| IBP4/SHBG + | EGLN | PTGDS | EGLN | PTGDS | IBP4/SHBG + ObRisk + | 0.676 | 0.683 | 0.731 |
| ObRisk+ | | | | | EGLN/PTGDS(nullip), EGLN/PTGDS(multip) | | | |
| IBP4/SHBG + | EGLN | TETN | EGLN | TETN | IBP4/SHBG + ObRisk + | 0.675 | 0.682 | 0.775 |
| ObRisk+ | | | | | EGLN/TETN(nullip), EGLN/TETN(multip) | | | |
| IBP4/SHBG + | EGLN | TETN | EGLN | A2GL | IBP4/SHBG + ObRisk + | 0.686 | 0.696 | 0.789 |
| ObRisk+ | | | | | EGLN/TETN(nullip), EGLN/A2GL(multip) | | | |
| IBP4/SHBG + | EGLN | TETN | EGLN | ALS | IBP4/SHBG + ObRisk + | 1.366 | 1.395 | 0.795 |
| ObRisk+ | | | | | EGLN/TETN(nullip), EGLN/ALS(multip) | | | |
| IBP4/SHBG + | EGLN | TETN | EGLN | ANGT | IBP4/SHBG + ObRisk + | 0.675 | 0.69 | 0.775 |
| ObRisk+ | | | | | EGLN/TETN(nullip), EGLN/ANGT(multip) | | | |
| IBP4/SHBG + | EGLN | TETN | EGLN | CLUS | IBP4/SHBG + ObRisk + | 0.684 | 0.693 | 0.795 |
| ObRisk+ | | | | | EGLN/TETN(nullip), EGLN/CLUS(multip) | | | |
| IBP4/SHBG + | EGLN | TETN | EGLN | CSH | IBP4/SHBG + ObRisk + | 0.68 | 0.693 | 0.788 |
| ObRisk+ | | | | | EGLN/TETN(nullip), EGLN/CSH(multip) | | | |
| IBP4/SHBG + | EGLN | TETN | EGLN | DEF1 | IBP4/SHBG + ObRisk + | 0.681 | 0.699 | 0.778 |
| ObRisk+ | | | | | EGLN/TETN(nullip), EGLN/DEF1(multip) | | | |
| IBP4/SHBG + | EGLN | TETN | EGLN | F13B | IBP4/SHBG + ObRisk + | 0.681 | 0.695 | 0.789 |
| ObRisk+ | | | | | EGLN/TETN(nullip), EGLN/F13B(multip) | | | |
| IBP4/SHBG + | EGLN | TETN | EGLN | FBLN3 | IBP4/SHBG + ObRisk + | 0.68 | 0.695 | 0.787 |
| ObRisk+ | | | | | EGLN/TETN(nullip), EGLN/FBLN3(multip) | | | |
| IBP4/SHBG + | EGLN | TETN | EGLN | FETUA | IBP4/SHBG + ObRisk + | 0.682 | 0.694 | 0.789 |
| ObRisk+ | | | | | EGLN/TETN(nullip), EGLN/FETUA(multip) | | | |
| IBP4/SHBG + | EGLN | TETN | EGLN | HEMO | IBP4/SHBG + ObRisk + | 0.682 | 0.696 | 0.789 |
| ObRisk+ | | | | | EGLN/TETN(nullip), EGLN/HEMO(multip) | | | |
| IBP4/SHBG + | EGLN | TETN | EGLN | IBP3 | IBP4/SHBG + ObRisk + | 0.684 | 0.698 | 0.795 |
| ObRisk+ | | | | | EGLN/TETN(nullip), EGLN/IBP3(multip) | | | |
| IBP4/SHBG + | EGLN | TETN | EGLN | IBP3 | IBP4/SHBG + ObRisk + | 0.681 | 0.695 | 0.794 |
| ObRisk+ | | | | | EGLN/TETN(nullip), EGLN/IBP3(multip) | | | |
| IBP4/SHBG + | EGLN | TETN | EGLN | IGF1 | IBP4/SHBG + ObRisk + | 0.684 | 0.697 | 0.787 |
| ObRisk+ | | | | | EGLN/TETN(nullip), EGLN/IGF1(multip) | | | |
| IBP4/SHBG + | EGLN | TETN | EGLN | IGF2 | IBP4/SHBG + ObRisk + | 0.68 | 0.696 | 0.793 |
| ObRisk+ | | | | | EGLN/TETN(nullip), EGLN/IGF2(multip) | | | |
| IBP4/SHBG + | EGLN | TETN | EGLN | PEDF | IBP4/SHBG + ObRisk + | 0.682 | 0.695 | 0.789 |
| ObRisk+ | | | | | EGLN/TETN(nullip), EGLN/PEDF(multip) | | | |
| IBP4/SHBG + | EGLN | TETN | EGLN | PRL | IBP4/SHBG + ObRisk + | | 0.697 | |
| ObRisk+ | | | | | EGLN/TETN(nullip), EGLN/PRL(multip) | | | |
| IBP4/SHBG + | EGLN | TETN | EGLN | PTGDS | IBP4/SHBG + ObRisk + | 0.678 | 0.697 | 0.771 |
| ObRisk+ | | | | | EGLN/TETN(nullip), EGLN/PTGDS(multip) | | | |
| IBP4/SHBG + | EGLN | TETN | EGLN | TENX | IBP4/SHBG + ObRisk + | 0.691 | 0.699 | 0.782 |
| ObRisk+ | | | | | EGLN/TETN(nullip), EGLN/TENX(multip) | | | |
| IBP4/SHBG + | EGLN | TETN | EGLN | TETN | IBP4/SHBG + ObRisk + | 0.676 | 0.69 | 0.789 |
| ObRisk+ | | | | | EGLN/TETN(nullip), EGLN/TETN(multip) | | | |
| IBP4/SHBG + | EGLN | VGFR1 | EGLN | VGFR1 | IBP4/SHBG + ObRisk + | | 0.691 | |
| ObRisk+ | | | | | EGLN/VGFR1(nullip), EGLN/VGFR1(multip) | | | |
| IBP4/SHBG + | HEMO | FA5 | EGLN | HEMO | IBP4/SHBG + ObRisk + | 0.688 | 0.683 | 0.756 |
| ObRisk+ | | | | | HEMO/FA5(nullip), EGLN/HEMO(multip) | | | |
| IBP4/SHBG + | LYAM1 | TETN | IBP2 | LYAMI | IBP4/SHBG + ObRisk + | 0.686 | 0.683 | 0.748 |
| ObRisk+ | | | | | LYAMI/TETN(nullip), IBP2/LYAMI(multip) | | | |
| IBP4/SHBG + | | | | | IBP4/SHBG + ObRisk | 0.647 | 0.645 | 0.706 |
| ObRisk+ | | | | | | | | |
| IBP4/SHBG + | PAPP2 | | PAPP2 | | IBP4/SHBG + ObRisk + PAPP2/(nullip), | | 0.669 | |
| ObRisk+ | | | | | PAPP2/(multip) | | | |
| IBP4/SHBG + | PAPP2 | TETN | PAPP2 | TETN | IBP4/SHBG + ObRisk + | 0.666 | 0.68 | 0.778 |
| ObRisk+ | | | | | PAPP2/TETN(nullip), PAPP2/TETN(multip) | | | |

Most models also include a second reversal (ratio of protein biomarker response ratios). The second reversal differs for nulliparous women vs. multiparous women. These second reversals are constrained to have one protein in common between the two parity levels. Thus, the model structure can be described as:

$$b 1 * log ( IBP 4 / SHBG ) + b 2 * ObRisk + b 3 * log ( AnalyteA ) - b 4 * log ( AnalyteB ) for nulliparous women [ new reversal : A / C ]$$

$$b 5 * log ( IBP 4 / SHBG ) + b 6 * ObRisk + b 7 * log ( AnalyteA ) - b 8 * log ( AnalyteC ) for nulliparous women [ new reversal : A / C ]$$

For example, the following model taken from Table 21:

$$IBP 4 / SHBG + ObRisk + EGLN / TETN ( nullip ) , EGLN / IBP 3 ( multip )$$

$$\text{becomes:}$$

$$log ( IBP 4 / SHBG ) + ObRisk + log ( EGLN ) - log ( TETN ) for nulliparous women ( with a coefficient implied for each term )$$

$$log ( IBP 4 / SHBG ) + ObRisk + log ( EGLN ) - log ( IBP 3 ) for mult iparous women ( with a coefficient implied for each term )$$

Note that log(EGLN)-log(IBP3) is mathematically equivalent to log(EGLN/IBP3), but provides opportunity to separately weight the numerator and denominator with different coefficients.

Two models form a triplet as shown in Table 21. Here, a third protein is incorporated into the numerator of the IBP4/SHBG reversal. For example, the following model taken from Table 21:

$$IBP 4 / SHBG + ObRisk + EGLN / ( nullip ) , EGLN / ( multip )$$

$$\text{becomes:}$$

$$log ( EGLN ) + log ( IBP 4 / SHBG ) + ObRisk for both nulliparous and multiparous women ( with nullip - and multip - specific coefficients implied for each term )$$

Note that log(EGLN)+log(IBP4/SHBG) is mathematically equivalent to log((EGLN*IBP4)/SHBG), but provides opportunity to separately weight the EGLN and IBP4 components of the numerator.

The ObRisk clinical variable is set to low (0) for nulliparous women with no prior miscarriage and for multiparous women with no prior preterm delivery. ObRisk is set to high (1) for nulliparous women with at least one prior miscarriage and for multiparous women with at least one prior preterm delivery.

TABLE 22
List of biomarker pairs measured in PBB study.

| No. | Biomarker Pair |
|---|---|
| 1. | EGLN/CSH |
| 2. | EGLN/DPEP2 |
| 3. | EGLN/FA5 |
| 4. | EGLN/A2GL |
| 5. | EGLN/ALS |
| 6. | EGLN/ANGT |
| 7. | EGLN/CLUS |
| 8. | EGLN/DEF1 |
| 9. | EGLN/F13B |
| 10. | EGLN/FBLN3 |
| 11. | EGLN/FETUA |
| 12. | EGLN/HEMO |
| 13. | EGLN/IBP3 |
| 14. | EGLN/IGF1 |
| 15. | EGLN/IGF2 |
| 16. | EGLN/PEDF |
| 17. | EGLN/PTGDS |
| 18. | EGLN/TENX |
| 19. | FA5/TENX |
| 20. | EGLN/FBLN3 |
| 21. | EGLN/IGF1 |
| 22. | EGLN/PAPP2 |
| 23. | EGLN/PSG2 |
| 24. | EGLN/TETN |
| 25. | EGLN/PRL |
| 26. | EGLN/VGFR1 |
| 27. | HEMO/FA5 |
| 28. | LYAM1/TETN |
| 29. | IBP2/LYAM1 |
| 30. | PAPP2/TETN |

What is claimed is:

1. A composition comprising at least three pairs of biomarkers selected from the group consisting of IBP4/SHBG, IBP4/TETN, EGLN/SHBG, EGLN/TETN, PRL/SHBG, and PRL/TETN, wherein said pairs of biomarkers exhibit a change in reversal value between pregnant females at risk for pre-term birth and term controls.

2. A composition comprising a reversal group of isolated biomarkers comprising:
(a) a reversal pair of isolated biomarkers consisting of IBP4/SHBG; and
(b) a reversal triplet of isolated biomarkers consisting of (EGLN+PRL)/TETN,
wherein said reversal group of isolated biomarkers exhibits a change in combined reversal value between pregnant females at risk for pre-term birth and term controls.

3. A composition comprising a reversal group of surrogate peptides for a reversal group of biomarkers comprising:
(a) a reversal pair of biomarkers consisting of IBP4/SHBG; and
(b) a reversal triplet of biomarkers consisting of (EGLN+PRL)/TETN,
wherein said reversal group of biomarkers exhibits a change in combined reversal value between pregnant females at risk for pre-term birth and term controls.

4. A composition comprising a reversal group of isolated biomarkers comprising:
(a) a reversal triplet of isolated biomarkers consisting of (EGLN+IBP4)/SHBG or (PAPP2+IBP4)/SHBG,
wherein said reversal group of isolated biomarkers exhibits a change in combined reversal value between pregnant females at risk for pre-term birth and term controls.

5. A composition comprising a reversal group of surrogate peptides for a reversal group of biomarkers comprising:
(a) a reversal triplet of isolated biomarkers consisting of (EGLN+IBP4)/SHBG or (PAPP2+IBP4)/SHBG,
wherein said reversal group of biomarkers exhibits a change in combined reversal value between pregnant females at risk for pre-term birth and term controls.

6. A composition comprising at least two pairs of biomarkers, wherein the at least two pairs of biomarkers comprise a first reversal pair of IBP4/SHBG; and a second reversal pair selected from Table 22, wherein said pairs of biomarkers exhibit a change in reversal value between pregnant females at risk for pre-term birth and term controls.

7. A composition comprising a reversal group of isolated biomarkers comprising:
(a) a first reversal pair of isolated biomarkers consisting of IBP4/SHBG; and
(b) a second reversal pair of isolated biomarkers selected from Table 22,
wherein said reversal group of isolated biomarkers exhibits a change in combined reversal value between pregnant females at risk for pre-term birth and term controls.

8. A composition comprising a reversal group of surrogate peptides for a reversal group of biomarkers comprising:
(a) a first reversal pair of isolated biomarkers consisting of IBP4/SHBG; and
(b) a second reversal pair of isolated biomarkers selected from Table 22,
wherein said reversal group of biomarkers exhibits a change in combined reversal value between pregnant females at risk for pre-term birth and term controls.

9. The composition of any one of claims 3, 5, or 8, further comprising stable isotope labeled standard peptides (SIS peptides) corresponding to each of the surrogate peptides.

10. The composition of claim 6, wherein the composition comprises a third reversal pair, wherein the third reversal pair is a reversal pair of isolated biomarkers selected from Table 22, except the third reversal pair and the second reversal pair are not the same.

11. The composition of claim 7 or 8, wherein the reversal group of isolated biomarkers of claim 7 or the reversal group of biomarkers of claim 8 further comprise a third reversal pair, wherein the third reversal pair is a reversal pair of isolated biomarkers is selected from Table 22, except the third reversal pair and the second reversal pair are not the same.

12. A panel comprising at least three pairs of biomarkers selected from the group consisting of IBP4/SHBG, IBP4/TETN, EGLN/SHBG, EGLN/TETN, PRL/SHBG, and PRL/TETN, wherein said pairs of biomarkers exhibit a change in reversal value between pregnant females at risk for pre-term birth and term controls.

13. A panel of biomarkers comprising a reversal group of biomarkers comprising:
(a) a reversal pair of biomarkers consisting of IBP4/SHBG; and
(b) a reversal triplet of biomarkers consisting of (EGLN+PRL)/TETN,
wherein said reversal group of biomarkers exhibits a change in combined reversal value between pregnant females at risk for pre-term birth and term controls.

14. A panel of surrogate peptides comprising a reversal group of surrogate peptides for a reversal group of biomarkers comprising:
(a) a reversal pair of biomarkers consisting of IBP4/SHBG; and
(b) a reversal triplet of biomarkers consisting of (EGLN+PRL)/TETN,
wherein said reversal group of biomarkers exhibits a change in combined reversal value between pregnant females at risk for pre-term birth and term controls.

15. A panel of biomarkers comprising a reversal group of biomarkers comprising:
(a) a reversal triplet of isolated biomarkers consisting of (EGLN+IBP4)/SHBG or (PAPP2+IBP4)/SHBG,
wherein said reversal group of isolated biomarkers exhibits a change in combined reversal value between pregnant females at risk for pre-term birth and term controls.

16. A panel of surrogate peptides comprising a reversal group of surrogate peptides for a reversal group of biomarkers comprising:
(a) a reversal triplet of isolated biomarkers consisting of (EGLN+IBP4)/SHBG or (PAPP2+IBP4)/SHBG,
wherein said reversal group of isolated biomarkers exhibits a change in combined reversal value between pregnant females at risk for pre-term birth and term controls.

17. A panel comprising at least two pairs of biomarkers, wherein the at least two pairs of biomarkers comprise a first reversal pair of IBP4/SHBG; and a second reversal pair selected from Table 22, wherein said pairs of biomarkers exhibits a change in reversal value between pregnant females at risk for pre-term birth and term controls.

18. A panel of biomarkers comprising a reversal group of biomarkers comprising:
(a) a first reversal pair of isolated biomarkers consisting of IBP4/SHBG; and
(b) a second reversal pair of biomarkers selected from Table 22,
wherein said reversal group of biomarkers exhibits a change in combined reversal value between pregnant females at risk for pre-term birth and term controls.

19. A panel of surrogate peptides comprising a reversal group of surrogate peptides for a reversal group of biomarkers comprising:
(a) a first reversal pair of isolated biomarkers consisting of IBP4/SHBG; and
(b) a second reversal pair of biomarkers selected from Table 22,
wherein said reversal group of biomarkers exhibits a change in combined reversal value between pregnant females at risk for pre-term birth and term controls.

20. The panel of any one of claims 14, 16, or 19, further comprising SIS peptides corresponding to each of the surrogate peptides.

21. The panel of claim 17, wherein the panel comprises a third pair of biomarkers, wherein the third pair of biomarkers is a reversal pair of isolated biomarkers selected from Table 22, except the third reversal pair and the second reversal pair are not the same.

22. The panel of claim 18 or 19, wherein the reversal group of biomarkers of claim 18 or the reversal group of biomarkers of claim 19 further comprise a third reversal pair, wherein the third reversal pair is a reversal pair of isolated biomarkers selected from Table 22, except the third reversal pair and the second reversal pair are not the same.

23. A method of determining probability for preterm birth in a pregnant female, the method comprising measuring in a biological sample obtained from said pregnant female, at least three pairs of biomarkers selected from the group consisting of IBP4/SHBG, IBP4/TETN, EGLN/SHBG, EGLN/TETN, PRL/SHBG, and PRL/TETN, wherein said pairs of biomarkers exhibit a change in reversal value between pregnant females at risk for pre-term birth and term controls.

24. A method of determining probability for preterm birth in a pregnant female, the method comprising measuring in a biological sample obtained from said pregnant female a reversal group of biomarkers comprising:
(a) a reversal pair of biomarker consisting of IBP4/SHBG; and
(b) a reversal triplet of biomarkers consisting of (EGLN+PRL)/TETN,
wherein said reversal group of biomarkers exhibits a change between pregnant females at risk for pre-term birth and term controls.

25. A method of determining probability for preterm birth in a pregnant female, the method comprising measuring in a biological sample obtained from said pregnant female a reversal group of surrogate peptides for a reversal group of biomarkers comprising:
(a) a reversal pair of biomarkers consisting of IBP4/SHBG; and
(b) a reversal triplet of biomarkers consisting of (EGLN+PRL)/TETN,
wherein said reversal group of biomarkers exhibits a change between pregnant females at risk for pre-term birth and term controls.

26. A method of determining probability for preterm birth in a pregnant female, the method comprising:
(a) obtaining a biological sample from the pregnant female;
(b) measuring a reversal group of biomarkers comprising:
(i) a reversal pair of biomarkers consisting of IBP4/SHBG; and
(ii) a reversal triplet of biomarkers consisting of (EGLN+PRL)/TETN; and
(c) determining the combined reversal value of said reversal group,
wherein said combined reversal value of said reversal group of biomarkers exhibits a change between pregnant females at risk for pre-term birth and term controls.

27. A method of determining probability for preterm birth in a pregnant female, the method comprising:
(a) obtaining a biological sample from the pregnant female;
(b) measuring a reversal pair of biomarkers consisting of IBP4/SHBG;
(c) determining a first reversal value of said reversal pair;
(d) measuring a reversal triplet of biomarkers consisting of (EGLN+PRL)/TETN;
(e) determining a second reversal value of said reversal triplet;
(f) combining the first reversal value and the second reversal value into a combined reversal value,
wherein said combined reversal value exhibits a change between pregnant females at risk for pre-term birth and term controls.

28. A method of determining probability for preterm birth in a pregnant female, the method comprising measuring in a biological sample obtained from said pregnant female a reversal group of biomarkers comprising:
(a) a reversal triplet of isolated biomarkers consisting of (EGLN+IBP4)/SHBG or (PAPP2+IBP4)/SHBG,
wherein said reversal group of biomarkers exhibits a change between pregnant females at risk for pre-term birth and term controls.

29. A method of determining probability for preterm birth in a pregnant female, the method comprising measuring in a biological sample obtained from said pregnant female a reversal group of surrogate peptides for a reversal group of biomarkers comprising:
(a) a reversal triplet of isolated biomarkers consisting of (EGLN+IBP4)/SHBG or (PAPP2+IBP4)/SHBG,
wherein said reversal group of biomarkers exhibits a change between pregnant females at risk for pre-term birth and term controls.

30. A method of determining probability for preterm birth in a pregnant female, the method comprising:
(a) obtaining a biological sample from the pregnant female;
(b) measuring a reversal group of biomarkers comprising:
(i) a reversal triplet of isolated biomarkers consisting of (EGLN+IBP4)/SHBG or (PAPP2+IBP4)/SHBG; and
(c) determining a combined reversal value of said reversal group,
wherein said combined reversal value of said reversal group of biomarkers exhibits a change between pregnant females at risk for pre-term birth and term controls.

31. A method of determining probability for preterm birth in a pregnant female, the method comprising:
(a) obtaining a biological sample from the pregnant female;
(b) measuring a reversal triplet of isolated biomarkers consisting of (EGLN+IBP4)/SHBG or (PAPP2+IBP4)/SHBG; and
(c) determining a combined reversal value of said reversal triplet;
wherein said combined reversal value exhibits a change between pregnant females at risk for pre-term birth and term controls.

32. A method of determining probability for preterm birth in a pregnant female, the method comprising measuring a biological sample obtained from said pregnant female, at least two pairs of biomarkers, wherein the at least two pairs of biomarkers comprise a first reversal pair of IBP4/SHBG; and a second reversal pair selected from Table 22, wherein said pairs of biomarkers exhibit a change in reversal value between pregnant females at risk for pre-term birth and term controls.

33. A method of determining probability for preterm birth in a pregnant female, the method comprising measuring a biological sample obtained from said pregnant female a reversal group of biomarkers comprising:
(a) a first reversal pair of isolated biomarkers consisting of IBP4/SHBG; and
(b) a second reversal pair of isolated biomarkers selected from Table 22;
wherein said reversal group of biomarkers exhibits a change between pregnant females at risk for pre-term birth and term controls.

34. A method of determining probability for preterm birth in a pregnant female, the method comprising measuring in a biological sample obtained from said pregnant female a reversal group of surrogate peptides for a reversal group of biomarkers comprising:
(a) a first reversal pair of isolated biomarkers consisting of IBP4/SHBG; and
(b) a second reversal pair of isolated biomarkers selected from Table 22,
wherein said reversal group of biomarkers exhibits a change between pregnant females at risk for pre-term birth and term controls.

35. A method of determining probability for preterm birth in a pregnant female, the method comprising:
(a) obtaining a biological sample from the pregnant female;
(b) measuring a reversal group of biomarkers comprising:
(i) a first reversal pair of isolated biomarkers consisting of IBP4/SHBG; and
(ii) a second reversal pair of isolated biomarkers selected from Table 22; and
(c) determining the reversal value of said reversal group,
wherein said reversal value of said reversal group of biomarkers exhibits a change between pregnant females at risk for pre-term birth and term controls.

36. A method of determining probability for preterm birth in a pregnant female, the method comprising:
(a) obtaining a biological sample from the pregnant female;
(b) measuring a reversal group of biomarkers comprising a first reversal pair of biomarkers consisting of IBP4/SHBG;
(c) determining a first reversal value of said first reversal pair;
(d) measuring a second reversal pair of biomarkers selected from Table 22;
(e) determining a second reversal value of said second reversal pair;
(f) combining the first reversal value and the second reversal value into a combined reversal value,
wherein said combined reversal value exhibits a change between pregnant females at risk for pre-term birth and term controls.

37. The method of claim 32, wherein the method comprises measuring a third pair of biomarkers, wherein the third pair of biomarkers is a reversal pair of isolated biomarkers selected from Table 22, except the third reversal pair and the second reversal pair are not the same.

38. The method of any one of claims 33 to 36, wherein the reversal group of biomarkers of claim 33, the reversal group of biomarkers of claim 34, the reversal group of biomarkers of claim 35, or the reversal group of biomarkers of claim 36 further comprise a third reversal pair, wherein the third reversal pair is a reversal pair of isolated biomarkers selected from Table 22, except the third reversal pair and the second reversal pair are not the same.

39. The method of any one of claims 23 to 38, further comprising a step of determining gestational age at blood draw (GABD).

40. The method of claim 39, wherein said step of determining GABD is performed before said obtaining step of 26(a), 27(a), 30(a), 31(a), 35(a), and 36(a).

41. The method of any one of claims 23 to 38, further comprising a step of determining body mass index (BMI).

42. The method of claim 41, wherein said step of determining BMI is performed before said obtaining step of 26(a), 27(a), 30(a), 31(a), 35(a), and 36(a).

43. The method of any one of claims 23 to 38, further comprising an initial step of detecting a measurable feature for one or more risk indicia.

44. The method of claim 23 or 32, wherein the one or more risk indicia are combined with the measurement of said pairs of biomarkers into a test score that, when compared to a reference score, exhibits a change in score between pregnant females at risk for pre-term birth and term controls.

45. The method of any one of claims 24, 25, 28, 29, 33, or 34, wherein the one or more risk indicia are combined with the measurement of said reversal group of biomarkers into a test score that, when compared to a reference score, exhibits a change in score between pregnant females at risk for pre-term birth and term controls.

46. The method of any one of claims 26, 30, or 35, wherein the one or more risk indicia are combined with said reversal value into a test score that, when compared to a reference score, exhibits a change in score between pregnant females at risk for pre-term birth and term controls.

47. The method of any one of claims 27, 31, or 36, wherein the one or more risk indicia are combined with said combined reversal value into a test score that, when compared to a reference score, exhibits a change in score between pregnant females at risk for pre-term birth and term controls.

48. The method of any one of claims 41 to 47, wherein said one or more risk indicia is selected from the group consisting of prior preterm birth, short cervical length, prior miscarriage, prior stillbirth, Body Mass Index (BMI), maternal age, parity, gravidity, fetal gender, height and weight separately from BMI, race and socioeconomic status.

49. The method of claim 48, wherein the risk indicium is BMI.

50. The method of any one of claims 23 to 47, wherein said method further comprises prediction of gestational age at birth (GAB) prior to said determining the probability for preterm birth.

51. The method of any one of claims 23 to 47, wherein the existence of a change in reversal value, combined reversal value, or final reversal value between the pregnant female and a term control indicates the probability for preterm birth in the pregnant female.

52. The method of any one of claims 23-24, 26-28, 30-33, and 35-47, wherein said measuring comprises measuring surrogate peptides of each of said reversal pair or reversal triplet in the biological sample obtained from said pregnant female.

53. The method of claim 52, wherein said measuring further comprises measuring stable isotope labeled standard peptides (SIS peptides) for each of the surrogate peptides.

54. The method of any one of claims 23 to 53, wherein said probability is expressed as a risk score.

55. The method of any one of claims 23 to 54, wherein said biological sample is selected from the group consisting of whole blood, plasma, serum, saliva, urine, amniotic fluid, cervical vaginal fluid.

56. The method of claim 55, wherein the biological sample is serum.

57. The method of any one of claims 23 to 56, wherein said measuring comprises mass spectrometry (MS).

58. The method of claim 57, wherein said MS is selected from the group consisting of matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n.

59. The method of claim 57, wherein said MS comprises co-immunoprecipitation-mass spectrometry (co-IP MS).

60. The method of claim 57, wherein said MS comprises liquid chromatography-mass spectrometry (LC-MS).

61. The method of claim 57, wherein said MS comprises multiple reaction monitoring (MRM) or selected reaction monitoring (SRM).

62. The method of any one of claims 23 to 61, wherein said measuring comprises an assay that utilizes a capture agent.

63. The method of claim 62, wherein said capture agent is selected from the group consisting of and antibody, antibody fragment, nucleic acid-based protein binding reagent, small molecule or variant thereof.

64. The method of claim 62, wherein said assay is selected from the group consisting of enzyme immunoassay (EIA), enzyme-linked immunosorbent assay (ELISA), and radioimmunoassay (RIA).

65. A method of detecting at least three pairs of biomarkers selected from the group consisting of IBP4/SHBG, IBP4/TETN, EGLN/SHBG, EGLN/TETN, PRL/SHBG, and PRL/TETN in a pregnant female, said method comprising,
(a) obtaining a biological sample from said pregnant female; and
(b) detecting whether the at least three pairs of biomarkers is present in the biological sample,
wherein said pairs of biomarkers exhibit a change in reversal value between pregnant females at risk for pre-term birth and term controls.

66. A method of detecting at least two pairs of biomarkers in a pregnant female, wherein the at least two pairs of biomarkers comprise a first reversal pair of IBP4/SHBG and a second reversal pair selected from Table 22, said method comprising,
(a) obtaining a biological sample from said pregnant female; and
(b) detecting whether the at least two pairs of biomarkers is present in the biological sample,
wherein said pairs of biomarkers exhibit a change in reversal value between pregnant females at risk for pre-term birth and term controls.

67. The method of claim 66, wherein the method comprises detecting a third biomarker pair, wherein the third biomarker pair is a biomarker pair selected from Table 22, except the third biomarker pair and the second biomarker pair are not the same.

68. The method of any one of claims 65 to 67, further comprising a step of determining gestational age at blood draw (GABD).

69. The method of any one of claims 65 to 67, wherein said step of determining GABD is performed before said obtaining step 65(a) or 66(a).

70. The method of any one of claims 65 to 67, further comprising a step of determining body mass index (BMI).

71. The method of any one of claims 65 to 67, wherein said step of determining BMI is performed before said obtaining step 65(a) or 66(a).

72. The method of any one of claims 65(a) or 66(a), further comprising an initial step of detecting a measurable feature for one or more risk indicia.

73. The method of claim 23 or 32, wherein the one or more risk indicia are combined with the measurement of said pairs of biomarkers into a test score that, when compared to a reference score, exhibits a change in score between pregnant females at risk for pre-term birth and term controls.

74. The method of any one of claims 24, 25, 28, 29, 33, or 34, wherein the one or more risk indicia are combined with the measurement of said reversal group of biomarkers into a test score that, when compared to a reference score, exhibits a change in score between pregnant females at risk for pre-term birth and term controls.

75. The method of any one of claims 26, 30 or 35, wherein the one or more risk indicia are combined with said reversal value or combined reversal value into a test score that, when compared to a reference score, exhibits a change in score between pregnant females at risk for pre-term birth and term controls.

76. The method of any one of claims 27, 31, or 36, wherein the one or more risk indicia are combined with said combined reversal value into a test score that, when compared to a reference score, exhibits a change in score between pregnant females at risk for pre-term birth and term controls.

77. The method of any one of claims 72 to 76, wherein said one or more risk indicia is selected from the group consisting of prior preterm birth, short cervical length, prior miscarriage, prior stillbirth, Body Mass Index (BMI), maternal age, parity, gravidity, fetal gender, height and weight separately from BMI, race and socioeconomic status.

78. The method of claim 77, wherein the risk indicium is BMI.

79. The method of any one of claims 23 to 78, wherein said method further comprises prediction of gestational age at birth (GAB) prior to said determining the probability for preterm birth.

80. The method of any one of claims 23 to 78, wherein the existence of a change in reversal value, combined reversal value, or final reversal value between the pregnant female and a term control indicates the probability for preterm birth in the pregnant female.

81. The method of any one of claims 23-24, 26-28, 30-33, and 35-78, wherein said measuring comprises measuring surrogate peptides of each of said reversal pair or reversal triplet in the biological sample obtained from said pregnant female.

82. The method of claim 81, wherein said measuring further comprises measuring stable isotope labeled standard peptides (SIS peptides) for each of the surrogate peptides.

83. The method of any one of claims 23 to 82, wherein said probability is expressed as a risk score.

84. The method of any one of claims 23 to 83, wherein said biological sample is selected from the group consisting of whole blood, plasma, serum, saliva, urine, amniotic fluid, cervical vaginal fluid.

85. The method of claim 84, wherein the biological sample is serum.

86. The method of any one of claims 23 to 85, wherein said measuring comprises mass spectrometry (MS).

87. The method of claim 86, wherein said MS is selected from the group consisting of matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n.

88. The method of claim 86, wherein said MS comprises co-immunoprecipitation-mass spectrometry (co-IP MS).

89. The method of claim 86, wherein said MS comprises liquid chromatography-mass spectrometry (LC-MS).

90. The method of claim 86, wherein said MS comprises multiple reaction monitoring (MRM) or selected reaction monitoring (SRM).

91. The method of any one of claims 23 to 85, wherein said measuring comprises an assay that utilizes a capture agent.

92. The method of claim 91, wherein said capture agent is selected from the group consisting of and antibody, antibody fragment, nucleic acid-based protein binding reagent, small molecule or variant thereof.

93. The method of claim 91, wherein said assay is selected from the group consisting of enzyme immunoassay (EIA), enzyme-linked immunosorbent assay (ELISA), and radioimmunoassay (RIA).

94. A method of detecting a reversal group of biomarkers comprising a reversal pair of biomarkers consisting of IBP4/SHBG and a reversal triplet of biomarkers consisting of (EGLN+PRL)/TETN in a pregnant female, said method comprising:
(a) obtaining a biological sample from said pregnant female; and
(b) detecting whether the reversal group of biomarkers is present in the biological sample,
wherein said detecting comprises subjecting said sample to a proteomics work-flow comprised of mass spectrometry (MS) quantification.

95. A method of detecting a reversal group of biomarkers comprising a reversal pair of biomarkers consisting of IBP4/SHBG and a reversal triplet consisting of (EGLN+PRL)/TETN in a pregnant female, said method comprising:
(a) obtaining a biological sample from the pregnant female;
(b) first detecting whether the reversal pair of biomarkers is present in the biological sample; and
(c) second detecting whether the reversal triplet of biomarkers is present in the biological sample,
wherein said first detecting step and/or said second detecting step comprises subjecting said sample to a proteomics work-flow comprised of mass spectrometry (MS) quantification.

96. A method of detecting at least three pairs of biomarkers selected from the group consisting of IBP4/SHBG, IBP4/TETN, EGLN/SHBG, EGLN/TETN, PRL/SHBG, and PRL/TETN in a pregnant female, said method comprising,
(a) obtaining a biological sample from said pregnant female; and
(b) detecting whether the pair of isolated biomarkers is present in the biological sample by contacting the biological sample with a first capture agent that specifically binds a first member of said pair and a second capture agent that specifically binds a second member of said pair; and,
(c) detecting binding between the first biomarker of said pair and the first capture agent and between the second member of said pair and the second capture agent,
wherein said detecting comprises an assay that utilizes the capture agent.

97. A method of detecting a reversal group of biomarkers comprising a reversal pair of biomarkers consisting of IBP4/SHBG and a reversal triplet of biomarkers consisting of (EGLN+PRL)/TETN in a pregnant female, said method comprising:
(a) obtaining a biological sample from the pregnant female;
(b) detecting whether the reversal group of biomarkers is present in the biological sample by contacting the biological sample with a capture agent that specifically binds an individual biomarker of the reversal group; and
(c) detecting binding between the capture agent and the individual biomarker of the reversal group,
wherein said detecting comprises an assay that utilizes the capture agent.

98. A method of detecting a reversal group of biomarkers comprising a reversal triplet of isolated biomarkers consisting of (EGLN+IBP4)/SHBG or (PAPP2+IBP4)/SHBG in a pregnant female, said method comprising:
(a) obtaining a biological sample from said pregnant female; and
(b) detecting whether the reversal group of biomarkers is present in the biological sample,
wherein said detecting comprises subjecting said sample to a proteomics work-flow comprised of mass spectrometry (MS) quantification.

99. A method of detecting a reversal group of biomarkers comprising a reversal triplet of biomarkers consisting of (EGLN+IBP4)/SHBG or (PAPP2+IBP4)/SHBG in a pregnant female, said method comprising:
(a) obtaining a biological sample from the pregnant female;
(b) detecting whether the reversal group of biomarkers is present in the biological sample by contacting the biological sample with a capture agent that specifically binds an individual biomarker of the reversal group; and
(c) detecting binding between the capture agent and the individual biomarker of the reversal group,
wherein said detecting comprises an assay that utilizes the capture agent.

100. A method of detecting a reversal group of biomarkers in a pregnant female comprising a first reversal pair of biomarkers consisting of IBP4/SHBG and a second reversal pair of biomarkers selected from Table 22, said method comprising:
(a) obtaining a biological sample from said pregnant female; and
(b) detecting whether the reversal group of biomarkers is present in the biological sample,
wherein said detecting comprises subjecting said sample to a proteomics work-flow comprised of mass spectrometry (MS) quantification.

101. A method of detecting a reversal group of biomarkers in a pregnant female comprising a first reversal pair of biomarkers consisting of IBP4/SHBG and a second reversal pair of biomarkers selected from Table 22, said method comprising:
(a) obtaining a biological sample from the pregnant female;
(b) first detecting whether the first reversal pair of biomarkers is present in the biological sample; and
(c) second detecting whether the second reversal pair of biomarkers is present in the biological sample,
wherein said first detecting step and/or said second detecting step comprises subjecting said sample to a proteomics work-flow comprised of mass spectrometry (MS) quantification.

102. A method of detecting at least two pairs of biomarkers in a pregnant female, wherein the at least two pairs of biomarkers comprise a first reversal pair of IBP4/SHBG and a second reversal pair selected from Table 22, said method comprising,
(a) obtaining a biological sample from said pregnant female; and
(b) detecting whether the pairs of isolated biomarkers are present in the biological sample by contacting the biological sample with a first capture agent that specifically binds a first member of said pairs and a second capture agent that specifically binds a second member of said pairs; and,
(c) detecting binding between the first biomarker of said pairs and the first capture agent and between the second member of said pairs and the second capture agent,
wherein said detecting comprises an assay that utilizes the capture agent.

103. A method of detecting a reversal group of biomarkers comprising a first reversal pair of biomarkers consisting of IBP4/SHBG and a second reversal pair of biomarkers selected from Table 22 in a pregnant female, said method comprising:
(a) obtaining a biological sample from the pregnant female;
(b) detecting whether the reversal group of biomarkers is present in the biological sample by contacting the biological sample with a capture agent that specifically binds an individual biomarker of the reversal group; and
(c) detecting binding between the capture agent and the individual biomarker of the reversal group,
wherein said detecting comprises an assay that utilizes the capture agent.

104. The method of any one of claims 100, 101, and 103, wherein the reversal group of biomarkers of claim 100, the reversal group of biomarkers of claim 101, or the reversal group of biomarkers of claim 103 further comprise a third reversal pair, wherein the third reversal pair is a reversal pair of isolated biomarkers selected from Table 22, except the third reversal pair and the second reversal pair are not the same.

105. The method of claim 102, wherein the method comprises detecting a third pair of biomarkers, wherein the third pair of biomarkers is a reversal pair of isolated biomarkers selected from Table 22, except the third reversal pair and the second reversal pair are not the same.

106. The method of any one of claims 94 to 105, further comprising an initial step of determining gestational age at blood draw (GABD).

107. The method of any one of claims 94 to 105, further comprising an initial step of determining Body Mass Index (BMI).

108. The method of any one of claims 94 to 105, further comprising detecting a measurable feature for one or more risk indicia.

109. The method of claim 108, wherein said risk indicium is selected from the group consisting of prior preterm birth, short cervical length, prior miscarriage, prior stillbirth, body mass index (BMI), maternal age, parity, gravidity, fetal gender, height and weight separately from BMI, race, and low socioeconomic status.

110. The method of claim 109, wherein the risk indicium is BMI.

111. The method of any one of claims 94 to 105, further comprising measuring a reversal value or combined reversal value for each of said pair of biomarkers or said reversal group of biomarkers.

112. The method of claim 111, wherein the existence of a change in said reversal value or combined reversal value between the pregnant female and a term control indicates the probability for preterm birth in the pregnant female.

113. The method of claim 112, wherein said probability is expressed as a risk score.

114. The method of any one of claims 94 to 105, wherein said biological sample is selected from the group consisting of whole blood, plasma, serum, saliva, urine, amniotic fluid, cervical vaginal fluid.

115. The method of claim 114, wherein said biological sample is serum.

116. The method of any one of claims 65 to 67, wherein said detecting comprises mass spectrometry (MS).

117. The method of any one of claims 65 to 67, wherein said detecting comprises an assay that utilizes a capture agent.

118. The method of any one of claims 96, 97, 99, 102, 103, and 117, wherein said capture agent is selected from the group consisting of and antibody, antibody fragment, nucleic acid-based protein binding reagent, small molecule or variant thereof.

119. The method of any one of claims 96, 97, 99, 102, 103, and 117, wherein said assay is selected from the group consisting of enzyme immunoassay (EIA), enzyme-linked immunosorbent assay (ELISA), and radioimmunoassay (RIA).

120. The method of any one of claims 94, 95, 98, 100, 101 and 116, wherein said MS is selected from the group consisting of matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n.

121. The method of any one of claims 94, 95, 98, 100, 101, and 116, wherein said MS comprises affinity-capture MS (AC-MS).

122. The method of any one of claims 94, 95, 98, 100, 101, and 116, wherein said MS comprises co-immunoprecipitation-mass spectrometry (co-IP MS).

123. The method of any one of claims 94, 95, 98, 100, 101, and 116, wherein said MS comprises liquid chromatography-mass spectrometry (LC-MS).

124. The method of any one of claims 94, 95, 98, 100, 101, and 116, wherein said MS comprises multiple reaction monitoring (MRM) or selected reaction monitoring (SRM).

125. A method of treating or preventing preterm birth in a pregnant female the method comprising:
(a) obtaining a biological sample from said pregnant female;
(b) detecting a reversal group of biomarkers in said sample;
(c) providing a risk score for said pregnant female;
(d) prognosing said pregnant female as having an increased risk of preterm birth; and
(e) administering one or more therapies to said pregnant female to prevent preterm birth.

126. The method of claim 125, wherein said reversal group comprises a reversal pair of and a reversal triplet of biomarkers.

127. The method of claim 126, wherein the reversal pair comprises IBP4/SHBG.

128. The method of claim 126, wherein the reversal triplet comprises (EGLN+PRL)/TETN.

129. The method of claim 125, wherein said reversal group comprises a reversal triplet of biomarkers consisting of (EGLN+IBP4)/SHBG or (PAPP2+IBP4)/SHBG.

130. The method of claim 125, wherein said reversal group comprises at least two reversal pairs of biomarkers, wherein the at least two reversal pairs comprise a first reversal pair and a second reversal pair of biomarkers.

131. The method of claim 130, wherein said first reversal pair comprises IBP4/SHBG.

132. The method of claim 130, wherein said second reversal pair comprises a reversal group of biomarkers selected from Table 22.

133. The method of claim 130, wherein said reversal group comprises a third reversal pair, wherein the third reversal pair is a reversal pair of isolated biomarkers selected from Table 22, except the third reversal pair and the second reversal pair are not the same.

134. The method of any one of claims 125 to 133, wherein said therapies comprise cervical cerclage, administration of 17-α hydroxyprogesterone caproate, vaginal progesterone gel, antenatal corticosteroids, cervical pessaries, or elevated care.

135. The method of any one of claims 125 to 134, wherein said method further comprises prediction of gestational age at birth (GAB) prior to said administrating step 125(e).

136. The method of any one of claims 125 to 135, further comprising an initial step of determining gestational age at blood draw (GABD).

137. The method of claim 136, wherein said step of determining GABD is performed before said obtaining step of 125(a).

138. The method of any one of claims 125 to 137, further comprising an initial step of determining Body Mass Index (BMI).

139. The method of claim 138 Error! Reference source not found., wherein said step of determining BMI is performed before said obtaining step of 125(a).

140. The method of any one of claims 125 to 139, further comprising detecting a measurable feature for one or more risk indicia.

141. The method of claim 140, wherein the one or more risk indicia are incorporated into said test risk score and said reference risk score.

142. The method of claim 140, wherein said risk indicium is selected from the group consisting of prior preterm birth, short cervical length, prior miscarriage, prior stillbirth, body mass index (BMI), maternal age, parity, gravidity, fetal gender, height and weight separately from BMI, race, and low socioeconomic status.

143. The method of claim 142, wherein the risk indicium is BMI.

144. The method of any one of claims 125 to 143, further comprising measuring a reversal value or combined reversal value for each of said pair of biomarkers or said reversal group of biomarkers.

145. The method of claim 144, wherein the existence of a change in said reversal value or combined reversal value between the pregnant female and a term control indicates the probability for preterm birth in the pregnant female.

146. The method of any one of claims 125 to 145, wherein said biological sample is selected from the group consisting of whole blood, plasma, serum, saliva, urine, amniotic fluid, cervical vaginal fluid.

147. The method of claim 146, wherein said biological sample is serum.

148. The method of any one of claims 125 to 147, wherein said detecting comprises an assay that utilizes a capture agent.

149. The method of claim 148, wherein said capture agent is selected from the group consisting of and antibody, antibody fragment, nucleic acid-based protein binding reagent, small molecule or variant thereof.

150. The method of claim 148, wherein said assay is selected from the group consisting of enzyme immunoassay (EIA), enzyme-linked immunosorbent assay (ELISA), and radioimmunoassay (RIA).

151. The method of any one of claims 125 to 147, wherein said detecting comprises mass spectrometry (MS).

152. The method of claim 151, wherein said MS is selected from the group consisting of matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n.

153. The method of claim 151, wherein said MS comprises affinity-capture MS (AC-MS).

154. The method of claim 151, wherein said MS comprises co-immunoprecipitation-mass spectrometry (co-IP MS).

155. The method of claim 151, wherein said MS comprises liquid chromatography-mass spectrometry (LC-MS).

156. The method of claim 151, wherein said MS comprises multiple reaction monitoring (MRM) or selected reaction monitoring (SRM).

157. A method of treating and/or preventing preterm birth, the method comprising:
(a) obtaining a biological sample from a pregnant female;
(b) detecting a reversal group of biomarkers in said sample;
(c) providing a test risk score for said pregnant female based at least in part on the detected level of said reversal group in said sample; and
(d) administering one or more preterm birth interventions to said pregnant female when the risk score exceeds a reference risk score.

158. The method of claim 157, wherein said reversal group comprises a reversal pair of and a reversal triplet of biomarkers.

159. The method of claim 158, wherein the reversal pair comprises IBP4/SHBG.

160. The method of claim 158, wherein the reversal triplet comprises (EGLN+PRL)/TETN.

161. The method of claim 157, wherein said reversal group comprises a reversal triplet of biomarkers consisting of (EGLN+IBP4)/SHBG or (PAPP2+IBP4)/SHBG.

162. The method of claim 157, wherein said reversal group comprises at least two reversal pairs of biomarkers, wherein the at least two reversal pairs comprise a first reversal pair and a second reversal pair of biomarkers.

163. The method of claim 162, wherein said first reversal pair comprises IBP4/SHBG.

164. The method of claim 162, wherein said second reversal pair comprises a reversal group of biomarkers selected from Table 22.

165. The method of claim 162, wherein said reversal group comprises a third reversal pair of biomarkers, wherein the third reversal pair of biomarkers is a reversal pair of isolated biomarkers selected from Table 22, except the third reversal pair and the second reversal pair are not the same.

166. The method of any one of claims 157 to 165, wherein said preterm interventions comprise cervical cerclage, administration of 17-α hydroxyprogesterone caproate, vaginal progesterone gel, antenatal corticosteroids, cervical pessaries, or elevated care.

167. The method of any one of claims 157 to 166, wherein said method further comprises prediction of gestational age at birth (GAB) prior to said administrating step 157(d).

168. The method of any one of claims 157 to 167, further comprising a step of determining gestational age at blood draw (GABD).

169. The method of claim 168, wherein said step of determining GABD is performed before said obtaining step 157(a).

170. The method of any one of claims 157 to 168, further comprising a step of determining Body Mass Index (BMI).

171. The method of claim 170, wherein said step of determining BMI is performed before said obtaining step 157(a).

172. The method of any one of claims 157 to 170, further comprising detecting a measurable feature for one or more risk indicia.

173. The method of claim 172, wherein the one or more risk indicia are incorporated into said test risk score and said reference risk score.

174. The method of claim 172, wherein said risk indicium is selected from the group consisting of prior preterm birth, short cervical length, prior miscarriage, prior stillbirth, body mass index (BMI), maternal age, parity, gravidity, fetal gender, height and weight separately from BMI, race, and low socioeconomic status.

175. The method of claim 174, wherein the risk indicium is BMI.

176. The method of claim 157, further comprising measuring a reversal value or combined reversal value for said reversal group of biomarkers.

177. The method of claim 176, wherein the existence of a change in said reversal value or combined reversal value between the pregnant female and a term control indicates the probability for preterm birth in the pregnant female.

178. The method of any one of claims 157 to 177, wherein said biological sample is selected from the group consisting of whole blood, plasma, serum, saliva, urine, amniotic fluid, cervical vaginal fluid.

179. The method of claim 178, wherein said biological sample is serum.

180. The method of any one of claims 157 to 179, wherein said detecting comprises an assay that utilizes a capture agent.

181. The method of claim 180, wherein said capture agent is selected from the group consisting of and antibody, antibody fragment, nucleic acid-based protein binding reagent, small molecule or variant thereof.

182. The method of claim 180, wherein said assay is selected from the group consisting of enzyme immunoassay (EIA), enzyme-linked immunosorbent assay (ELISA), and radioimmunoassay (RIA).

183. The method of any one of claims 157 to 179, wherein said detecting comprises mass spectrometry (MS).

184. The method of claim 183, wherein said MS is selected from the group consisting of matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n.

185. The method of claim 183, wherein said MS comprises affinity-capture MS (AC-MS).

186. The method of claim 183, wherein said MS comprises co-immunoprecipitation-mass spectrometry (co-IP MS).

187. The method of claim 183, wherein said MS comprises liquid chromatography-mass spectrometry (LC-MS).

188. The method of claim 183, wherein said MS comprises multiple reaction monitoring (MRM) or selected reaction monitoring (SRM).


SEQUENCE LISTING (67 sequences)

SEQ ID NO: 1 - protein, 14 aa, Homo sapiens
IALGGLLFPASNLR

SEQ ID NO: 2 - protein, 10 aa, Homo sapiens
QCHPALDGQR

SEQ ID NO: 3 - protein, 18 aa, Homo sapiens
GPITSAAELNDPQSILLR

SEQ ID NO: 4 - protein, 10 aa, Homo sapiens
TQILEWAAER

SEQ ID NO: 5 - protein, 12 aa, Homo sapiens
LSAYYNLLHCLR

SEQ ID NO: 6 - protein, 14 aa, Homo sapiens
SWNEPLYHLVTEVR

SEQ ID NO: 7 - protein, 9 aa, Homo sapiens
CFLAFTQTK

SEQ ID NO: 8 - protein, 12 aa, Homo sapiens
LDTLAQEVALLK

SEQ ID NO: 9 - protein, 13 aa, Homo sapiens
FGFGGSTDSGPIR

SEQ ID NO: 10 - protein, 11 aa, Homo sapiens
GWVTDGFSSLK

SEQ ID NO: 11 - protein, 12 aa, Homo sapiens
YGSQLAPETFYR

SEQ ID NO: 12 - protein, 11 aa, Homo sapiens
YGLVTYATYPK

SEQ ID NO: 13 - protein, 9 aa, Homo sapiens
VIAVNEVGR

SEQ ID NO: 14 - protein, 7 aa, Homo sapiens
VVESLAK

SEQ ID NO: 15 - protein, 14 aa, Homo sapiens
AGLLRPDYALLGHR

SEQ ID NO: 16 - protein, 16 aa, Homo sapiens
VSAPSGTGHLPGLNPL

SEQ ID NO: 17 - protein, 7 aa, Homo sapiens
HLACLPR

SEQ ID NO: 18 - protein, 10 aa, Homo sapiens
DLLLPQPDLR

SEQ ID NO: 19 - protein, 13 aa, Homo sapiens
IRPHTFTGLSGLR

SEQ ID NO: 20 - protein, 12 aa, Homo sapiens
DPTFIPAPIQAK

SEQ ID NO: 21 - protein, 12 aa, Homo sapiens
ASSIIDELFQDR

SEQ ID NO: 22 - protein, 19 aa, Homo sapiens
AHQLAIDTYQEFEETYIPK

SEQ ID NO: 23 - protein, 14 aa, Homo sapiens
ISLLLIESWLEPVR

SEQ ID NO: 24 - protein, 9 aa, Homo sapiens
YGTCIYQGR

SEQ ID NO: 25 - protein, 17 aa, Homo sapiens
ALEVSQAPVIFSHSAAR

SEQ ID NO: 26 - protein, 16 aa, Homo sapiens
GDTYPAELYITGSILR

SEQ ID NO: 27 - protein, 16 aa, Homo sapiens
LSEGASYLDHTFPAEK

SEQ ID NO: 28 - protein, 8 aa, Homo sapiens
IPSNPSHR

SEQ ID NO: 29 - protein, 10 aa, Homo sapiens
HTLNQIDEVK

SEQ ID NO: 30 - protein, 11 aa, Homo sapiens
NFPSPVDAAFR

SEQ ID NO: 31 - protein, 9 aa, Homo sapiens
LIQGAPTIR

SEQ ID NO: 32 - protein, 8 aa, Homo sapiens
FLNVLSPR

SEQ ID NO: 33 - protein, 11 aa, Homo sapiens
YGQPLPGYTTK

SEQ ID NO: 34 - protein, 15 aa, Homo sapiens
GFYFNKPTGYGSSSR

SEQ ID NO: 35 - protein, 9 aa, Homo sapiens
GIVEECCFR

SEQ ID NO: 36 - protein, 8 aa, Homo sapiens
SYYWIGIR

SEQ ID NO: 37 - protein, 10 aa, Homo sapiens
DIPHWLNPTR

SEQ ID NO: 38 - protein, 13 aa, Homo sapiens
LLLRPEVLAEIPR

SEQ ID NO: 39 - protein, 10 aa, Homo sapiens
TVQAVLTVPK

SEQ ID NO: 40 - protein, 9 aa, Homo sapiens
IHPSYTNYR

SEQ ID NO: 41 - protein, 17 aa, Homo sapiens
AQGFTEDTIVFLPQTDK

SEQ ID NO: 42 - protein, 15 aa, Homo sapiens
LSQLSVTDVTTSSLR

SEQ ID NO: 43 - protein, 8 aa, Homo sapiens
YLAVPTSK

SEQ ID NO: 44 - protein, 11 aa, Homo sapiens
ITLPDFTGDLR

SEQ ID NO: 45 - protein, 8 aa, Homo sapiens
ILDDLSPR

SEQ ID NO: 46 - protein, 7 aa, Homo sapiens
FQLPGQK

SEQ ID NO: 47 - protein, 14 aa, Homo sapiens
LEQGENVFLQATDK

SEQ ID NO: 48 - protein, 9 aa, Homo sapiens
ATVVYQGER

SEQ ID NO: 49 - protein, 12 aa, Homo sapiens
TNQVNSGGVLLR

SEQ ID NO: 50 - protein, 10 aa, Homo sapiens
LLPAQLPAEK

SEQ ID NO: 51 - protein, 13 aa, Homo sapiens
LLSDFPVVPTATR

SEQ ID NO: 52 - protein, 8 aa, Homo sapiens
ETLLQDFR

SEQ ID NO: 53 - protein, 8 aa, Homo sapiens
SSDANLYR

SEQ ID NO: 54 - protein, 7 aa, Homo sapiens
SLLQPNK

SEQ ID NO: 55 - protein, 8 aa, Homo sapiens
TASDFITK

SEQ ID NO: 56 - protein, 9 aa, Homo sapiens
NYGLLYCFR

SEQ ID NO: 57 - protein, 8 aa, Homo sapiens
VGFAEAAR

SEQ ID NO: 58 - protein, 7 aa, Homo sapiens
HFQNLGK

SEQ ID NO: 59 - protein, 9 aa, Homo sapiens
SALVLQYLR

SEQ ID NO: 60 - protein, 10 aa, Homo sapiens
AEVDDVIQVR

SEQ ID NO: 61 - protein, 9 aa, Homo sapiens
ITGFLKPGK

SEQ ID NO: 62 - protein, 7 aa, Homo sapiens
IAFSATR

SEQ ID NO: 63 - protein, 18 aa, Homo sapiens
FNAVLTNPQGDYDTSTGK

SEQ ID NO: 64 - protein, 10 aa, Homo sapiens
ELLALIQLER

SEQ ID NO: 65 - protein, 8 aa, Homo sapiens
AIDGINQR

SEQ ID NO: 66 - protein, 22 aa, Homo sapiens
AQPVQVAEGSEPDGFWEALGGK

SEQ ID NO: 67 - protein, 11 aa, Homo sapiens
FGSGYVSGWGR